SEQUENCE LISTING (14 sequences)

SEQ ID NO: 1 - DNA, 40 bp, synthetic construct
  source 1..40: mol_type = other DNA
cagagagcgaagcgggaggctgcgggctcaatttatagaa

SEQ ID NO: 2 - DNA, 107 bp, synthetic construct
  source 1..107: mol_type = other DNA
gcctcccgcttcgctctctgcatgtaatgcacgtactttcaggtatgtcggaaggtgtgcaggctaccgcttgtcaactggagaatgttctataaattgagcccgca

SEQ ID NO: 3 - DNA, 24 bp, synthetic construct
  source 1..24: mol_type = other DNA
catgtaatgcacgtactttcaggt

SEQ ID NO: 4 - DNA, 35 bp, synthetic construct
  source 1..35: mol_type = other DNA
atgtcggaaggtgtgcaggctaccgcttgtcaact

SEQ ID NO: 5 - skipped

SEQ ID NO: 6 - DNA, 93 bp, synthetic construct
  source 1..93: mol_type = other DNA
cagccgcatcttcttttgcgtcctctatgattactgactgcgtctatttagtggagccnnnnnnnnnnncttttgctcctcctgttcgacagt

SEQ ID NO: 7 - DNA, 38 bp, synthetic construct
  source 1..38: mol_type = other DNA
tcctctatgattactgactgcgtctatttagtggagcc

SEQ ID NO: 8 - DNA, 11 bp, synthetic construct
  source 1..11: mol_type = other DNA
gctactatctt

SEQ ID NO: 9 - DNA, 11 bp, synthetic construct
  source 1..11: mol_type = other DNA
ttagtagataa

SEQ ID NO: 10 - DNA, 11 bp, synthetic construct
  source 1..11: mol_type = other DNA
caccagcgagg

SEQ ID NO: 11 - DNA, 11 bp, synthetic construct
  source 1..11: mol_type = other DNA
aggtgctcgcc

SEQ ID NO: 12 - DNA, 35 bp, synthetic construct; identical to SEQ ID NO: 4
  source 1..35: mol_type = other DNA

SEQ ID NO: 13 - DNA, 28 bp, synthetic construct
  source 1..28: mol_type = other DNA
ttactgactgcgtctatttagtggagcc

SEQ ID NO: 14 - DNA, 53 bp, synthetic construct
  source 1..53: mol_type = other DNA
catgtaatgcacgtactttcagggtaaacatgtaatgcacgtactttcagggt

(19) United States
(12) Patent Application Publication
(10) Pub. No.: US 2025/0146068 A1
(43) Pub. Date: May 8, 2025

(54) REITERATIVE SHORT READ SEQUENCING INSIDE A CELLULAR SAMPLE

(71) Applicant: Element Biosciences, Inc., San Diego, CA (US)

(72) Inventors: Tsung-Li LIU, San Diego, CA (US); Sinan ARSLAN, San Diego, CA (US); Michael PREVITE, San Diego, CA (US); Tuval BEN-YEHEZKEL, Pleasanton, CA (US); Connor THOMPSON, San Diego, CA (US); Matthew KELLINGER, San Diego, CA (US); Hui Zhen MAH, San Diego, CA (US); Molly HE, San Diego, CA (US); Andrew PRICE, San Diego, CA (US)

(21) Appl. No.: 18/919,127

(22) Filed: Oct. 17, 2024

Related U.S. Application Data
(63) Continuation of application No. PCT/US23/65972, filed on Apr. 19, 2023.
(60) Provisional application No. 63/334,023, filed on Apr. 22, 2022.
(60) Provisional application No. 63/332,690, filed on Apr. 20, 2022.

(51) Int. Cl. C12Q 1/6874; C12N 15/10; C12Q 1/6804; C12Q 1/6841; C12Q 1/6844; G01N 33/58

(52) U.S. Cl. CPC C12Q 1/6874; C12N 15/1096; C12Q 1/6804; C12Q 1/6841; C12Q 1/6844; G01N 33/582

(57) ABSTRACT

The present disclosure provides methods for conducting in situ multiplex and multi-omics detection and identification using coded padlocks probes. The methods comprise simultaneous use of RNA-specific padlock probes and polypeptide-specific padlock probes to detect both RNA and polypeptides in a cellular sample. Both types of probes include a barcode that unique identifies the RNA or polypeptide that that padlock probe detects. Both types of probes also include a batch-specific sequencing primer binding site to enable sequencing a desired subset of concatemer template molecules. Use of the batch-specific sequencing primers reduces overcrowding signals and images, to produces optical images that are intense and resolvable. By conducting multiple rounds of sequencing on the same cellular sample using different batch-specific sequencing primers enables multiplex and multi-omics sequencing to reveal numerous target RNAs and their encoded polypeptides.

### CROSS-REFERENCE

This application is a continuation of International Patent Application No. PCT/US23/65972, filed Apr. 19, 2023 claims the benefit of U.S. Provisional Application No. 63/332,690, filed Apr. 20, 2022, and U.S. Provisional Application No. 63/334,023, filed Apr. 22, 2022, each of which is incorporated herein by reference in its entirety.

### SEQUENCE LISTING

The present application is being filed along with a Sequence Listing in electronic format. The Sequence Listing is provided as a file entitled 52933-752_301SL.xml, created on Apr. 17, 2023, which is 12,931 bytes in size. The information in the electronic format of the Sequence Listing is incorporated by reference in its entirety.

### TECHNICAL FIELD

The present disclosure provides compositions, apparatus and methods for conducting reiterative short read sequencing inside a cellular sample. In some embodiments, the reiterative short read sequencing can be used to discover the RNA content of the cellular sample.

### SUMMARY

Provided herein, in one aspect, is a method for detecting in situ at least two target nucleic acid sequences in a biological sample, wherein the at least two target nucleic acid sequences comprise a first target nucleic acid sequence and a second target nucleic acid sequence, the method comprising: (a) providing the biological sample comprising: (i) a first nucleic acid molecule, comprising the first target nucleic acid sequence or a portion thereof, or the reverse complement thereof or a portion thereof; and (ii) a second nucleic acid molecule, comprising the second target nucleic acid sequence or a portion thereof, or the reverse complement thereof or a portion thereof; (b) determining in situ the sequence of: (i) the first nucleic acid molecule or a portion thereof in the biological sample to generate a first sequencing product nucleic acid molecule that is complementary and binds to the first nucleic acid molecule or a portion thereof; and (ii) the second nucleic acid molecule or a portion thereof in the biological sample to generate a second sequencing product nucleic acid molecule that is complementary and binds to the second nucleic acid molecule or a portion thereof, wherein the full sequence of the first target nucleic acid sequence and the full sequence of the second target nucleic acid sequence have at least one nucleotide of difference; (c) removing the first sequencing product nucleic acid molecule from the first nucleic acid molecule and the second sequencing product nucleic acid molecule from the second nucleic acid molecule, wherein the first nucleic acid molecule and the second nucleic acid molecule are positioned inside the biological sample after the removing; and (d) repeating (b) and (c) to detect in situ the at least two target nucleic acid sequences in the biological sample. In some embodiments, the biological sample is fixed and permeabilized. In some embodiments, the method further comprises generating in situ: (a) a first complementary DNA (cDNA) molecule through reverse transcription of a first messenger RNA (mRNA) molecule inside the biological sample, wherein the first cDNA molecule or the first mRNA molecule comprises the first target nucleic acid sequence, or the reverse complement of the first target nucleic acid sequence; and/or (b) a second cDNA molecule through reverse transcription of a second mRNA molecule inside the biological sample, wherein the second cDNA molecule or the second mRNA molecule comprises the second target nucleic acid sequence, or the reverse complement of the second target nucleic acid sequence. In some embodiments, the method further comprises contacting in situ: (a) the first target nucleic acid sequence or a reverse complement thereof with a first oligonucleotide comprising a first end portion and a second end portion, wherein the first end portion and second end portion of the first oligonucleotide are complementary and bind to two neighboring segments of the first target nucleic acid sequence or the reverse complement thereof so that the first oligonucleotide forms a circular structure with a gap between the first end portion and the second end portion; and/or (b) the second target nucleic acid sequence or a reverse complement thereof with a second oligonucleotide comprising a first end portion and a second end portion, wherein the first end portion and second end portion of the second oligonucleotide are complementary and bind to two neighboring segments of the second target nucleic acid sequence or a reverse complement thereof so that the second oligonucleotide forms a circular structure with a gap between the first end portion and the second end portion. In some embodiments, wherein: (a) the first target nucleic acid sequence comprises the first cDNA molecule or the first mRNA molecule; and/or (b) the second target nucleic acid sequence comprises the second cDNA molecule or the second mRNA molecule. In some embodiments, wherein the gap of the first oligonucleotide or the second oligonucleotide has a size of one nucleotide. In some embodiments, wherein the gap of the first oligonucleotide or the second oligonucleotide has a size of at least two nucleotides. In some embodiments, wherein the first oligonucleotide further comprises a first identification sequence that identifies the first target nucleic acid sequence and the second oligonucleotide further comprises a second identification sequence that identifies the second target nucleic acid sequence. In some embodiments, wherein the first oligonucleotide and the second oligonucleotide further comprise a nucleic acid sequence that is complementary sequence to a nucleic acid sequence of a sequencing primer. In some embodiments, wherein the first oligonucleotide and the second oligonucleotides further comprise a nucleic acid sequence that is complementary sequence to a nucleic acid sequence of a primer for nucleic acid amplification. In some embodiments, wherein the primer for nucleic acid amplification is a primer for rolling circle amplification (RCA) that produces a concatemer, wherein the concatemer comprises at least two repeats of a target nucleic acid sequence of the at least two target nucleic acid sequences or a portion thereof, or a reverse complement thereof. In some embodiments, wherein the first oligonucleotide and the second oligonucleotide further comprise a reverse complement for a compaction oligonucleotide, wherein: (a) a first segment of the compaction oligonucleotide is complementary and binds to a first portion of the concatemer; and (b) a second segment of the compaction oligonucleotide is complementary and binds to a second portion of the concatemer, to result in a reduction in the size or a change in the shape of the concatemer.

In some embodiments, the method further comprises: (a) joining in situ the first and second end portions of the first oligonucleotide to produce a first circular oligonucleotide inside the biological sample; and (b) joining in situ the first and second end portions of the second oligonucleotide to produce a second circular oligonucleotide inside the biological sample. In some embodiments, wherein: (a) the joining the first and second end portions of the first oligonucleotide comprises joining the first and second end portions of the first oligonucleotides through a first nucleic acid enzyme; and (b) the joining the first and second end portions of the second oligonucleotide comprises joining the first and second end portions of the second oligonucleotide through a second nucleic acid enzyme, wherein the first nucleic acid enzyme and the second nucleic acid enzyme are the same type of enzyme. In some embodiments, wherein the first and second nucleic acid enzymes comprise a nucleic acid ligase, a nucleic acid ligation enzyme, a nucleic acid polymerase, a nucleic acid polymerization enzyme, or combinations thereof. In some embodiments, the method further comprises amplifying in situ: (a) the first circular oligonucleotide to produce the first nucleic acid molecule; and/or (b) the second circular oligonucleotide to produce the second nucleic acid molecule. In some embodiments, wherein the amplifying comprises rolling circle amplification (RCA), wherein the first nucleic acid molecule comprises a first concatemer and the second nucleic acid molecule comprises a second concatemer. In some embodiments, wherein: (a) the first concatemer comprises at least two repeats of a first unit nucleic acid sequence comprising the first target nucleic acid sequence or a portion thereof, or the reverse complement of the first target nucleic acid sequence or a portion thereof; and/or (b) the second concatemer comprises at least two repeats of a second unit nucleic acid sequence comprising the second target nucleic acid sequence or a portion thereof, or the reverse complement of the second target nucleic acid sequence or a portion thereof. In some embodiments, wherein the first concatemer further comprises the first identification sequence that identifies the first target nucleic acid sequence, or a sequencing primer or a reverse complement thereof, wherein the second concatemer further comprises the second identification sequence that identifies the second target nucleic acid sequence, or a sequencing primer or a reverse complement thereof. In some embodiments, wherein the first concatemer further comprises a first compaction oligonucleotide, wherein: (a) a first segment of the first compaction oligonucleotide is complementary and binds to a first portion of the first concatemer; and (b) a second segment of the first compaction oligonucleotide is complementary and binds to a second portion of the first concatemer, to result in a reduction in the size or a change in the shape of the first concatemer. In some embodiments, wherein the second concatemer further comprises a second compaction oligonucleotide, wherein: (a) a first segment of the second compaction oligonucleotide is complementary and binds to a first portion of the second concatemer; and (b) a second segment of the second compaction oligonucleotide is complementary and binds to a second portion of the second concatemer, to result in a reduction in the size or a change in the shape of the second concatemer. In some embodiments, wherein the determining comprises: (a) determining the sequence of the first nucleic acid molecule or the portion thereof, wherein the first nucleic acid molecule or the portion thereof consists of 2-30 nucleotides; and/or (b) determining the sequence of the second nucleic acid molecule or the portion thereof, wherein the second nucleic acid molecule or the portion thereof consists of 2-30 nucleotides. In some embodiments, wherein the 2-30 nucleotides of the first nucleic acid molecule or the portion thereof comprises the first identification sequence and the 2-30 nucleotides of the second nucleic acid molecule or the portion thereof comprises the second identification sequence. In some embodiments, wherein the 2-30 nucleotides of the first nucleic acid molecule or the portion thereof further comprises at least a portion of the sequence of the first cDNA molecule or the first mRNA molecule and the 2-30 nucleotides of the second nucleic acid molecule or the portion thereof further comprises at least a portion of the sequence of the second cDNA molecule or the second mRNA molecule. In some embodiments, wherein the determining comprises: (a) contacting the first concatemer with a polymerizing enzyme, a plurality of nucleotides, and a primer sequence that is complementary to a portion of the first concatemer under conditions sufficient to form a binding complex comprising the polymerizing enzyme, a nucleotide of the plurality of nucleotides that is complementary and binds to a nucleotide unit of the first concatemer, and the first concatemer hybridized to the primer sequence, wherein the nucleotide comprises a fluorescent label and a removable blocking group at the 3′ carbon position of the sugar moiety; (b) incorporating the nucleotide into the 3′ end of the primer sequence; and (c) identifying the nucleobase of the incorporated nucleotide by imaging the fluorescent label of the incorporated nucleotide. In some embodiments, wherein the determining comprises: (a) contacting the second concatemer with a polymerizing enzyme, a plurality of nucleotides, and a primer sequence that is complementary to a portion of the second concatemer under conditions sufficient to form a binding complex comprising the polymerizing enzyme, a nucleotide of the plurality of nucleotides that is complementary and binds to a nucleotide unit of the second concatemer, and the second concatemer hybridized to the primer sequence, wherein the nucleotide comprises a fluorescent label and a removable blocking group at the 3′ carbon position of the sugar moiety; (b) incorporating the nucleotide into the 3′ end of the primer sequence; and (c) identifying the nucleobase of the incorporated nucleotide by imaging the fluorescent label of the incorporated nucleotide.

In some embodiments, wherein the determining further comprises contacting the nucleotide with an agent to remove the blocking group from the nucleotide and generate a 3′ OH group on the sugar moiety. In some embodiments, wherein the blocking group comprises an alkyl group, an alkenyl group, an alkynyl group, an allyl group, an aryl group, a benzyl group, an azide group, an azido group, an O-azidomethyl group, an amine group, an amide group, a keto group, an isocyanate group, a phosphate group, a thiol group, a disulfide group, a carbonate group, a urea group, or a silyl group. In some embodiments, wherein the plurality of nucleotides consist of at least two of the same type of nucleotide, wherein the same type of nucleotide is selected from the group comprising dATP, dGTP, dCTP, dTTP, and dUTP, wherein the fluorescent label of one type of nucleotide of the group emits light at a wavelength that is different from the wavelength of light emitted from the fluorescent label of another type of nucleotide of the group. In some embodiments, wherein the plurality of nucleotides comprise at least two types of nucleotides, wherein the at least two types of nucleotides are selected from the group comprising dATP, dGTP, dCTP, dTTP, and dUTP, wherein the fluorescent label of one type of nucleotide of the group emits light at a wavelength that is different from the wavelength of light emitted from the fluorescent label of another type of nucleotide of the group. In some embodiments, wherein the determining comprises: (a) contacting two of the first concatemer with two of a polymerizing enzyme, a plurality of nucleotide conjugates, and two of a primer sequence that is complementary to a portion of the first concatemer under conditions sufficient to form a multivalent binding complex comprising each of the two of the polymerizing enzyme, a nucleotide conjugate of the plurality of nucleotide conjugates, and each of the two of the first concatemer hybridized to each of the two of the primer sequence, wherein the nucleotide conjugate comprises a label and at least two of a nucleotide moiety, wherein two of the at least two of the nucleotide moiety are each complementary and bind to a nucleotide of each of the two of the first concatemer; (b) detecting the multivalent binding complex through the label of the nucleotide conjugate; (c) identifying the nucleobases of the nucleotides of the two of the first concatemer that are each complementary and bind to each of the two of the at least two of the nucleotide moiety of the nucleotide conjugate. In some embodiments, wherein the determining comprises: (a) contacting two of the second concatemer with two of a polymerizing enzyme, a plurality of nucleotide conjugates, and two of a primer sequence that is complementary to a portion of the second concatemer under conditions sufficient to form a multivalent binding complex comprising each of the two of the polymerizing enzyme, a nucleotide conjugate of the plurality of nucleotide conjugates, and each of the two of the second concatemer hybridized to each of the two of the primer sequence, wherein the nucleotide conjugate comprises a label and at least two of a nucleotide moiety, wherein two of the at least two of the nucleotide moiety are each complementary and bind to a nucleotide of each of the two of the second concatemer; (b) detecting the multivalent binding complex through the label of the nucleotide conjugate; (c) identifying the nucleobases of the nucleotides of the two of the second concatemer that are each complementary and bind to each of the two of the at least two of the nucleotide moiety of the nucleotide conjugate. In some embodiments, wherein the conditions inhibit incorporation of the at least two of the nucleotide moiety of the nucleotide conjugate into the two of the first or second concatemer. In some embodiments, wherein the nucleotide conjugate comprises a core coupled to a plurality of nucleotide arms, wherein each of the nucleotide moiety is attached to one nucleotide arm of the plurality of nucleotide arms. In some embodiments, wherein the detectable label comprises a fluorescent label. In some embodiments, wherein the detecting comprises imaging the fluorescent label. In some embodiments, wherein the determining further comprises: (a) removing the two of the polymerizing enzyme and the nucleotide conjugate from the two of the first concatemer; (b) contacting each of the two of the first concatemer hybridized to each of the two of the primer sequence with two of a second polymerizing enzyme and a plurality of unlabeled nucleotides under conditions suitable for forming a binding complex comprising each of the two of the second polymerizing enzyme, each of the two of the first concatemer hybridized to each of the two of the primer sequence, and two of the plurality of the unlabeled nucleotides; and (c) incorporating each of the two of the plurality of the unlabeled nucleotides into each of the two of the primer sequence, wherein each of the two of the plurality of the unlabeled nucleotides is complementary and binds to a nucleotide of each of the two of the first concatemer, wherein an unlabeled nucleotide of the plurality of unlabeled nucleotides comprises a removable blocking group at the 3′ carbon of the sugan moiety. In some embodiments, wherein the determining further comprises: (a) removing the two of the polymerizing enzyme and the nucleotide conjugate from the two of the second concatemer; (b) contacting each of the two of the second concatemer hybridized to each of the two of the primer sequence with two of a second polymerizing enzyme and a plurality of unlabeled nucleotides under conditions sufficient for forming a binding complex comprising each of the two of the second polymerizing enzyme, each of the two of the second concatemer hybridized to each of the two of the primer sequence, and two of the plurality of the unlabeled nucleotides and incorporating each of the two of the plurality of the unlabeled nucleotides into each of the two of the primer sequence, wherein each of the two of the plurality of the unlabeled nucleotides is complementary and binds to a nucleotide of each of the two of the second concatemer, wherein an unlabeled nucleotide of the plurality of unlabeled nucleotides comprises a removable blocking group at the 3′ carbon of the sugan moiety.

In some embodiments, wherein the determining comprises: (a) contacting two of the first concatemer with two of a polymerizing enzyme, a plurality of nucleotide conjugates, a first primer sequence that is complementary to a first portion of the first concatemer, and a second primer sequence that is complementary to a second portion of the first concatemer under conditions sufficient to form a multivalent binding complex comprising each of the two of the polymerizing enzyme, a nucleotide conjugate of the plurality of nucleotide conjugates, the first portion of the first concatemer hybridized to the first primer sequence and a second portion of the first concatemer hybridized to the second primer sequence, wherein the nucleotide conjugate comprises a label and at least two of a nucleotide moiety, wherein two of the at least two of the nucleotide moiety are each complementary and bind to a nucleotide of a first portion and a second portion of the first concatemer; (b) detecting the multivalent binding complex through the label of the nucleotide conjugate; (c) identifying the nucleobases of the nucleotides of the first and second portions of the first concatemer that are each complementary and bind to each of the two of the at least two of the nucleotide moiety of the nucleotide conjugate. In some embodiments, the determining comprises: (a) contacting two of the second concatemer with two of a polymerizing enzyme, a plurality of nucleotide conjugates, a first primer sequence that is complementary to a first portion of the second concatemer, and a second primer sequence that is complementary to a second portion of the second concatemer under conditions sufficient to form a multivalent binding complex comprising each of the two of the polymerizing enzyme, a nucleotide conjugate of the plurality of nucleotide conjugates, the first portion of the second concatemer hybridized to the first primer sequence and a second portion of the second concatemer hybridized to the second primer sequence, wherein the nucleotide conjugate comprises a label and at least two of a nucleotide moiety, wherein two of the at least two of the nucleotide moiety are each complementary and bind to a nucleotide of a first portion and a second portion of the second concatemer; (b) detecting the multivalent binding complex through the label of the nucleotide conjugate; (c) identifying the nucleobases of the nucleotides of the first and second portions of the second concatemer that are each complementary and bind to each of the two of the at least two of the nucleotide moiety of the nucleotide conjugate.

In some embodiments, the conditions inhibit incorporation of the at least two of the nucleotide moiety of the nucleotide conjugate into the two of the first or second concatemer. In some embodiments, wherein the nucleotide conjugate comprises a core coupled to a plurality of nucleotide arms, wherein each of the nucleotide moiety is attached to one nucleotide arm of the plurality of nucleotide arms. In some embodiments, the detectable label comprises a fluorescent label. In some embodiments, wherein the detecting comprises imaging the fluorescent label. In some embodiments, the plurality of nucleotides consist of at least two of the same type of nucleotide, wherein the same type of nucleotide is selected from the group comprising dATP, dGTP, dCTP, dTTP, and dUTP, wherein the fluorescent label of one type of nucleotide of the group emits light at a wavelength that is different from the wavelength of light emitted from the fluorescent label of another type of nucleotide of the group. In some embodiments, the plurality of nucleotides comprise at least two types of nucleotides, wherein the at least two types of nucleotides are selected from the group comprising dATP, dGTP, dCTP, dTTP, and dUTP, wherein the fluorescent label of one type of nucleotide of the group emits light at a wavelength that is different from the wavelength of light emitted from the fluorescent label of another type of nucleotide of the group. In some embodiments, wherein the determining further comprises contacting the nucleotide with an agent to remove the blocking group from the nucleotide and generate a 3′ OH group on the sugar moiety. In some embodiments, wherein the blocking group comprises an alkyl group, an alkenyl group, an alkynyl group, an allyl group, an aryl group, a benzyl group, an azide group, an azido group, an O-azidomethyl group, an amine group, an amide group, a keto group, an isocyanate group, a phosphate group, a thiol group, a disulfide group, a carbonate group, a urea group, or a silyl group. In some embodiments, wherein the first target nucleic acid sequence and the second target nucleic acid sequence correspond to two separate portions of the same mRNA or cDNA molecule. In some embodiments, wherein the first target nucleic acid sequence and the second target nucleic acid sequence correspond to two different mRNA or cDNA molecules. In some embodiments, wherein the repeating comprises repeating at least 2 times, 5 times, 10 times, 15 times, 20 times, 25 times, 30 times, 35 times, 40 times, 45 times, or at least 50 times. In some embodiments, the determining comprises detecting in situ the first or second sequencing product nucleic acid molecule inside the biological sample through imaging. In some embodiments, the determining comprises detecting in situ simultaneously the first and second sequencing product nucleic acid molecules inside the biological sample through imaging. In some embodiments, the imaging comprises fluorescent imaging. In some embodiments, the biological sample comprises a cellular organelle, a cell, a whole cell, a group of whole cells, a tissue, an intact tissue, a tumor, an intact tumor, an organ, an organism, a protozoa, an algae, a bacteria, a virus, a plant, a fungus, an insect, or an animal. In some embodiments, the biological sample comprises a fresh sample, a processed sample, a freshly-frozen sample, a sectioned sample, or a formalin-fixed and paraffin-embedded (FFPE) sample. In some embodiments, the biological sample comprises a fresh cellular sample, a freshly-frozen cellular sample, a sectioned cellular sample, or an FFPE cellular sample. In some embodiments, the at least two target nucleic acid sequences comprise a target DNA sequence. In some embodiments, the at least two target nucleic acid sequences comprise a target RNA sequence. In some embodiments, the at least two target nucleic acid sequences comprises a first target RNA sequence and a second target RNA sequence. In some embodiments, the first target RNA sequence comprises coding RNA, non-coding RNA, messenger RNA (mRNA), transfer RNA (tRNA), ribosomal RNA (rRNA), microRNA (miRNA), guide RNA (gRNA), small nuclear RNA (snRNA), small interference RNA (siRNA), anti-sense RNA, mature microRNA, or immature microRNA. In some embodiments, the second target RNA sequence comprises coding RNA, non-coding RNA, mRNA, tRNA, rRNA, miRNA, gRNA, snRNA, siRNA, anti-sense RNA, mature microRNA, or immature microRNA. In some embodiments, the at least two target nucleic acid sequences comprise a first target DNA sequence and a second target DNA sequence. In some embodiments, the first target DNA sequence comprises complementary DNA (cDNA), genomic DNA (gDNA), non-coding DNA, or coding DNA. In some embodiments, the second target DNA sequence comprises cDNA, gDNA, non-coding DNA, or coding DNA. In some embodiments, the at least two target nucleic acid sequences comprise a target RNA sequence and a target DNA sequence. In some embodiments, the target RNA sequence comprises coding RNA, non-coding RNA, mRNA, tRNA, rRNA, miRNA, gRNA, snRNA, siRNA, anti-sense RNA, mature microRNA, or immature microRNA. In some embodiments, the target DNA sequence comprises cDNA, gDNA, non-coding DNA, or coding DNA. In some embodiments, the biological sample comprises a human sample, a simian sample, an ape sample, a canine sample, a feline sample, a bovine sample, an equine sample, a murine sample, a porcine sample, a caprine sample, a lupine sample, a ranine sample, a piscine sample, a plant sample, an insect sample, a bacteria sample, an algae sample, a viral sample, a protozoa sample, or a fungus sample. In some embodiments, the biological sample comprises a cellular organelle, a cell, a whole cell, a group of whole cells, a tissue, an intact tissue, a tumor, an intact tumor, an organ, or an organism. In some embodiments, the biological sample is immobilized on a surface. In some embodiments, the surface comprises an interior surface of a flow cell.

Provided herein, in another aspect, is a method for detecting in situ at least two target nucleic acid molecules and at least two target polypeptides in a biological sample, wherein the at least two target nucleic acid sequences comprise a first target nucleic acid sequence and a second target nucleic acid sequence, wherein the at least two target polypeptides comprise a first target polypeptide encoded by the first target nucleic acid sequence or a reverse complement thereof and a second target polypeptide encoded by the second target nucleic acid sequence or a reverse complement thereof, the method comprising: (a) providing the biological sample comprising: (i) a first nucleic acid molecule, comprising the first target nucleic acid sequence or a portion thereof, or the reverse complement of the first target nucleic acid sequence or a portion thereof; (ii) a second nucleic acid molecule, comprising the second target nucleic acid sequence or a portion thereof, or the reverse complement of the second target nucleic acid sequence or a portion thereof; (iii) a third nucleic acid molecule, comprising a third target nucleic acid sequence or a portion thereof, or the reverse complement of the third target nucleic acid sequence or a portion thereof, wherein the presence of third target nucleic acid sequence or the reverse complement thereof identifies the presence of the first target polypeptide in the biological sample; and (iv) a fourth nucleic acid molecule, comprising a fourth target nucleic acid sequence or a portion thereof, or the reverse complement of the fourth target nucleic acid sequence or a portion thereof, wherein the presence of the fourth target nucleic acid sequence or the reverse complement thereof identifies the presence of the second target polypeptide in the biological sample; (b) determining in situ the sequence of: (i) the first nucleic acid molecule or a portion thereof in the biological sample to generate a first sequencing product nucleic acid molecule that is complementary and binds to the first nucleic acid molecule or a portion thereof; and (ii) the third nucleic acid molecule or a portion thereof in the biological sample to generate a third sequencing product nucleic acid molecule that is complementary and binds to the third nucleic acid molecule or a portion; and (c) identifying in situ the sequence of: (i) the second nucleic acid molecule or a portion thereof in the biological sample to generate a second sequencing product nucleic acid molecule that is complementary and binds to the second nucleic acid molecule or a portion thereof; and (ii) the fourth nucleic acid molecule or a portion thereof in the biological sample to generate a fourth sequencing product nucleic acid molecule that is complementary and binds to the fourth nucleic acid molecule or a portion, wherein the full sequence of the first target nucleic acid sequence and the full sequence of the second target nucleic acid sequence have at least one nucleotide of difference, wherein the full sequence of the first target polypeptide and the full sequence of the second target polypeptide have at least one amino acid of difference, and wherein the performance of step (b) is under a condition that prevents the performance of the step (c).

Provided herein, in another aspect, is a method for detecting in situ at least two target nucleic acid sequences and at least two target polypeptides in a biological sample, wherein the at least two target nucleic acid sequences comprise a first target nucleic acid sequence and a second target nucleic acid sequence, wherein the at least two target polypeptides comprise a first target polypeptide encoded by the first target nucleic acid sequence or a reverse complement thereof and a second target polypeptide encoded by the second target nucleic acid sequence or a reverse complement thereof, the method comprising: (a) providing the biological sample comprising: (i) a first nucleic acid molecule, comprising the first target nucleic acid sequence or a portion thereof, or the reverse complement of the first target nucleic acid sequence or a portion thereof; (ii) a second nucleic acid molecule, comprising the second target nucleic acid sequence or a portion thereof, or the reverse complement of the second target nucleic acid sequence or a portion thereof; (iii) a third nucleic acid molecule, comprising a third target nucleic acid sequence or a portion thereof, or the reverse complement of the third target nucleic acid sequence or a portion thereof, wherein the presence of third target nucleic acid sequence or the reverse complement thereof identifies the presence of the first target polypeptide in the biological sample; and (iv) a fourth nucleic acid molecule, comprising a fourth target nucleic acid sequence or a portion thereof, or the reverse complement of the fourth target nucleic acid sequence or a portion thereof, wherein the presence of the fourth target nucleic acid sequence or the reverse complement thereof identifies the presence of the second target polypeptide in the biological sample; (b) determining in situ the sequence of: (i) the first nucleic acid molecule or a portion thereof in the biological sample to generate a first sequencing product nucleic acid molecule that is complementary and binds to the first nucleic acid molecule or a portion thereof, wherein the first nucleic acid molecule or the portion thereof consists of 2-30 nucleotides; and (ii) the third nucleic acid molecule or a portion thereof in the biological sample to generate a third sequencing product nucleic acid molecule that is complementary and binds to the third nucleic acid molecule or a portion, wherein the third nucleic acid molecule or the portion thereof consists of 2-30 nucleotides; and (c) removing the first sequencing product nucleic acid molecule from the first nucleic acid molecule and the third sequencing product nucleic acid molecule from the third nucleic acid molecule, wherein the first nucleic acid molecule and the third nucleic acid molecule are positioned in the biological sample after the removing; (d) repeating (b) and (c); (e) identifying in situ the sequence of: (i) the second nucleic acid molecule or a portion thereof in the biological sample to generate a second sequencing product nucleic acid molecule that is complementary and binds to the second nucleic acid molecule or a portion thereof, wherein the second nucleic acid molecule or the portion thereof consists of 2-30 nucleotides; and (ii) the fourth nucleic acid molecule or a portion thereof in the biological sample to generate a fourth sequencing product nucleic acid molecule that is complementary and binds to the fourth nucleic acid molecule or a portion, wherein the fourth nucleic acid molecule or the portion thereof consists of 2-30 nucleotides; (f) removing the second sequencing product nucleic acid molecule from the second nucleic acid molecule and the fourth sequencing product nucleic acid molecule from the fourth nucleic acid molecule, wherein the second nucleic acid molecule and the fourth nucleic acid molecule are located in the biological sample after the removing; and (g) repeating (e) and (f), wherein the full sequence of the first target nucleic acid sequence and the full sequence of the second target nucleic acid sequence have at least one nucleotide of difference, wherein the full sequence of the first target polypeptide and the full sequence of the second target polypeptide have at least one amino acid of difference, and wherein the performance of step (b) is under a condition that prevents the performance of the step (e).

Provided herein, in another aspect, is a method for detecting in situ at least two target RNA sequences and at least two target polypeptides in a biological sample, the method comprising:(a) providing the biological sample that is immobilized on a surface, permeabilized and fixed, wherein the biological sample comprises: (i) a first target RNA sequence of at least two target RNA sequences and a first target polypeptide of the at least two polypeptides, wherein the first target polypeptide is encoded by the first target RNA sequence or a reverse complement thereof; and (ii) a second target RNA sequence of at least two target RNA sequences and a second target polypeptide of the at least two polypeptides, wherein the second target polypeptide is encoded by the second target RNA sequence or a reverse complement thereof; (b) producing a first target cDNA sequence through reverse transcription of the first target RNA sequence and a second target cDNA sequence through reverse transcription of the second target RNA sequence; (c) contacting: (i) the first target cDNA sequence with a first oligonucleotide comprising a first end portion and a second end portion, wherein the first end portion and second end portion of the first oligonucleotide are complementary and bind to two neighboring segments of the first target cDNA sequence so that the first oligonucleotide forms a circular structure with a gap between the first end portion and the second end portion, wherein the first oligonucleotide comprises a first identification sequence that identifies the first target RNA sequence, a first sequencing primer, and a nucleic acid amplification primer; (ii) the second target cDNA sequence with a second oligonucleotide comprising a first end portion and a second end portion, wherein the first end portion and second end portion of the second oligonucleotide are complementary and bind to two neighboring segments of the second target cDNA sequence so that the second oligonucleotide forms a circular structure with a gap between the first end portion and the second end portion, wherein the second oligonucleotide comprises a second identification sequence that identifies the second target RNA sequence, a second sequencing primer, and a nucleic acid amplification primer, where the first sequencing primer and the second sequencing primer have at least one nucleotide of difference; (iii) the first target polypeptide with a first oligonucleotide conjugate comprising a first short nucleic acid and a first binding moiety that binds specifically to the first target polypeptide, wherein the first short nucleic acid comprises a first tag sequence, a second tag sequence, wherein the first oligonucleotide conjugate binds specifically to the first target polypeptide through the first binding moiety to form a first binding complex, wherein the first and second tag sequences identify the first binding moiety; and (iv) the second target polypeptide with a second oligonucleotide conjugate comprising a second short nucleic acid and a second binding moiety that binds specifically to the second target polypeptide, wherein the second short nucleic acid comprises a third tag sequence, a fourth tag sequence, wherein the second oligonucleotide conjugate binds specifically to the second target polypeptide through the second binding moiety to form a second binding complex, wherein the third and fourth tag sequences identify the second binding moiety; (d) contacting: (i) the first binding complex with a third oligonucleotide comprising a first end portion and a second end portion, wherein the first end portion and second end portion of the third oligonucleotide are complementary and bind to the first tag sequence and the second tag sequence of the first oligonucleotide conjugate so that the third oligonucleotide forms a circular structure with a gap between the first end portion and the second end portion; and (ii) the second binding complex with a fourth oligonucleotide comprising a first end portion and a second end portion, wherein the first end portion and second end portion of the fourth oligonucleotide are complementary and bind to the third tag sequence and the fourth tag sequence so that the fourth oligonucleotide forms a circular structure with a gap between the first end portion and the second end portion; (e) joining the first and second end portions of the first oligonucleotide to produce a first circular oligonucleotide, the first and second end portions of the second oligonucleotides to produce a second circular oligonucleotide, the first and second end portions of the third oligonucleotide to produce a third circular oligonucleotide and the first and second end portions of the fourth oligonucleotide to produce a fourth circular oligonucleotide, wherein the first circular oligonucleotide and the third circular oligonucleotide comprise a first sequencing primer or the reverse complement thereof, wherein the second circular oligonucleotide and the fourth circular oligonucleotide comprise a second sequencing primer or the reverse complement thereof, wherein the sequences of the first and second sequencing primers have at least one nucleotide of difference; (f) amplifying: (i) the first circular oligonucleotide through rolling circle amplification to produce a first concatemer comprising a plurality of the first circular oligonucleotides; (ii) the second circular oligonucleotide through rolling circle amplification to produce a second concatemer comprising a plurality of the second circular oligonucleotides; (iii) the third circular oligonucleotide through rolling circle amplification to produce a third concatemer comprising a plurality of the third circular oligonucleotides; and (iv) the fourth circular oligonucleotide through rolling circle amplification to produce a fourth concatemer comprising a plurality of the fourth circular oligonucleotides; (g) determining in situ the sequence of: (i) the first concatemer or a portion thereof to generate a first sequencing product nucleic acid molecule that is complementary and binds to the first concatemer, wherein the sequence of the first concatemer or the portion thereof consists of 2-30 nucleotides; and (ii) the third concatemer or a portion thereof to generate a third sequencing product nucleic acid molecule that is complementary and binds to the third concatemer, wherein the sequence of the third concatemer or the portion thereof consists of 2-30 nucleotides, wherein the performance of step (g) is under a condition that prevents the performance of the step (j); (h) removing the first sequencing product nucleic acid molecule from the first concatemer and the third sequencing product nucleic acid molecule from the third concatemer, wherein the first concatemer and the third concatemer are positioned in the biological sample after the removing; (i) repeating (g) and (h) at least once; (j) determining in situ the sequence of: (i) the second concatemer or a portion thereof to generate a second sequencing product nucleic acid molecule that is complementary and binds to the second concatemer, wherein the sequence of the second concatemer or the portion thereof consists of 2-30 nucleotides; and (ii) the fourth concatemer or a portion thereof to generate a fourth sequencing product nucleic acid molecule that is complementary and binds to the fourth concatemer, wherein the sequence of the fourth concatemer or the portion thereof consists of 2-30 nucleotides, wherein the performance of steps (j) is under a condition that prevents the performance of the step (g), wherein the full sequence of the first target RNA sequence and the full sequence of the second target RNA sequence have at least one nucleotide of difference, wherein the full sequence of the first target polypeptide and the full sequence of the second target polypeptide have at least one amino acid of difference; (k) removing the second sequencing product nucleic acid molecule from the second concatemer and the fourth sequencing product nucleic acid molecule from the fourth concatemer, wherein the second concatemer and the fourth concatemer are positioned in the biological sample after the removing; (1) repeating (j) and (k) at least once.

In some embodiments, the determining comprises imaging the first, or third sequencing product nucleic acid molecule, wherein the identifying comprises imaging the second or fourth sequencing product nucleic acid molecule and the fourth sequencing product nucleic acid molecule. In some embodiments, the method further comprises imaging simultaneously the first sequencing product nucleic acid molecule and the second sequencing product nucleic acid molecule to analyze the spatial distribution of the first and second sequencing product nucleic acid molecules inside the biological sample. In some embodiments, the biological sample comprises a cellular organelle, a cell, a whole cell, a group of whole cells, a tissue, an intact tissue, a tumor, an intact tumor, an organ, an organism, a protozoa, an algae, a bacteria, a virus, a plant, a fungus, an insect, or an animal. In some embodiments, the biological sample comprises a fresh sample, a processed sample, a freshly-frozen sample, a sectioned sample, or a formalin-fixed and paraffin-embedded (FFPE) sample. In some embodiments, the first target nucleic acid sequence comprises DNA, cDNA, RNA, coding RNA, non-coding RNA, mRNA, tRNA, rRNA, miRNA, gRNA, snRNA, siRNA, anti-sense RNA, mature microRNA, or immature microRNA, and/or wherein the first target RNA sequence comprises coding RNA, non-coding RNA, mRNA, tRNA, rRNA, miRNA, gRNA, snRNA, siRNA, anti-sense RNA, mature microRNA, or immature microRNA. In some embodiments, the second target nucleic acid sequence comprises DNA, cDNA, RNA, coding RNA, non-coding RNA, mRNA, tRNA, rRNA, miRNA, gRNA, snRNA, siRNA, anti-sense RNA, mature microRNA, or immature microRNA, and/or wherein the second target RNA sequence comprises coding RNA, non-coding RNA, mRNA, tRNA, rRNA, miRNA, gRNA, snRNA, siRNA, anti-sense RNA, mature microRNA, or immature microRNA. In some embodiments, the biological sample comprises a human sample, a simian sample, an ape sample, a canine sample, a feline sample, a bovine sample, an equine sample, a murine sample, a porcine sample, a caprine sample, a lupine sample, a ranine sample, a piscine sample, a plant sample, an insect sample, a bacteria sample, an algae sample, a viral sample, a protozoa sample, or a fungus sample. In some embodiments, the first, second, third or fourth concatemer further comprises a compaction oligonucleotide, wherein: (a) a first segment of the compaction oligonucleotide is complementary and binds to a first portion of the first, second, third, or fourth concatemer; and (b) a second segment of the compaction oligonucleotide is complementary and binds to a second portion of the first, second, third, or fourth concatemer, to result in a reduction in the size or a change in the shape of the second concatemer. In some embodiments, the first sequencing product nucleic acid molecule comprises a first identification sequence that identifies the first target RNA sequence and the second sequencing product nucleic acid molecule comprises a second identification sequence that identifies the second target RNA sequence. In some embodiments, the first sequencing product nucleic acid molecule comprises a first identification sequence that identifies the first target RNA sequence and a portion of the first target RNA sequence, wherein the second sequencing product nucleic acid molecule comprises a second identification sequence that identifies the second target RNA sequence and a portion of the second target RNA sequence. In some embodiments, the determining comprises: (a) contacting the first second, third, or fourth concatemer with a polymerizing enzyme, a plurality of nucleotides, and a primer sequence that is complementary to a portion of the first second, third, or fourth concatemer under conditions sufficient to form a binding complex comprising the polymerizing enzyme, a nucleotide of the plurality of nucleotides that is complementary and binds to a nucleotide unit of the first second, third, or fourth concatemer, and the first second, third, or fourth concatemer hybridized to the primer sequence, wherein the nucleotide comprises a fluorescent label and a removable blocking group at the 3′ carbon position of the sugar moiety; (b) incorporating the nucleotide into the 3′ end of the primer sequence; and (c) identifying the nucleobase of the incorporated nucleotide by imaging the fluorescent label of the incorporated nucleotide. In some embodiments, the determining further comprises contacting the nucleotide with an agent to remove a blocking group from the nucleotide and generate a 3′ OH group on the sugar moiety. In some embodiments, the blocking group comprises an alkyl group, an alkenyl group, an alkynyl group, an allyl group, an aryl group, a benzyl group, an azide group, an azido group, an O-azidomethyl group, an amine group, an amide group, a keto group, an isocyanate group, a phosphate group, a thiol group, a disulfide group, a carbonate group, a urea group, or a silyl group. In some embodiments, the plurality of nucleotides comprise one type of nucleotide selected from a group comprising dATP, dGTP, dCTP, dTTP and dUTP. In some embodiments, the plurality of nucleotides comprise a mixture of any combination of two or more types of nucleotides selected from a group consisting of dATP, dGTP, dCTP, dTTP and/or dUTP. In some embodiments, the determining comprises: (a) contacting two of the first, second, third, or fourth concatemer with two of a polymerizing enzyme, a plurality of nucleotide conjugates, and two of a primer sequence that is complementary to a portion of the first, second, third, or fourth concatemer under conditions sufficient to form a multivalent binding complex comprising each of the two of the polymerizing enzyme, a nucleotide conjugate of the plurality of nucleotide conjugates, and each of the two of the first, second, third, or fourth concatemer hybridized to each of the two of the primer sequence, wherein the nucleotide conjugate comprises a label and at least two of a nucleotide moiety, wherein two of the at least two of the nucleotide moiety are each complementary and bind to a nucleotide of each of the two of the first concatemer; (b) detecting the multivalent binding complex through the label of the nucleotide conjugate; (c) identifying the nucleobases of the nucleotides of the two of the first concatemer that are each complementary and bind to each of the two of the at least two of the nucleotide moiety of the nucleotide conjugate.

In some embodiments, the determining further comprises: (a) removing the two of the polymerizing enzyme and the nucleotide conjugate from the two of the first, second, third, or fourth concatemer; (b) contacting each of the two of the first, second, third, or fourth concatemer hybridized to each of the two of the primer sequence with two of a second polymerizing enzyme and a plurality of unlabeled nucleotides under conditions suitable for forming a binding complex comprising each of the two of the second polymerizing enzyme, each of the two of the first, second, third, or fourth concatemer hybridized to each of the two of the primer sequence, and two of the plurality of the unlabeled nucleotides and incorporating each of the two of the plurality of the unlabeled nucleotides into each of the two of the primer sequence, wherein each of the two of the plurality of the unlabeled nucleotides is complementary and binds to a nucleotide of each of the two of the first, second, third, or fourth concatemer, wherein an unlabeled nucleotide of the plurality of unlabeled nucleotides comprises a removable blocking group at the 3′ carbon of the sugan moiety.

In some embodiments, the determining comprises: (a) contacting two of the first, second, third, or fourth concatemer with two of a polymerizing enzyme, a plurality of nucleotide conjugates, a first primer sequence that is complementary to a first portion of the first, second, third, or fourth concatemer, and a second primer sequence that is complementary to a second portion of the first, second, third, or fourth concatemer under conditions sufficient to form a multivalent binding complex comprising each of the two of the polymerizing enzyme, a nucleotide conjugate of the plurality of nucleotide conjugates, the first portion of the first, second, third, or fourth concatemer hybridized to the first primer sequence and a second portion of the first, second, third, or fourth concatemer hybridized to the second primer sequence, wherein the nucleotide conjugate comprises a label and at least two of a nucleotide moiety, wherein two of the at least two of the nucleotide moiety are each complementary and bind to a nucleotide of a first portion and a second portion of the first, second, third, or fourth concatemer; (b) detecting the multivalent binding complex through the label of the nucleotide conjugate; (c) identifying the nucleobases of the nucleotides of the first and second portions of the first, second, third, or fourth concatemer that are each complementary and bind to each of the two of the at least two of the nucleotide moiety of the nucleotide conjugate. In some embodiments, the conditions inhibit incorporation of the at least two of the nucleotide moiety of the nucleotide conjugate into the two of the first, second, third, or fourth concatemer. In some embodiments, the nucleotide conjugate comprises a core coupled to a plurality of nucleotide arms, wherein each of the nucleotide moiety is attached to one nucleotide arm of the plurality of nucleotide arms. In some embodiments, the detectable label comprises a fluorescent label. In some embodiments, the detecting comprises imaging the fluorescent label. In some embodiments, the plurality of nucleotides consist of at least two of the same type of nucleotide, wherein the same type of nucleotide is selected from the group comprising dATP, dGTP, dCTP, dTTP, and dUTP, wherein the fluorescent label of one type of nucleotide of the group emits light at a wavelength that is different from the wavelength of light emitted from the fluorescent label of another type of nucleotide of the group. In some embodiments, the plurality of nucleotides comprise at least two types of nucleotides, wherein the at least two types of nucleotides are selected from the group comprising dATP, dGTP, dCTP, dTTP, and dUTP, wherein the fluorescent label of one type of nucleotide of the group emits light at a wavelength that is different from the wavelength of light emitted from the fluorescent label of another type of nucleotide of the group. In some embodiments, the determining further comprises contacting the nucleotide with an agent to remove the blocking group from the nucleotide and generate a 3′ OH group on the sugar moiety.

In some embodiments, the blocking group comprises an alkyl group, an alkenyl group, an alkynyl group, an allyl group, an aryl group, a benzyl group, an azide group, an azido group, an O-azidomethyl group, an amine group, an amide group, a keto group, an isocyanate group, a phosphate group, a thiol group, a disulfide group, a carbonate group, a urea group, or a silyl group. In some embodiments, the plurality of nucleotides consist of at least two of the same type of nucleotide, wherein the same type of nucleotide is selected from the group comprising dATP, dGTP, dCTP, dTTP, and dUTP, wherein the fluorescent label of one type of nucleotide of the group emits light at a wavelength that is different from the wavelength of light emitted from the fluorescent label of another type of nucleotide of the group. In some embodiments, the plurality of nucleotides comprise at least two types of nucleotides, wherein the at least two types of nucleotides are selected from the group comprising dATP, dGTP, dCTP, dTTP, and dUTP, wherein the fluorescent label of one type of nucleotide of the group emits light at a wavelength that is different from the wavelength of light emitted from the fluorescent label of another type of nucleotide of the group. In some embodiments, the first target RNA sequence comprises coding RNA, non-coding RNA, messenger RNA (mRNA), transfer RNA (tRNA), ribosomal RNA (rRNA), microRNA (miRNA), guide RNA (gRNA), small nuclear RNA (snRNA), small interference RNA (siRNA), anti-sense RNA, mature microRNA, or immature microRNA. In some embodiments, the second target RNA sequence comprises coding RNA, non-coding RNA, mRNA, tRNA, rRNA, miRNA, gRNA, snRNA, siRNA, anti-sense RNA, mature microRNA, or immature microRNA. In some embodiments, the biological sample comprises a human sample, a simian sample, an ape sample, a canine sample, a feline sample, a bovine sample, an equine sample, a murine sample, a porcine sample, a caprine sample, a lupine sample, a ranine sample, a piscine sample, a plant sample, an insect sample, a bacteria sample, an algae sample, a viral sample, a protozoa sample, or a fungus sample. In some embodiments, the biological sample comprises a cellular organelle, a cell, a whole cell, a group of whole cells, a tissue, an intact tissue, a tumor, an intact tumor, an organ, or an organism. In some embodiments, the biological sample is immobilized on a surface. In some embodiments, the surface comprises an interior surface of a flow cell.

Provided herein, in another aspect, is a system for detecting in situ at least two target nucleic acid sequences in a biological sample, wherein the at least two target nucleic acid sequences comprise a first target nucleic acid sequence and a second target nucleic acid sequence, the system comprising: (a) a biological sample comprising a first nucleic acid molecule and a second nucleic acid molecule, wherein the first nucleic acid molecule comprises the first target nucleic acid sequence or a reverse complement thereof, or a portion thereof, and wherein the second nucleic acid molecule comprises the second target nucleic acid sequence or a reverse complement thereof, or a portion thereof; (b) a first oligonucleotide comprising a first end portion and a second end portion, wherein the first end portion and second end portion of the first oligonucleotide are complementary and bind to two neighboring segments of the first target nucleic acid sequence or the reverse complement thereof, or the portion thereof, so that the first oligonucleotide forms a first circular oligonucleotide within the biological sample, wherein the first circular oligonucleotide comprises a gap between the first end portion and the second end portion; and (c) a second oligonucleotide comprising a first end portion and a second end portion, wherein the first end portion and second end portion of the second oligonucleotide are complementary and bind to two neighboring segments of the second target nucleic acid sequence or the reverse complement thereof, or the portion thereof, so that the second oligonucleotide forms a second circular oligonucleotide within the biological sample, wherein the second circular oligonucleotide comprises a gap between the first end portion and the second end portion. In some embodiments, the first circular oligonucleotide further comprises a first nucleic acid enzyme configured to join the first and second end portions of the first oligonucleotide, and the second circular oligonucleotide further comprises a second nucleic acid enzyme configured to join the first and second end portions of the second oligonucleotide. In some embodiments, the first and second nucleic acid enzymes comprises a nucleic acid ligase, a nucleic acid ligation enzyme, a nucleic acid polymerase, a nucleic acid polymerization enzyme, or combinations thereof. In some embodiments, the system further comprises a first amplicon of the first circular oligonucleotide, and a second amplicon of the second circular oligonucleotide.

In some embodiments, the first amplicon comprises a first concatemer and/or the second amplicon comprises a second concatemer. In some embodiments, (a) the first concatemer comprises at least two repeats of a first unit nucleic acid sequence comprising the first target nucleic acid sequence or a portion thereof, or the reverse complement thereof or a portion thereof; and/or (b) the second concatemer comprises at least two repeats of a second unit nucleic acid sequence comprising the second target nucleic acid sequence or a portion thereof, or the reverse complement thereof or a portion thereof.

In some embodiments, the first concatemer further comprises the first identification sequence that identifies the first target nucleic acid sequence, or a sequencing primer or a reverse complement thereof, wherein the second concatemer further comprises the second identification sequence that identifies the second target nucleic acid sequence, or a sequencing primer or a reverse complement thereof. In some embodiments, the first concatemer further comprises a first compaction oligonucleotide, wherein: (a) a first segment of the first compaction oligonucleotide is complementary and binds to a first portion of the first concatemer; and (b) a second segment of the first compaction oligonucleotide is complementary and binds to a second portion of the first concatemer, to result in a reduction in the size or a change in the shape of the first concatemer. In some embodiments, the second concatemer further comprises a second compaction oligonucleotide, wherein:(a) a first segment of the second compaction oligonucleotide is complementary and binds to a first portion of the second concatemer; and (b) a second segment of the second compaction oligonucleotide is complementary and binds to a second portion of the second concatemer, to result in a reduction in the size or a change in the shape of the second concatemer. In some embodiments, the system further comprises a polymerizing enzyme, a plurality of nucleotides, and a primer sequence that is complementary to at least a portion of the first concatemer or the second concatemer under conditions sufficient to form a binding complex. In some embodiments, the system further comprises an agent configured to remove a blocking group from a nucleotide of the plurality of nucleotides and generate a 3′ OH group on a sugar moiety of the nucleotide. In some embodiments, the blocking group comprises an alkyl group, an alkenyl group, an alkynyl group, an allyl group, an aryl group, a benzyl group, an azide group, an azido group, an O-azidomethyl group, an amine group, an amide group, a keto group, an isocyanate group, a phosphate group, a thiol group, a disulfide group, a carbonate group, a urea group, or a silyl group. In some embodiments, the plurality of nucleotides comprises a fluorescent label. In some embodiments, the plurality of nucleotides consists of at least two of the same type of nucleotide, wherein the same type of nucleotide is selected from the group comprising dATP, dGTP, dCTP, dTTP, and dUTP, wherein the fluorescent label of one type of nucleotide of the group emits light at a wavelength that is different from the wavelength of light emitted from the fluorescent label of another type of nucleotide of the group. In some embodiments, the plurality of nucleotides comprises at least two types of nucleotides, wherein the at least two types of nucleotides are selected from the group comprising dATP, dGTP, dCTP, dTTP, and dUTP, wherein the fluorescent label of one type of nucleotide of the group emits light at a wavelength that is different from the wavelength of light emitted from the fluorescent label of another type of nucleotide of the group. In some embodiments, the system further comprises a plurality of nucleotide conjugates, wherein a nucleotide conjugate of the plurality of nucleotide conjugates is configured to form a multivalent binding complex comprising two or more of the polymerizing enzyme, the nucleotide conjugate, and the at least two target nucleic acid sequences, wherein the nucleotide conjugate comprises a label and at least two nucleotide moieties that are each complementary and bind to a nucleotide of each of the at least two target nucleic acid sequences. In some embodiments, the system further comprises a first oligonucleotide conjugate comprising a first short nucleic acid and a first binding moiety that binds specifically to a first target polypeptide, wherein the first short nucleic acid comprises a first tag sequence and a second tag sequence, wherein the first oligonucleotide conjugate binds specifically to the first target polypeptide in the biological sample through the first binding moiety to form a first binding complex, wherein the first and second tag sequences identify the first binding moiety in a nucleic acid sequence reaction. In some embodiments, the system further comprises a second oligonucleotide conjugate comprising a second short nucleic acid and a second binding moiety that binds specifically to a second target polypeptide, wherein the second short nucleic acid comprises a third tag sequence and a fourth tag sequence, wherein the second oligonucleotide conjugate binds specifically to the second target polypeptide in the biological sample through the second binding moiety to form a second binding complex, wherein the third and fourth tag sequences identify the second binding moiety in a nucleic acid sequence reaction. In some embodiments, the first or second binding moiety is an antibody or an antigen-binding fragment thereof. In some embodiments, the system further comprises a third oligonucleotide comprising a first end portion and a second end portion, wherein the first end portion and second end portion of the third oligonucleotide are complementary and bind to the first tag sequence and the second tag sequence of the first oligonucleotide conjugate so that the third oligonucleotide forms a circular structure with a gap between the first end portion and the second end portion. In some embodiments, the system further comprises a fourth oligonucleotide comprising a first end portion and a second end portion, wherein the first end portion and second end portion of the fourth oligonucleotide are complementary and bind to the third tag sequence and the fourth tag sequence so that the fourth oligonucleotide forms a circular structure with a gap between the first end portion and the second end portion. In some embodiments, the system further comprises a third circular oligonucleotide that results from joining the first and second end portions of the third oligonucleotide and a fourth circular oligonucleotide that results from joining the first and second end portions of the fourth oligonucleotide, wherein the first circular oligonucleotide and the third circular oligonucleotide comprise a first sequencing primer or the reverse complement thereof, wherein the second circular oligonucleotide and the fourth circular oligonucleotide comprise a second sequencing primer or the reverse complement thereof, wherein the sequences of the first and second sequencing primers have at least one nucleotide of difference, wherein the joining is carried out by the first or second nucleic acid enzyme. In some embodiments, the system further comprises a third amplicon of the third circular oligonucleotide, and a fourth amplicon of the fourth circular oligonucleotide. In some embodiments, the third amplicon comprises a third concatemer and/or the fourth amplicon comprises a fourth concatemer. In some embodiments, (a) the third concatemer comprises at least two repeats of a third unit nucleic acid sequence comprising the third circular oligonucleotide or a portion thereof, or the reverse complement thereof or a portion thereof; and/or (b) the fourth concatemer comprises at least two repeats of a second unit nucleic acid sequence comprising the fourth circular oligonucleotide or a portion thereof, or the reveres complement thereof or a portion thereof. In some embodiments, the third concatemer further comprises the first or second tag sequence or a reverse complement thereof that identifies the first binding moiety, wherein the fourth concatemer further comprises the third or fourth tag sequence or a reverse complement thereof that identifies the second binding moiety. In some embodiments, the third concatemer further comprises a third compaction oligonucleotide, wherein: (a) a first segment of the third compaction oligonucleotide is complementary and binds to a first portion of the third concatemer; and (b) a second segment of the third compaction oligonucleotide is complementary and binds to a second portion of the third concatemer, to result in a reduction in the size or a change in the shape of the third concatemer.

In some embodiments, the fourth concatemer further comprises a fourth compaction oligonucleotide, wherein: (a) a first segment of the fourth compaction oligonucleotide is complementary and binds to a first portion of the fourth concatemer; and (b) a second segment of the fourth compaction oligonucleotide is complementary and binds to a second portion of the fourth concatemer, to result in a reduction in the size or a change in the shape of the fourth concatemer. In some embodiments, the system further comprises a second polymerizing enzyme, a second plurality of nucleotides, and a second primer sequence that is complementary to at least a portion of the third concatemer or the fourth concatemer under conditions sufficient to form a binding complex comprising the third or fourth concatemer hybridized to the second primer sequence, the second polymerizing enzyme, and a second nucleotide of the second plurality of nucleotides that is complementary and binds to the a nucleotide of the third or fourth concatemer. In some embodiments, the system further comprises a second agent configured to remove a second blocking group from a second nucleotide of the second plurality of nucleotides, and generate a 3′ OH group on a sugar moiety of the second nucleotide. In some embodiments, the second blocking group comprises an alkyl group, an alkenyl group, an alkynyl group, an allyl group, an aryl group, a benzyl group, an azide group, an azido group, an O-azidomethyl group, an amine group, an amide group, a keto group, an isocyanate group, a phosphate group, a thiol group, a disulfide group, a carbonate group, a urea group, or a silyl group. In some embodiments, the second plurality of nucleotides comprises a second fluorescent label. In some embodiments, the second plurality of nucleotides consists of at least two of the same type of nucleotide, wherein the same type of nucleotide is selected from the group comprising dATP, dGTP, dCTP, dTTP, and dUTP, wherein the second fluorescent label of one type of nucleotide of the group emits light at a wavelength that is different from the wavelength of light emitted from the second fluorescent label of another type of nucleotide of the group. In some embodiments, the second plurality of nucleotides comprises at least two types of nucleotides, wherein the at least two types of nucleotides are selected from the group comprising dATP, dGTP, dCTP, dTTP, and dUTP, wherein the second fluorescent label of one type of nucleotide of the group emits light at a wavelength that is different from the wavelength of light emitted from the second fluorescent label of another type of nucleotide of the group. In some embodiments, the system further comprises a second plurality of nucleotide conjugates, wherein a second nucleotide conjugate of the second plurality of nucleotide conjugates is configured to form a multivalent binding complex comprising two or more of the second polymerizing enzyme, the second nucleotide conjugate of the a second plurality of nucleotide conjugates plurality of nucleotide conjugates, and the at least two of the first short nucleic acid or the short nucleic acid or a portion thereof, wherein the second nucleotide conjugate comprises a label and at least two nucleotide moieties that are each complementary and bind to a nucleotide of each of the at least two of the first short nucleic acid or the short nucleic acid or a portion thereof. In some embodiments, the system further comprises a solid surface comprising the biological sample immobilized to the solid surface. In some embodiments, the biological sample is permeabilized. In some embodiments, the solid surface further comprises a hydrophilic polymer coating layer coupled thereto. In some embodiments, the hydrophilic polymer coating layer has a water contact angle that is less than 50 degrees. In some embodiments, the system further comprises an optical imaging module configured to image the biological sample coupled to the solid surface to detecting in situ the at least two target nucleic acid sequences and/or the at least two target polypeptides in the biological sample. In some embodiments, the first target polypeptide is encoded by the first target nucleic acid molecule or a reverse complement thereof and the second target polypeptide is encoded by the second target nucleic acid molecule or a reverse complement thereof.

Provided herein, in another aspect, is a computer-implemented system comprising a computing device comprising at least one processor, an operating system configured to perform executable instructions, a memory, and a computer program including instructions executable by the computing device wherein the instructions comprise a method disclosed herein.

Provided herein, in another aspect, is non-transitory computer-readable storage media encoded with a computer program including instructions executable by one or more processors, wherein the instructions comprise a method disclosed herein.

Provided herein, in another aspect, is a kit for detecting in situ at least two target nucleic acid sequences in a biological sample, wherein the at least two target nucleic acid sequences comprise a first target nucleic acid sequence and a second target nucleic acid sequence, the kit comprising: (a) a first oligonucleotide comprising a first end portion and a second end portion, wherein the first end portion and second end portion of the first oligonucleotide are complementary and bind to two neighboring segments of the first target nucleic acid sequence or the reverse complement thereof, or a portion thereof, so that the first oligonucleotide forms a first circular oligonucleotide within the biological sample, wherein the first circular oligonucleotide comprises a gap between the first end portion and the second end portion, wherein the first oligonucleotide comprises a first identification sequence that identifies the first target nucleic acid sequence, a first sequencing primer or a reverse complement thereof, a compaction oligonucleotide or a reverse complement thereof, or a primer for nucleic acid amplification or a reverse complement thereof, (b) a second oligonucleotide comprising a first end portion and a second end portion, wherein the first end portion and second end portion of the second oligonucleotide are complementary and bind to two neighboring segments of the second target nucleic acid sequence or the reverse complement thereof, or the portion thereof, so that the second oligonucleotide forms a second circular oligonucleotide within the biological sample, wherein the second circular oligonucleotide comprises a gap between the first end portion and the second end portion, wherein the second oligonucleotide comprises a second identification sequence that identifies the second target nucleic acid sequence, a second sequencing primer or a reverse complement thereof, a compaction oligonucleotide or a reverse complement thereof, or a primer for nucleic acid amplification or a reverse complement thereof; (c) a first nucleic acid enzyme configured to join the first and second end portions of the first oligonucleotide and generate a first circular oligonucleotide, or the first and second end portions of the second oligonucleotide and generate a second circular oligonucleotide; (d) a second nucleic acid enzyme configured to amplify the first circular oligonucleotide to produce a first concatemer or the second circular oligonucleotide to product a second concatemer; (e) a third nucleic acid enzyme, a sequencing primer complementary to a portion of the first concatemer or a portion of the second concatemer, a plurality of nucleotides, or a plurality of nucleotide conjugates, wherein a nucleotide conjugate of the plurality of nucleotide conjugates comprises a core and two of nucleotide moieties attached to the core, (i) wherein the third nucleic acid enzyme, a nucleotide of the plurality of nucleotides, and the sequencing primer are configured to form a binding complex comprising the third nucleic acid enzyme, the nucleotide of the plurality of nucleotides that is complementary and binds to a nucleotide unit of the first or the second concatemer, and the first concatemer or the second concatemer hybridized to the sequencing primer; or (ii) wherein at least two of the third nucleic acid enzyme, the nucleotide conjugate, the sequencing primer, and the first or the second concatemer are configured to form a multivalent binding complex comprising the third nucleic acid enzyme, the nucleotide conjugate, and the first or the second concatemer hybridized to the sequencing primer, wherein the two nucleotide moieties are complementary and bind to two nucleotide units the first or the second concatemer, and each of the two of the first concatemer or the second concatemer hybridized to each of the two of the sequencing primer; (f) a fourth nucleic acid enzyme configured to: (i) add a nucleotide of the plurality of nucleotides to the end of the sequencing primer hybridized to the first concatemer and generate a first sequencing product nucleic acid molecule, wherein the nucleotide is unlabeled and comprises a blocking group; or (ii) add a nucleotide of the plurality of nucleotides to the end of the sequencing primer hybridized to the second concatemer and generate a second sequencing product nucleic acid molecule, wherein the nucleotide is unlabeled and comprises a blocking group; and (g) a dissociation reagent configured to remove the first sequencing product nucleic acid molecule from the first concatemer, or the second sequencing product nucleic acid molecule from the second concatemer. In some embodiments, the kit is further configured for detecting in situ at least two target RNA sequences and at least two target polypeptides comprising a first target polypeptide and a second target polypeptide in a biological sample, the kit comprising: the first target polypeptide encoded by the first target nucleic acid molecule or a reverse complement thereof, wherein the second target polypeptide is encoded by the second target nucleic acid molecule or a reverse complement thereof, wherein the full sequence of the first target RNA sequence and the full sequence of the second target RNA sequence have at least one nucleotide of difference, wherein the full sequence of the first target polypeptide and the full sequence of the second target polypeptide have at least one amino acid of difference. In some embodiments, the first, second, third, or fourth nucleic acid enzyme comprises a nucleic acid ligase, a nucleic acid ligation enzyme, a nucleic acid polymerase, a nucleic acid polymerization enzyme, or combinations thereof. In some embodiments, the nucleotide of the plurality of nucleotides comprises a fluorescent label. In some embodiments, the nucleotide of the plurality of nucleotides comprises a removable blocking group at the 3′ carbon position of the sugar moiety. In some embodiments, the plurality of nucleotides consist of at least two of the same type of nucleotide comprising a fluorescent label, wherein the same type of nucleotide is selected from the group comprising dATP, dGTP, dCTP, dTTP, and dUTP, wherein the fluorescent label of one type of nucleotide of the group emits light at a wavelength that is different from the wavelength of light emitted from the fluorescent label of another type of nucleotide of the group. In some embodiments, the plurality of nucleotides comprise at least two types of nucleotides, wherein a type of the at least two types of nucleotides comprises a fluorescent label, wherein the at least two types of nucleotides are selected from the group comprising dATP, dGTP, dCTP, dTTP, and dUTP, wherein the fluorescent label of one type of nucleotide of the group emits light at a wavelength that is different from the wavelength of light emitted from the fluorescent label of another type of nucleotide of the group. In some embodiments, the nucleotide conjugate comprises a detectable label. In some embodiments, the nucleotide conjugate comprises a fluorescent label. In some embodiments, the biological sample comprises a human sample, a simian sample, an ape sample, a canine sample, a feline sample, a bovine sample, an equine sample, a murine sample, a porcine sample, a caprine sample, a lupine sample, a ranine sample, a piscine sample, a plant sample, an insect sample, a bacteria sample, an algae sample, a viral sample, a protozoa sample, or a fungus sample. In some embodiments, the biological sample comprises a cellular organelle, a cell, a whole cell, a group of whole cells, a tissue, an intact tissue, a tumor, an intact tumor, an organ, or an organism. In some embodiments, the biological sample comprises a cellular organelle, a cell, a whole cell, a group of whole cells, a tissue, an intact tissue, a tumor, an intact tumor, an organ, an organism, a protozoa, an algae, a bacteria, a virus, a plant, a fungus, an insect, or an animal. In some embodiments, the biological sample comprises a fresh sample, a processed sample, a freshly-frozen sample, a sectioned sample, or a formalin-fixed and paraffin-embedded (FFPE) sample. In some embodiments, the first target nucleic acid sequence comprises DNA, cDNA, RNA, coding RNA, non-coding RNA, mRNA, tRNA, rRNA, miRNA, gRNA, snRNA, siRNA, anti-sense RNA, mature microRNA, or immature microRNA, and/or, wherein the first target RNA sequence comprises coding RNA, non-coding RNA, mRNA, tRNA, rRNA, miRNA, gRNA, snRNA, siRNA, anti-sense RNA, mature microRNA, or immature microRNA. In some embodiments, the second target nucleic acid sequence comprises DNA, cDNA, RNA, coding RNA, non-coding RNA, mRNA, tRNA, rRNA, miRNA, gRNA, snRNA, siRNA, anti-sense RNA, mature microRNA, or immature microRNA, and/or wherein the second target RNA sequence comprises coding RNA, non-coding RNA, mRNA, tRNA, rRNA, miRNA, gRNA, snRNA, siRNA, anti-sense RNA, mature microRNA, or immature microRNA. In some embodiments, the blocking group comprises an alkyl group, an alkenyl group, an alkynyl group, an allyl group, an aryl group, a benzyl group, an azide group, an azido group, an O-azidomethyl group, an amine group, an amide group, a keto group, an isocyanate group, a phosphate group, a thiol group, a disulfide group, a carbonate group, a urea group, or a silyl group. In some embodiments, the kit further comprises: (a) a first oligonucleotide conjugate comprising a first short nucleic acid and a first binding moiety that binds specifically to the first target polypeptide, wherein the first short nucleic acid comprises a first tag sequence, a second tag sequence, wherein the first oligonucleotide conjugate binds specifically to the first target polypeptide through the first binding moiety to form a first binding complex, wherein the first and second tag sequences identify the first binding moiety; or (b) a second oligonucleotide conjugate comprising a second short nucleic acid and a second binding moiety that binds specifically to the second target polypeptide, wherein the second short nucleic acid comprises a third tag sequence, a fourth tag sequence, wherein the second oligonucleotide conjugate binds specifically to the second target polypeptide through the second binding moiety to form a second binding complex, wherein the third and fourth tag sequences identify the second binding moiety. In some embodiments, the kit further comprises: (a) a third oligonucleotide comprising a first end portion and a second end portion, wherein the first end portion and second end portion of the third oligonucleotide are complementary and bind to the first tag sequence and the second tag sequence of the first oligonucleotide conjugate so that the third oligonucleotide forms a circular structure with a gap between the first end portion and the second end portion; or (b) a fourth oligonucleotide comprising a first end portion and a second end portion, wherein the first end portion and second end portion of the fourth oligonucleotide are complementary and bind to the third tag sequence and the fourth tag sequence so that the fourth oligonucleotide forms a circular structure with a gap between the first end portion and the second end portion. In some embodiments, the third oligonucleotide comprises a third identification sequence that identifies the first binding moiety, and wherein the fourth oligonucleotide comprises a fourth identification sequence that identifies the second binding moiety. In some embodiments, the first oligonucleotide and third oligonucleotide comprise the same first sequencing primer or the reverse complement thereof, wherein the second oligonucleotide and the fourth oligonucleotide comprise the same second sequencing primer or the reverse complement thereof. In some embodiments, the kit further comprises an agent that reacts with the reactive group at the 3′ carbon of the sugar moiety in the nucleotide moiety of the nucleotide conjugate. In some embodiments, the kit further comprises a reagent for use in the nucleotide binding reaction. In some embodiments, the reagent comprises a cation. In some embodiments, the kit further comprises a reverse transcriptase, a primer for reverse transcription, a sequencing primer, a reagent configured to permeabilize the biological sample, a reagent configured to fix the biological sample, an unblocked nucleotide, a blocked nucleotide. a reagent for use in the nucleotide incorporation reaction, a solution comprising a cation, one or more unlabeled nucleotides, one or more buffers for reverse transcription, one or more buffers for nucleic acid binding, one or more buffers for nucleic acid amplification, or one or more buffers for nucleic acid dissociation. In some embodiments, the kit further comprises instructions for use of the kit to detect in situ the at least two target nucleic acid sequences in the biological sample. In some embodiments, the kit further comprises instructions for use of the kit to detect in situ the at least two target nucleic acid sequences and the at least two target polypeptides in the biological sample. In some embodiments, the instructions comprise performing a sequencing by synthesis reaction. In some embodiments, the instructions comprise performing a sequencing by binding reaction in which detection in situ is not contemporaneous with a nucleotide incorporation step. In some embodiments, the kit further comprises instructions for use of the kit using a method disclosed herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### DESCRIPTION OF THE DRAWINGS

The novel features of the inventive concepts are set forth with particularity in the appended claims. A better understanding of the features and advantages of the present inventive concepts will be obtained by reference to the following detailed description that sets forth illustrative embodiments, in which the principles of the inventive concepts are utilized, and the accompanying drawings of which:

FIG. 1 is a schematic showing a non-limiting example workflow for generating concatemers inside a cellular sample. Target RNA harbored by a cellular sample is hybridized to a reverse transcription primer (RT primer) and reverse transcription is conducted to synthesize a first strand cDNA. The first strand cDNA is hybridized to a target-specific padlock probe to generate a circularized padlock probe having a nick (solid downward triangle) between the first and second ends of the hybridized padlock probe. The padlock probe carries an adaptor for a universal compaction oligonucleotide binding site, an adaptor for a universal sequencing primer binding site, and a target barcode sequence that corresponds to a given target cDNA. The nick in the circularized padlock probe is ligated to form covalently closed circular padlock probe which carries a cDNA sequence that corresponds to the target RNA. The sequence of an non-limiting example padlock probe which binds selectively to GAPDH cDNA is shown at the bottom of FIG. 1. The covalently closed circular padlock probe can be subjected to rolling circle amplification inside the cellular sample to generate a concatemer molecule. The concatemer molecule can be reiteratively sequenced inside the cellular sample.

FIG. 2 is a schematic showing a non-limiting example workflow for sequencing a concatemer that is generated inside the cell as shown in FIG. 1. The concatemer depicted in FIG. 2 includes tandem repeat units where each unit comprises: (i) a universal sequencing primer binding site (Seq), (ii) universal compaction oligonucleotide binding site (CO), (iii) an insert sequence that corresponds to a given target cDNA, and (iv) a target barcode sequence that corresponds to the given target cDNA (BC). In some embodiments, universal sequencing primers (solid arrows) hybridize to the universal sequencing primer binding sites and no more than 30 sequencing cycles are conducted to generate a plurality of first sequencing read products (dashed arrows), where the first sequencing read products include only the target barcode sequence. The plurality of first sequencing read products are removed from the concatemer, and the sequencing is repeated where no more than 30 sequencing cycles are conducted to generate another plurality of first sequencing read products (dashed arrows), where the first sequencing read products include only the target barcode sequence. The plurality of first sequencing read products are removed from the concatemer, and the sequencing is once again repeated where no more than 30 sequencing cycles are conducted to generate another plurality of first sequencing read products (dashed arrows), where the first sequencing read products include only the target barcode sequence. In some embodiments, the reiterative sequencing can be conducted up to 50 times. The sequences of all of the first sequencing read products can be determined and aligned with a first reference sequence (e.g., reference barcode sequence) to confirm the presence of the first target RNA molecules inside the cellular sample.

FIG. 3 is a schematic showing a non-limiting example workflow for sequencing a concatemer that is generated inside the cell as shown in FIG. 1. The concatemer depicted in FIG. 3 includes tandem repeat units where each unit comprises: (i) a universal sequencing primer binding site (Seq), (ii) universal compaction oligonucleotide binding site (CO), (iii) an insert sequence that corresponds to a given target cDNA, and (iv) a target barcode sequence that corresponds to the given target cDNA (BC). In some embodiments, universal sequencing primers (solid arrows) hybridize to the universal sequencing primer binding sites and no more than 30 sequencing cycles are conducted to generate a plurality of first sequencing read products (dashed arrows), where the first sequencing read products include the target barcode sequence and a portion of the insert sequence. The plurality of first sequencing read products are removed from the concatemer, and the sequencing is repeated where no more than 30 sequencing cycles are conducted to generate another plurality of first sequencing read products (dashed arrows), where the first sequencing read products include the target barcode sequence and a portion of the insert sequence. The plurality of first sequencing read products are removed from the concatemer, and the sequencing is once again repeated where no more than 30 sequencing cycles are conducted to generate another plurality of first sequencing read products (dashed arrows), where the first sequencing read products include the target barcode sequence and a portion of the insert sequence. In some embodiments, the reiterative sequencing can be conducted up to 50 times. The sequences of all the first sequencing read products can be determined and aligned with a first reference sequence (e.g., reference barcode sequence and the insert sequence that corresponds to the target RNA) to confirm the presence of the first target RNA molecules inside the cellular sample.

FIG. 4 is a schematic showing a non-limiting example workflow for sequencing a concatemer that is generated inside the cell as shown in FIG. 1. The concatemer depicted in FIG. 4 includes tandem repeat units where each unit comprises: (i) a universal sequencing primer binding site (Seq), (ii) universal compaction oligonucleotide binding site (CO), and (iii) an insert sequence that corresponds to a given target cDNA. In some embodiments, universal sequencing primers (solid arrows) hybridize to the universal sequencing primer binding sites and no more than 30 sequencing cycles are conducted to generate a plurality of first sequencing read products (dashed arrows), where the first sequencing read products include a portion of the insert sequence. The plurality of first sequencing read products are removed from the concatemer, and the sequencing is repeated where no more than 30 sequencing cycles are conducted to generate another plurality of first sequencing read products (dashed arrows), where the first sequencing read products include a portion of the insert sequence. The plurality of first sequencing read products are removed from the concatemer, and the sequencing is once again repeated where no more than 30 sequencing cycles are conducted to generate another plurality of first sequencing read products (dashed arrows), where the first sequencing read products include a portion of the insert sequence. In some embodiments, the reiterative sequencing can be conducted up to 50 times. The sequences of all of the first sequencing read products can be determined and aligned with a first reference sequence (e.g., the insert sequence that corresponds to the target RNA) to confirm the presence of the first target RNA molecules inside the cellular sample.

FIG. 5 is a schematic showing a non-limiting example workflow for sequencing a concatemer that is generated inside the cell as shown in FIG. 1. The concatemer depicted in FIG. 5 includes tandem repeat units where each unit comprises: (i) a universal sequencing primer binding site (Seq) and (ii) an insert sequence that corresponds to a given target cDNA. In some embodiments, universal sequencing primers (solid arrows) hybridize to the universal sequencing primer binding sites and no more than 30 sequencing cycles are conducted to generate a plurality of first sequencing read products (dashed arrows), where the first sequencing read products include a portion of the insert sequence. The plurality of first sequencing read products are removed from the concatemer, and the sequencing is repeated where no more than 30 sequencing cycles are conducted to generate another plurality of first sequencing read products (dashed arrows), where the first sequencing read products include a portion of the insert sequence. The plurality of first sequencing read products are removed from the concatemer, and the sequencing is once again repeated where no more than 30 sequencing cycles are conducted to generate another plurality of first sequencing read products (dashed arrows), where the first sequencing read products include a portion of the insert sequence. In some embodiments, the reiterative sequencing can be conducted up to 50 times. The sequences of all of the first sequencing read products can be determined and aligned with a first reference sequence (e.g., the insert sequence that corresponds to the target RNA) to confirm the presence of the first target RNA molecules inside the cellular sample.

FIG. 6 shows images of fluorescent sequencing signals emitted from HEK 293 cells cultured on a poly-lysine coated flow cell. The cultured cells were fixed, permeabilized, subjected to reverse transcription reactions, and subjected to rolling circle amplification on the flow cell, all under conditions suitable for retaining the cellular nucleic acids inside the cells. The coated flow cell lacked surface capture primers. The retained nucleic acids in the cells were sequenced using a two-stage sequencing method that employed detectably labeled multivalent molecules and unlabeled nucleotide analogs. Thirty cycles of the two-stage sequencing reactions were conducted using multivalent molecules labeled with one of four fluorophores and unlabeled nucleotide analogs. FIG. 6 shows results from the second in situ sequencing experiment (see Example 1). FIG. 6 shows fluorescent signals from cycles 20-25 where the sequence reads CCTCCT. A total of 4620 fluorescent spots were detected with significant signals in all cycles. More than 90% of the fluorescent spots detected the sample index sequences.

FIG. 7 is a schematic of various non-limiting example configurations of multivalent molecules. Left (Class I): schematics of multivalent molecules having a “starburst” or “helter-skelter” configuration. Center (Class II): a schematic of a multivalent molecule having a dendrimer configuration. Right (Class III): a schematic of multiple multivalent molecules formed by reacting streptavidin with 4-arm or 8-arm PEG-NHS with biotin and dNTPs. Nucleotide units are designated ‘N’, biotin is designated 13′, and streptavidin is designated ‘SA’.

FIG. 8 is a schematic of an non-limiting example multivalent molecule comprising a generic core attached to a plurality of nucleotide-arms.

FIG. 9 is a schematic of an non-limiting example multivalent molecule comprising a dendrimer core attached to a plurality of nucleotide-arms.

FIG. 10 shows a schematic of a non-limiting example multivalent molecule comprising a core attached to a plurality of nucleotide-arms, where the nucleotide arms comprise biotin, spacer, linker and a nucleotide unit.

FIG. 11 is a schematic of a non-limiting example nucleotide-arm comprising a core attachment moiety, spacer, linker and nucleotide unit.

FIG. 12 shows the chemical structure of a non-limiting example spacer (top), and the chemical structures of various non-limiting example linkers, including an 11-atom Linker, 16-atom Linker, 23-atom Linker and an N3 Linker (bottom).

FIG. 13 shows the chemical structures of various non-limiting example linkers, including Linkers 1-9.

FIG. 14 shows the chemical structures of various non-limiting example linkers joined/attached to nucleotide units.

FIG. 15 shows the chemical structures of various non-limiting example linkers joined/attached to nucleotide units.

FIG. 16 shows the chemical structures of various non-limiting example linkers joined/attached to nucleotide units.

FIG. 17 shows the chemical structure of a non-limiting example biotinylated nucleotide-arm. In this example, the nucleotide unit is connected to the linker via a propargyl amine attachment at the 5 position of a pyrimidine base or the 7 position of a purine base.

FIG. 18 is a schematic of a guanine tetrad (e.g., G-tetrad).

FIG. 19 is a schematic of a non-limiting example intramolecular G-quadruplex structure.

FIG. 20 is a schematic showing a workflow for generating circularized padlock probes, comprising generating first and second cDNAs from first and second target RNA molecules (respectively), hybridizing first and second padlock probes to the first and second cDNA molecules (respectively) to generate first and second circularized padlock probes (respectively). The first padlock probe comprises (i) a first target barcode sequence (target BC-1) that uniquely identifies the first target RNA, (ii) a first batch-specific sequencing primer binding site (Batch Seq-1) (or a complementary sequence thereof), (iii) a universal binding site for an amplification primer (universal RCA) (or a complementary sequence thereof), and (iv) a universal binding site for a compaction oligonucleotide (or a complementary sequence thereof). The second padlock probe comprises (i) a second target barcode sequence (target BC-2) that uniquely identifies the second target RNA, (ii) a second batch-specific sequencing primer binding site (Batch Seq-2) (or a complementary sequence thereof), (iii) a universal binding site for an amplification primer (universal RCA) (or a complementary sequence thereof), and (iv) a universal binding site for a compaction oligonucleotide (or a complementary sequence thereof).

FIG. 21 is a schematic showing a rolling circle and sequencing workflow comprising generating first and second concatemers by conducting rolling circle amplification using first and second covalently closed circular molecules (respectively). The first and second concatemers are subjected to a first sequencing workflow using first batch-specific sequencing primers, sequencing polymerases, and a plurality of nucleotide reagents. The first concatemers undergo reiterative sequencing but the second concatemers do not. The first and second concatemers are subjected to a second sequencing workflow using second batch-specific sequencing primers, sequencing polymerases, and a plurality of nucleotide reagents. The second concatemers undergo reiterative sequencing but the first concatemers do not.

FIG. 22 is a schematic showing first and second antibody-oligonucleotide conjugates. The first antibody-oligonucleotide conjugate comprises a first antibody that selectively binds a first target polypeptide. The first antibody is linked to a first oligonucleotide, where the first oligonucleotide carries a first and second tag sequence. The second antibody-oligonucleotide conjugate comprises a second antibody that selectively binds a second target polypeptide. The second antibody is linked to a second oligonucleotide, where the second oligonucleotide carries a third and fourth tag sequence. The first, second, third and fourth tag sequences differ from each other.

FIG. 23 is a schematic showing first and second antibody-oligonucleotide conjugates each bound to their respective target polypeptides and their respective padlock probes.

The first padlock probe comprises (i) a sequence that can hybridize to the first tag sequence, (ii) a third target barcode sequence (target BC-3) that uniquely identifies the first antibody which selectively binds the first target polypeptide, (iii) a first batch-specific sequencing primer binding site (Batch Seq-1) (or a complementary sequence thereof), (iv) a universal binding site for an amplification primer (universal RCA) (or a complementary sequence thereof), (v) a universal binding site for a compaction oligonucleotide (or a complementary sequence thereof), and (vi) a sequence that can hybridize to the second tag sequence. The second padlock probe comprises (i) a sequence that can hybridize to the third tag sequence, (ii) a fourth target barcode sequence (target BC-4) that uniquely identifies the second antibody which selectively binds the second target polypeptide, (iii) a second batch-specific sequencing primer binding site (Batch Seq-2) (or a complementary sequence thereof), (iv) a universal binding site for an amplification primer (universal RCA) (or a complementary sequence thereof), (v) a universal binding site for a compaction oligonucleotide (or a complementary sequence thereof), and (vi) a sequence that can hybridize to the fourth tag sequence.

FIG. 24 is a schematic showing a rolling circle amplification and sequencing workflow for a first target RNA and first target polypeptide. A first target cDNA molecule (left top schematic) hybridizes with target-specific padlock probe for a first cDNA carrying (i) first and second binding arms that hybridize to the first target cDNA, (ii) a first barcode sequence which uniquely identifies the first target cDNA (BC-1), (iii) a first batch-specific sequencing primer binding site (batch Seq-1), (iv) a universal binding site for an amplification primer, and (v) a universal binding site for a compaction oligonucleotide. The hybridized first padlock probe includes a nick or gap, the nick or gap are enzymatically closed to generate a first covalently closed circular molecule carrying first cDNA sequences. The covalently closed circular molecule undergoes rolling circle amplification to generate a first cDNA concatemer molecule (top concatemer molecule). The first cDNA concatemer molecule undergoes sequencing using first batch-specific sequencing primers. A first antibody-oligonucleotide conjugate (right top schematic) binds with a first target polypeptide. The oligonucleotide of the first antibody-oligonucleotide conjugate binds a target-specific padlock probe for a first target polypeptide-oligonucleotide conjugate carrying (i) first and second binding arms that hybridize to a first and second tag sequence, (ii) a third barcode sequence (BC-3) which uniquely identifies the first antibody-oligonucleotide conjugate which selectively binds a first target polypeptide, and (iii) a first batch-specific sequencing primer binding site (batch Seq-1), (iv) a universal binding site for an amplification primer, and (v) a universal binding site for a compaction oligonucleotide. The hybridized first padlock probe includes a nick or gap, the nick or gap are enzymatically closed to generate a third covalently closed circular molecule carrying first and second tag sequences. The covalently closed circular molecule undergoes rolling circle amplification to generate a third oligonucleotide-tagged concatemer molecule (bottom concatemer molecule). The third oligonucleotide-tagged concatemer molecule undergoes sequencing using first batch-specific sequencing primers.

FIG. 25 is a schematic showing a rolling circle amplification and sequencing workflow for a second target RNA and second target polypeptide. A second target cDNA molecule (left top schematic) hybridizes with target-specific padlock probe for a second cDNA carrying (i) first and second binding arms that hybridize to the second target cDNA, (ii) a second barcode sequence which uniquely identifies the second target cDNA (BC-2), (iii) a second batch-specific sequencing primer binding site (batch Seq-2), (iv) a universal binding site for an amplification primer, and (v) a universal binding site for a compaction oligonucleotide. The hybridized second padlock probe includes a nick or gap, the nick or gap are enzymatically closed to generate a second covalently closed circular molecule carrying second cDNA sequences. The covalently closed circular molecule undergoes rolling circle amplification to generate a second cDNA concatemer molecule (top concatemer molecule). The second cDNA concatemer molecule undergoes sequencing using second batch-specific sequencing primers. A second antibody-oligonucleotide conjugate (right top schematic) binds with a second target polypeptide. The oligonucleotide of the second antibody-oligonucleotide conjugate binds a target-specific padlock probe for a second target polypeptide-oligonucleotide conjugate carrying (i) first and second binding arms that hybridize to a third and fourth tag sequence, (ii) a fourth barcode sequence (BC-4) which uniquely identifies the second antibody-oligonucleotide conjugate which selectively binds a second target polypeptide, and (iii) a second batch-specific sequencing primer binding site (batch Seq-2), (iv) a universal binding site for an amplification primer, and (v) a universal binding site for a compaction oligonucleotide. The hybridized fourth padlock probe includes a nick or gap, the nick or gap are enzymatically closed to generate a fourth covalently closed circular molecule carrying third and fourth tag sequences. The covalently closed circular molecule undergoes rolling circle amplification to generate a fourth oligonucleotide-tagged concatemer molecule (bottom concatemer molecule). The fourth oligonucleotide-tagged concatemer molecule undergoes sequencing using second batch-specific sequencing primers.

FIG. 26 shows a computer system that is programmed or otherwise configured to implement methods provided herein.

FIG. 27 depicts a non-limiting example method of an antibody conjugated to multiple oligonucleotides via multiple linkers. Detection efficiency may be increased by adding linker moieties directly or via multivalent structures as described herein. This may reduce non detection due to rolling circle amplification.

FIG. 28 depicts a non-limiting example method as disclosed herein. A first antibody-oligonucleotide conjugate comprising a first antibody and a first oligonucleotide tag and a second antibody-oligonucleotide conjugate comprising a second antibody and a second oligonucleotide tag can be used against the same target protein corresponding to a target RNA. The first oligonucleotide tag and the second oligonucleotide tag can be used to circularize a padlock probe as disclosed herein. This method can increase binding specificity.

FIG. 29 depicts a non-limiting example method as disclosed herein. A first antibody-oligonucleotide conjugate comprising a first antibody and a first oligonucleotide tag and a second antibody-oligonucleotide conjugate comprising a second antibody and a second oligonucleotide tag can be used against the same target protein corresponding to a target RNA. The first oligonucleotide tag and the second oligonucleotide tag can be used to circularize a padlock probe as disclosed herein. This method can increase binding specificity.

### DETAILED DESCRIPTION

### Definitions

The headings provided herein are not limitations of the various aspects of the disclosure, which aspects can be understood by reference to the specification as a whole.

Unless defined otherwise, technical and scientific terms used herein have meanings that are commonly understood by those of ordinary skill in the art unless defined otherwise. Generally, terminologies pertaining to techniques of molecular biology, nucleic acid chemistry, protein chemistry, genetics, microbiology, transgenic cell production, and hybridization described herein are those well-known and commonly used in the art. Techniques and procedures described herein are generally performed according to conventional methods well known in the art and as described in various general and more specific references that are cited and discussed throughout the instant specification. For example, see Sambrook et al., Molecular Cloning: A Laboratory Manual (Third ed., Cold Spring Harbor Laboratory Press, Cold Spring Harbor, N.Y. 2000), which is incorporated by reference in its entirety. See also Ausubel et al., Current Protocols in Molecular Biology, Greene Publishing Associates (1992), which is incorporated by reference in its entirety. The nomenclatures utilized in connection with, and the laboratory procedures and techniques described herein are those well-known and commonly used in the art.

Unless otherwise required by context herein, singular terms shall include pluralities and plural terms shall include the singular. Singular forms “a”, “an” and “the”, and singular use of any word, include plural referents unless expressly and unequivocally limited on one referent.

It is understood the use of the alternative term (e.g., “or”) is taken to mean either one or both or any combination thereof of the alternatives.

The term “and/or” used herein is to be taken mean specific disclosure of each of the specified features or components with or without the other. For example, the term “and/or” as used in a phrase such as “A and/or B” herein is intended to include: “A and B”; “A or B”; “A” (A alone); or “B” (B alone). In a similar manner, the term “and/or” as used in a phrase such as “A, B, and/or C” is intended to encompass each of the following aspects: “A, B, and C”; “A, B, or C”; “A or C”; “A or B”; “B or C”; “A and B”; “B and C”; “A and C”; “A” (A alone); “B” (B alone); or “C” (C alone).

As used herein and in the appended claims, terms “comprising”, “including”, “having” and “containing”, and their grammatical variants, as used herein are intended to be non-limiting so that one item or multiple items in a list do not exclude other items that can be substituted or added to the listed items. It is understood that wherever aspects are described herein with the language “comprising,” otherwise analogous aspects described in terms of “consisting of and/or “consisting essentially of are also provided.

As used herein, the terms “about” and “approximately” refer to a value or composition that is within an acceptable error range for the particular value or composition as determined by one of ordinary skill in the art, which will depend in part on how the value or composition is measured or determined, i.e., the limitations of the measurement system. For example, “about” or “approximately” can mean within one or more than one standard deviation per the practice in the art. Alternatively, “about” or “approximately” can mean a range of up to 10% (i.e., ±10%) or more depending on the limitations of the measurement system. For example, about 5 mg can include any number between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “approximately” should be assumed to be within an acceptable error range for that particular value or composition. Also, where ranges and/or subranges of values are provided, the ranges and/or subranges can include the endpoints of the ranges and/or subranges.

The term “polymerase” and its variants, as used herein, comprises an enzyme comprising a domain that binds a nucleotide (or nucleoside) where the polymerase can form a complex having a template nucleic acid and a complementary nucleotide. The polymerase can have one or more activities including, but not limited to, base analog detection activities, DNA polymerization activity, reverse transcriptase activity, DNA binding, strand displacement activity, and nucleotide binding and recognition. A polymerase can be any enzyme that can catalyze polymerization of nucleotides (including analogs thereof) into a nucleic acid strand. Typically, but not necessarily, such nucleotide polymerization can occur in a template-dependent fashion. Typically, a polymerase comprises one or more active sites at which nucleotide binding and/or catalysis of nucleotide polymerization can occur. In some embodiments, a polymerase includes other enzymatic activities, such as for example, 3′ to 5′ exonuclease activity or 5′ to 3′ exonuclease activity. In some embodiments, a polymerase has strand displacing activity. A polymerase can include without limitation naturally occurring polymerases and any subunits and truncations thereof, mutant polymerases, variant polymerases, recombinant, fusion or otherwise engineered polymerases, chemically modified polymerases, synthetic molecules or assemblies, and any analogs, derivatives or fragments thereof that retain the ability to catalyze nucleotide polymerization (e.g., catalytically active fragment). The polymerase includes catalytically inactive polymerases, catalytically active polymerases, reverse transcriptases, and other enzymes comprising a nucleotide binding domain. In some embodiments, a polymerase can be isolated from a cell, or generated using recombinant DNA technology or chemical synthesis methods. In some embodiments, a polymerase can be expressed in prokaryote, eukaryote, viral, or phage organisms. In some embodiments, a polymerase can be post-translationally modified proteins or fragments thereof. A polymerase can be derived from a prokaryote, eukaryote, virus or phage. A polymerase comprises DNA-directed DNA polymerase and RNA-directed DNA polymerase.

As used herein, the term “strand displacing” refers to the ability of a polymerase to locally separate strands of double-stranded nucleic acids and synthesize a new strand in a template-based manner. Strand displacing polymerases displace a complementary strand from a template strand and catalyze new strand synthesis. Strand displacing polymerases include mesophilic and thermophilic polymerases. Strand displacing polymerases include wild type enzymes, and variants including exonuclease minus mutants, mutant versions, chimeric enzymes and truncated enzymes. Examples of strand displacing polymerases include phi29 DNA polymerase, large fragment of Bst DNA polymerase, large fragment of Bsu DNA polymerase (exo-), Bca DNA polymerase (exo-), Klenow fragment of E. coli DNA polymerase, T5 polymerase, M-MuLV reverse transcriptase, HIV viral reverse transcriptase, Deep Vent DNA polymerase and KOD DNA polymerase. The phi29 DNA polymerase can be wild type phi29 DNA polymerase (e.g., MagniPhi from Expedeon), or variant EquiPhi29 DNA polymerase (e.g., from Thermo Fisher Scientific), or chimeric QualiPhi DNA polymerase (e.g., from 4basebio).

The terms “nucleic acid”, “polynucleotide” and “oligonucleotide” and other related terms used herein are used interchangeably and refer to polymers of nucleotides and are not limited to any particular length. Nucleic acids include recombinant and chemically-synthesized forms. Nucleic acids can be isolated. Nucleic acids include DNA molecules (e.g., cDNA or genomic DNA), RNA molecules (e.g., mRNA), analogs of the DNA or RNA generated using nucleotide analogs (e.g., peptide nucleic acids (PNA) and non-naturally occurring nucleotide analogs), and chimeric forms containing DNA and RNA. Nucleic acids can be single-stranded or double-stranded. Nucleic acids comprise polymers of nucleotides, where the nucleotides include natural or non-natural bases and/or sugars. Nucleic acids comprise naturally-occurring internucleosidic linkages, for example phosphodiester linkages. Nucleic acids can lack a phosphate group. Nucleic acids comprise non-natural internucleoside linkages, including phosphorothioate, phosphorothiolate, or peptide nucleic acid (PNA) linkages. In some embodiments, nucleic acids comprise a one type of polynucleotides or a mixture of two or more different types of polynucleotides.

The term “operably linked” and “operably joined” or related terms as used herein refers to juxtaposition of components. The juxtaposed components can be linked together covalently. For example, two nucleic acid components can be enzymatically ligated together where the linkage that joins together the two components comprises phosphodiester linkage. A first and second nucleic acid component can be linked together, where the first nucleic acid component can confer a function on a second nucleic acid component. For example, linkage between a primer binding sequence and a sequence of interest forms a nucleic acid library molecule having a portion that can bind to a primer. In another example, a transgene (e.g., a nucleic acid encoding a polypeptide or a nucleic acid sequence of interest) can be ligated to a vector where the linkage permits expression or functioning of the transgene sequence contained in the vector. In some embodiments, a transgene is operably linked to a host cell regulatory sequence (e.g., a promoter sequence) that affects expression of the transgene. In some embodiments, the vector comprises at least one host cell regulatory sequence, including a promoter sequence, enhancer, transcription and/or translation initiation sequence, transcription and/or translation termination sequence, polypeptide secretion signal sequences, and the like. In some embodiments, the host cell regulatory sequence controls expression of the level, timing and/or location of the transgene.

The terms “linked”, “joined”, “attached”, “appended” and variants thereof comprise any type of fusion, bond, adherence or association between any combination of compounds or molecules that is of sufficient stability to withstand use in a particular procedure. The procedure can include but is not limited to: nucleotide binding; nucleotide incorporation; de-blocking (e.g., removal of chain-terminating moiety); washing; removing; flowing; detecting; imaging and/or identifying; or any combination thereof. Such linkage can comprise, for example, covalent, ionic, hydrogen, dipole-dipole, hydrophilic, hydrophobic, or affinity bonding, bonds or associations involving van der Waals forces, mechanical bonding, the like, or any combination thereof. In some embodiments, such linkage occurs intramolecularly, for example linking together the ends of a single-stranded or double-stranded linear nucleic acid molecule to form a circular molecule. In some embodiments such linkage can occur between a combination of different molecules, or between a molecule and a non-molecule, including but not limited to: linkage between a nucleic acid molecule and a solid surface; linkage between a protein and a detectable reporter moiety; linkage between a nucleotide and detectable reporter moiety; and the like. Some examples of linkages can be found, for example, in Hermanson, G., “Bioconjugate Techniques”, Second Edition (2008); Aslam, M., Dent, A., “Bioconjugation: Protein Coupling Techniques for the Biomedical Sciences”, London: Macmillan (1998); Aslam, M., Dent, A., “Bioconjugation: Protein Coupling Techniques for the Biomedical Sciences”, London: Macmillan (1998), which is incorporated herein by reference in its entirety.

The term “primer” and related terms used herein refers to an oligonucleotide that is capable of hybridizing with a DNA and/or RNA polynucleotide template to form a duplex molecule. Primers comprise natural nucleotides and/or nucleotide analogs. Primers can be recombinant nucleic acid molecules. Primers may have any length, but typically range from 4-50 nucleotides. A typical primer comprises a 5′ end and 3′ end. The 3′ end of the primer can include a 3′ OH moiety which serves as a nucleotide polymerization initiation site in a polymerase-catalyzed primer extension reaction. Alternatively, the 3′ end of the primer can lack a 3′ OH moiety, or can include a terminal 3′ blocking group that inhibits nucleotide polymerization in a polymerase-catalyzed reaction. Any one nucleotide, or more than one nucleotide, along the length of the primer can be labeled with a detectable reporter moiety. A primer can be in solution (e.g., a soluble primer) or can be immobilized to a support (e.g., a capture primer).

The terms “template nucleic acid”, “template polynucleotide”, “target nucleic acid” “target polynucleotide”, “template strand” and other variations refer to a nucleic acid strand that serves as the basis nucleic acid molecule for any of the reiterative sequencing methods described herein. The template nucleic acid can be single-stranded or double-stranded, or the template nucleic acid can have single-stranded or double-stranded portions. The template nucleic acid can be obtained from a naturally-occurring source, recombinant form, or chemically synthesized to include any type of nucleic acid analog. The template nucleic acid can be linear, circular, or other forms. The template nucleic acids can include an insert portion having an insert sequence. The template nucleic acids can also include at least one adaptor sequence. The insert portion can be isolated in any form, including chromosomal, genomic, organellar (e.g., mitochondrial, chloroplast or ribosomal), recombinant molecules, cloned, amplified, cDNA, RNA such as precursor mRNA or mRNA, oligonucleotides, whole genomic DNA, obtained from fresh frozen paraffin embedded tissue, needle biopsies, circulating tumor cells, cell free circulating DNA, or any type of nucleic acid library. The insert portion can be isolated from any source including from organisms such as prokaryotes, eukaryotes (e.g., humans, plants and animals), fungus, viruses cells, tissues, normal or diseased cells or tissues, body fluids including blood, urine, serum, lymph, tumor, saliva, anal and vaginal secretions, amniotic samples, perspiration, semen, environmental samples, culture samples, or synthesized nucleic acid molecules prepared using recombinant molecular biology or chemical synthesis methods. The insert portion can be isolated from any organ, including, head, neck, brain, breast, ovary, cervix, colon, rectum, endometrium, gallbladder, intestines, bladder, prostate, testicles, liver, lung, kidney, esophagus, pancreas, thyroid, pituitary; thymus, skin, heart, larynx, or other organs. The template nucleic acid can be subjected to nucleic acid analysis, including sequencing and composition analysis.

The term “adaptor” and related terms refers to oligonucleotides that can be operably linked to a target polynucleotide, where the adaptor confers a function to the co-joined adaptor-target molecule. Adaptors may comprise DNA, RNA, chimeric DNA/RNA, or analogs thereof. Adaptors can include at least one ribonucleoside residue. Adaptors can be single-stranded, double-stranded, or have single-stranded and/or double-stranded portions. Adaptors can be configured to be linear, stem-looped, hairpin, or Y-shaped forms. Adaptors can be any length, including 4-100 nucleotides or longer. Adaptors can have blunt ends, overhang ends, or a combination of both. Overhang ends include 5′ overhang and 3′ overhang ends. The 5′ end of a single-stranded adaptor, or one strand of a double-stranded adaptor, can have a 5′ phosphate group or lack a 5′ phosphate group. Adaptors can include a 5′ tail that does not hybridize to a target polynucleotide (e.g., tailed adaptor), or adaptors can be non-tailed. An adaptor can include a sequence that is complementary to at least a portion of a primer, such as an amplification primer, a sequencing primer, or a capture primer (e.g., soluble or immobilized capture primers). Adaptors can include a random sequence or degenerate sequence. Adaptors can include at least one inosine residue. Adaptors can include at least one phosphorothioate, phosphorothiolate and/or phosphoramidate linkage. Adaptors can include a barcode sequence which can be used to distinguish polynucleotides (e.g., insert sequences) from different sample sources in a multiplex assay. Adaptors can include a unique identification sequence (e.g., unique molecular index, UMI; or a unique molecular tag) that can be used to uniquely identify a nucleic acid molecule to which the adaptor is appended. In some embodiments, a unique identification sequence can be used to increase error correction and accuracy, reduce the rate of false-positive variant calls and/or increase sensitivity of variant detection. Adaptors can include at least one restriction enzyme recognition sequence, including any one or any combination of two or more selected from a group consisting of type I, type II, type III, type IV, type Hs or type IIB.

In some embodiments, any of the amplification primer sequences, sequencing primer sequences, capture primer sequences, target capture sequences, circularization anchor sequences, sample barcode sequences, spatial barcode sequences, or anchor region sequences can be about 3-50 nucleotides in length, or about 5-40 nucleotides in length, or about 5-25 nucleotides in length.

The term “universal sequence” and related terms refers to a sequence in a nucleic acid molecule that is common among two or more polynucleotide molecules. For example, an adaptor having a universal sequence can be operably joined to a plurality of polynucleotides so that the population of co-joined molecules carry the same universal adaptor sequence. Examples of universal adaptor sequences include an amplification primer sequence, a sequencing primer sequence or a capture primer sequence (e.g., soluble or immobilized capture primers).

When used in reference to nucleic acid molecules, the terms “hybridize” or “hybridizing” or “hybridization” or other related terms refers to hydrogen bonding between two different nucleic acids to form a duplex nucleic acid. Hybridization also includes hydrogen bonding between two different regions of a single nucleic acid molecule to form a self-hybridizing molecule having a duplex region. Hybridization can comprise Watson-Crick or Hoogsteen binding to form a duplex double-stranded nucleic acid, or a double-stranded region within a nucleic acid molecule. The double-stranded nucleic acid, or the two different regions of a single nucleic acid, may be wholly complementary, or partially complementary. Complementary nucleic acid strands need not hybridize with each other across their entire length. The complementary base pairing can be the standard A-T or C-G base pairing, or can be other forms of base-pairing interactions. Duplex nucleic acids can include mismatched base-paired nucleotides.

When used in reference to nucleic acids, the terms “extend”, “extending”, “extension” and other variants, refers to incorporation of one or more nucleotides into a nucleic acid molecule. Nucleotide incorporation comprises polymerization of one or more nucleotides into the terminal 3′ OH end of a nucleic acid strand, resulting in extension of the nucleic acid strand. Nucleotide incorporation can be conducted with natural nucleotides and/or nucleotide analogs. Typically, but not necessarily, nucleotide incorporation occurs in a template-dependent fashion. Any suitable method of extending a nucleic acid molecule may be used, including primer extension catalyzed by a DNA polymerase or RNA polymerase.

The term “nucleotides” and related terms refers to a molecule comprising an aromatic base, a five carbon sugar (e.g., ribose or deoxyribose), and at least one phosphate group. Canonical or non-canonical nucleotides are consistent with use of the term. The phosphate in some embodiments comprises a monophosphate, diphosphate, or triphosphate, or corresponding phosphate analog. The term “nucleoside” refers to a molecule comprising an aromatic base and a sugar. Nucleotides and nucleosides can be non-labeled or labeled with a detectable reporter moiety.

Nucleotides (and nucleosides) typically comprise a hetero cyclic base including substituted or unsubstituted nitrogen-containing parent heteroaromatic ring which are commonly found in nucleic acids, including naturally-occurring, substituted, modified, or engineered variants, or analogs of the same. The base of a nucleotide (or nucleoside) is capable of forming Watson-Crick and/or Hoogsteen hydrogen bonds with an appropriate complementary base. Non-limiting example bases include, but are not limited to, purines and pyrimidines such as: 2-aminopurine, 2,6-diaminopurine, adenine (A), ethenoadenine, N6-Δ2-isopentenyladenine (6iA), N6-Δ2-isopentenyl-2-methylthioadenine (2ms6iA), N6-methyladenine, guanine (G), isoguanine, N2-dimethylguanine (dmG), 7-methylguanine (7mG), 2-thiopyrimidine, 6-thioguanine (6sG), hypoxanthine and 06-methylguanine; 7-deaza-purines such as 7-deazaadenine (7-deaza-A) and 7-deazaguanine (7-deaza-G); pyrimidines such as cytosine (C), 5-propynylcytosine, isocytosine, thymine (T), 4-thiothymine (4sT), 5,6-dihydrothymine, 04-methylthymine, uracil (U), 4-thiouracil (4sU) and 5,6-dihydrouracil (dihydrouracil; D); indoles such as nitroindole and 4-methylindole; pyrroles such as nitropyrrole; nebularine; inosines; hydroxymethylcytosines; 5-methycytosines; base (Y); as well as methylated, glycosylated, and acylated base moieties; and the like. Additional non-limiting example bases can be found in Fasman, 1989, in “Practical Handbook of Biochemistry and Molecular Biology”, pp. 385-394, CRC Press, Boca Raton, Fla., which is incorporated herein by reference in its entirety.

Nucleotides (and nucleosides) typically comprise a sugar moiety, such as carbocyclic moiety (Ferraro and Gotor 2000 Chem. Rev. 100: 4319-48, which is incorporated herein by reference in its entirety), acyclic moieties (Martinez, et al., 1999 Nucleic Acids Research 27: 1271-1274; Martinez, et al., 1997 Bioorganic & Medicinal Chemistry Letters vol. 7: 3013-3016, which are both incorporated herein by reference in their entirety), and other sugar moieties (Joeng, et al., 1993 J. Med. Chem. 36: 2627-2638; Kim, et al., 1993 J. Med. Chem. 36: 30-7; Eschenmosser 1999 Science 284:2118-2124; and U.S. Pat. No. 5,558,991, which are all incorporated herein by reference in their entireties). The sugar moiety comprises: ribosyl; 2′-deoxyribosyl; 3′-deoxyribosyl; 2′,3′-dideoxyribosyl; 2′,3′-didehydrodideoxyribosyl; 2′-alkoxyribosyl; 2′-azidoribosyl; 2′-aminoribosyl; 2′-fluororibosyl; 2′-mercaptoriboxyl; 2′-alkylthioribosyl; 3′-alkoxyribosyl; 3′-azidoribosyl; 3′-aminoribosyl; 3′-fluororibosyl; 3′-mercaptoriboxyl; 3′-alkylthioribosyl carbocyclic; acyclic or other modified sugars.

In some embodiments, nucleotides comprise a chain of one, two or three phosphorus atoms where the chain is typically attached to the 5′ carbon of the sugar moiety via an ester or phosphoramide linkage. In some embodiments, the nucleotide is an analog having a phosphorus chain in which the phosphorus atoms are linked together with intervening O, S, NH, methylene or ethylene. In some embodiments, the phosphorus atoms in the chain include substituted side groups including 0, S or BH3. In some embodiments, the chain includes phosphate groups substituted with analogs including phosphoramidate, phosphorothioate, phosphorodithioate, and O-methyl phosphoramidite groups.

The terms “reporter moiety”, “reporter moieties” or related terms refers to a compound that generates, or causes to generate, a detectable signal. A reporter moiety is sometimes called a “label”. Any suitable reporter moiety may be used, including luminescent, photoluminescent, electroluminescent, bioluminescent, chemiluminescent, fluorescent, phosphorescent, chromophore, radioisotope, electrochemical, mass spectrometry, Raman, hapten, affinity tag, atom, or an enzyme. A reporter moiety generates a detectable signal resulting from a chemical or physical change (e.g., heat, light, electrical, pH, salt concentration, enzymatic activity, or proximity events). A proximity event includes two reporter moieties approaching each other, or associating with each other, or binding each other In some embodiments, reporter moieties may be selected so that each absorbs excitation radiation and/or emits fluorescence at a wavelength distinguishable from the other reporter moieties to permit monitoring the presence of different reporter moieties in the same reaction or in different reactions. Two or more different reporter moieties can be selected having spectrally distinct emission profiles, or having minimal overlapping spectral emission profiles. Reporter moieties can be linked (e.g., operably linked) to nucleotides, nucleosides, nucleic acids, enzymes (e.g., polymerases or reverse transcriptases), or support (e.g., surfaces).

A reporter moiety (or label) may comprise a fluorescent label or a fluorophore. Non-limiting example fluorescent moieties which may serve as fluorescent labels or fluorophores include, but are not limited to, fluorescein and fluorescein derivatives such as carboxyfluorescein, tetrachlorofluorescein, hexachlorofluorescein, carboxynapthofluorescein, fluorescein isothiocyanate, NHS-fluorescein, iodoacetamidofluorescein, fluorescein maleimide, SAMSA-fluorescein, fluorescein thiosemicarbazide, carbohydrazinomethylthioacetyl-amino fluorescein, rhodamine and rhodamine derivatives such as TRITC, TMR, lissamine rhodamine, Texas Red, rhodamine B, rhodamine 6G, rhodamine 10, NHS-rhodamine, TMR-iodoacetamide, lissamine rhodamine B sulfonyl chloride, lissamine rhodamine B sulfonyl hydrazine, Texas Red sulfonyl chloride, Texas Red hydrazide, coumarin and coumarin derivatives such as AMCA, AMCA-NHS, AMCA-sulfo-NHS, AMCA-HPDP, DCIA, AMCE-hydrazide, BODIPY and derivatives such as BODIPY FL C3-SE, BODIPY 530/550 C3, BODIPY 530/550 C3-SE, BODIPY 530/550 C3 hydrazide, BODIPY 493/503 C3 hydrazide, BODIPY FL C3 hydrazide, BODIPY FL IA, BODIPY 530/551 IA, Br-BODIPY 493/503, Cascade Blue and derivatives such as Cascade Blue acetyl azide, Cascade Blue cadaverine, Cascade Blue ethylenediamine, Cascade Blue hydrazide, Lucifer Yellow and derivatives such as Lucifer Yellow iodoacetamide, Lucifer Yellow CH, cyanine and derivatives such as indolium based cyanine dyes, benzo-indolium based cyanine dyes, pyridium based cyanine dyes, thiozolium based cyanine dyes, quinolinium based cyanine dyes, imidazolium based cyanine dyes, Cy 3, Cy5, lanthanide chelates and derivatives such as BCPDA, TBP, TMT, BHHCT, BCOT, Europium chelates, Terbium chelates, Alexa Fluor dyes, DyLight dyes, Atto dyes, LightCycler Red dyes, CAL Flour dyes, JOE and derivatives thereof, Oregon Green dyes, WellRED dyes, IRD dyes, phycoerythrin and phycobilin dyes, Malachite green, stilbene, DEG dyes, NR dyes, near-infrared dyes and others known in the art such as those described in Haugland, Molecular Probes Handbook, (Eugene, Oreg.) 6th Edition; Lakowicz, Principles of Fluorescence Spectroscopy, 2nd Ed., Plenum Press New York (1999), or Hermanson, Bioconjugate Techniques, 2nd Edition, all references which are incorporated herein in their entirety; or derivatives thereof, or any combination thereof. Cyanine dyes may exist in either sulfonated or non-sulfonated forms, and consist of two indolenin, benzo-indolium, pyridium, thiozolium, and/or quinolinium groups separated by a polymethine bridge between two nitrogen atoms. Commercially available cyanine fluorophores include, for example, Cy3, (which may comprise 1-[6-(2,5-dioxopyrrolidin-1-yloxy)-6-oxohexyl]-2-(3-{1-[6-(2,5-dioxopyrrolidin-1-yloxy)-6-oxohexyl]-3,3-dimethyl-1,3-dihydro-2H-indol-2-ylidene}prop-1-en-1-yl)-3,3-dimethyl-3H-indolium or 1-[6-(2,5-dioxopyrrolidin-1-yloxy)-6-oxohexyl]-2-(3-{1-[6-(2,5-dioxopyrrolidin-1-yloxy)-6-oxohexyl]-3,3-dimethyl-5-sulfo-1,3-dihydro-2H-indol-2-ylidene}prop-1-en-1-yl)-3,3-dimethyl-3H-indolium-5-sulfonate), Cy5 (which may comprise 1-(6-((2,5-dioxopyrrolidin-1-yl)oxy)-6-oxohexyl)-2-((1E,3E)-5-((E)-1-(6-((2,5-dioxopyrrolidin-1-yl)oxy)-6-oxohexyl)-3,3-dimethyl-5-indolin-2-ylidene)penta-1,3-dien-1-yl)-3,3-dimethyl-3H-indolium or 1-(6-((2,5-dioxopyrrolidin-1-yl)oxy)-6-oxohexyl)-2-((1E,3E)-5-((E)-1-(6-((2,5-dioxopyrrolidin-1-y 1)oxy)-6-oxohexyl)-3,3-dimethyl-5-sulfoindolin-2-ylidene)penta-1,3-dien-1-yl)-3,3-dimethyl-3H-indolium-5-sulfonate), and Cy7 (which may comprise 1-(5-carboxypenty1)-2-[(1E,3E,5E,7Z)-7-(1-ethyl-1,3-dihydro-2H-indol-2-ylidene)hepta-1,3,5-trien-1-y1]-3H-indolium or 1-(5-carboxypenty1)-2-[((1E,3E,5E,7Z)-7-(1-ethyl-5-sulfo-1,3-dihydro-2H-indol-2-ylidene)hepta-1,3,5-trien-1-y1]-3H-indolium-5-sulfonate), where “Cy” stands for ‘cyanine’, and the first digit identifies the number of carbon atoms between two indolenine groups. Cy2, which is an oxazole derivative rather than an indolenin, and the benzo-derivatized Cy3.5, Cy5.5 and Cy7.5 are non-limiting examples of exceptions to this rule.

In some embodiments, the reporter moiety can be a FRET pair, such that multiple classifications can be performed under a single excitation and imaging step. As used herein, FRET may comprise excitation exchange (Forster) transfers, or electron-exchange (Dexter) transfers.

When used in reference to nucleic acids, the terms “amplify”, “amplifying”, “amplification”, and other related terms include producing multiple copies of an original polynucleotide template molecule, where the copies comprise a sequence that is complementary to the template sequence, or the copies comprise a sequence that is the same as the template sequence. In some embodiments, the copies comprise a sequence that is substantially identical to a template sequence, or is substantially identical to a sequence that is complementary to the template sequence.

The term “support” as used herein refers to a substrate that is designed for deposition of biological molecules or biological samples for assays and/or analyses. Examples of biological molecules to be deposited onto a support include nucleic acids (e.g., DNA, RNA), polypeptides, saccharides, lipids, a single cell or multiple cells. Examples of biological samples include but are not limited to saliva, phlegm, mucus, blood, plasma, serum, urine, stool, sweat, tears and fluids from tissues or organs.

In some embodiments, the support is solid, semi-solid, or a combination of both. In some embodiments, the support is porous, semi-porous, non-porous, or any combination of porosity.

In some embodiments, the support can be substantially planar, concave, convex, or any combination thereof. In some embodiments, the support can be cylindrical, for example comprising a capillary or interior surface of a capillary.

In some embodiments, the surface of the support can be substantially smooth. In some embodiments, the support can be regularly or irregularly textured, including bumps, etched, pores, three-dimensional scaffolds, or any combination thereof.

In some embodiments, the support comprises a bead having any shape, including spherical, hemi-spherical, cylindrical, barrel-shaped, toroidal, disc-shaped, rod-like, conical, triangular, cubical, polygonal, tubular or wire-like.

The support can be fabricated from any material, including but not limited to glass, fused-silica, silicon, a polymer (e.g., polystyrene (PS), macroporous polystyrene (1VIPPS), polymethylmethacrylate (PMMA), polycarbonate (PC), polypropylene (PP), polyethylene (PE), high density polyethylene (HDPE), cyclic olefin polymers (COP), cyclic olefin copolymers (COC), polyethylene terephthalate (PET)), or any combination thereof. Various compositions of both glass and plastic substrates are contemplated.

The support can have a plurality (e.g., two or more) of nucleic acid templates immobilized thereon. The plurality of immobilized nucleic acid templates may have the same sequence or have different sequences. In some embodiments, individual nucleic acid template molecules in the plurality of nucleic acid templates are immobilized to a different site on the support. In some embodiments, two or more individual nucleic acid template molecules in the plurality of nucleic acid templates are immobilized to a site on the support.

The term “array” refers to a support comprising a plurality of sites located at pre-determined locations on the support to form an array of sites. The sites can be discrete and separated by interstitial regions. In some embodiments, the pre-determined sites on the support can be arranged in one dimension in a row or a column, or arranged in two dimensions in rows and columns. In some embodiments, the plurality of pre-determined sites is arranged on the support in an organized fashion. In some embodiments, the plurality of pre-determined sites is arranged in any organized pattern, including rectilinear, hexagonal patterns, grid patterns, patterns having reflective symmetry, patterns having rotational symmetry, or the like. The pitch between different pairs of sites can be that same or can vary. In some embodiments, the support comprises at least 102 sites, at least 103 sites, at least 104 sites, at least 105 sites, at least 106 sites, at least 107 sites, at least 108 sites, at least 109 sites, at least 1010 sites, at least 1011 sites, at least 1012 sites, at least 1013 sites, at least 1014 sites, at least 1015 sites, or more, where the sites are located at pre-determined locations on the support. In some embodiments, a plurality of pre-determined sites on the support (e.g., 102-1015 sites or more) are immobilized with nucleic acid templates to form a nucleic acid template array. In some embodiments, the nucleic acid templates that are immobilized at a plurality of pre-determined sites by hybridization to immobilized surface capture primers, or the nucleic acid templates are covalently attached to the surface capture primer. In some embodiments, the nucleic acid templates that are immobilized at a plurality of pre-determined sites, for example immobilized at 102-1015 sites or more. In some embodiments, the immobilized nucleic acid templates are clonally-amplified to generate immobilized nucleic acid clusters at the plurality of pre-determined sites. In some embodiments, individual immobilized nucleic acid clusters comprise linear clusters, or comprise single-stranded or double-stranded concatemers.

In some embodiments, a support comprising a plurality of sites located at random locations on the support is referred to herein as a support having randomly located sites thereon. The location of the randomly located sites on the support are not pre-determined. The plurality of randomly-located sites is arranged on the support in a disordered and/or unpredictable fashion. In some embodiments, the support comprises at least 102 sites, at least 103 sites, at least 104 sites, at least 105 sites, at least 106 sites, at least 107 sites, at least 108 sites, at least 109 sites, at least 1010 sites, at least 1011 sites, at least 1012 sites, at least 1013 sites, at least 1014 sites, at least 1015 sites, or more, where the sites are randomly located on the support. In some embodiments, a plurality of randomly located sites on the support (e.g., 102-1015 sites or more) are immobilized with nucleic acid templates to form a support immobilized with nucleic acid templates. In some embodiments, the nucleic acid templates that are immobilized at a plurality of randomly located sites by hybridization to immobilized surface capture primers, or the nucleic acid templates are covalently attached to the surface capture primer. In some embodiments, the nucleic acid templates that are immobilized at a plurality of randomly located sites, for example immobilized at 102-1015 sites or more. In some embodiments, the immobilized nucleic acid templates are clonally-amplified to generate immobilized nucleic acid clusters at the plurality of randomly located sites. In some embodiments, individual immobilized nucleic acid clusters comprise linear clusters, or comprise single-stranded or double-stranded concatemers.

In some embodiment, the plurality of immobilized surface capture primers on the support are in fluid communication with each other to permit flowing a solution of reagents (e.g., nucleic acid template molecules, soluble primers, enzymes, nucleotides, divalent cations, buffers, and the like) onto the support so that the plurality of immobilized surface capture primers on the support can be essentially simultaneously reacted with the reagents in a massively parallel manner. In some embodiments, the fluid communication of the plurality of immobilized surface capture primers can be used to conduct nucleic acid amplification reactions (e.g., RCA, MDA, PCR and bridge amplification) essentially simultaneously on the plurality of immobilized surface capture primers.

In some embodiment, the plurality of immobilized nucleic acid clusters on the support are in fluid communication with each other to permit flowing a solution of reagents (e.g., enzymes, nucleotides, divalent cations, and the like) onto the support so that the plurality of immobilized nucleic acid clusters on the support can be essentially simultaneously reacted with the reagents in a massively parallel manner. In some embodiments, the fluid communication of the plurality of immobilized nucleic acid clusters can be used to conduct nucleotide binding assays and/or conduct nucleotide polymerization reactions (e.g., primer extension or sequencing) essentially simultaneously on the plurality of immobilized nucleic acid clusters, and optionally to conduct detection and imaging for massively parallel sequencing.

When used in reference to immobilized enzymes, the term “immobilized” and related terms refer to enzymes (e.g., polymerases) that are attached to a support through covalent bond or non-covalent interaction, or attached to a coating on the support, or buried within a matrix formed by a coating on the support.

When used in reference to immobilized nucleic acids, the term “immobilized” and related terms refer to nucleic acid molecules that are attached to a support through covalent bond or non-covalent interaction, or attached to a coating on the support, or buried within a matrix formed by a coating on the support, where the nucleic acid molecules include surface capture primers, nucleic acid template molecules and extension products of capture primers. Extension products of capture primers includes nucleic acid concatemers (e.g., nucleic acid clusters).

In some embodiments, one or more nucleic acid templates are immobilized on the support, for example immobilized at the sites on the support. In some embodiments, the one or more nucleic acid templates are clonally-amplified. In some embodiments, the one or more nucleic acid templates are clonally-amplified off the support (e.g., in-solution) and then deposited onto the support and immobilized on the support. In some embodiments, the clonal amplification reaction of the one or more nucleic acid templates is conducted on the support resulting in immobilization on the support. In some embodiments, the one or more nucleic acid templates are clonally-amplified (e.g., in solution or on the support) using a nucleic acid amplification reaction, including any one or any combination of: polymerase chain reaction (PCR), multiple displacement amplification (MDA), transcription-mediated amplification (TMA), nucleic acid sequence-based amplification (NASBA), strand displacement amplification (SDA), real-time SDA, bridge amplification, isothermal bridge amplification, rolling circle amplification (RCA), circle-to-circle amplification, helicase-dependent amplification, recombinase-dependent amplification, and/or single-stranded binding (SSB) protein-dependent amplification.

The term “persistence time” and related terms refers to the length of time that a binding complex, which is formed between the target nucleic acid, a polymerase, a conjugated or unconjugated nucleotide, remains stable without any binding component dissociates from the binding complex. The persistence time is indicative of the stability of the binding complex and strength of the binding interactions. Persistence time can be measured by observing the onset and/or duration of a binding complex, such as by observing a signal from a labeled component of the binding complex. For example, a labeled nucleotide or a labeled reagent comprising one or more nucleotides may be present in a binding complex, thus allowing the signal from the label to be detected during the persistence time of the binding complex. One non-limiting example of a label is a fluorescent label.

### INTRODUCTION

The present disclosure provides methods, compositions, systems and kits for use in processing or analyzing a biomolecule. The biomolecule may comprise, e.g., a nucleic acid, a polypeptide or protein, a lipid, or a carbohydrate. Methods, compositions, systems and kits as described herein may be used to obtain information about a location of a nucleic acid sequence, such as an RNA or a DNA, and a protein encoded by the nucleic acid sequence. For example, target nucleic acid sequences may be identified and sequenced as disclosed herein. For example, a target RNA or DNA molecule associated with a target protein or polypeptide may be identified by a padlock probe as described herein, and may be further amplified and/or sequenced. In this manner, information can be gathered about the location and/or sequence of a target protein, RNA sequence, and DNA sequence.

Disclosed herein, in some embodiments, are methods, compositions, systems and kits for use in processing and/or analyzing a nucleic acid sequence. Methods, systems kit and compositions as described herein may be used in in situ reiterative short read sequencing. The sequencing may be performed in one or more cells, tissues, or tumors. The nucleic acid sequence may be, e.g., a DNA sequence or an RNA sequence. Methods, systems, kits and compositions as described herein may be useful in leveraging massively parallel sequencing technologies. Such methods, composition, kits, and systems can also be found in US Publication No. US20210139884, which is incorporated herein by reference in its entirety.

### Methods

The present disclosure provides methods for analyzing a nucleic acid sequence. Methods as disclosed herein may comprise introducing one or more reagents to the biological sample under conditions sufficient to identify a DNA encoding an RNA, the RNA encoding a protein, or the protein in a single nucleic acid sequencing. The identifying may comprise running any of the methods disclosed herein. In some embodiments, the methods disclosed herein do not require the use of a barcoded nucleic acid, and instead, can utilize spatially encoded reagents. For example, primers spatially encoded on a solid surface may be identified by its relative position on the solid surface. The methods described herein may employ in situ reiterative short read sequencing within a cellular sample including a single cell, multiple cells, a tissue or a tumor. In some embodiments, the methods comprise repeatedly conducting a short number of sequencing cycles of the same region of the template molecules. By conducting reiterative short sequencing cycles, the RNA content of the cellular sample can be discovered. Methods as described herein may be employed in conducting transcriptomics workflows which leverages massively parallel sequencing technologies. Compared to long read sequencing workflows, the reiterative short sequencing cycles described herein use a reduced amount of sequencing reagents which reduces cost and saves time. Methods for conducting reiterative short sequencing cycles has many uses, including, but not limited to, detecting specific RNAs of interest, mutant RNA sequences, splice variants, and their abundance levels thereof.

One non-limiting example of a purpose of the methods described herein is to detect and image the spatial localization of RNAs within a cellular sample using massively parallel sequencing. The workflow may comprise the following general steps: a cellular sample may be placed on a solid support, which may be positioned on a fluorescent microscope configured to detect fluorescent signals. The cellular may be is treated with a chemical fixation reagent to retain the target RNAs inside the cells. The cellular sample may also be permeabilized to permit manipulation of the target RNAs inside the cells. The target RNAs may be converted into first strand cDNAs, which may be selectively hybridized to target-specific padlock probes for generating circularized padlock probes that correspond to particular cDNAs. The padlock probes may carry at least a universal adaptor for a sequencing primer binding site. The padlock probes can also carry a target barcode sequence each corresponding to a given target cDNA. The circularized padlock probes may be subjected to a ligation and/or fill-in reaction to form covalently closed circular padlock probes, which may be amplified inside the cellular sample via rolling circle amplification to generate single-stranded concatemers. The rolling circle amplification may be conducted in the presence of one or more compaction oligonucleotides. The concatemers may carry tandem repeat units of a cDNA-of-interest, the universal sequencing primer binding site, and the target barcode sequence. The concatemers may be sequenced inside the cellular sample where a short number of sequencing cycles are conducted for each round and multiple rounds of short read sequencing is conducted. The full length of the target barcode and cDNA region may not be sequenced. Instead, at least a portion of the target barcode region may be reiteratively sequenced. In some embodiments, it is not necessary to sequence the cDNA region. In some embodiments, the target barcode and a portion of the cDNA region are reiteratively sequenced. It may not be necessary to sequence the entire length of the cDNA region. It may not be necessary to assemble the sequencing reads or to obtain a full-length sequence of the cDNAs-of-interest. The redundant sequencing information obtained from the short sequencing reads may obviate the need to sequence the complementary strand of the concatemer. Thus, pairwise sequencing may not be necessary.

The methods described herein may offer several advantages over other in situ transcriptomics workflows. For example, the cellular sample may be placed on a solid support, for example a planar support comprising glass or plastic which can be fabricated into any shape and size. Assembly of a hybridization chamber on the support may not be needed. Preparation of chemically-washed glass beads for cell adherence may also not be needed. The support can be passivated with a coating that promotes cell adhesion to the support. The coating may not need to be formulated to include tethered capture primers. The support, having a cell sample adhered thereon, can be easily adapted to fit into an existing flow cell holder/cradle which is fluidically connected to an automated fluid dispensing system and configured on a fluorescent microscope. Any combination of the steps for conducting in situ reiterative short read sequencing can be performed in an automated mode using a fluid dispensing system, including cell seeding, cell fixation, cell permeabilization, reverse transcription reactions, padlock probe hybridization, padlock probe ligation reaction, rolling circle amplification, and sequencing.

Another advantage of the methods described herein is the formation of concatemers inside the cellular sample. The single-stranded concatemers may collapse into compact DNA nanoballs, where each nanoball carries numerous tandem copies of a polynucleotide unit along their lengths, where the polynucleotide unit includes a cDNA sequence-of-interest and at least a universal sequencing primer binding site. Each polynucleotide unit can bind a sequencing primer, a sequencing polymerase and a detectably-labeled nucleotide reagent (e.g., detectably labeled multivalent molecules), to form a detectable sequencing complex (e.g., a detectable ternary complex). Each nanoball carries numerous detectable sequencing complexes. Thus, the compact nature of the nanoballs may increase the local concentration of detectably-labeled nucleotide reagents that are used during the sequencing workflow, which may thereby increase the signal intensity emitted from a nanoball. This may give a discrete detectable signal, which can be imaged as a fluorescent spot inside the cellular sample. Each spot corresponds to a concatemer and each concatemer corresponds to a target RNA molecule in the cellular sample. Multiple spots can be detected and imaged simultaneously in the cellular sample.

Additionally, a short portion of the cDNA region in the concatemer may be re-sequenced at least once (e.g., reiterative sequencing) from the same start position to generate overlapping sequencing reads that can be aligned to a reference sequence. For example, the same portion of the concatemer molecule can be sequenced at least two, three, four, five, or up to 50 times. The start sequencing site can be any location of the concatemer and may be dictated by the sequencing primers which are designed to anneal to a selected position within the concatemer. The reiterative short sequencing reads may increase the redundancy of sequencing information for individual bases in the cDNA region. Reiteratively sequencing one strand of the concatemer template molecule may provide enough base coverage to reveal the presence of target RNAs in the cellular sample so that pairwise sequencing of the complementary strand is not necessary.

A concatemer template molecule may include multiple sequencing primer binding sites along the same concatemer molecule which can be used to generate multiple usable sequencing reads for increased sequencing depth. Together, reiteratively sequencing one strand of the concatemer templates may increase sequencing base coverage and sequencing depth compared to sequencing a one-copy template molecule.

The methods described herein can be conducted in uniplex or multiplex modes. Two or more different target RNAs can be detected and imaged simultaneously inside a cellular sample using different reverse transcription primers, different target-specific padlock probes, and universal sequencing primers. For example, the presence of a housekeeping RNA and at least one target RNA in a cellular sample can be simultaneously detected and imaged using any of the reiterative short read sequencing methods described herein.

In the methods described herein, the RNA may not be extracted from the cellular sample, and sequencing information may not need to be tracked and mapped back to an image of the cellular sample. Rather, RNA may be retained inside the cellular sample to permit direct imaging of the spatial location of target RNAs within the cells. Additionally, RNA within the cellular sample may not be fragmented and enrichment of target RNA may not be necessary. Use of target-specific and/or random-sequence reverse transcription primers enables detection of both poly-A and non-poly-A RNAs in either uniplex or multiplex modes.

The methods described herein offer several advantages over other in situ transcriptomics workflows, including a simpler workflow, fewer reagents, lower cost, less time, gentler conditions on the cellular sample, and no requirement for specialized equipment.

Methods for Conducting in situ Reiterative Short Read Sequencing

The present disclosure provides methods for conducting in situ sequencing in a biological sample. One or more nucleic acids may be analyzed in a biological sample, e.g., a cell. The nucleic acid may comprise, e.g., deoxyribonucleic acid (DNA) or ribonucleic acid (RNA). The sequencing may be conducted reiteratively, so that multiple sequencing cycles are conducted of a nucleic acid.

The present disclosure provides methods for detecting in situ at least two nucleic acid sequences in a biological sample. The present disclosure provides methods for detecting in situ at least two target RNA molecules in a biological sample. The at least two target nucleic acid sequences may comprise a first target nucleic acid sequence and a second target nucleic acid sequence. The biological sample may be a cellular sample. The method may comprise conducting sequencing reactions inside the cellular sample, where the cDNA amplicons can be the concatemer molecules. The biological sample may comprise a first nucleic acid molecule, a second nucleic acid molecule, or a combination thereof. The first nucleic acid molecule may comprise a first target nucleic acid sequence or portion thereof, or the reverse complement thereof or a portion thereof. The second nucleic acid molecule may comprise the second target nucleic acid sequence or a portion thereof, or the reverse complement thereof or a portion thereof. The method may comprise determining in situ the sequence of the first nucleic acid molecule or a portion thereof in the biological sample to generate a first sequencing product nucleic acid molecule that is complementary and binds to the first nucleic acid molecule or a portion thereof. The method may comprise determining in situ the sequence of the second nucleic acid molecule or a portion thereof in the biological sample to generate a second sequencing product nucleic acid molecule that is complementary and binds to the second nucleic acid molecule or a portion thereof. The full sequence of the first target nucleic acid sequence and the full sequence of the second target nucleic acid sequence may have at least one nucleotide of difference. The method may further comprise removing the first sequencing product nucleic acid molecule from the first nucleic acid molecule and the second sequencing product nucleic acid molecule from the second nucleic acid molecule. The first nucleic acid molecule and the second nucleic acid molecule may be positioned inside the biological sample after the removing. The method may further comprise repeating the determining in situ the sequence of the first and second nucleic acids. In some embodiments, the repeating comprises repeating at least 2 times, 5 times, 10 times, 15 times, 20 times, 25 times, 30 times, 35 times, 40 times, 45 times, or at least 50 times. The method may further comprise repeating the removing the first sequencing product nucleic acid molecule from the first nucleic acid molecule and the second sequencing product nucleic acid molecule from the second nucleic acid molecule. In some embodiments, the repeating comprises repeating at least 2 times, 5 times, 10 times, 15 times, 20 times, 25 times, 30 times, 35 times, 40 times, 45 times, or at least 50 times.

The present disclosure provides methods for detecting in situ at least two different target nucleic acid molecules in a biological sample. The present disclosure provides methods for detecting in situ at least two different target RNA molecules in a cellular sample comprising step (a): providing a cellular sample deposited on a solid support, wherein the cellular sample harbors at least a first plurality of DNA amplicons that correspond to a first target RNA molecule and the cellular sample harbors a second plurality of DNA amplicons that correspond to a second target RNA molecule. In some embodiments, the cellular sample can comprise a first target RNA molecule. In some embodiments, the cellular sample can comprise at least a first plurality of DNA amplicons that can correspond to a first target RNA molecule. In some embodiments, the cellular sample can comprise a second target RNA molecule. In some embodiments, the cellular sample can comprise at least a second plurality of DNA amplicons that can correspond to a second target RNA. In some embodiments, the cellular sample can be deposited on a solid support.

In some embodiments, the cellular sample harbors 2-25 different target RNA molecules, or harbors 25-50 different target RNA molecules, or harbors 50-75 different target RNA molecules, or harbors 75-100 different target RNA molecules. In some embodiments, the cellular sample harbors more than 100 different target RNA molecules, or more than 250 different target RNA molecules, or more than 500 different target molecules, or more than 1000 different target RNA molecules, or more. In some embodiments, the cellular sample harbors more than 10,000 different target RNA molecules. In some embodiments, the cellular sample comprises a whole cell, a plurality of whole cells, an intact tissue or an intact tumor. In some embodiments, the cellular sample comprises a fresh cellular sample, a freshly-frozen cellular sample, a sectioned cellular sample, or an FFPE cellular sample. In some embodiments, the cellular sample is deposited onto a solid support. In some embodiments, the cellular sample is deposited onto a solid support which is passivated with a coating that promotes cell adhesion. In some embodiments, the cellular sample is deposited on a support that lacks immobilized capture oligonucleotides. In some embodiments, the cellular sample comprises an expanded cellular sample that has been cultured in a simple or complex cell culture media.

In some embodiments, the first plurality of DNA amplicons comprises a first plurality of concatemers. In some embodiments, the second plurality of DNA amplicons comprises a second plurality of concatemers.

In some embodiments, the first plurality of DNA amplicons comprises a first target DNA sequence that corresponds to a first target RNA molecule. In some embodiments, the first plurality of DNA amplicons comprises a first target DNA sequence that corresponds to a first target RNA molecule and a first target barcode sequence that corresponds to the first target RNA molecule. In some embodiments, the first plurality of DNA amplicons comprises a first target DNA sequence that corresponds to a first target RNA molecule and at least one universal adaptor sequence, such as for example a universal sequencing primer binding site (or a complementary sequence thereof). In some embodiments, the first plurality of DNA amplicons comprises a first target DNA sequence that corresponds to a first target RNA molecule and a universal primer binding site for a rolling circle amplification primer (or a complementary sequence thereof). In some embodiments, the first plurality of DNA amplicons comprises a first target DNA sequence that corresponds to a first target RNA molecule and a universal compaction oligonucleotide binding site (or a complementary sequence thereof).

In some embodiments, the second plurality of DNA amplicons comprises a second target DNA sequence that corresponds to a second target RNA molecule. In some embodiments, the second plurality of DNA amplicons comprises a second target DNA sequence that corresponds to a second target RNA molecule and a second target barcode sequence that corresponds to the second target RNA molecule. In some embodiments, the second plurality of DNA amplicons comprises a second target DNA sequence that corresponds to a second target RNA molecule and at least one universal adaptor sequence, such as for example a universal sequencing primer binding site (or a complementary sequence thereof). In some embodiments, the second plurality of DNA amplicons comprises a second target DNA sequence that corresponds to a second target RNA molecule and a universal primer binding site for a rolling circle amplification primer (or a complementary sequence thereof). In some embodiments, the second plurality of DNA amplicons comprises a second target DNA sequence that corresponds to a second target RNA molecule and a universal compaction oligonucleotide binding site (or a complementary sequence thereof).

In some embodiments, the methods for detecting in situ at least two different target RNA molecules in a cellular sample further comprise step (b): sequencing the first plurality of DNA amplicons inside the cellular sample. The sequencing may comprise conducting no more than 2-30 sequencing cycles to generate a plurality of first sequencing read products, and sequencing the second plurality of DNA amplicons inside the cellular sample which comprises conducting no more than 2-30 sequencing cycles to generate a plurality of second sequencing read products. In some embodiments, (b) can comprise sequencing the first plurality of DNA amplicons inside the cellular sample which can comprise conducting no more than 2-30 sequencing cycles. In some embodiments, the 2-30 sequencing cycles can generate a plurality of first sequencing read products. In some embodiments, (b) can comprise sequencing the second plurality of DNA amplicons inside the cellular sample which can comprise conducting no more than 2-30 sequencing cycles. In some embodiments, the 2-30 sequencing cycles can generate a plurality of second sequencing read products. In some embodiments, the sequences of the first sequencing read products can be aligned with a first target reference sequence to confirm the presence of the first target RNA in the cellular product. In some embodiments, the sequences of the second sequencing read products can be aligned with a second target reference sequence to confirm the presence of the second target RNA in the cellular sample. In some embodiments, the sequences of the first sequencing read products are aligned with a first target reference sequence to confirm the presence of the first target RNA in the cellular sample, and the sequences of the second sequencing read products are aligned with a second target reference sequence to confirm the presence of the second target RNA in the cellular sample.

In some embodiments, the first target reference sequence comprises the first target barcode sequence. In some embodiments, the first target reference sequence comprises the first target barcode sequence and at least a portion of the first target RNA sequence. In some embodiments, the first target reference sequence comprises at least a portion of the first target RNA sequence.

In some embodiments, the second target reference sequence comprises the second target barcode sequence. In some embodiments, the second target reference sequence comprises the second target barcode sequence and at least a portion of the second target RNA sequence. In some embodiments, the second target reference sequence comprises at least a portion of the second target RNA sequence.

In some embodiments, the methods for detecting in situ at least two different target RNA molecules in a cellular sample can further comprise (c): removing the plurality of first sequencing read products from the first DNA amplicons and retaining the first DNA amplicons inside the cellular sample, and removing the plurality of second sequencing read products from the second DNA amplicons and retaining the second DNA amplicons inside the cellular sample. In some embodiments, (c) can comprise removing the plurality of first sequencing read products from the first DNA amplicons. In some embodiments, (c) can comprise retaining the first DNA amplicons inside the cellular sample. In some embodiments, (c) can comprise removing the plurality of second sequence ready products from the second DNA amplicons. In some embodiments, (c) can comprise retaining the second DNA amplicons inside the cellular sample.

In some embodiments, the methods for detecting in situ at least two different target RNA molecules in a cellular sample further comprise step (c): removing the plurality of first sequencing read products from the first DNA amplicons and retaining the first DNA amplicons inside the cellular sample, and removing the plurality of second sequencing read products from the first DNA amplicons and retaining the second DNA amplicons inside the cellular sample.

In some embodiments, the methods for detecting in situ at least two different target RNA molecules in a cellular sample further can comprise (d): reiteratively sequencing the first and second plurality of DNA amplicons by repeating (b) and (c) at least once. In some embodiments, the repeating comprises repeating at least 2 times, 5 times, 10 times, 15 times, 20 times, 25 times, 30 times, 35 times, 40 times, 45 times, or at least 50 times. In some embodiments, (d) can comprise reiteratively sequencing the first plurality of DNA amplicons by repeating (b) and (c)at least once. In some embodiments, (d) can comprise reiteratively sequencing the second plurality of DNA amplicons by repeating (b) and (c) at least once. In some embodiments, (b) and (c) can be repeated at least 2 times, at least 3 times, at least 4 times, at least 5 times, at least 6 times, at least 7 times, at least 8 times, at least 9 times, or at least 10 times. In some embodiments, (b) and (c) can be repeated up to 10 times, up to 20 times, up to 30 time, up to 40 times, or up to 50 times.

In some embodiments, the methods for detecting in situ at least two different target RNA molecules in a cellular sample further comprise step (d): reiteratively sequencing the first and second plurality of DNA amplicons by repeating steps (b) and (c) at least once. In some embodiments, steps (b) and (c) can be repeated at least 2 times, at least 3 times, at least 4 times, at least 5 times, at least 6 times, at least 7 times, at least 8 times, at least 9 times, or at least 10 times. In some embodiments, steps (b) and (c) can be repeated up to 10 times, up to 20 times, up to 30 time, up to 40 times, or up to 50 times.

In some embodiments, the sequences of the first and second sequencing read products can be aligned after each round of generating the first and second sequencing read products which are no more than 30 bases in length (e.g., after step (b)), or after generating a set of reiterative sequencing read products wherein the first and second sequencing read products which are no more than 30 bases in length (e.g., after step (d)).

The present disclosure provides methods for detecting in situ at least two different target RNA molecules in a cellular sample comprising step (a): providing a cellular sample harboring a plurality of RNA which comprises at least a first target RNA molecule and a second target RNA molecule, wherein the cellular sample is fixed and permeabilized. In some embodiments, the cellular sample harbors 2-25 different target RNA molecules, or harbors 25-50 different target RNA molecules, or harbors 50-75 different target RNA molecules, or harbors 75,100 different target RNA molecules. In some embodiments, the cellular sample harbors more than 100 different target RNA molecules, or more than 250 different target RNA molecules, or more than 500 different target molecules, or more than 1000 different target RNA molecules, or more. In some embodiments, the cellular sample harbors more than 10,000 different target RNA molecules. In some embodiments, the cellular sample comprises a whole cell, a plurality of whole cells, an intact tissue or an intact tumor. In some embodiments, the cellular sample comprises a fresh cellular sample, a freshly-frozen cellular sample, a sectioned cellular sample, or an FFPE cellular sample. In some embodiments, the cellular sample is deposited onto a solid support. In some embodiments, the cellular sample is deposited onto a solid support which is passivated with a coating that promotes cell adhesion. In some embodiments, the cellular sample is deposited on a support that lacks immobilized capture oligonucleotides. In some embodiments, the plurality of RNA can comprise at least a first target RNA molecule. In some embodiments, the plurality of RNA can comprise at least a second target RNA molecule. In some embodiments, the cellular sample is cultured prior to conducting step (b) which is described below.

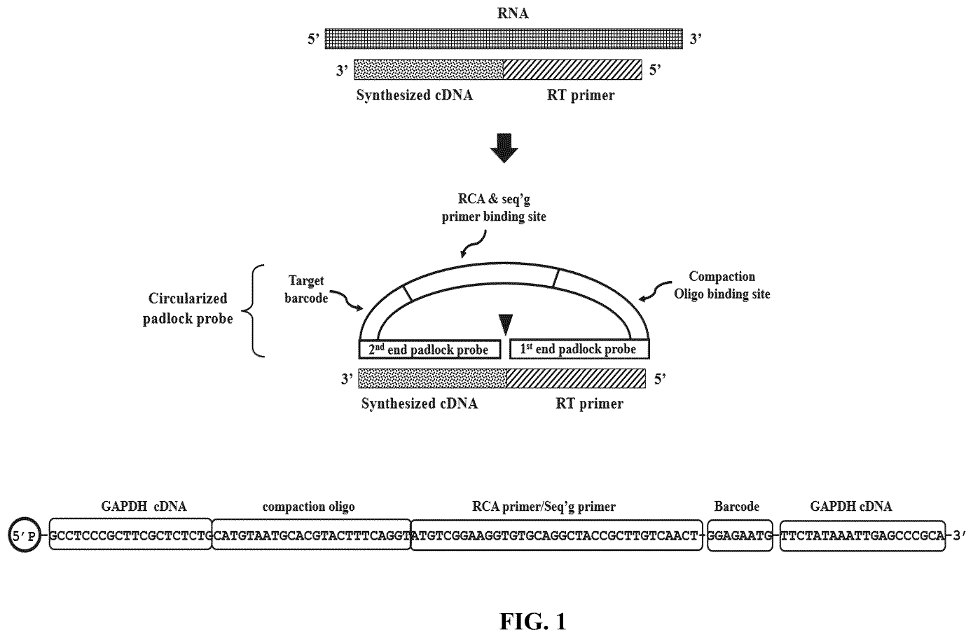

In some embodiments, methods for detecting at least two different target RNA molecules in a cellular sample further comprising step (b): generating inside the cellular sample a plurality of cDNA molecules which include at least a first target cDNA molecule that corresponds to the first target RNA molecule, and the plurality of cDNA molecules includes a second target cDNA molecule that corresponds to the second target RNA molecule. A cDNA molecule of the plurality of cDNA molecules may be generated through reverse transcription of a messenger RNA (mRNA) molecule inside the biological sample, wherein the cDNA molecule or mRNA molecule comprise a target nucleic acid sequence, or the reverse complement of the target nucleic acid sequence. A plurality of cDNA molecules may be generated through reverse transcription of one or more messenger RNA (mRNA) molecule(s) inside the biological sample, wherein the plurality of cDNA molecules or the one or more mRNA molecules comprise a target nucleic acid sequence, or the reverse complement of the target nucleic acid sequence. The first target cDNA molecule or the second target cDNA molecule may be generated through reverse transcription of one or more messenger RNA (mRNA) molecule(s) inside the biological sample, wherein the plurality of cDNA molecules or the one or more mRNA molecules comprise a target nucleic acid sequence, or the reverse complement of the target nucleic acid sequence. In some embodiments, the method comprises generating at least 2-10,000 different target cDNA molecules that correspond to 2-10,000 different target RNA molecules. In some embodiments, the generating of step (b) comprises contacting the plurality of RNA inside the cellular sample with (i) a plurality of reverse transcription primers, (ii) a plurality of reverse transcriptase enzymes, and (iii) a plurality of nucleotides, under a condition suitable for conducting a reverse transcription reaction to generate a plurality of cDNA molecules (e.g., a plurality of first strand cDNA molecules) in the cellular sample (e.g., FIG. 1). In some embodiments, the plurality of reverse transcription primers comprises a first sub-population of target-specific reverse transcription primers that hybridize selectively to the first target RNA, and comprises a second sub-population of target-specific reverse transcription primers that hybridize selectively to the second target RNA. In some embodiments, the plurality of reverse transcription primers comprises a first sub-population of random-sequence reverse transcription primers that hybridize to the first target RNA, and comprises a second sub-population of random-sequence reverse transcription primers that hybridize to the second target RNA.

In some embodiments, methods for detecting at least two different target RNA molecules in a cellular sample can comprise step (b): generating inside the cellular sample a plurality of cDNA molecules which can include at least a first target cDNA molecule that can corresponds to a first target RNA molecule. The plurality of cDNA molecules can include a second target cDNA molecule that can correspond to the second target RNA molecule. In some embodiments, the plurality of cDNA molecules can include at least a first target cDNA molecule. In some embodiments, the first target cDNA molecule can correspond to the first target RNA molecule. In some embodiments, the plurality of cDNA molecules can include at least a second target cDNA molecule. In some embodiments, the second target cDNA molecule can correspond to the second target RNA molecule. In some embodiments, the method can comprise generating at least 2-10,000 different target cDNA molecules that can correspond to 2-10,000 different target RNA molecules. In some embodiments, the generating of step (b) can comprises contacting the plurality of RNA inside the cellular sample with (i) a plurality of reverse transcription primers, (ii) a plurality of reverse transcriptase enzymes, and (iii) a plurality of nucleotides. In some embodiments, the generating of step (b) can be conducted under a condition suitable for conducting a reverse transcription reaction to generate a plurality of cDNA molecules (e.g., a plurality of first strand cDNA molecules) in the cellular sample (e.g., FIG. 1). In some embodiments, the generating of step (b) can comprise contacting the plurality of RNA inside the cellular sample with a plurality of reverse transcription primers. In some embodiments, the generating of step (b) can comprise contacting the plurality of RNA inside the cellular sample with a plurality of reverse transcription enzymes. In some embodiments, the generating of step (b) can comprise contacting the plurality of RNA inside the cellular sample with a plurality of nucleotides. In some embodiments, the plurality of reverse transcription primers can comprise a first sub-population of target-specific reverse transcription primers that can hybridize selectively to the first target RNA. In some embodiments, the plurality of reverse transcription primers can comprise a second sub-population of target-specific reverse transcription primers that can hybridize selectively to the second target RNA. In some embodiments, the plurality of reverse transcription primers can comprise a first sub-population of target-specific reverse transcription primers. In some embodiments, the first sub-population of target-specific reverse transcription primers can hybridize selectively to the first target RNA. In some embodiments, the plurality of reverse transcription primers can comprise a second sub-population of target-specific reverse transcription primers. In some embodiments, the second sub-population of target-specific reverse transcription primers can hybridize selectively to the second target RNA. In some embodiments, the plurality of reverse transcription primers can comprise a first sub-population of random-sequence reverse transcription primers that can hybridize to the first target RNA, and can comprise a second sub-population of random-sequence reverse transcription primers that can hybridize to the second target RNA. In some embodiments, the plurality of reverse transcription primers can comprise a first sub-population of random-sequence reverse transcription primers. In some embodiments, the first sub-population of random-sequence reverse transcription primers can hybridize to the first target RNA. In some embodiments, the plurality of reverse transcription primers can comprise a second sub-population of random-sequence reverse transcription primers. In some embodiments, the second sub-population of random-sequence reverse transcription primers can hybridize to the second target RNA.

In some embodiments, methods for detecting at least two different target RNA molecules in a cellular sample further comprise step (c): contacting the plurality of cDNA molecule in the cellular sample with a plurality of target-specific padlock probes which includes at least a first plurality of target-specific padlock probes and a second plurality of target-specific padlock probes. In some embodiments, the plurality of target-specific padlock probes can include at least a first plurality of target-specific padlock probes. In some embodiments, the plurality of target-specific padlock probes can include at least a second plurality of target-specific padlock probes. In some embodiments, the method comprises contacting the plurality of cDNA molecule in the cellular sample with at least 2-10,000 different target-specific padlock probes.

In some embodiments, individual padlock probes in the plurality of first target-specific padlock probes comprise a first and second end (e.g., first and second padlock binding arms), wherein the first end selectively hybridizes to a first region of the first target cDNA molecule and the second end selectively hybridizes to a second region of the first target cDNA molecule. In some embodiments, the contacting of step (c) comprises: hybridizing the first and second ends of the first target-specific padlock probes to proximal positions on the first target cDNA molecule to form a circularized first target-specific padlock probe having a nick or gap between the hybridized first and second ends (e.g., FIG. 1). In some embodiments, the first target-specific padlock probe comprises a first target barcode sequence that corresponds to the first target cDNA sequence. In some embodiments, the first target-specific padlock probe comprises a first target barcode sequence that is located adjacent to one of the regions of the first target-specific padlock probe that selectively hybridizes to the first target cDNA molecule. In some embodiments, the first target-specific padlock probe comprises at least one universal adaptor sequence, such as for example a universal sequencing primer binding site (or a complementary sequence thereof). In some embodiments, the first target-specific padlock probe comprises a universal primer binding site for a rolling circle amplification primer (or a complementary sequence thereof). In some embodiments, the first target-specific padlock probe comprises a universal compaction oligonucleotide binding site (or a complementary sequence thereof).

In some embodiments, the method comprises contacting in situ: the first target nucleic acid sequence or a reverse complement thereof with a first oligonucleotide comprising a first end portion and a second end portion. The first end portion and second end portion of the first oligonucleotide may be complementary. The first end portion and second end portion of the first oligonucleotide may bind to two neighboring segments of the first target nucleic acid sequence or the reverse complement thereof so that the first oligonucleotide forms a circular structure with a gap between the first end portion and the second end portion. In some embodiments, the method further comprises contacting in situ: the second target nucleic acid sequence or a reverse complement thereof with a second oligonucleotide comprising a first end portion and a second end portion. In some embodiments, the first end portion and second end portion of the second oligonucleotide are complementary. In some embodiments, the first end portion and second end portion bind to two neighboring segments of the second target nucleic acid sequence or a reverse complement thereof so that the second oligonucleotide forms a circular structure with a gap between the first end portion and the second end portion. In some embodiments, the first target nucleic acid sequence comprises the first cDNA molecule or the first mRNA molecule. In some embodiments, the second target nucleic acid sequence comprises the second cDNA molecule or the second mRNA molecule. In some embodiments, the gap of the first oligonucleotide or the second oligonucleotide has a size of one nucleotide. In some embodiments, the gap of the first oligonucleotide and the second oligonucleotide has a size of one nucleotide. In some embodiments, the gap of the first oligonucleotide has a size of 1, 2, 3, 4, 5, 6, 7, 8, 9, 10, 11, 12, 13, 14, 15, 16, 17, 18, 19, or more nucleotides. In some embodiments, the gap of the second oligonucleotide has a size of 1, 2, 3, 4, 5, 6, 7, 8, 9, 10, 11, 12, 13, 14, 15, 16, 17, 18, 19, or more nucleotides. In some embodiments, the gap of the first oligonucleotide or the second oligonucleotide has a size of at least two nucleotides. In some embodiments, the gap of the first oligonucleotide and the second oligonucleotide has a size of at least two nucleotides. In some embodiments, the first oligonucleotide further comprises a first identification sequence that identifies the first target nucleic acid sequence. In some embodiments, the second oligonucleotide further comprises a second identification sequence that identifies the second target nucleic acid sequence. In some embodiments, the first oligonucleotide further comprises a nucleic acid sequence that is complementary sequence to a nucleic acid sequence of a sequencing primer. In some embodiments, the second oligonucleotide further comprises a nucleic acid sequence that is complementary sequence to a nucleic acid sequence of a sequencing primer. In some embodiments, the first oligonucleotide further comprises a nucleic acid sequence that is complementary sequence to a nucleic acid sequence of a primer for nucleic acid amplification. In some embodiments, the second oligonucleotide further comprises a nucleic acid sequence that is complementary sequence to a nucleic acid sequence of a primer for nucleic acid amplification. In some embodiments, the primer for nucleic acid amplification is a primer for rolling circle amplification (RCA). In some embodiments, the primer for RCA produces a concatemer. In some embodiments, the concatemer comprises at least two repeats of a target nucleic acid sequence of the at least two target nucleic acid sequences or a portion thereof, or a reverse complement thereof. In some embodiments, the first oligonucleotide further comprises a reverse complement for a compaction oligonucleotide. In some embodiments, the second oligonucleotide further comprises a reverse complement for a compaction oligonucleotide. In some embodiments, a first segment of the compaction oligonucleotide is complementary and binds to a first portion of the concatemer. In some embodiments, a second segment of the compaction oligonucleotide is complementary and binds to a second portion of the concatemer. In some embodiments, the binding to a first and/or a second portion of the concatemer results in a reduction in the size or a change in the shape of the concatemer. In some embodiments, the method further comprises joining in situ the first and second end portions of the first oligonucleotide to produce a first circular oligonucleotide inside the biological sample. In some embodiments, the method further comprises joining in situ the first and second end portions of the second oligonucleotide to produce a second circular oligonucleotide inside the biological sample. In some embodiments, the joining the first and second end portions of the first oligonucleotide comprises joining the first and second end portions of the first oligonucleotides through a first nucleic acid enzyme. In some embodiments, the joining the first and second end portions of the second oligonucleotide comprises joining the first and second end portions of the second oligonucleotide through a second nucleic acid enzyme. In some embodiments, the first nucleic acid enzyme and the second nucleic acid enzyme are the same type of enzyme. In some embodiments, the joining the first and second end portions of the second oligonucleotide comprises joining the first and second end portions of the second oligonucleotide through a second nucleic acid enzyme, wherein the first nucleic acid enzyme and the second nucleic acid enzyme are a different type of enzyme. In some embodiments, the first nucleic acid enzyme comprises a nucleic acid ligase, a nucleic acid ligation enzyme, a nucleic acid polymerase, a nucleic acid polymerization enzyme, or combinations thereof. In some embodiments, the second nucleic acid enzyme comprises a nucleic acid ligase, a nucleic acid ligation enzyme, a nucleic acid polymerase, a nucleic acid polymerization enzyme, or combinations thereof. In some embodiments, the method further comprises amplifying in situ the first circular oligonucleotide to produce the first nucleic acid molecule. In some embodiments, the method further comprises amplifying in situ the second circular oligonucleotide to produce the second nucleic acid molecule. In some embodiments, the amplifying comprises rolling circle amplification (RCA), wherein the first nucleic acid molecule comprises a first concatemer and the second nucleic acid molecule comprises a second concatemer. In some embodiments, the first concatemer comprises at least two repeats of a first unit nucleic acid sequence comprising the first target nucleic acid sequence or a portion thereof, or the reverse complement of the first target nucleic acid sequence or a portion thereof. In some embodiments, the second concatemer comprises at least two repeats of a second unit nucleic acid sequence comprising the second target nucleic acid sequence or a portion thereof, or the reverse complement of the second target nucleic acid sequence or a portion thereof. In some embodiments, the first concatemer further comprises the first identification sequence that identifies the first target nucleic acid sequence, or a sequencing primer or a reverse complement thereof, wherein the second concatemer further comprises the second identification sequence that identifies the second target nucleic acid sequence, or a sequencing primer or a reverse complement thereof. In some embodiments, the first concatemer further comprises a first compaction oligonucleotide. In some embodiments, a first segment of the first compaction oligonucleotide is complementary and binds to a first portion of the first concatemer. In some embodiments, a second segment of the first compaction oligonucleotide is complementary and binds to a second portion of the first concatemer, to result in a reduction in the size or a change in the shape of the first concatemer. In some embodiments, the second concatemer further comprises a second compaction oligonucleotide. In some embodiments, a first segment of the second compaction oligonucleotide is complementary and binds to a first portion of the second concatemer. In some embodiments, a second segment of the second compaction oligonucleotide is complementary and binds to a second portion of the second concatemer, to result in a reduction in the size or a change in the shape of the second concatemer.

In some embodiments, individual padlock probes in the plurality of second target-specific padlock probes comprise a first and second end, wherein the first end selectively hybridizes to a first region of the second target cDNA molecule and the second end selectively hybridizes to a second region of the second target cDNA molecule. In some embodiments, the contacting of step (c) comprises: hybridizing the first and second ends of the second target-specific padlock probes to proximal positions on the second target cDNA molecule to form a circularized second target-specific padlock probe having a nick or gap between the hybridized first and second ends. In some embodiments, the second target-specific padlock probe comprises a second target barcode sequence that corresponds to the second target cDNA sequence. In some embodiments, the second target-specific padlock probe comprises a second target barcode sequence that is located adjacent to one of the regions of the second target-specific padlock probe that selectively hybridizes to the second target cDNA molecule. In some embodiments, the second target-specific padlock probe at least one universal adaptor sequence, such as for example a comprises at least one universal sequencing primer binding site (or a complementary sequence thereof). In some embodiments, the second target-specific padlock probe comprises a universal primer binding site for a rolling circle amplification primer (or a complementary sequence thereof). In some embodiments, the second target-specific padlock probe comprises a universal compaction oligonucleotide binding site (or a complementary sequence thereof).

In some embodiments, the target-specific padlock probes comprise a universal sequencing primer binding site and a target barcode sequence that are adjacent to each other so that the target barcode region of the concatemer is sequenced first. The target barcode sequence can be any length, for example 3-15 bases, or 15-25 bases, or 25-40 bases, or longer.

In some embodiments, methods for detecting at least two different target RNA molecules in a cellular sample further can comprise step (d): closing the nick or gap in the at least first and second circularized target-specific padlock probes by conducting an enzymatic reaction, thereby generating at least a first covalently closed circular padlock probe and a second covalently closed circular padlock probe inside the cellular sample. In some embodiments, step (d) can comprise closing the nick or gap in the at least first circularized target-specific padlock probe by conducting an enzymatic reaction, thereby generating at least a first covalently closed circular padlock probe. In some embodiments, step (d) can comprise closing the nick or gap in the at least second circularized target-specific padlock probe by conducting an enzymatic reaction, thereby generating at least a second covalently closed circular padlock probe. In some embodiments, the closing the nick can comprise conducting an enzymatic ligation reaction. In some embodiments, closing the gap can comprise conducting a polymerase-catalyzed fill-in reaction using the first or second target cDNA molecule as a template, and conducting an enzymatic ligation reaction. In some embodiments, closing the gap can comprise conducting a polymerase-catalyzed fill-in reaction using the first target cDNA molecule as a template. In some embodiments, closing the gap can comprise conducting a polymerase-catalyzed fill-in reaction using the second target cDNA molecule as a template. In some embodiments, the method can comprise closing the nick or gap in at least 2-10,000 circularized target-specific padlock probes by conducting an enzymatic reaction, thereby generating at least 2-10,000 covalently closed circular padlock probes inside the cellular sample.

In some embodiments, methods for detecting at least two different target RNA molecules in a cellular sample can further comprise step (e): conducting a rolling circle amplification reaction inside the cellular sample using the first and second covalently closed circular padlock probes as template molecules, thereby generating a plurality of concatemer molecules including at least a first concatemer molecule that corresponds to a first target RNA molecule, and the plurality of concatemer molecules can include at least a second concatemer molecule that corresponds to a second target RNA molecule. In some embodiments, step (e) can comprise conducting a rolling circle amplification reaction inside the cellular sample using the first covalently closed circular padlock probe as a template molecule. In some embodiments, the rolling circle amplification of the first covalently closed circular padlock probe can generate a plurality of concatemer molecule. In some embodiments, the plurality of concatemer molecule can include a first concatemer molecule. In some embodiments, the first concatemer molecule can correspond to a first target RNA molecule. In some embodiments, step (e) can comprise conducting a rolling circle amplification reaction inside the cellular sample using the second covalently closed circular padlock probe as a template molecule. In some embodiments, the rolling circle amplification of the second covalently closed circular padlock probe can generate a plurality of concatemer molecules. In some embodiments, the plurality of concatemer molecule can include a second concatemer molecule. In some embodiments, the second concatemer molecule can correspond to a second target RNA molecule. In some embodiments, the first concatemer molecule can comprise tandem repeat units, wherein a unit can comprise the sequence of the first target cDNA and the universal sequencing primer binding site (or a complementary sequence thereof). In some embodiments, the second concatemer molecule can comprises tandem repeat units, wherein a unit can comprise the sequence of the second target cDNA and the universal sequencing primer binding site (or a complementary sequence thereof).

In some embodiments, the method can comprise conducting a rolling circle amplification reaction inside the cellular sample using the at least 2-10,000 covalently closed circular padlock probes as template molecules, thereby generating at least 2-10,000 concatemer molecules that correspond to at least 2-10,000 target RNA molecules. In some embodiments, the rolling circle amplification can be conducted in the presence of a plurality of compaction oligonucleotides. In some embodiments, each compaction oligonucleotide can comprise a single stranded oligonucleotide. In some embodiments, the single stranded oligonucleotide can have a first region at one end and a second region at the other end. In some embodiments, the first region can hybridize to a portion of the concatemer molecule, and the second region can hybridize to another portion of the concatemer molecule. In some embodiments, the hybridization of the first region and the second region of the compaction oligonucleotide can compact the concatemer molecule. In some embodiments, the compaction oligonucleotide can compact the size of the concatemer molecule. In some embodiments, the compaction oligonucleotide can compact the shape of the concatemer molecule. In some embodiments, the compaction oligonucleotide can compact the size and shape of the concatemer molecule. In some embodiments, the compaction oligonucleotide can compact the concatemer molecule to form a compact nanoball.

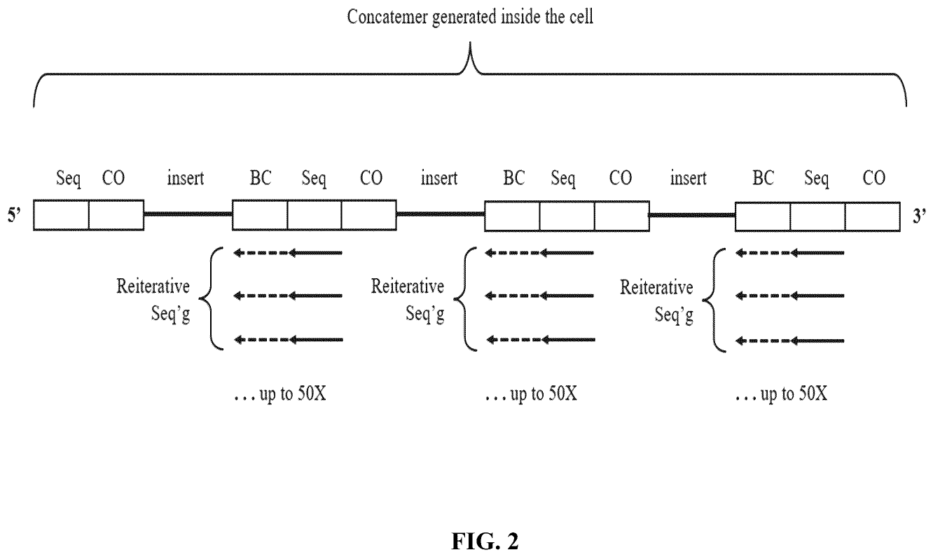

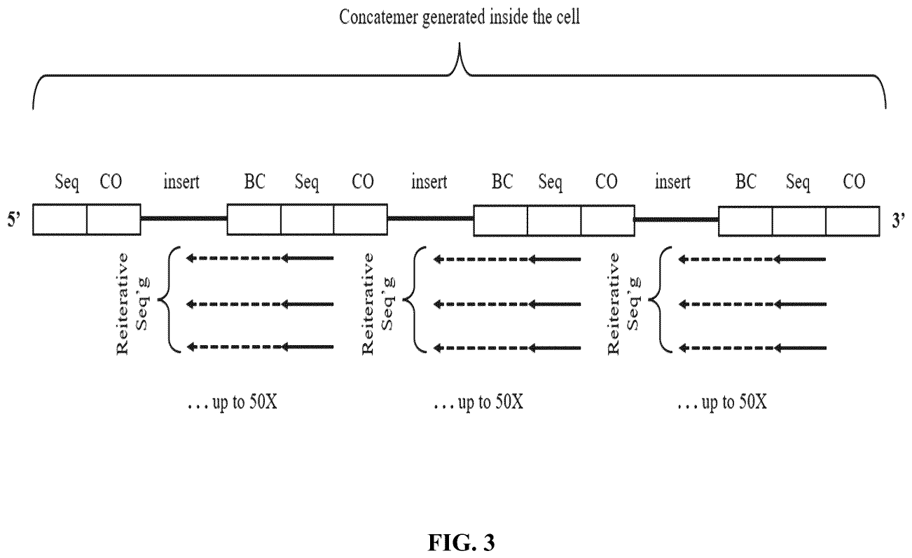

In some embodiments, methods for detecting at least two different target RNA molecules in a cellular sample can further comprise step (f): sequencing the plurality of concatemer molecules inside the cellular sample, which can comprise sequencing the first concatemer molecule by conducting no more than 2-30 sequencing cycles to generate a plurality of first sequencing read products, and sequencing the second concatemer molecule by conducting no more than 2-30 sequencing cycles to generate a plurality of second sequencing read products. In some embodiments, step (f) can comprise sequencing the plurality of concatemer molecules inside the cellular sample. In some embodiments, sequencing the plurality of concatemer molecules inside the cellular sample can comprise sequencing the first concatemer molecule by conducting no more than 2-30 sequencing cycles to generate a plurality of first sequencing read products. In some embodiments, step (f) can comprise sequencing the plurality of concatemer molecules inside the cellular sample. In some embodiments, sequencing the plurality of concatemer molecules inside the cellular sample can comprise sequencing the second concatemer molecule by conducting no more than 2-30 sequencing cycles to generate a plurality of second sequencing read products. In some embodiments, the sequencing of step (f) can comprise sequencing no more than 2-30 bases of the first concatemer molecules to generate a plurality of first sequencing read products, and which can comprise sequencing no more than 2-30 bases of the second concatemer molecules to generate a plurality of second sequencing read products. In some embodiments, the sequencing of step (f) can comprise sequencing no more than 2-30 bases of the first concatemer molecules to generate a plurality of first sequencing read products. In some embodiments, the sequencing of step (f) can comprise sequencing no more than 2-30 bases of the second concatemer molecules to generate a plurality of second sequencing read products. In some embodiments, the method can comprise sequencing the at least 2-10,000 concatemer molecules inside the cellular sample, which can comprise conducting no more than 2-30 sequencing cycles on the 2-10,000 concatemer molecules to generate a plurality of sequencing read products. In some embodiments, only the first target barcode region of the first concatemer molecules are sequenced (e.g., FIG. 2). In some embodiments, at least a portion or the full length of the first target barcode of the first concatemer molecules are sequenced (e.g., FIG. 2). In some embodiments, the first target barcode is sequenced and a portion of the first cDNA region of the first concatemer molecules are sequenced (e.g., FIG. 3).

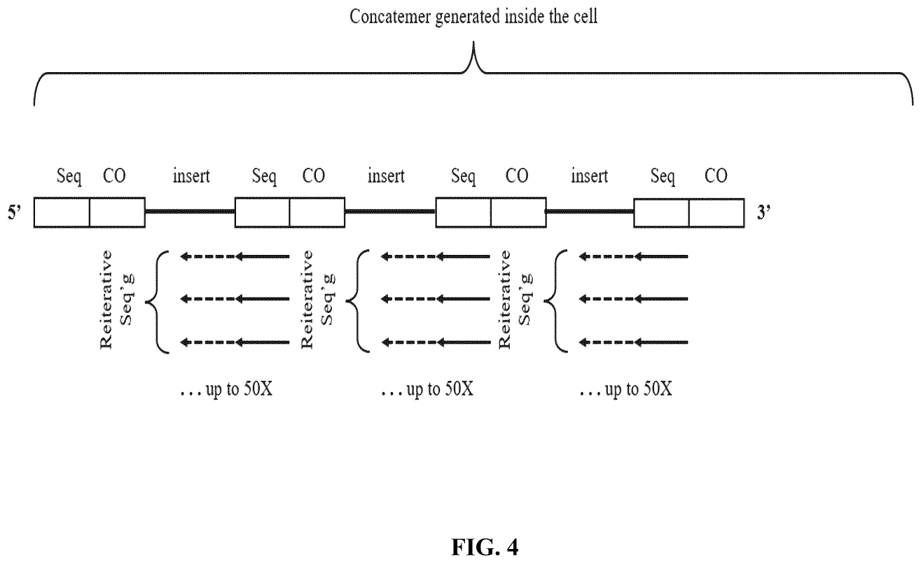

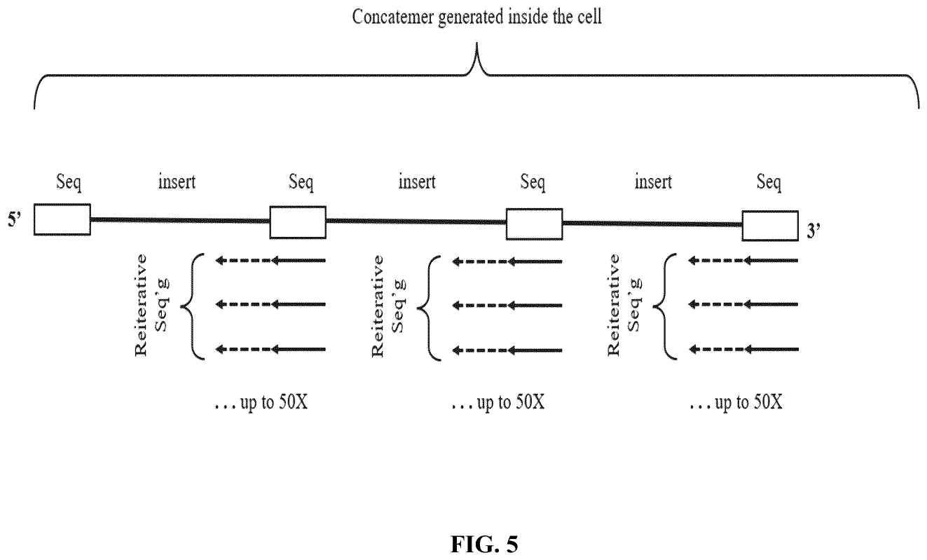

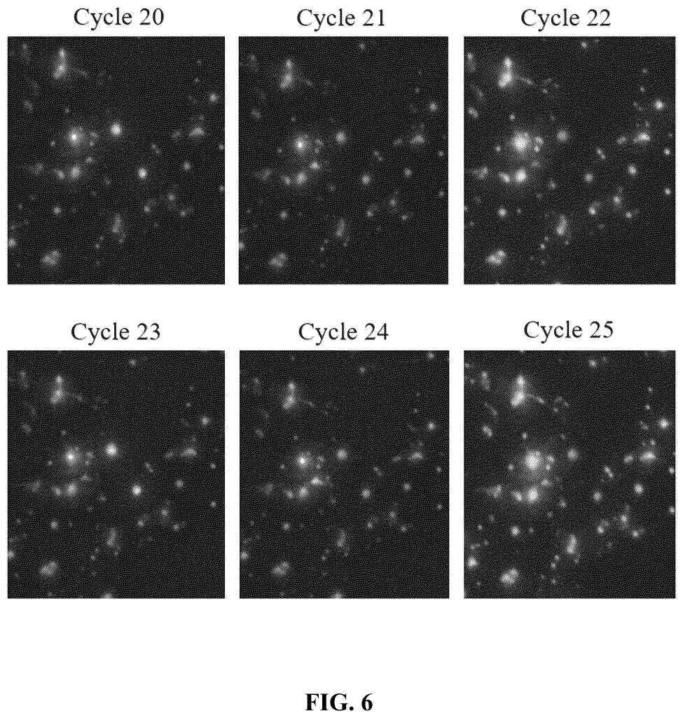

In some embodiments, at least a portion of the first cDNA region of the first concatemer molecules are sequenced (e.g., FIG. 4 or 5). In some embodiments, only the second target barcode region of the second concatemer molecules are sequenced (e.g., FIG. 2). In some embodiments, at least a portion or the full length of the second target barcode of the second concatemer molecules are sequenced (e.g., FIG. 2). In some embodiments, the second target barcode is sequenced and a portion of the second cDNA region of the second concatemer molecules are sequenced (e.g., FIG. 3). In some embodiments, at least a portion of the second cDNA region of the second concatemer molecules are sequenced (e.g., FIG. 4 or 5).

In some embodiments, the sequencing of step (f) comprises sequencing at least a portion of the first and second nucleic acid concatemers using an optical imaging system comprising a field-of-view (FOV) greater than 1.0 mm².

In some embodiments, in the sequencing of step (f), the plurality of first and second sequencing read products can be detectable by imaging, and wherein the sequencing can comprise decoding the plurality of first and second sequencing read products from the images obtained during the no more than 2-30 sequencing cycles. In some embodiments, in the sequencing of step (f), the plurality first sequencing read products can be detectable by imaging. In some embodiments, in the sequencing of step (f), the plurality of first sequencing read products can be detectable by imaging, and wherein the sequencing can comprise decoding the plurality of first sequencing read products from the images obtained during the no more than 2-30 sequencing cycles. In some embodiments, in the sequencing of step (f), the plurality of second sequencing read products can be detectable by imaging. In some embodiments, in the sequencing of step (f), the plurality of second sequencing read products can be detectable by imaging, and wherein the sequencing can comprise decoding the plurality of second sequencing read products from the images obtained during the no more than 2-30 sequencing cycles.

In some embodiments, in the sequencing of step (f), the plurality of first and second sequencing read products can be detectable by imaging, and wherein the sequencing can comprise decoding the plurality of first and second sequencing read products from the images obtained during the no more than 2-30 sequencing cycles. In some embodiments, in the sequencing of step (f), the plurality first sequencing read products can be detectable by imaging. In some embodiments, in the sequencing of step (f), the plurality of first sequencing read products can be detectable by imaging, and wherein the sequencing can comprise decoding the plurality of first sequencing read products from the images obtained during the no more than 2-30 sequencing cycles. In some embodiments, in the sequencing of step (f), the plurality of second sequencing read products can be detectable by imaging. In some embodiments, in the sequencing of step (f), the plurality of second sequencing read products can be detectable by imaging, and wherein the sequencing can comprise decoding the plurality of second sequencing read products from the images obtained during the no more than 2-30 sequencing cycles.

In some embodiments, the sequences of the first sequencing read products can be aligned with a first target reference sequence to confirm the presence of the first target RNA in the cellular product. In some embodiments, the sequences of the second sequencing read products can be aligned with a second target reference sequence to confirm the presence of the second target RNA in the cellular sample.

In some embodiments, the sequencing of step (f) can comprise (1) contacting the plurality of concatemer molecules inside the cellular sample with (i) a plurality of universal sequencing primers, (ii) a plurality of sequencing polymerases, and (iii) a plurality of nucleotide reagents, under a condition suitable for hybridizing the plurality of universal sequencing primers to their respective universal sequencing primer binding sites on the concatemers. In some embodiments, the sequencing of step (f) can comprise (1) contacting the plurality of concatemer molecules inside the cellular sample with a plurality of universal sequencing primers. In some embodiments, the sequencing of step (f) can comprise (1) contacting the plurality of concatemer molecules inside the cellular sample with a plurality of sequencing polymerases. In some embodiments, the sequencing of step (f) can comprise (1) contacting the plurality of concatemer molecules inside the cellular sample with a plurality of nucleotide reagents. In some embodiments, the sequencing can further comprise (2) conducting no more than 2-30 sequencing cycles to generate at least a first plurality of sequencing read products. In some embodiments, the sequencing can further comprise (2) conducting no more than 2-30 sequencing cycles to generate at least a first plurality of sequencing read products and a second plurality of sequencing read products. In some embodiments, the sequencing can further comprise (3) removing the first plurality of sequencing read products from the concatemers and retaining the plurality of concatemers inside the cellular sample. In some embodiments, the sequencing can further comprise (3) removing the first plurality of sequencing read products from the first concatemer molecules. In some embodiments, the sequencing of (3) can further comprise retaining the first concatemer molecules inside the cellular sample. In some embodiments, the sequencing further can comprise (3) removing the first plurality of sequencing read products from the concatemers and retaining the first concatemer molecules inside the cellular sample, and removing the second plurality of sequencing read products from the second concatemer molecules and retaining the second concatemer molecules inside the cellular sample. In some embodiments, the sequencing can further comprise (3) removing the second plurality of sequencing read products from the second concatemer molecules. In some embodiments, the sequencing of (3) can further comprise retaining the second concatemer molecules inside the cellular sample. In some embodiments, the sequencing can further comprise (4) repeating (1)-(3) at least once. In some embodiments, (4) can comprise repeating (1) -(3) at least 2 times, at least 3 times, at least 4 times, at least 5 times, at least 6 times, at least 7 times, at least 8 times, at least 9 times, or at least 10 times. In some embodiments, (4) can comprise repeating (1) -(3) up to 10 times, up to 20 times, up to 30 time, up to 40 times, or up to 50 times.

In some embodiments, the reiterative sequencing can be conducting using a sequencing-by-binding procedure, labeled and/or non-labeled chain-terminating nucleotides, or multivalent molecules. Descriptions of these three sequencing methods is described below.

In some embodiments, the plurality of universal sequencing primers can be hybridized to concatemer template molecules with a hybridization reagent comprising an SSC buffer (e.g., 2× saline-sodium citrate) buffer with formamide (e.g., 10-20% formamide). The hybridization conditions comprise a temperature of about 20-30° C., for about 10-60 minutes.

In some embodiments, the plurality of sequencing read products can be removed from the concatemers and the plurality of concatemers can be retained inside the cellular sample using a de-hybridization reagent comprising an SSC buffer (e.g., saline-sodium citrate) buffer, with or without formamide, at a temperature that promotes nucleic acid denaturation such as for example 30-90° C.

In some embodiments, the plurality of nucleotide reagents of step (f) comprises a plurality of nucleotides that are detectably labeled or non-labeled. In some embodiments, individual nucleotides are linked to a detectable reporter moiety. In some embodiments, the detectable reporter moiety comprises a fluorophore. In some embodiments, the plurality of detectably labeled nucleotide analogs comprise a plurality of chain terminating nucleotides, where the chain terminating moiety is linked to the 3′ nucleotide sugar position to form a 3′ blocked nucleotide analog. In some embodiments, the chain terminating moiety can be removed to convert the 3′ blocked nucleotide analog to an extendible nucleotide having a 3′ OH group on the sugar. In some embodiments, the labeled nucleotide analogs are linked to a different fluorophore that corresponds to the nucleobases adenine, cytosine, guanine, thymine or uracil, where the different fluorophores emit a fluorescent signal during the sequencing of step (f). In some embodiments, a sequencing cycle comprises (1) contacting the concatemer/sequencing primer duplex with a sequencing polymerase and a detectably labeled chain terminating nucleotide under a condition suitable for polymerase-catalyzed incorporation of the detectably labeled chain terminating nucleotide into the terminal end of the sequencing primer, (2) detecting and imaging the fluorescent signal and color emitted by the incorporated chain terminating nucleotide, and (3) removing the chain terminating moiety (e.g., unblocking) and retaining the concatemer/sequencing primer duplex. In some embodiments, no more than 2-30 sequencing cycles can be conducted on the plurality of concatemers inside the cellular sample to generate a plurality of first sequencing read products and a plurality of second sequencing read products. In some embodiments, no more than 2-30 sequencing cycles can be conducted on the plurality of concatemers inside the cellular sample to generate a plurality of first sequencing read. In some embodiments, no more than 2-30 sequencing cycles can be conducted on the plurality of concatemers inside the cellular sample to generate a plurality of second sequencing read products. In some embodiments, the sequence of the first sequencing read product can be determined and aligned with a first reference sequence to confirm the presence of the first target RNA molecules inside the cellular sample. In some embodiments, the sequence of the second sequencing read product can be determined and aligned with a second reference sequence to confirm the presence of the first target polypeptides inside the cellular sample. In some embodiments, the sequences of the first and second sequencing read products can be aligned after each round of generating the first and second sequencing read products. In some embodiments, the first and second sequencing read products can be no more than 30 bases in length. In some embodiments, the sequences of the first and second sequencing read products can be aligned after generating a set of reiterative sequencing read products. In some embodiments, wherein the first and second sequencing read products can be no more than 30 bases in length. In some embodiments, the sequences of the first sequencing read products can be aligned after each round of generating the first sequencing read products. In some embodiments, the first sequencing read products can be no more than 30 bases in length. In some embodiments, the sequences of the first sequencing read products can be aligned after generating a set of reiterative sequencing read products. In some embodiments, wherein the first sequencing read products can be no more than 30 bases in length. In some embodiments, the sequences of the second sequencing read products can be aligned after each round of generating the second sequencing read products. In some embodiments, the second sequencing read products can be no more than 30 bases in length. In some embodiments, the sequences of the second sequencing read products can be aligned after generating a set of reiterative sequencing read products. In some embodiments, wherein the second sequencing read products can be no more than 30 bases in length. In some embodiments, the sequencing reactions can be conducted on a sequencing apparatus having a detector that captures fluorescent signals from the sequencing reactions inside the cellular sample. The sequencing apparatus can be configured to relay the fluorescent signal data captured by the detector to a computer system that is programmed to display images of different fluorescent spots which are co-located in the cellular sample, where individual fluorescent spots correspond to different target RNA molecules or different target polypeptides. In some embodiments, when the sequencing is conducted using different fluorescently-labeled nucleotide reagents that correspond to different nucleobases (e.g., A, G, C, T/U), then the images can have different color fluorescent spots co-located in the same cellular sample at different sequencing cycles.

In some embodiments, out-of-sync phasing and/or pre-phasing events can occur during synchronized sequencing reactions on clonally amplified template amplicons, where the sequencing reactions comprise polymerase-catalyzed sequencing reactions employing detectably labeled chain terminator nucleotides. In some embodiments, a sequencing reaction on one template molecule in the clonally-amplified template molecules moves ahead (e.g., pre-phasing) or fall behind (e.g., phasing) of the sequencing of the other template molecules within the clonally-amplified template molecules. During sequencing, a fluorescent signal is typically detected which corresponds to incorporation of a labeled chain terminator nucleotide. Thus, phasing and pre-phasing events can be detected and monitored using incorporation of a labeled chain terminator nucleotide.

In some embodiments, the plurality of nucleotide reagents of step (f) comprises a plurality of multivalent molecules each comprising a core attached to a plurality of nucleotide-arms, wherein the nucleotide-arms are attached to a nucleotide unit. In some embodiments, individual multivalent molecules are labeled with a detectably reporter moiety. In some embodiments, the detectable reporter moiety comprises a fluorophore. In some embodiments, the core of the multivalent molecule is labeled with a fluorophore, and wherein the fluorophore which is attached to a given core of the multivalent molecule corresponds to the nucleotide base (e.g., adenine, guanine, cytosine, thymine or uracil) of the nucleotide arm. In some embodiments, at least one of the nucleotide arms of the multivalent molecule comprises a linker and/or nucleotide base that is attached to a fluorophore, and wherein the fluorophore which is attached to a given nucleotide base corresponds to the nucleotide base (e.g., adenine, guanine, cytosine, thymine or uracil) of the nucleotide arm. In some embodiments, a sequencing cycle comprises (1) contacting the concatemer/sequencing primer duplex with a first sequencing polymerase to form a complexed polymerase, (2) contacting the complexed polymerase with a detectably labeled multivalent molecule under a condition suitable for binding a complementary nucleotide unit of the multivalent molecule to the complexed polymerase thereby forming a multivalent-binding complex, and the condition is suitable for inhibiting incorporation of the complementary nucleotide unit into the terminal end of the sequencing primer, (3) detecting and imaging the fluorescent signal and color emitted by the bound detectably labeled multivalent molecule, (4) removing the first sequencing polymerase and the bound detectably labeled multivalent molecule, and retaining the concatemer/sequencing primer duplex, (5) contacting the retained concatemer/sequencing primer duplex with a second sequencing polymerase and a non-labeled chain terminating nucleotide under a condition suitable for polymerase-catalyzed incorporation of the non-labeled chain terminating nucleotide into the terminal end of the sequencing primer, and (6) removing the chain terminating moiety (e.g., unblocking) and retaining the concatemer/sequencing primer duplex. In some embodiments, a sequencing cycle can comprise contacting the concatemer/sequencing primer duplex with a first sequencing polymerase to form a complexed polymerase. In some embodiments, a sequencing cycle can comprise contacting the complexed polymerase with a detectably labeled multivalent molecule under a condition suitable for binding a complementary nucleotide unit of the multivalent molecule to the complexed polymerase thereby forming a multivalent-binding complex, and the condition is suitable for inhibiting incorporation of the complementary nucleotide unit into the terminal end of the sequencing primer. In some embodiments, a sequencing cycle can comprise detecting and imaging the fluorescent signal and color emitted by the bound detectably labeled multivalent molecule. In some embodiments, a sequencing cycle can comprise removing the first sequencing polymerase and the bound detectably labeled multivalent molecule, and retaining the concatemer/sequencing primer duplex. In some embodiments, a sequencing cycle can comprise contacting the retained concatemer/sequencing primer duplex with a second sequencing polymerase and a non-labeled chain terminating nucleotide under a condition suitable for polymerase-catalyzed incorporation of the non-labeled chain terminating nucleotide into the terminal end of the sequencing primer. In some embodiments, a sequencing cycle can comprise removing the chain terminating moiety (e.g., unblocking) and retaining the concatemer/sequencing primer duplex. In some embodiments, individual cycle times can be achieved in less than 30 minutes. In some embodiments, the field of view (FOV) can exceed 1 mm² and the cycle time for scanning large area (>10 mm²) can be less than 5 minutes. In some embodiments, no more than 2-30 sequencing cycles can be conducted on the plurality of concatemers inside the cellular sample to generate a plurality of first sequencing read products and a plurality of second sequencing read products. In some embodiments, no more than 2-30 sequencing cycles can be conducted on the plurality of concatemers inside the cellular sample to generate a plurality of first sequencing read products. In some embodiments, no more than 2-30 sequencing cycles can be conducted on the plurality of concatemers inside the cellular sample to generate a plurality of second sequencing read products. In some embodiments, the sequence of the first sequencing read product can be determined and aligned with a first reference sequence to confirm the presence of the first target RNA molecules inside the cellular sample. In some embodiments, the sequence of the second sequencing read product can be determined and aligned with a second reference sequence to confirm the presence of the second target RNA molecules inside the cellular sample. In some embodiments, the sequences of the first and second sequencing read products can be aligned after each round of generating the first and second sequencing read products which are no more than 30 bases in length, or after generating a set of reiterative sequencing read products wherein the first and second sequencing read products which are no more than 30 bases in length. In some embodiments, the sequences of the first and second sequencing read products can be aligned after each round of generating the first and second sequencing read products. In some embodiments, the first and second sequencing read products can be no more than 30 bases in length. In some embodiments, the sequences of the first and second sequencing read products can be aligned after generating a set of reiterative sequencing read products. In some embodiments, wherein the first and second sequencing read products can be no more than 30 bases in length. In some embodiments, the sequences of the first sequencing read products can be aligned after each round of generating the first sequencing read products. In some embodiments, the first sequencing read products can be no more than 30 bases in length. In some embodiments, the sequences of the first sequencing read products can be aligned after generating a set of reiterative sequencing read products. In some embodiments, wherein the first sequencing read products can be no more than 30 bases in length. In some embodiments, the sequences of the second sequencing read products can be aligned after each round of generating the second sequencing read products. In some embodiments, the second sequencing read products can be no more than 30 bases in length. In some embodiments, the sequences of the second sequencing read products can be aligned after generating a set of reiterative sequencing read products. In some embodiments, wherein the second sequencing read products can be no more than 30 bases in length. In some embodiments, the sequencing reactions can be conducted on a sequencing apparatus having a detector that can capture fluorescent signals from the sequencing reactions inside the cellular sample. The sequencing apparatus can be configured to relay the fluorescent signal data captured by the detector to a computer system that can be programmed to display images of different fluorescent spots which can be co-located in the cellular sample, where individual fluorescent spots can correspond to different target RNA molecules (e.g., the first target RNA molecule, the second target RNA molecule, or a combination of the first RNA molecule and the second RNA molecule.

In some embodiments, when sequencing with detectably labeled multivalent molecules, step (2) in which multivalent-binding complexes are formed and step (3) in which the bound detectably labeled multivalent molecules are imaged and detected, the conditions are gentle compared to sequencing workflows that employ detectable labeled chain terminating nucleotides. For example, steps (2) and (3) can be conducted at a gentle temperature of about 35-45° C., or about 39-42° C. Steps (2) and (3) can be conducted at a gentle temperature which can help retain the compact size and shape of a DNA nanoball during multiple sequencing cycles (e.g., up to 30 cycles) which can improve FWHM (full width half maximum) of a spot image of the DNA nanoball inside a cellular sample. In some embodiments, the DNA nanoball does not unravel during multiple sequencing cycles. In some embodiments, the spot image of the DNA nanoball does not enlarge during multiple sequencing cycles. In some embodiments, the spot image of the DNA nanoball remains a discrete spot during multiple sequencing cycles. The spot image can be represented as a Gaussian spot and the size can be measured as a FWHM. A smaller spot size as indicated by a smaller FWHM typically correlates with an improved image of the spot. In some embodiments, the FWHM of a nanoball spot can be about 10 μm or smaller.

In some embodiments, out-of-sync phasing and/or pre-phasing events can occur during synchronized polymerase-catalyzed sequencing reactions employing detectably labeled multivalent molecules. During sequencing, a fluorescent signal can be detected which corresponds to binding of complementary nucleotide unit of a multivalent molecule to the complexed polymerase thereby forming a multivalent-binding complex. Thus, phasing and pre-phasing events can be detected and monitored using binding of labeled multivalent molecules. In some embodiments, when conducting up to 30 sequencing cycles with detectably labeled multivalent molecules, the phasing and/or pre-phasing rate can be less than about 5%, or less than about 1%, or less than about 0.01%, or less than about 0.001%. By contrast, the phasing and/or pre-phasing rates for conducting up to 30 sequencing cycles using labeled chain terminator nucleotides can be about 5%.

In some embodiments, the sequencing of (f) can comprise determining the sequence of the first nucleic acid molecule or the portion thereof, wherein the first nucleic acid molecule or the portion thereof consists of 2-30 nucleotides. In some embodiments, the sequence of (f) can comprise determining the sequence of the second nucleic acid molecule or the portion thereof, wherein the second nucleic acid molecule or the portion thereof consists of 2-30 nucleotides. In some embodiments, the 2-30 nucleotides of the first nucleic acid molecule or the portion thereof comprises the first identification sequence and the 2-30 nucleotides of the second nucleic acid molecule or the portion thereof comprises the second identification sequence. In some embodiments, the 2-30 nucleotides of the first nucleic acid molecule or the portion thereof further comprises at least a portion of the sequence of the first cDNA molecule or the first mRNA molecule and the 2-30 nucleotides of the second nucleic acid molecule or the portion thereof further comprises at least a portion of the sequence of the second cDNA molecule or the second mRNA molecule.

In some embodiments, the determining comprises contacting the first concatemer with a polymerizing enzyme, a plurality of nucleotides, and a primer sequence that is complementary to a portion of the first concatemer under conditions sufficient to form a binding complex comprising the polymerizing enzyme, a nucleotide of the plurality of nucleotides that is complementary and binds to a nucleotide unit of the first concatemer, and the first concatemer hybridized to the primer sequence, wherein the nucleotide comprises a fluorescent label and a removable blocking group at the 3′ carbon position of the sugar moiety. In some embodiments, the method further comprises incorporating the nucleotide into the 3′ end of the primer sequence. In some embodiments, the method further comprises identifying the nucleobase of the incorporated nucleotide by imaging the fluorescent label of the incorporated nucleotide.

In some embodiments, the determining comprises contacting the second concatemer with a polymerizing enzyme, a plurality of nucleotides, and a primer sequence that is complementary to a portion of the second concatemer under conditions sufficient to form a binding complex comprising the polymerizing enzyme, a nucleotide of the plurality of nucleotides that is complementary and binds to a nucleotide unit of the second concatemer, and the second concatemer hybridized to the primer sequence, wherein the nucleotide comprises a fluorescent label and a removable blocking group at the 3′ carbon position of the sugar moiety. In some embodiments, the method further comprises incorporating the nucleotide into the 3′ end of the primer sequence. In some embodiments, the method further comprises identifying the nucleobase of the incorporated nucleotide by imaging the fluorescent label of the incorporated nucleotide. In some embodiments, the determining further comprises contacting the nucleotide with an agent to remove the blocking group from the nucleotide and generate a 3′ OH group on the sugar moiety. In some embodiments, the blocking group comprises an alkyl group, an alkenyl group, an alkynyl group, an allyl group, an aryl group, a benzyl group, an azide group, an azido group, an O-azidomethyl group, an amine group, an amide group, a keto group, an isocyanate group, a phosphate group, a thiol group, a disulfide group, a carbonate group, a urea group, a silyl group, or any combination thereof. In some embodiments, the plurality of nucleotides consists of at least two of the same type of nucleotide, wherein the same type of nucleotide is selected from the group comprising dATP, dGTP, dCTP, dTTP, and dUTP. In some embodiments, the fluorescent label of one type of nucleotide of the group emits light at a wavelength that is different from the wavelength of light emitted from the fluorescent label of another type of nucleotide of the group. In some embodiments, the plurality of nucleotides comprises at least two types of nucleotides, wherein the at least two types of nucleotides are selected from the group comprising dATP, dGTP, dCTP, dTTP, and dUTP. In some embodiments, the fluorescent label of one type of nucleotide of the group emits light at a wavelength that is different from the wavelength of light emitted from the fluorescent label of another type of nucleotide of the group.

In some embodiments, the determining comprises: contacting two of the first concatemer with two of a polymerizing enzyme, a plurality of nucleotide conjugates, and two of a primer sequence that is complementary to a portion of the first concatemer under conditions sufficient to form a multivalent binding complex comprising each of the two of the polymerizing enzyme, a nucleotide conjugate of the plurality of nucleotide conjugates, and each of the two of the first concatemer hybridized to each of the two of the primer sequence, wherein the nucleotide conjugate comprises a label and at least two of a nucleotide moiety, wherein two of the at least two of the nucleotide moiety are each complementary and bind to a nucleotide of each of the two of the first concatemer. In some embodiments, the determining comprises detecting the multivalent binding complex through the label of the nucleotide conjugate; and identifying the nucleobases of the nucleotides of the two of the first concatemer that are each complementary and bind to each of the two of the at least two of the nucleotide moiety of the nucleotide conjugate. In some embodiments, the conditions inhibit incorporation of the at least two of the nucleotide moiety of the nucleotide conjugate into the two of the first or second concatemer. In some embodiments, the nucleotide conjugate comprises a core coupled to a plurality of nucleotide arms, wherein each of the nucleotide moiety is attached to one nucleotide arm of the plurality of nucleotide arms. In some embodiments, the detectable label comprises a fluorescent label. In some embodiments, the detecting comprises imaging the fluorescent label. In some embodiments, the determining further comprises: removing the two of the polymerizing enzyme and the nucleotide conjugate from the two of the first concatemer; contacting each of the two of the first concatemer hybridized to each of the two of the primer sequence with two of a second polymerizing enzyme and a plurality of unlabeled nucleotides under conditions suitable for forming a binding complex comprising each of the two of the second polymerizing enzyme, each of the two of the first concatemer hybridized to each of the two of the primer sequence, and two of the plurality of the unlabeled nucleotides; and incorporating each of the two of the plurality of the unlabeled nucleotides into each of the two of the primer sequence, wherein each of the two of the plurality of the unlabeled nucleotides is complementary and binds to a nucleotide of each of the two of the first concatemer, wherein an unlabeled nucleotide of the plurality of unlabeled nucleotides comprises a removable blocking group at the 3′ carbon of the sugan moiety.

In some embodiments, the determining comprises: contacting two of the second concatemer with two of a polymerizing enzyme, a plurality of nucleotide conjugates, and two of a primer sequence that is complementary to a portion of the second concatemer under conditions sufficient to form a multivalent binding complex comprising each of the two of the polymerizing enzyme, a nucleotide conjugate of the plurality of nucleotide conjugates, and each of the two of the second concatemer hybridized to each of the two of the primer sequence, wherein the nucleotide conjugate comprises a label and at least two of a nucleotide moiety, wherein two of the at least two of the nucleotide moiety are each complementary and bind to a nucleotide of each of the two of the second concatemer; detecting the multivalent binding complex through the label of the nucleotide conjugate; and identifying the nucleobases of the nucleotides of the two of the second concatemer that are each complementary and bind to each of the two of the at least two of the nucleotide moiety of the nucleotide conjugate. In some embodiments, wherein the conditions inhibit incorporation of the at least two of the nucleotide moiety of the nucleotide conjugate into the two of the first or second concatemer. In some embodiments, the nucleotide conjugate comprises a core coupled to a plurality of nucleotide arms, wherein each of the nucleotide moiety is attached to one nucleotide arm of the plurality of nucleotide arms. In some embodiments, the detectable label comprises a fluorescent label. In some embodiments, the detecting comprises imaging the fluorescent label. In some embodiments, the determining further comprises: removing the two of the polymerizing enzyme and the nucleotide conjugate from the two of the first concatemer; contacting each of the two of the first concatemer hybridized to each of the two of the primer sequence with two of a second polymerizing enzyme and a plurality of unlabeled nucleotides under conditions suitable for forming a binding complex comprising each of the two of the second polymerizing enzyme, each of the two of the first concatemer hybridized to each of the two of the primer sequence, and two of the plurality of the unlabeled nucleotides; and incorporating each of the two of the plurality of the unlabeled nucleotides into each of the two of the primer sequence, wherein each of the two of the plurality of the unlabeled nucleotides is complementary and binds to a nucleotide of each of the two of the first concatemer, wherein an unlabeled nucleotide of the plurality of unlabeled nucleotides comprises a removable blocking group at the 3′ carbon of the sugan moiety.

In some embodiments, the determining further comprises: removing the two of the polymerizing enzyme and the nucleotide conjugate from the two of the second concatemer; contacting each of the two of the second concatemer hybridized to each of the two of the primer sequence with two of a second polymerizing enzyme and a plurality of unlabeled nucleotides under conditions sufficient for forming a binding complex comprising each of the two of the second polymerizing enzyme, each of the two of the second concatemer hybridized to each of the two of the primer sequence, and two of the plurality of the unlabeled nucleotides and incorporating each of the two of the plurality of the unlabeled nucleotides into each of the two of the primer sequence, wherein each of the two of the plurality of the unlabeled nucleotides is complementary and binds to a nucleotide of each of the two of the second concatemer, wherein an unlabeled nucleotide of the plurality of unlabeled nucleotides comprises a removable blocking group at the 3′ carbon of the sugan moiety. In some embodiments, wherein the conditions inhibit incorporation of the at least two of the nucleotide moiety of the nucleotide conjugate into the two of the first or second concatemer. In some embodiments, the nucleotide conjugate comprises a core coupled to a plurality of nucleotide arms, wherein each of the nucleotide moiety is attached to one nucleotide arm of the plurality of nucleotide arms. In some embodiments, the detectable label comprises a fluorescent label. In some embodiments, the detecting comprises imaging the fluorescent label. In some embodiments, the determining further comprises: removing the two of the polymerizing enzyme and the nucleotide conjugate from the two of the first concatemer; contacting each of the two of the first concatemer hybridized to each of the two of the primer sequence with two of a second polymerizing enzyme and a plurality of unlabeled nucleotides under conditions suitable for forming a binding complex comprising each of the two of the second polymerizing enzyme, each of the two of the first concatemer hybridized to each of the two of the primer sequence, and two of the plurality of the unlabeled nucleotides; and incorporating each of the two of the plurality of the unlabeled nucleotides into each of the two of the primer sequence, wherein each of the two of the plurality of the unlabeled nucleotides is complementary and binds to a nucleotide of each of the two of the first concatemer, wherein an unlabeled nucleotide of the plurality of unlabeled nucleotides comprises a removable blocking group at the 3′ carbon of the sugan moiety.

In some embodiments, the determining comprises: contacting two of the first concatemer with two of a polymerizing enzyme, a plurality of nucleotide conjugates, a first primer sequence that is complementary to a first portion of the first concatemer, and a second primer sequence that is complementary to a second portion of the first concatemer under conditions sufficient to form a multivalent binding complex comprising each of the two of the polymerizing enzyme, a nucleotide conjugate of the plurality of nucleotide conjugates, the first portion of the first concatemer hybridized to the first primer sequence and a second portion of the first concatemer hybridized to the second primer sequence, wherein the nucleotide conjugate comprises a label and at least two of a nucleotide moiety, wherein two of the at least two of the nucleotide moiety are each complementary and bind to a nucleotide of a first portion and a second portion of the first concatemer; detecting the multivalent binding complex through the label of the nucleotide conjugate; and identifying the nucleobases of the nucleotides of the first and second portions of the first concatemer that are each complementary and bind to each of the two of the at least two of the nucleotide moiety of the nucleotide conjugate. In some embodiments, wherein the conditions inhibit incorporation of the at least two of the nucleotide moiety of the nucleotide conjugate into the two of the first or second concatemer. In some embodiments, the nucleotide conjugate comprises a core coupled to a plurality of nucleotide arms, wherein each of the nucleotide moiety is attached to one nucleotide arm of the plurality of nucleotide arms. In some embodiments, the detectable label comprises a fluorescent label. In some embodiments, the detecting comprises imaging the fluorescent label. In some embodiments, the determining further comprises: removing the two of the polymerizing enzyme and the nucleotide conjugate from the two of the first concatemer; contacting each of the two of the first concatemer hybridized to each of the two of the primer sequence with two of a second polymerizing enzyme and a plurality of unlabeled nucleotides under conditions suitable for forming a binding complex comprising each of the two of the second polymerizing enzyme, each of the two of the first concatemer hybridized to each of the two of the primer sequence, and two of the plurality of the unlabeled nucleotides; and incorporating each of the two of the plurality of the unlabeled nucleotides into each of the two of the primer sequence, wherein each of the two of the plurality of the unlabeled nucleotides is complementary and binds to a nucleotide of each of the two of the first concatemer, wherein an unlabeled nucleotide of the plurality of unlabeled nucleotides comprises a removable blocking group at the 3′ carbon of the sugan moiety.

In some embodiments, the determining comprises: contacting two of the second concatemer with two of a polymerizing enzyme, a plurality of nucleotide conjugates, a first primer sequence that is complementary to a first portion of the second concatemer, and a second primer sequence that is complementary to a second portion of the second concatemer under conditions sufficient to form a multivalent binding complex comprising each of the two of the polymerizing enzyme, a nucleotide conjugate of the plurality of nucleotide conjugates, the first portion of the second concatemer hybridized to the first primer sequence and a second portion of the second concatemer hybridized to the second primer sequence, wherein the nucleotide conjugate comprises a label and at least two of a nucleotide moiety, wherein two of the at least two of the nucleotide moiety are each complementary and bind to a nucleotide of a first portion and a second portion of the second concatemer; detecting the multivalent binding complex through the label of the nucleotide conjugate; and identifying the nucleobases of the nucleotides of the first and second portions of the second concatemer that are each complementary and bind to each of the two of the at least two of the nucleotide moiety of the nucleotide conjugate. In some embodiments, the conditions inhibit incorporation of the at least two of the nucleotide moiety of the nucleotide conjugate into the two of the first or second concatemer. In some embodiments, the nucleotide conjugate comprises a core coupled to a plurality of nucleotide arms, wherein each of the nucleotide moiety is attached to one nucleotide arm of the plurality of nucleotide arms. In some embodiments, the detectable label comprises a fluorescent label. In some embodiments, the detecting comprises imaging the fluorescent label. In some embodiments, the plurality of nucleotides consists of at least two of the same type of nucleotide, wherein the same type of nucleotide is selected from the group comprising dATP, dGTP, dCTP, dTTP, and dUTP, wherein the fluorescent label of one type of nucleotide of the group emits light at a wavelength that is different from the wavelength of light emitted from the fluorescent label of another type of nucleotide of the group. In some embodiments, the plurality of nucleotides comprises at least two types of nucleotides, wherein the at least two types of nucleotides are selected from the group comprising dATP, dGTP, dCTP, dTTP, and dUTP, wherein the fluorescent label of one type of nucleotide of the group emits light at a wavelength that is different from the wavelength of light emitted from the fluorescent label of another type of nucleotide of the group. In some embodiments, the determining further comprises contacting the nucleotide with an agent to remove the blocking group from the nucleotide and generate a 3′ OH group on the sugar moiety. In some embodiments, the blocking group comprises an alkyl group, an alkenyl group, an alkynyl group, an allyl group, an aryl group, a benzyl group, an azide group, an azido group, an O-azidomethyl group, an amine group, an amide group, a keto group, an isocyanate group, a phosphate group, a thiol group, a disulfide group, a carbonate group, a urea group, or a silyl group.

In methods as disclosed herein, the first target nucleic acid sequence or the second target nucleic acid sequence may correspond to at least a portion of a messenger RNA (mRNA) molecule. In methods as disclosed herein, the first target nucleic acid sequence and the second target nucleic acid sequence may correspond to at least a portion of a messenger RNA (mRNA) molecule. In methods as disclosed herein, the first target nucleic acid sequence or the second target nucleic acid sequence may correspond to at least a portion of a complementary DNA (cDNA) molecule. In methods as disclosed herein, the first target nucleic acid sequence and the second target nucleic acid sequence may correspond to at least a portion of a complementary DNA (cDNA) molecule. In some embodiments, the first target nucleic acid sequence and/or the second target nucleic acid sequence may correspond to two separate portions of the same mRNA or cDNA molecule. In some embodiments, the first target nucleic acid sequence and the second target nucleic acid sequence may correspond to two different mRNA or cDNA molecules. In some embodiments, the determining comprises detecting in situ the first or second sequencing product nucleic acid molecule inside the biological sample through imaging. In some embodiments, the determining comprises detecting in situ simultaneously the first and second sequencing product nucleic acid molecules inside the biological sample through imaging. In some embodiments, the imaging comprises fluorescent imaging. In some embodiments, the biological sample comprises a cellular organelle, a cell, a whole cell, a group of whole cells, a tissue, an intact tissue, a tumor, an intact tumor, an organ, an organism, a protozoa, an algae, a bacteria, a virus, a plant, a fungus, an insect, or an animal. In some embodiments, the biological sample comprises a fresh sample, a processed sample, a freshly-frozen sample, a sectioned sample, or a formalin-fixed and paraffin-embedded (FFPE) sample. In some embodiments, the biological sample comprises a fresh cellular sample, a freshly-frozen cellular sample, a sectioned cellular sample, or an FFPE cellular sample. In some embodiments, the at least two target nucleic acid sequences comprise a target DNA sequence. In some embodiments, the at least two target nucleic acid sequences comprise a target RNA sequence. In some embodiments, the at least two target nucleic acid sequences comprise a first target RNA sequence and a second target RNA sequence. In some embodiments, the first target RNA sequence comprises coding RNA, non-coding RNA, messenger RNA (mRNA), transfer RNA (tRNA), ribosomal RNA (rRNA), microRNA (miRNA), guide RNA (gRNA), small nuclear RNA (snRNA), small interference RNA (siRNA), anti-sense RNA, mature microRNA, or immature microRNA. In some embodiments, the second target RNA sequence comprises coding RNA, non-coding RNA, mRNA, tRNA, rRNA, miRNA, gRNA, snRNA, siRNA, anti-sense RNA, mature microRNA, or immature microRNA. In some embodiments, the at least two target nucleic acid sequences comprise a first target DNA sequence and a second target DNA sequence. In some embodiments, the first target DNA sequence comprises complementary DNA (cDNA), genomic DNA (gDNA), non-coding DNA, or coding DNA. In some embodiments, the second target DNA sequence comprises cDNA, gDNA, non-coding DNA, or coding DNA. In some embodiments, the at least two target nucleic acid sequences comprise a target RNA sequence and a target DNA sequence. In some embodiments, the target RNA sequence comprises coding RNA, non-coding RNA, mRNA, tRNA, rRNA, miRNA, gRNA, snRNA, siRNA, anti-sense RNA, mature microRNA, or immature microRNA. In some embodiments, the target DNA sequence comprises cDNA, gDNA, non-coding DNA, or coding DNA.

In some embodiments, methods for detecting at least two different target RNA molecules in a cellular sample can further comprise step (g): removing the plurality of first sequencing read products from the first concatemer molecules and retaining the first concatemer molecules in the cellular sample, and removing the plurality of second sequencing read products from the second concatemer molecules and retaining the second concatemer molecules in the cellular sample. In some embodiments, (g) can comprise removing the plurality of first sequence read products from the first concatemer molecule. In some embodiments, step (g) can further comprise retaining the first concatemer molecule in the cellular sample. In some embodiments, step (g) can comprise removing the plurality of second sequence read products from the second concatemer molecule. In some embodiments, step (g) can further comprise retaining the second concatemer molecule in the cellular sample.

In some embodiments, methods for detecting at least two different target RNA molecules in a cellular sample further comprising step (h): reiteratively sequencing the plurality of concatemers by repeating steps (f) and (g) at least once, wherein the sequences of the plurality of first sequencing read products confirms the presence of the first target RNA molecules in the cellular sample, and wherein the sequences of the plurality of second sequencing read products confirms the presence of the second target RNA molecules in the cellular sample. In some embodiments, the repeating comprises repeating at least 2 times, 5 times, 10 times, 15 times, 20 times, 25 times, 30 times, 35 times, 40 times, 45 times, or at least 50 times. In some embodiments, step (h) can comprise reiteratively sequencing the plurality of concatemers by repeating steps (f) and (g) at least once. In some embodiments, the repeating comprises repeating at least 2 times, 5 times, 10 times, 15 times, 20 times, 25 times, 30 times, 35 times, 40 times, 45 times, or at least 50 times. In some embodiments, the sequences of the plurality of first sequencing read products can confirm the presence of the first target RNA molecules in the cellular sample. In some embodiments, the sequences of the plurality of second sequencing read products can confirm the presence of the second target RNA molecules in the cellular sample. In some embodiments, the sequences of the plurality of first sequencing read products can confirm the presence of the first target RNA molecules in the cellular sample and the sequence of the plurality of the second sequencing read products can confirm the presence of the second target RNA molecules in the sample.

The present disclosure provides a method for detecting in situ at least two target nucleic acid molecules and at least two polypeptides in a biological sample, wherein the at least two target nucleic acid sequences comprise a first target nucleic acid sequence and a second target nucleic acid sequence, wherein the at least two target polypeptides comprise a first target polypeptide encoded by the first target nucleic acid sequence or a reverse complement thereof and a second target polypeptide encoded by the second target nucleic acid sequence or a reverse complement thereof. In some embodiments, the method comprises step (a) providing the biological sample. In some embodiments, the biological sample comprises a first nucleic acid molecule, comprising the first target nucleic acid sequence or a portion thereof, or the reverse complement of the first target nucleic acid sequence or a portion thereof. In some embodiments, the biological sample comprises a second nucleic acid molecule, comprising the second target nucleic acid sequence or a portion thereof, or the reverse complement of the second target nucleic acid sequence or a portion thereof. In some embodiments, the biological sample comprises a third nucleic acid molecule, comprising a third target nucleic acid sequence or a portion thereof, or the reverse complement of the third target nucleic acid sequence or a portion thereof, wherein the presence of third target nucleic acid sequence or the reverse complement thereof identifies the presence of the first target polypeptide in the biological sample. In some embodiments, the biological sample comprises a fourth nucleic acid molecule, comprising a fourth target nucleic acid sequence or a portion thereof, or the reverse complement of the fourth target nucleic acid sequence or a portion thereof, wherein the presence of the fourth target nucleic acid sequence or the reverse complement thereof identifies the presence of the second target polypeptide in the biological sample. In some embodiments, the method further comprises step (b) determining in situ the sequence of the first nucleic acid molecule or a portion thereof in the biological sample to generate a first sequencing product nucleic acid molecule that is complementary and binds to the first nucleic acid molecule or a portion thereof. In some embodiments, step (b) further comprises determining in situ the sequence of the third nucleic acid molecule or a portion thereof in the biological sample to generate a third sequencing product nucleic acid molecule that is complementary and binds to the third nucleic acid molecule or a portion. In some embodiments, the method further comprises step (c) identifying in situ the sequence of the second nucleic acid molecule or a portion thereof in the biological sample to generate a second sequencing product nucleic acid molecule that is complementary and binds to the second nucleic acid molecule or a portion thereof. In some embodiments, step (c) further comprises identifying in situ the sequence of the fourth nucleic acid molecule or a portion thereof in the biological sample to generate a fourth sequencing product nucleic acid molecule that is complementary and binds to the fourth nucleic acid molecule or a portion. In some embodiments, the full sequence of the first target nucleic acid sequence and the full sequence of the second target nucleic acid sequence have at least one nucleotide of difference. In some embodiments, the full sequence of the first target polypeptide and the full sequence of the second target polypeptide have at least one amino acid of difference. In some embodiments, the performance of step (b) is under a condition that prevents the performance of the step (c).

The present disclosure provides a method for method for detecting in situ at least two target nucleic acid sequences and at least two polypeptides in a biological sample. In some embodiments, at least two target nucleic acid sequences comprise a first target nucleic acid sequence and a second target nucleic acid sequence. In some embodiments, the at least two target polypeptides comprise a first target polypeptide encoded by the first target nucleic acid sequence or a reverse complement thereof. In some embodiments, the at least two target polypeptides comprise a second target polypeptide encoded by the second target nucleic acid sequence or a reverse complement thereof. In some embodiments, the method comprises (a) providing the biological sample comprising: (i) a first nucleic acid molecule, comprising the first target nucleic acid sequence or a portion thereof, or the reverse complement of the first target nucleic acid sequence or a portion thereof. In some embodiments, the biological sample comprises (ii) a second nucleic acid molecule, comprising the second target nucleic acid sequence or a portion thereof, or the reverse complement of the second target nucleic acid sequence or a portion thereof. In some embodiments, the biological sample comprises (iii)a third nucleic acid molecule, comprising a third target nucleic acid sequence or a portion thereof, or the reverse complement of the third target nucleic acid sequence or a portion thereof. In some embodiments, the presence of third target nucleic acid sequence or the reverse complement thereof identifies the presence of the first target polypeptide in the biological sample. In some embodiments, the biological sample further comprises (iv) a fourth nucleic acid molecule, comprising a fourth target nucleic acid sequence or a portion thereof, or the reverse complement of the fourth target nucleic acid sequence or a portion thereof, wherein the presence of the fourth target nucleic acid sequence or the reverse complement thereof identifies the presence of the second target polypeptide in the biological sample. In some embodiments, the method further comprises the step of (b) determining in situ the sequence of: (i) the first nucleic acid molecule or a portion thereof in the biological sample to generate a first sequencing product nucleic acid molecule that is complementary and binds to the first nucleic acid molecule or a portion thereof, wherein the first nucleic acid molecule or the portion thereof consists of 2-30 nucleotides. In some embodiments, step (b) further comprises determining in situ the sequence of (ii) the third nucleic acid molecule or a portion thereof in the biological sample to generate a third sequencing product nucleic acid molecule that is complementary and binds to the third nucleic acid molecule or a portion, wherein the third nucleic acid molecule or the portion thereof consists of 2-30 nucleotides. In some embodiments, the method further comprises (c) removing the first sequencing product nucleic acid molecule from the first nucleic acid molecule and the third sequencing product nucleic acid molecule from the third nucleic acid molecule. In some embodiments, the first nucleic acid molecule and the third nucleic acid molecule are positioned in the biological sample after the removing. In some embodiments, the method further comprises step (d): repeating (b) and (c). In some embodiments, the method further comprises step (e): identifying in situ the sequence of: (i) the second nucleic acid molecule or a portion thereof in the biological sample to generate a second sequencing product nucleic acid molecule that is complementary and binds to the second nucleic acid molecule or a portion thereof, wherein the second nucleic acid molecule or the portion thereof consists of 2-30 nucleotides. In some embodiments, step (e) further comprises identifying in situ the sequence of (ii) the fourth nucleic acid molecule or a portion thereof in the biological sample to generate a fourth sequencing product nucleic acid molecule that is complementary and binds to the fourth nucleic acid molecule or a portion. In some embodiments, the fourth nucleic acid molecule or the portion thereof consists of 2-30 nucleotides. In some embodiments, the method further comprises step (f): removing the second sequencing product nucleic acid molecule from the second nucleic acid molecule and the fourth sequencing product nucleic acid molecule from the fourth nucleic acid molecule, wherein the second nucleic acid molecule and the fourth nucleic acid molecule are located in the biological sample after the removing. In some embodiments, the method further comprises step (g): repeating steps (e) and (f). In some embodiments, the full sequence of the first target nucleic acid sequence and the full sequence of the second target nucleic acid sequence have at least one nucleotide of difference. In some embodiments, the full sequence of the first target polypeptide and the full sequence of the second target polypeptide have at least one amino acid of difference. In some embodiments, the performance of step (b) is under a condition that prevents the performance of the step (e).

The present disclosure provides a method for detecting in situ at least two target RNA sequences and at least two polypeptides in a biological sample. In some embodiments, the method comprises a step (a) providing the biological sample that is immobilized on a surface, permeabilized and fixed. In some embodiments, the biological sample comprises (i) a first target RNA sequence of at least two target RNA sequences and a first target polypeptide of the at least two polypeptides, wherein the first target polypeptide is encoded by the first target RNA sequence or a reverse complement thereof. In some embodiments, the biological sample comprises (ii) a second target RNA sequence of at least two target RNA sequences and a second target polypeptide of the at least two polypeptides, wherein the second target polypeptide is encoded by the second target RNA sequence or a reverse complement thereof. In some embodiments, the method further comprises a step (b) producing a first target cDNA sequence through reverse transcription of the first target RNA sequence and a second target cDNA sequence through reverse transcription of the second target RNA sequence. In some embodiments, the method further comprises a step (c) contacting the first target cDNA sequence with a first oligonucleotide comprising a first end portion and a second end portion, wherein the first end portion and second end portion of the first oligonucleotide are complementary and bind to two neighboring segments of the first target cDNA sequence so that the first oligonucleotide forms a circular structure with a gap between the first end portion and the second end portion, wherein the first oligonucleotide comprises a first identification sequence that identifies the first target RNA sequence, a first sequencing primer, and a nucleic acid amplification primer. In some embodiments, step (c) further comprises contacting the second target cDNA sequence with a second oligonucleotide comprising a first end portion and a second end portion, wherein the first end portion and second end portion of the second oligonucleotide are complementary and bind to two neighboring segments of the second target cDNA sequence, so that the second oligonucleotide forms a circular structure with a gap between the first end portion and the second end portion. In some embodiments, the second oligonucleotide comprises a second identification sequence that identifies the second target RNA sequence, a second sequencing primer, and a nucleic acid amplification primer. In some embodiments, the first sequencing primer and the second sequencing primer have at least one nucleotide of difference. In some embodiments, step (c) comprises contacting the first target polypeptide with a first oligonucleotide conjugate comprising a first short nucleic acid and a first binding moiety that binds specifically to the first target polypeptide, wherein the first short nucleic acid comprises a first tag sequence, a second tag sequence, wherein the first oligonucleotide conjugate binds specifically to the first target polypeptide through the first binding moiety to form a first binding complex, wherein the first and second tag sequences identify the first binding moiety. In some embodiments, step (c) comprises contacting the second target polypeptide with a second oligonucleotide conjugate comprising a second short nucleic acid and a second binding moiety that binds specifically to the second target polypeptide, wherein the second short nucleic acid comprises a third tag sequence, a fourth tag sequence, wherein the second oligonucleotide conjugate binds specifically to the second target polypeptide through the second binding moiety to form a second binding complex, wherein the third and fourth tag sequences identify the second binding moiety. In some embodiments, the method further comprises step (d) contacting the first binding complex with a third oligonucleotide comprising a first end portion and a second end portion, wherein the first end portion and second end portion of the third oligonucleotide are complementary and bind to the first tag sequence and the second tag sequence of the first oligonucleotide conjugate so that the third oligonucleotide forms a circular structure with a gap between the first end portion and the second end portion. In some embodiments, step (d) comprises contacting the second binding complex with a fourth oligonucleotide comprising a first end portion and a second end portion, wherein the first end portion and second end portion of the fourth oligonucleotide are complementary and bind to the third tag sequence and the fourth tag sequence so that the fourth oligonucleotide forms a circular structure with a gap between the first end portion and the second end portion. In some embodiments, the method further comprises step (e) joining the first and second end portions of the first oligonucleotide to produce a first circular oligonucleotide, the first and second end portions of the second oligonucleotides to produce a second circular oligonucleotide, the first and second end portions of the third oligonucleotide to produce a third circular oligonucleotide and the first and second end portions of the fourth oligonucleotide to produce a fourth circular oligonucleotide, wherein the first circular oligonucleotide and the third circular oligonucleotide comprise a first sequencing primer or the reverse complement thereof, wherein the second circular oligonucleotide and the fourth circular oligonucleotide comprise a second sequencing primer or the reverse complement thereof, wherein the sequences of the first and second sequencing primers have at least one nucleotide of difference. In some embodiments, the method further comprises step (f) amplifying the first circular oligonucleotide through rolling circle amplification to produce a first concatemer comprising a plurality of the first circular oligonucleotides. In some embodiments, step (f) further comprises amplifying the second circular oligonucleotide through rolling circle amplification to produce a second concatemer comprising a plurality of the second circular oligonucleotides. In some embodiments, step (f) further comprises amplifying the third circular oligonucleotide through rolling circle amplification to produce a third concatemer comprising a plurality of the third circular oligonucleotides. In some embodiments, step (f) further comprises amplifying the fourth circular oligonucleotide through rolling circle amplification to produce a fourth concatemer comprising a plurality of the fourth circular oligonucleotides. In some embodiments, the method further comprises step (g) determining in situ the sequence of the first concatemer or a portion thereof to generate a first sequencing product nucleic acid molecule that is complementary and binds to the first concatemer, wherein the sequence of the first concatemer or the portion thereof consists of 2-30 nucleotides. In some embodiments, step (g) further comprises determining in situ the sequence of the third concatemer or a portion thereof to generate a third sequencing product nucleic acid molecule that is complementary and binds to the third concatemer, wherein the sequence of the third concatemer or the portion thereof consists of 2-30 nucleotides. In some embodiments, the performance of step (g) is under a condition that prevents the performance of the step (j). In some embodiments, the method further comprises step (h) removing the first sequencing product nucleic acid molecule from the first concatemer and the third sequencing product nucleic acid molecule from the third concatemer, wherein the first concatemer and the third concatemer are positioned in the biological sample after the removing. In some embodiments, the method further comprises step (i) repeating (g) and (h) at least once. In some embodiments, the method further comprises step (j) determining in situ the sequence of the second concatemer or a portion thereof to generate a second sequencing product nucleic acid molecule that is complementary and binds to the second concatemer, wherein the sequence of the second concatemer or the portion thereof consists of 2-30 nucleotides. In some embodiments, step (j) further comprises determining in situ the sequence of the fourth concatemer or a portion thereof to generate a fourth sequencing product nucleic acid molecule that is complementary and binds to the fourth concatemer, wherein the sequence of the fourth concatemer or the portion thereof consists of 2-30 nucleotides. In some embodiments, the performance of steps (j) is under a condition that prevents the performance of the step (g). In some embodiments, the full sequence of the first target RNA sequence and the full sequence of the second target RNA sequence have at least one nucleotide of difference. In some embodiments, the full sequence of the first target polypeptide and the full sequence of the second target polypeptide have at least one amino acid of difference. In some embodiments, the method further comprises step (k) removing the second sequencing product nucleic acid molecule from the second concatemer and the fourth sequencing product nucleic acid molecule from the fourth concatemer. In some embodiments, the second concatemer and the fourth concatemer are positioned in the biological sample after the removing. In some embodiments, the method further comprises step (i) repeating (j) and (k) at least once.

In some embodiments, the determining comprises imaging the first, or third sequencing product nucleic acid molecule, wherein the identifying comprises imaging the second or fourth sequencing product nucleic acid molecule and the fourth sequencing product nucleic acid molecule. In some embodiments, the method further comprises imaging simultaneously the first sequencing product nucleic acid molecule and the second sequencing product nucleic acid molecule to analyze the spatial distribution of the first and second sequencing product nucleic acid molecules inside the biological sample. In some embodiments, the biological sample comprises a cellular organelle, a cell, a whole cell, a group of whole cells, a tissue, an intact tissue, a tumor, an intact tumor, an organ, an organism, a protozoa, an algae, a bacteria, a virus, a plant, a fungus, an insect, or an animal. In some embodiments, the biological sample comprises a fresh sample, a processed sample, a freshly-frozen sample, a sectioned sample, or a formalin-fixed and paraffin-embedded (FFPE) sample. In some embodiments, the first target nucleic acid sequence comprises DNA, cDNA, RNA, coding RNA, non-coding RNA, mRNA, tRNA, rRNA, niRNA, gRNA, snRNA, siRNA, anti-sense RNA, mature microRNA, or immature microRNA, and/or wherein the first target RNA sequence comprises coding RNA, non-coding RNA, mRNA, tRNA, rRNA, miRNA, gRNA, snRNA, siRNA, anti-sense RNA, mature microRNA, or immature microRNA. In some embodiments, the second target nucleic acid sequence comprises DNA, cDNA, RNA, coding RNA, non-coding RNA, mRNA, tRNA, rRNA, miRNA, gRNA, snRNA, siRNA, anti-sense RNA, mature microRNA, or immature microRNA, and/or wherein the second target RNA sequence comprises coding RNA, non-coding RNA, mRNA, tRNA, rRNA, miRNA, gRNA, snRNA, siRNA, anti-sense RNA, mature microRNA, or immature microRNA. In some embodiments, the biological sample comprises a human sample, a simian sample, an ape sample, a canine sample, a feline sample, a bovine sample, an equine sample, a murine sample, a porcine sample, a caprine sample, a lupine sample, a ranine sample, a piscine sample, a plant sample, an insect sample, a bacteria sample, an algae sample, a viral sample, a protozoa sample, or a fungus sample. In some embodiments, the first, second, third or fourth concatemer further comprises a compaction oligonucleotide, wherein: a first segment of the compaction oligonucleotide is complementary and binds to a first portion of the first, second, third, or fourth concatemer. In some embodiments, a second segment of the compaction oligonucleotide is complementary and binds to a second portion of the first, second, third, or fourth concatemer, to result in a reduction in the size or a change in the shape of the second concatemer. In some embodiments, the first sequencing product nucleic acid molecule comprises a first identification sequence that identifies the first target RNA sequence and the second sequencing product nucleic acid molecule comprises a second identification sequence that identifies the second target RNA sequence. In some embodiments, the first sequencing product nucleic acid molecule comprises a first identification sequence that identifies the first target RNA sequence and a portion of the first target RNA sequence, wherein the second sequencing product nucleic acid molecule comprises a second identification sequence that identifies the second target RNA sequence and a portion of the second target RNA sequence. In some embodiments, the determining comprises contacting the first second, third, or fourth concatemer with a polymerizing enzyme, a plurality of nucleotides, and a primer sequence that is complementary to a portion of the first second, third, or fourth concatemer under conditions sufficient to form a binding complex comprising the polymerizing enzyme, a nucleotide of the plurality of nucleotides that is complementary and binds to a nucleotide unit of the first second, third, or fourth concatemer, and the first second, third, or fourth concatemer hybridized to the primer sequence. In some embodiments, the nucleotide comprises a fluorescent label and a removable blocking group at the 3′ carbon position of the sugar moiety. In some embodiments, the determining further comprises incorporating the nucleotide into the 3′ end of the primer sequence. In some embodiments, the determining further comprises identifying the nucleobase of the incorporated nucleotide by imaging the fluorescent label of the incorporated nucleotide. In some embodiments, the determining further comprises contacting the nucleotide with an agent to remove a blocking group from the nucleotide and generate a 3′ OH group on the sugar moiety. In some embodiments, the blocking group comprises an alkyl group, an alkenyl group, an alkynyl group, an allyl group, an aryl group, a benzyl group, an azide group, an azido group, an O-azidomethyl group, an amine group, an amide group, a keto group, an isocyanate group, a phosphate group, a thiol group, a disulfide group, a carbonate group, a urea group, or a silyl group. In some embodiments, the plurality of nucleotides comprise one type of nucleotide selected from a group comprising dATP, dGTP, dCTP, dTTP and dUTP. In some embodiments, the plurality of nucleotides comprises a mixture of any combination of two or more types of nucleotides selected from a group consisting of dATP, dGTP, dCTP, dTTP and/or dUTP.

In some embodiments, the determining comprises contacting two of the first, second, third, or fourth concatemer with two of a polymerizing enzyme, a plurality of nucleotide conjugates, and two of a primer sequence that is complementary to a portion of the first, second, third, or fourth concatemer under conditions sufficient to form a multivalent binding complex comprising each of the two of the polymerizing enzyme, a nucleotide conjugate of the plurality of nucleotide conjugates, and each of the two of the first, second, third, or fourth concatemer hybridized to each of the two of the primer sequence. In some embodiments, the nucleotide conjugate comprises a label and at least two of a nucleotide moiety. In some embodiments, two of the at least two of the nucleotide moiety are each complementary and bind to a nucleotide of each of the two of the first concatemer. In some embodiments, the determining comprises detecting the multivalent binding complex through the label of the nucleotide conjugate. In some embodiments, the determining further comprises identifying the nucleobases of the nucleotides of the two of the first concatemer that are each complementary and bind to each of the two of the at least two of the nucleotide moiety of the nucleotide conjugate.

In some embodiments, the determining further comprises: removing the two of the polymerizing enzyme and the nucleotide conjugate from the two of the first, second, third, or fourth concatemer. In some embodiments, the determining further comprises contacting each of the two of the first, second, third, or fourth concatemer hybridized to each of the two of the primer sequence with two of a second polymerizing enzyme and a plurality of unlabeled nucleotides under conditions suitable for forming a binding complex comprising each of the two of the second polymerizing enzyme, each of the two of the first, second, third, or fourth concatemer hybridized to each of the two of the primer sequence, and two of the plurality of the unlabeled nucleotides and incorporating each of the two of the plurality of the unlabeled nucleotides into each of the two of the primer sequence. In some embodiments, each of the two of the plurality of the unlabeled nucleotides is complementary and binds to a nucleotide of each of the two of the first, second, third, or fourth concatemer. In some embodiments, an unlabeled nucleotide of the plurality of unlabeled nucleotides comprises a removable blocking group at the 3′ carbon of the sugan moiety.

In some embodiments, the determining comprises: contacting two of the first, second, third, or fourth concatemer with two of a polymerizing enzyme, a plurality of nucleotide conjugates, a first primer sequence that is complementary to a first portion of the first, second, third, or fourth concatemer, and a second primer sequence that is complementary to a second portion of the first, second, third, or fourth concatemer under conditions sufficient to form a multivalent binding complex comprising each of the two of the polymerizing enzyme, a nucleotide conjugate of the plurality of nucleotide conjugates, the first portion of the first, second, third, or fourth concatemer hybridized to the first primer sequence and a second portion of the first, second, third, or fourth concatemer hybridized to the second primer sequence. In some embodiments, the nucleotide conjugate comprises a label and at least two of a nucleotide moiety. In some embodiments, two of the at least two of the nucleotide moiety are each complementary and bind to a nucleotide of a first portion and a second portion of the first, second, third, or fourth concatemer. In some embodiments, the determining further comprises detecting the multivalent binding complex through the label of the nucleotide conjugate. In some embodiments, the determining further comprises identifying the nucleobases of the nucleotides of the first and second portions of the first, second, third, or fourth concatemer that are each complementary and bind to each of the two of the at least two of the nucleotide moiety of the nucleotide conjugate. In some embodiments, the conditions inhibit incorporation of the at least two of the nucleotide moiety of the nucleotide conjugate into the two of the first, second, third, or fourth concatemer. In some embodiments, the nucleotide conjugate comprises a core coupled to a plurality of nucleotide arms, wherein each of the nucleotide moiety is attached to one nucleotide arm of the plurality of nucleotide arms. In some embodiments, the detectable label comprises a fluorescent label. In some embodiments, the detecting comprises imaging the fluorescent label. In some embodiments, the plurality of nucleotides consists of at least two of the same type of nucleotide, wherein the same type of nucleotide is selected from the group comprising dATP, dGTP, dCTP, dTTP, and dUTP, wherein the fluorescent label of one type of nucleotide of the group emits light at a wavelength that is different from the wavelength of light emitted from the fluorescent label of another type of nucleotide of the group. In some embodiments, the plurality of nucleotides comprises at least two types of nucleotides, wherein the at least two types of nucleotides are selected from the group comprising dATP, dGTP, dCTP, dTTP, and dUTP, wherein the fluorescent label of one type of nucleotide of the group emits light at a wavelength that is different from the wavelength of light emitted from the fluorescent label of another type of nucleotide of the group. In some embodiments, the determining further comprises contacting the nucleotide with an agent to remove the blocking group from the nucleotide and generate a 3′ OH group on the sugar moiety. In some embodiments, the blocking group comprises an alkyl group, an alkenyl group, an alkynyl group, an allyl group, an aryl group, a benzyl group, an azide group, an azido group, an O-azidomethyl group, an amine group, an amide group, a keto group, an isocyanate group, a phosphate group, a thiol group, a disulfide group, a carbonate group, a urea group, or a silyl group. In some embodiments, the plurality of nucleotides consists of at least two of the same type of nucleotide, wherein the same type of nucleotide is selected from the group comprising dATP, dGTP, dCTP, dTTP, and dUTP, wherein the fluorescent label of one type of nucleotide of the group emits light at a wavelength that is different from the wavelength of light emitted from the fluorescent label of another type of nucleotide of the group. In some embodiments, the plurality of nucleotides comprises at least two types of nucleotides, wherein the at least two types of nucleotides are selected from the group comprising dATP, dGTP, dCTP, dTTP, and dUTP, wherein the fluorescent label of one type of nucleotide of the group emits light at a wavelength that is different from the wavelength of light emitted from the fluorescent label of another type of nucleotide of the group. In some embodiments, the first target RNA sequence comprises coding RNA, non-coding RNA, messenger RNA (mRNA), transfer RNA (tRNA), ribosomal RNA (rRNA), microRNA (miRNA), guide RNA (gRNA), small nuclear RNA (snRNA), small interference RNA (siRNA), anti-sense RNA, mature microRNA, or immature microRNA. In some embodiments, the second target RNA sequence comprises coding RNA, non-coding RNA, mRNA, tRNA, rRNA, miRNA, gRNA, snRNA, siRNA, anti-sense RNA, mature microRNA, or immature microRNA.

In some embodiments, the biological sample comprises a human sample, a simian sample, an ape sample, a canine sample, a feline sample, a bovine sample, an equine sample, a murine sample, a porcine sample, a caprine sample, a lupine sample, a ranine sample, a piscine sample, a plant sample, an insect sample, a bacteria sample, an algae sample, a viral sample, a protozoa sample, or a fungus sample. In some embodiments, the biological sample comprises a cellular organelle, a cell, a whole cell, a group of whole cells, a tissue, an intact tissue, a tumor, an intact tumor, an organ, or an organism. In some embodiments, the biological sample is immobilized on a surface. In some embodiments, the surface comprises an interior surface of a flow cell.

In situ Batch Sequencing RNA and Polypeptides

The present disclosure provides methods for conducting in situ multiplex and multi-omics detection and identification using coded padlocks probes. The padlock probes are designed to selectively detect target RNA or polypeptides. The padlock probe may comprise one or more batch-specific primer-binding sites, which are specific to a primer. The primer may be spatially localized, and the padlock probe may therefore not require a barcode sequence. In some embodiments, however, the padlock probe may comprise a barcode sequence.

The RNA-specific padlock probes selectively hybridize to cDNA that corresponds to target RNA. The RNA-specific probes carry barcodes that uniquely identify the cDNA. The RNA-specific padlock probes also carry batch-specific sequencing primer binding sites. The padlock probe may comprise one or more batch-specific primer-binding sites, which are specific to a primer. The primer may be spatially localized, and the padlock probe may therefore not require a barcode sequence. In some embodiments, however, the padlock probe may comprise a barcode sequence.

The target polypeptides are detected using antibody-oligonucleotide conjugates and polypeptide-specific padlock probes which selectively hybridize to the oligonucleotide which is conjugated to the antibody. The polypeptide-specific padlock probes carry barcodes that uniquely identify the antibody that selectively binds a target polypeptides. The polypeptide-specific padlock probes also carry a batch-specific sequencing primer which is the same batch-specific sequencing primer carried by a corresponding RNA-specific padlock probe to enable simultaneous sequencing and detection of a target RNA and the polypeptide encoded by that target RNA. Thus, the padlock probes enable simultaneous detection and identification of RNA and their encoded polypeptides.

Both types of padlock probes are used to generate concatemers which having multiple copies of batch-specific sequencing binding sites and barcodes. The concatemers can collapse into DNA nanoballs having compact shape and size that produce increased signal intensity and color differentiation during sequencing.

For in situ sequencing, the limit of optical resolution impedes the ability to perform highly multiplex sequencing. The batch-specific sequencing primer binding sites on the padlock probes enables sequencing a desired subset (e.g., a batch) of the concatemers using selected batch-specific sequencing primers to reduce over-crowding signals and images. The use of batch-specific sequencing primers produces optical images that are intense and resolvable. By conducting multiple rounds of sequencing on the same cellular sample using different batch-specific sequencing primers enables multiplex and multi-omics sequencing to reveal numerous target RNAs and their encoded polypeptides.

The batch-specific sequencing methods described herein have many uses. For example, the number of spots that are imaged and associated with sequencing can be counted. The counted spots can be used as a measure or RNA and polypeptide levels in a cellular sample.

For example, a pairwise sequencing kit that can be used to conduct 150 forward sequencing cycles and 150 reverse sequencing cycles (e.g., a total 300 sequencing cycles per kit) can be used to reveal more than 300,000 molecules. A non-limiting example cell has approximately 30 copies of 5,000 different transcripts for a total of approximately 150,000 transcripts, and this call has approximately 15,000 polypeptides encoded by the transcripts for a total of 300,000 total RNA and polypeptide molecules. The pairwise kit and be used to conduct 300 sequencing cycles (not in pairwise mode) to reveal the 300,000 different RNA and polypeptide molecules in the cellular sample. See Table 1. Table 1 Table 1 lists the estimated total sequencing cycles and total time to decode RNA and polypeptides in a cellular sample.

TABLE 1
Estimated total sequencing cycles and total time to decode RNA and polypeptides in a cellular sample

| | B: | C: | | E: | | | | | | K: |
|---|---|---|---|---|---|---|---|---|---|---|
| A: | Cell | Polonies | | Copy # | | G: | H: | I: | J: | Total |
| Resl'n | Vol. | per cell | D: | per | F: | Copy#/ | Total | Decod. | Total | time |
| (um) | (um²) | (4 color) | Transc. | transc. | Prtns | prtn | molecls | cycles | cycles | (hrs) |
| 0.4 | 1000 | 10000 | 100 | 30 | 100 | 30 | 6000 | 10 | 0.13 | 0.01516 |
| 0.4 | 1000 | 10000 | 200 | 30 | 200 | 30 | 12000 | 10 | 12 | 1.4 |
| 0.4 | 1000 | 10000 | 500 | 30 | 500 | 30 | 30000 | 10 | 30 | 3.5 |
| 0.4 | 1000 | 10000 | 1000 | 30 | 1000 | 30 | 60000 | 10 | 60 | 7 |
| 0.4 | 1000 | 10000 | 2000 | 30 | 2000 | 30 | 120000 | 10 | 120 | 14 |
| 0.4 | 1000 | 10000 | 5000 | 30 | 5000 | 30 | 300000 | 10 | 300 | 35 |
| 0.4 | 1000 | 10000 | 10000 | 30 | 10000 | 30 | 600000 | 10 | 600 | 70 |

Column A: resolution (um)
Column B: Cell volume (um²)
Column C: resolved polonies/cell (4-color)
Column D: transcripts
Column E: copy number/transcript
Column F: proteins
Column G: copy #/protein
Column H: total molecules
Column I: Decoding cycles
Column J: Total cycles to decode all molecules
Column K: Total time to decode (hrs)

The present disclosure provides methods for detecting in situ at least two different target RNA molecules and two different polypeptides encoded by the at least two different target RNA molecules, comprising step (a): providing a cellular sample deposited on a solid support, wherein the cellular sample harbors (i) a first plurality of DNA amplicons that correspond to a first target RNA molecule, (ii) a second plurality of DNA amplicons that correspond to a second target RNA molecule, (iii) a third plurality of DNA amplicons that correspond to a first polypeptide which is encoded by the first target RNA molecule, and (iv) a fourth plurality of DNA amplicons that correspond to a second polypeptide which is encoded by the second target RNA molecule.

In some embodiments, the method further comprises step (b): sequencing the first and third plurality of DNA amplicons inside the cellular sample under a condition that inhibits sequencing the second and fourth plurality of DNA amplicons, wherein sequencing the first plurality of DNA amplicons inside the cellular sample comprises generating a plurality of first sequencing read products, wherein the sequences of the first sequencing read products are aligned with a first target reference sequence to confirm the presence of the first target RNA in the cellular sample, and wherein sequencing the third plurality of DNA amplicons inside the cellular sample comprises generating a plurality of second sequencing read products, wherein the sequences of the second sequencing read products are aligned with a second target reference sequence to confirm the presence of the first target polypeptides in the cellular sample.

In some embodiments, the method further comprises step (c): sequencing the second and fourth plurality of DNA amplicons inside the cellular sample under a condition that inhibits sequencing the first and third plurality of DNA amplicons, wherein sequencing the second plurality of DNA amplicons inside the cellular sample comprises generating a plurality of third sequencing read products, wherein the sequences of the third sequencing read products are aligned with a third target reference sequence to confirm the presence of the second target RNA in the cellular sample, and wherein sequencing the fourth plurality of DNA amplicons inside the cellular sample comprises generating a plurality of fourth sequencing read products, wherein the sequences of the fourth sequencing read products are aligned with a fourth target reference sequence to confirm the presence of the second target polypeptides in the cellular sample.

The present disclosure provides methods for detecting in situ at least two different target RNA molecules and two different polypeptides encoded by the at least two different target RNA molecules, comprising step (a): providing a cellular sample deposited on a solid support, wherein the cellular sample harbors (i) a first plurality of DNA amplicons that correspond to a first target RNA molecule, (ii) a second plurality of DNA amplicons that correspond to a second target RNA molecule, (iii) a third plurality of DNA amplicons that correspond to a first polypeptide which is encoded by the first target RNA molecule, and (iv) a fourth plurality of DNA amplicons that correspond to a second polypeptide which is encoded by the second target RNA molecule.

In some embodiments, the methods further comprise step (b): sequencing the first and third plurality of DNA amplicons inside the cellular sample under a condition that inhibits sequencing the second and fourth plurality of DNA amplicons. In some embodiments, step (b) comprises sequencing the first plurality of DNA amplicons inside the cellular sample comprises conducting no more than 2-30 sequencing cycles to generate a plurality of first sequencing read products, wherein the sequences of the first sequencing read products are aligned with a first target reference sequence to confirm the presence of the first target RNA in the cellular sample.

In some embodiments, step (b) comprises sequencing the first plurality of DNA amplicons inside the cellular sample comprises conducting 1-250 sequencing cycles to generate a plurality of first sequencing read products, wherein the sequences of the first sequencing read products are aligned with a first target reference sequence to confirm the presence of the first target RNA in the cellular sample. In some embodiments, step (b) comprises sequencing the third plurality of DNA amplicons inside the cellular sample comprises conducting no more than 2-30 sequencing cycles to generate a plurality of second sequencing read products, wherein the sequences of the second sequencing read products are aligned with a second target reference sequence to confirm the presence of the first target polypeptide in the cellular sample. In some embodiments, step (b) comprises sequencing the third plurality of DNA amplicons inside the cellular sample comprises conducting 1-250 sequencing cycles to generate a plurality of second sequencing read products, wherein the sequences of the second sequencing read products are aligned with a second target reference sequence to confirm the presence of the first target polypeptide in the cellular sample.

In some embodiments, the methods further comprise step (c): removing the plurality of first sequencing read products from the first DNA amplicons and retaining the first DNA amplicons inside the cellular sample, and removing the plurality of second sequencing read products from the third DNA amplicons and retaining the third DNA amplicons inside the cellular sample. In some embodiments, a 3′ blocking moiety can be added to the first and second sequencing read products to inhibit further sequencing reactions. For example, a nucleotide analog can be incorporated where the nucleotide analog inhibits incorporation of a subsequent nucleotide. Non-limiting example blocking nucleotide analogs include dideoxynucleotide or a nucleotide having a 2′ or 3′ chain terminating moiety.

In some embodiments, the methods further comprise step (d): reiteratively sequencing the first and third plurality of DNA amplicons by repeating steps (b) and (c) at least once. In some embodiments, the repeating comprises repeating at least 2 times, 5 times, 10 times, 15 times, 20 times, 25 times, 30 times, 35 times, 40 times, 45 times, or at least 50 times.

In some embodiments, the methods further comprise step (e): sequencing the second and fourth plurality of DNA amplicons inside the cellular sample under a condition that inhibits sequencing the first and third plurality of DNA amplicons. In some embodiments, step (e) comprises sequencing the second plurality of DNA amplicons inside the cellular sample comprises conducting no more than 2-30 sequencing cycles to generate a plurality of third sequencing read products, wherein the sequences of the third sequencing read products are aligned with a third target reference sequence to confirm the presence of the second target RNA in the cellular sample. In some embodiments, step (e) comprises sequencing the second plurality of DNA amplicons inside the cellular sample comprises conducting 1-250 sequencing cycles to generate a plurality of third sequencing read products, wherein the sequences of the third sequencing read products are aligned with a third target reference sequence to confirm the presence of the second target RNA in the cellular sample. In some embodiments, step (e) comprises sequencing the fourth plurality of DNA amplicons inside the cellular sample comprises conducting no more than 2-30 sequencing cycles to generate a plurality of fourth sequencing read products, wherein the sequences of the fourth sequencing read products are aligned with a fourth target reference sequence to confirm the presence of the second target polypeptide in the cellular sample. In some embodiments, step (e) comprises sequencing the fourth plurality of DNA amplicons inside the cellular sample comprises conducting 1-250 sequencing cycles to generate a plurality of fourth sequencing read products, wherein the sequences of the fourth sequencing read products are aligned with a fourth target reference sequence to confirm the presence of the second target polypeptide in the cellular sample.

In some embodiments, the methods comprise step (f): removing the plurality of third sequencing read products from the second DNA amplicons and retaining the second DNA amplicons inside the cellular sample, and removing the plurality of fourth sequencing read products from the fourth DNA amplicons and retaining the fourth DNA amplicons inside the cellular sample. In some embodiments, a 3′ blocking moiety can be added to the third and fourth sequencing read products to inhibit further sequencing reactions. For example, a nucleotide analog can be incorporated where the nucleotide analog inhibits incorporation of a subsequent nucleotide. Non-limiting example blocking nucleotide analogs include dideoxynucleotide or a nucleotide having a 2′ or 3′ chain terminating moiety.

In some embodiments, the methods comprise step (g): reiteratively sequencing the second and fourth plurality of DNA amplicons by repeating steps (e) and (f) at least once. In some embodiments, the repeating comprises repeating at least 2 times, 5 times, 10 times, 15 times, 20 times, 25 times, 30 times, 35 times, 40 times, 45 times, or at least 50 times.

The present disclosure provides methods for detecting in situ at least two different target RNA molecules and two different polypeptides encoded by the at least two different target RNA molecules, comprising step (a): providing a cellular sample deposited on a solid support, wherein the cellular sample is fixed and permeabilized, wherein the cellular sample harbors a first plurality of target RNA and a first plurality of target polypeptides which are encoded by the first plurality of target RNA, and the cellular sample harbors a second plurality of target RNA and a second plurality of target polypeptides which are encoded by the second plurality of target RNA.

In some embodiments, the cellular sample harbors 2-25 different target RNA molecules, or harbors 25-50 different target RNA molecules, or harbors 50-75 different target RNA molecules, or harbors 75-100 different target RNA molecules. In some embodiments, the cellular sample harbors more than 100 different target RNA molecules, or more than 250 different target RNA molecules, or more than 500 different target molecules, or more than 1000 different target RNA molecules, or more. In some embodiments, the cellular sample harbors more than 10,000 different target RNA molecules. In some embodiments, the cellular sample comprises a whole cell, a plurality of whole cells, an intact tissue or an intact tumor. In some embodiments, the cellular sample comprises a fresh cellular sample, a freshly-frozen cellular sample, a sectioned cellular sample, or an FFPE cellular sample. In some embodiments, the cellular sample is deposited onto a solid support. In some embodiments, the cellular sample is deposited onto a solid support which is passivated with a coating that promotes cell adhesion. In some embodiments, the cellular sample is deposited on a support that lacks immobilized capture oligonucleotides. In some embodiments, the cellular sample is cultured prior to conducting step (b) which is described below.

In some embodiments, the cellular sample harbors 2-25 different target polypeptide molecules, or harbors 25-50 different target polypeptide molecules, or harbors 50-75 different target polypeptide molecules, or harbors 75-100 different target polypeptide molecules. In some embodiments, the cellular sample harbors more than 100 different target polypeptide molecules, or more than 250 different target polypeptide molecules, or more than 500 different target molecules, or more than 1000 different target polypeptide molecules, or more. In some embodiments, the cellular sample harbors more than 10,000 different target polypeptide molecules. The target polypeptide molecules are encoded by the target RNA molecules.

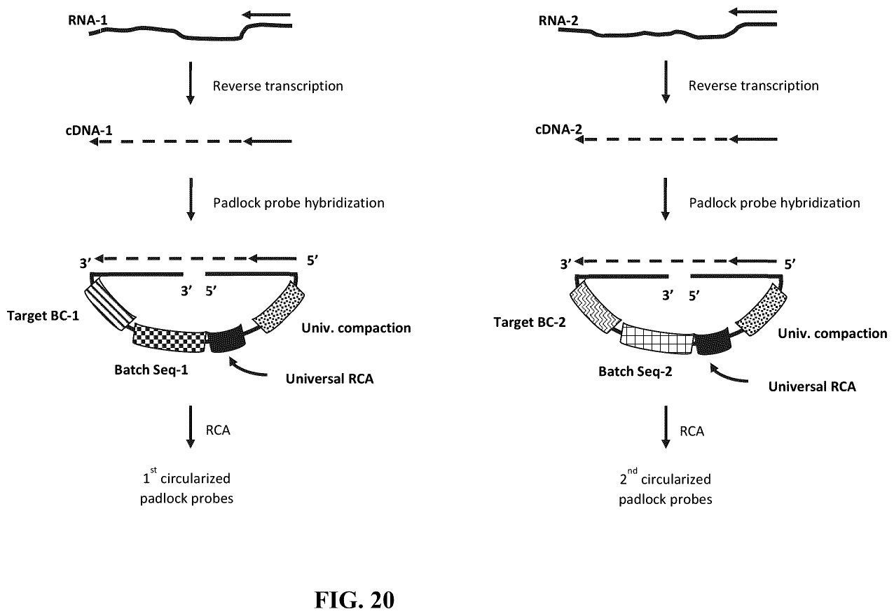

In some embodiments, the methods comprise step (b): generating inside the cellular sample a plurality of cDNA by (i) generating at least a first plurality of target cDNA from the first plurality of target RNA, and (ii) generating at least a second plurality of target cDNA from the second plurality of target RNA (e.g., FIG. 20). In some embodiments, the first target cDNAs correspond to the first target RNA molecules. In some embodiments, the second target cDNAs correspond to the second target RNA molecules. In some embodiments, the method comprises generating at least 2-10,000 different target cDNA molecules that correspond to 2-10,000 different target RNA molecules. In some embodiments, the generating of step (b) comprises contacting the plurality of RNA inside the cellular sample with (i) a plurality of reverse transcription primers, (ii) a plurality of reverse transcriptase enzymes, and (iii) a plurality of nucleotides, under a condition suitable for conducting a reverse transcription reaction to generate a plurality of cDNA molecules (e.g., a plurality of first strand cDNA molecules) in the cellular sample. In some embodiments, the plurality of reverse transcription primers comprises a first sub-population of target-specific reverse transcription primers that hybridize selectively to the first target RNA, and comprises a second sub-population of target-specific reverse transcription primers that hybridize selectively to the second target RNA. In some embodiments, the plurality of reverse transcription primers comprises a first sub-population of random-sequence reverse transcription primers that hybridize to the first target RNA, and comprises a second sub-population of random-sequence reverse transcription primers that hybridize to the second target RNA.

In some embodiments, the methods comprise step (c): generating inside the cellular sample a plurality of DNA concatemers which correspond to the first and second plurality of target RNA molecules, comprising: (1) generating a first plurality of covalently closed circular padlock probes by contacting the first plurality of target cDNA with a first plurality of padlock probes, wherein the contacting is conducted under a condition suitable for hybridizing the first and second binding arms of the first padlock probes to proximal positions on their respective first target cDNA molecules to form a first plurality of circularized padlock probes each having a nick or gap between the hybridized first and second binding arms, wherein the first padlock probes include a (i) a first target barcode sequence (target BC-1) that uniquely identifies the first target RNA, (ii) a first batch-specific sequencing primer binding site (Batch Seq-1) (or a complementary sequence thereof), and (iii) a universal binding site for an amplification primer (universal RCA) (or a complementary sequence thereof) (e.g., FIG. 20, left side); (2) enzymatically closing the nick or gap in the first plurality of covalently closed circular padlock probes to form a first plurality of covalently closed padlock probes; and (3) conducting rolling circle amplification inside the cellular sample using the first covalently closed circular padlock probes as template molecules, thereby generating a first plurality of concatemer molecules that correspond to the first plurality of target RNA molecules. In some embodiments, the rolling circle amplification reaction can be conducted in the presence or absence of a plurality of compaction oligonucleotides. In some embodiments, the method comprises contacting the plurality of cDNA molecule in the cellular sample with at least 2-10,000 different target-specific padlock probes. In some embodiments, the first padlock probe further comprises a universal compaction oligonucleotide binding site (or a complementary sequence thereof). In some embodiments, the closing the nick in the first circularized padlock probes comprises conducting an enzymatic ligation reaction. In some embodiments, closing the gap in the first circularized padlock probes comprises conducting a polymerase-catalyzed fill-in reaction using the first target cDNA molecule as a template, and conducting an enzymatic ligation reaction. In some embodiments, the method comprises closing the nick or gap in at least 2-10,000 circularized target-specific padlock probes by conducting an enzymatic reaction, thereby generating at least 2-10,000 covalently closed circular padlock probes inside the cellular sample. In some embodiments, each concatemer molecule in the first plurality comprises tandem repeat units, wherein a unit comprises the sequence of the first target cDNA and (i) the first target barcode sequence (target BC-1) that uniquely identifies the first target RNA, (ii) the first batch-specific sequencing primer binding site (Batch Seq-1) (or a complementary sequence thereof), and (iii) the universal binding site for an amplification primer (universal RCA) (or a complementary sequence thereof). In some embodiments, the unit further comprises the universal compaction oligonucleotide binding site (or a complementary sequence thereof).

In some embodiments, step (c) further comprises: (1) generating a second plurality of covalently closed circular padlock probes by contacting the second plurality of target cDNA with a second plurality of padlock probes, wherein the contacting is conducted under a condition suitable for hybridizing the first and second binding arms of the second padlock probes to proximal positions on their respective second target cDNA molecules to form a second plurality of circularized padlock probes each having a nick or gap between the hybridized first and second binding arms, wherein the second padlock probes include a (i) a second barcode sequence (target BC-2) that uniquely identifies the second target RNA, (ii) a second batch-specific sequencing primer binding site (Batch Seq-2) (or a complementary sequence thereof) wherein the sequence of the second batch-specific sequencing primer binding site differs from the sequence of the first batch-specific sequencing primer binding site, and (iii) the universal binding site for an amplification primer (universal RCA) (or a complementary sequence thereof) (e.g., FIG. 20, right side); (2) enzymatically closing the nick or gap in the second plurality of covalently closed circular padlock probes to form a second plurality of covalently closed padlock probes; and (3) conducting rolling circle amplification inside the cellular sample using the second covalently closed circular padlock probes as template molecules, thereby generating a second plurality of concatemer molecules that correspond to the second plurality of target RNA molecules. In some embodiments, the rolling circle amplification reaction can be conducted in the presence or absence of a plurality of compaction oligonucleotides. In some embodiments, the method comprises contacting the plurality of cDNA molecule in the cellular sample with at least 2-10,000 different target-specific padlock probes. In some embodiments, the second padlock probe further comprises a universal compaction oligonucleotide binding site (or a complementary sequence thereof). In some embodiments, the closing the nick in the second circularized padlock probes comprises conducting an enzymatic ligation reaction. In some embodiments, closing the gap in the second circularized padlock probes comprises conducting a polymerase-catalyzed fill-in reaction using the second target cDNA molecule as a template, and conducting an enzymatic ligation reaction. In some embodiments, the method comprises closing the nick or gap in at least 2-10,000 circularized target-specific padlock probes by conducting an enzymatic reaction, thereby generating at least 2-10,000 covalently closed circular padlock probes inside the cellular sample. In some embodiments, each concatemer molecule in the second plurality comprises tandem repeat units, wherein a unit comprises the sequence of the second target cDNA and (i) the second target barcode sequence (target BC-2) that uniquely identifies the second target RNA, (ii) the second batch-specific sequencing primer binding site (Batch Seq-2) (or a complementary sequence thereof), and (iii) the universal binding site for an amplification primer (universal RCA) (or a complementary sequence thereof). In some embodiments, the unit further comprises the universal compaction oligonucleotide binding site (or a complementary sequence thereof).

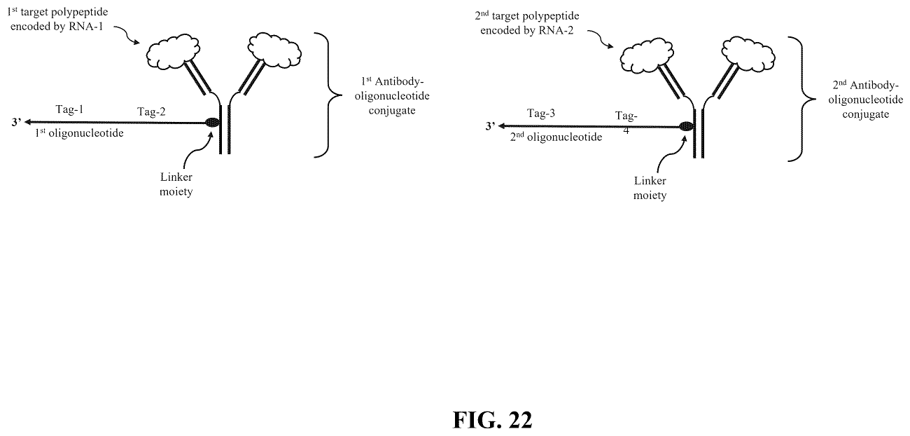

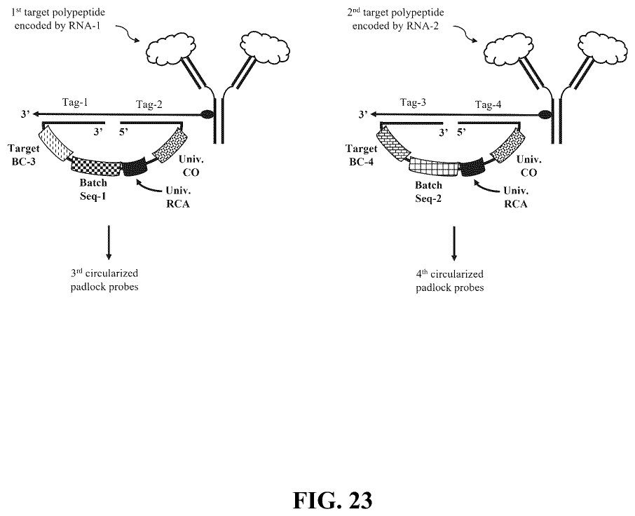

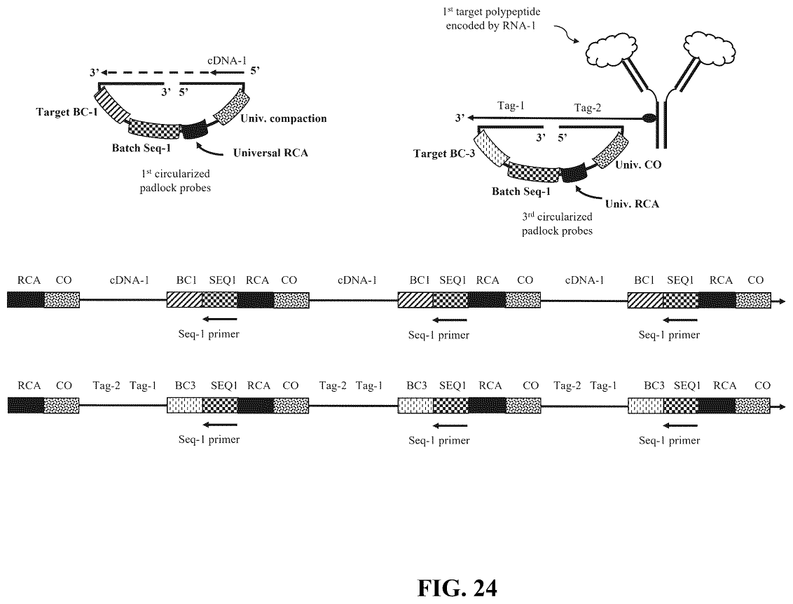

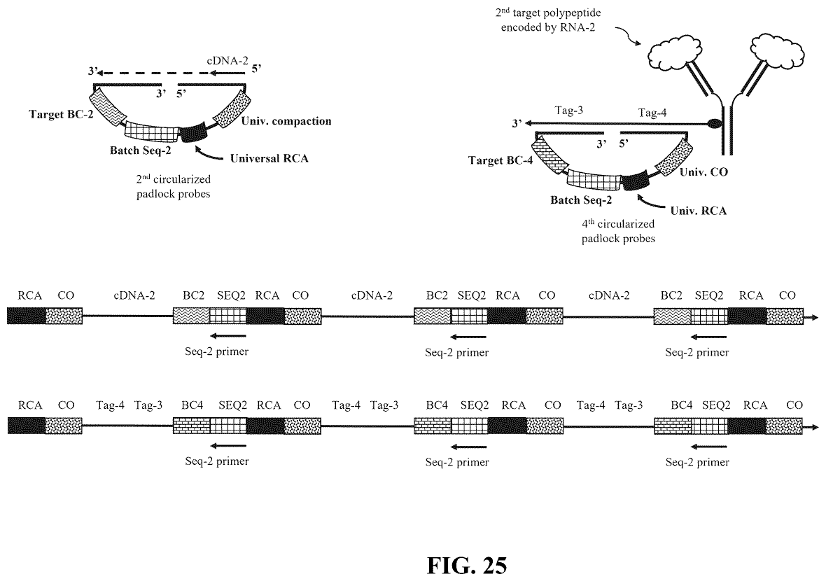

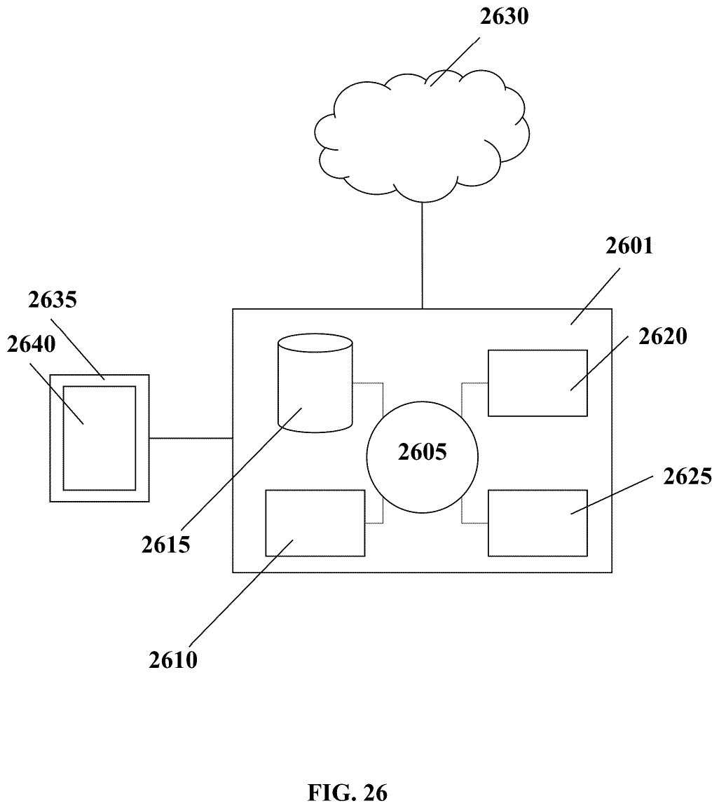

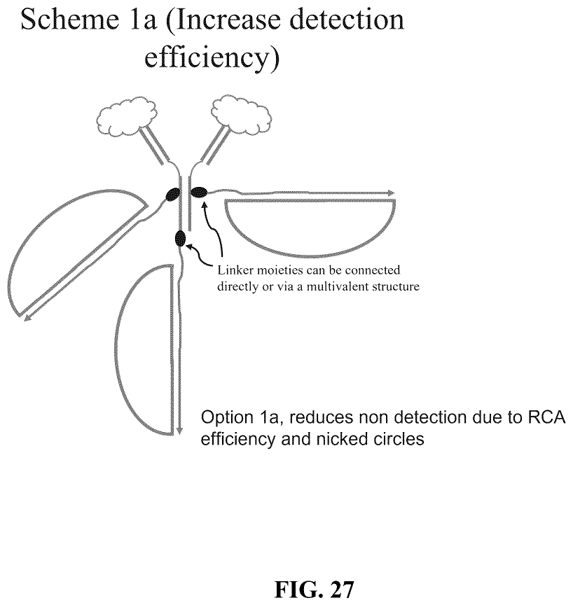

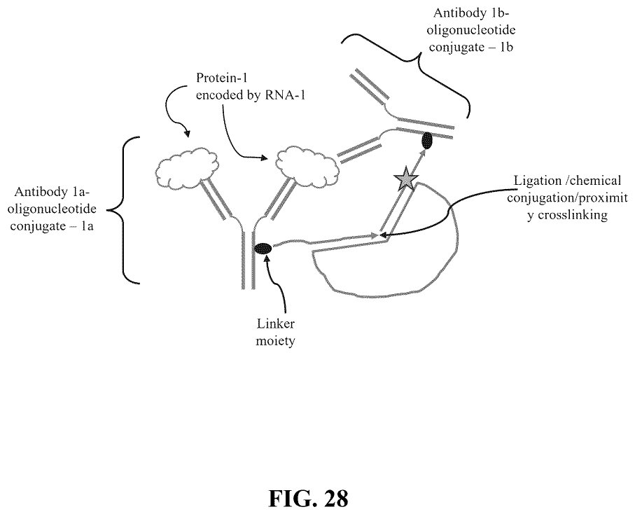

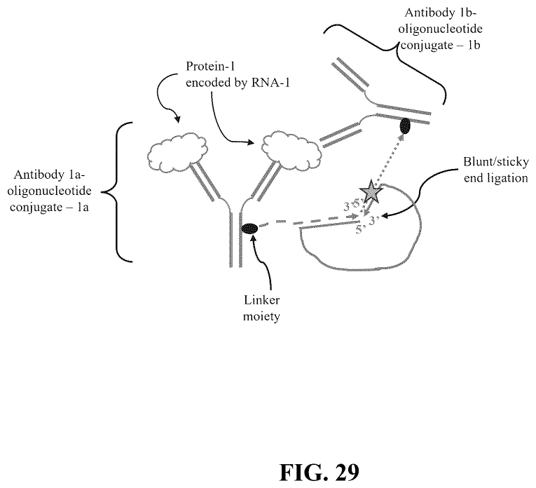

In some embodiments, the method further comprise step (d): generating inside the cellular sample a plurality of DNA concatemers which correspond to the first and second plurality of target polypeptide molecules, comprising: (1) generating a third plurality of covalently closed circular padlock probes by contacting the first plurality of target polypeptides with a first plurality of antibody-oligonucleotide conjugate molecules each comprising a first antibody linked to a first oligonucleotide carrying a first tag sequence (Tag-1) and a second tag sequence (Tag-2) wherein the first and second tag sequences uniquely identify the first antibodies that selective bind the first target polypeptides (e.g., FIG. 22, left side), wherein the contacting is conducted under a condition suitable for selectively binding the first antibody-oligonucleotide conjugate molecules to the first target polypeptides; (2) contacting the first oligonucleotides (of the first antibody-oligonucleotide conjugate molecules) with a third plurality of padlock probes, wherein the contacting is conducted under a condition suitable for hybridizing the first and second binding arms of the third padlock probes to proximal positions on their respective first oligonucleotides to form a third plurality of circularized padlock probes each having a nick or gap between the hybridized first and second binding arms, wherein the third padlock probes include a (i) a sequence that binds the first tag sequence of the first oligonucleotide, (ii) a third target barcode sequence (target BC-3) that uniquely identifies the first antibody-oligonucleotide conjugate which selectively binds the first target polypeptide, (iii) a first batch-specific sequencing primer binding site (Batch Seq-1) (or a complementary sequence thereof), (iv) a universal binding site for an amplification primer (universal RCA) (or a complementary sequence thereof), and (v) a sequence that bind the second tag sequence of the first oligonucleotide (e.g., FIG. 23, left side); (3) enzymatically closing the nick or gap in the third plurality of covalently closed circular padlock probes to form a third plurality of covalently closed padlock probes; and (4) conducting rolling circle amplification inside the cellular sample using the third covalently closed circular padlock probes as template molecules, thereby generating a third plurality of concatemer molecules that correspond to the first plurality of target polypeptides. In some embodiments, the rolling circle amplification reaction can be conducted in the presence or absence of a plurality of compaction oligonucleotides. In some embodiments, the third padlock probe further comprises a universal compaction oligonucleotide binding site (or a complementary sequence thereof). In some embodiments, the closing the nick in the third circularized padlock probes comprises conducting an enzymatic ligation reaction. In some embodiments, closing the gap in the third circularized padlock probes comprises conducting a polymerase-catalyzed fill-in reaction using the first oligonucleotide as a template, and conducting an enzymatic ligation reaction. In some embodiments, the method comprises closing the nick or gap in at least 2-10,000 circularized padlock probes by conducting an enzymatic reaction, thereby generating at least 2-10,000 covalently closed circular padlock probes inside the cellular sample. In some embodiments, each concatemer molecule in the third plurality comprises tandem repeat units, wherein a unit comprises (i) the sequence that binds the first tag sequence of the first oligonucleotide, (ii) the third target barcode sequence (target BC-3) that uniquely identifies the first antibody-oligonucleotide conjugate which selectively binds the first target polypeptide, (iii) the first batch-specific sequencing primer binding site (Batch Seq-1) (or a complementary sequence thereof), (iv) the universal binding site for an amplification primer (universal RCA) (or a complementary sequence thereof), and (v) the sequence that bind the second tag sequence of the first oligonucleotide. In some embodiments, the unit further comprises the universal compaction oligonucleotide binding site (or a complementary sequence thereof).

In some embodiments, step (d) further comprises: (1) generating a fourth plurality of covalently closed circular padlock probes by contacting the second plurality of target polypeptides with a second plurality of antibody-oligonucleotide conjugate molecules each comprising a second antibody linked to a second oligonucleotide carrying a third tag sequence (Tag-3) and a fourth tag sequence (Tag-4) wherein the third and fourth tag sequences uniquely identify the second antibodies that selective bind the second target polypeptides (e.g., FIG. 22, right side), wherein the contacting is conducted under a condition suitable for selectively binding the second antibody-oligonucleotide conjugate molecules to the second target polypeptides; (2) contacting the second oligonucleotides (of the second antibody-oligonucleotide conjugate molecules) with a fourth plurality of padlock probes, wherein the contacting is conducted under a condition suitable for hybridizing the first and second binding arms of the fourth padlock probes to proximal positions on their respective second oligonucleotides to form a fourth plurality of circularized padlock probes each having a nick or gap between the hybridized first and second binding arms, wherein the fourth padlock probes include a (i) sequence that binds the third tag sequence of the second oligonucleotide, (ii) a fourth target barcode sequence (target BC-4) that uniquely identifies the second antibody-oligonucleotide conjugate which selectively binds the second target polypeptide, (iii) a second batch-specific sequencing primer binding site (Batch Seq-2) (or a complementary sequence thereof), (iv) a universal binding site for an amplification primer (universal RCA) (or a complementary sequence thereof), and (v) a sequence that binds the fourth tag sequence of the second oligonucleotide (e.g., FIG. 23, right side); (3) enzymatically closing the nick or gap in the fourth plurality of covalently closed circular padlock probes to form a fourth plurality of covalently closed padlock probes; and (4) conducting rolling circle amplification inside the cellular sample using the fourth covalently closed circular padlock probes as template molecules, thereby generating a fourth plurality of concatemer molecules that correspond to the second plurality of target polypeptides. In some embodiments, the rolling circle amplification reaction can be conducted in the presence or absence of a plurality of compaction oligonucleotides. In some embodiments, the fourth padlock probe further comprises a universal compaction oligonucleotide binding site (or a complementary sequence thereof). In some embodiments, the closing the nick in the fourth circularized padlock probes comprises conducting an enzymatic ligation reaction. In some embodiments, closing the gap in the fourth circularized padlock probes comprises conducting a polymerase-catalyzed fill-in reaction using the second oligonucleotide as a template, and conducting an enzymatic ligation reaction. In some embodiments, the method comprises closing the nick or gap in at least 2-10,000 circularized padlock probes by conducting an enzymatic reaction, thereby generating at least 2-10,000 covalently closed circular padlock probes inside the cellular sample. In some embodiments, each concatemer molecule in the fourth plurality comprises tandem repeat units, wherein a unit comprises (i) the sequence that binds the third tag sequence of the second oligonucleotide, (ii) the fourth target barcode sequence (target BC-4) that uniquely identifies the second antibody-oligonucleotide conjugate which selectively binds the second target polypeptide, (iii) the second batch-specific sequencing primer binding site (Batch Seq-2) (or a complementary sequence thereof), (iv) the universal binding site for an amplification primer (universal RCA) (or a complementary sequence thereof), and (v) the sequence that bind the fourth tag sequence of the second oligonucleotide. In some embodiments, the unit further comprises the universal compaction oligonucleotide binding site (or a complementary sequence thereof).

In some embodiments, the methods further comprise step (e): sequencing the first and third plurality of concatemer molecules inside the cellular sample under a condition that inhibits sequencing the second and fourth plurality of concatemers (e.g., FIG. 24). In some embodiments, step (e) comprises sequencing the first plurality of concatemers inside the cellular sample comprises conducting no more than 2-30 sequencing cycles to generate a plurality of first sequencing read products, wherein the sequences of the first sequencing read products are aligned with a first target reference sequence to confirm the presence of the first target RNA in the cellular sample. In some embodiments, step (e) comprises sequencing the first plurality of concatemers inside the cellular sample comprises conducting 1-250 sequencing cycles to generate a plurality of first sequencing read products, wherein the sequences of the first sequencing read products are aligned with a first target reference sequence to confirm the presence of the first target RNA in the cellular sample. In some embodiments, step (e) comprises sequencing the third plurality of concatemers inside the cellular sample comprises conducting no more than 2-30 sequencing cycles to generate a plurality of second sequencing read products, wherein the sequences of the second sequencing read products are aligned with a second target reference sequence to confirm the presence of the first target polypeptides in the cellular sample. In some embodiments, step (e) comprises sequencing the third plurality of concatemers inside the cellular sample comprises conducting 1-250 sequencing cycles to generate a plurality of second sequencing read products, wherein the sequences of the second sequencing read products are aligned with a second target reference sequence to confirm the presence of the first target polypeptides in the cellular sample.

In some embodiments in step (e), in the first concatemer molecules, only the first target barcode region (target BC-1) is sequenced. In some embodiments, in the first concatemer molecules, at least a portion or the full length of the first target barcode (target BC-1) is sequenced. In some embodiments, in the first concatemer molecules, the first target barcode (target BC-1) is sequenced and a portion of the first cDNA region is sequenced.

In some embodiments in step (e), in the third concatemer molecules, only the third target barcode region (target BC-3) is sequenced. In some embodiments, in the third concatemer molecules, at least a portion or the full length of the third target barcode (target BC-3) is sequenced. In some embodiments, in the third concatemer molecules, the third target barcode (target BC-3) is sequenced and a portion of the first oligonucleotide is sequenced.

In some embodiments, the sequencing of step (e) comprises sequencing at least a portion of the first and third nucleic acid concatemers using an optical imaging system comprising a field-of-view (FOV) greater than 1.0 mm².

In some embodiments, in the sequencing of step (e), the plurality of first and second sequencing read products are detectable by imaging, and wherein the sequencing comprises decoding the plurality of first and second sequencing read products from the images obtained during the no more than 2-30 sequencing cycles, or from the images obtained during the 1-250 sequence cycles.

In some embodiments, in the sequencing of step (e), the plurality of the first and second sequencing read products are detectable by imaging, and wherein the sequencing comprises simultaneously imaging the plurality of first and second detectable sequencing read products in the cellular sample (co-localization of the first and second sequencing read products).

In some embodiments, the methods further comprise step (f): removing the plurality of first sequencing read products from the first concatemer molecules and retaining the first concatemer molecules inside the cellular sample, and removing the plurality of second sequencing read products from the third concatemer molecules and retaining the third concatemer molecules inside the cellular sample. In some embodiments, a 3′ blocking moiety can be added to the first and second sequencing read products to inhibit further sequencing reactions. For example, a nucleotide analog can be incorporated where the nucleotide analog inhibits incorporation of a subsequent nucleotide. Non-limiting example blocking nucleotide analogs include dideoxynucleotide or a nucleotide having a 2′ or 3′ chain terminating moiety.

In some embodiments, the methods further comprise step (g): reiteratively sequencing the plurality of first and third concatemers by repeating steps (e) and (f) at least once. In some embodiments, the repeating comprises repeating at least 2 times, 5 times, 10 times, 15 times, 20 times, 25 times, 30 times, 35 times, 40 times, 45 times, or at least 50 times.

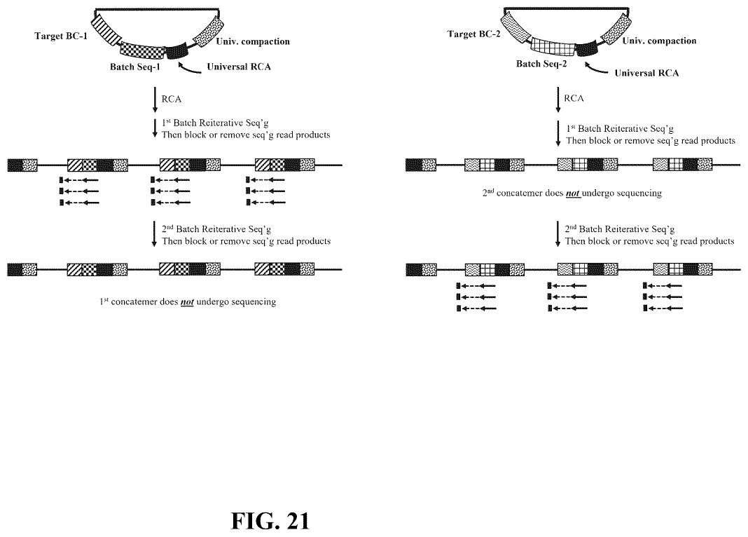

In some embodiments, the sequencing the first concatemers of step (g) comprises step (1) contacting the first plurality of concatemer molecules inside the cellular sample with (i) a plurality of first batch-specific sequencing primers, (ii) a plurality of sequencing polymerases, and (iii) a plurality of nucleotide reagents, under a condition suitable for hybridizing the plurality of first batch-specific sequencing primers to their respective first batch-specific sequencing primer binding sites on the first concatemers. In some embodiments, the sequencing further comprises step (2) conducting no more than 2-30 sequencing cycles to generate a first plurality of sequencing read products. In some embodiments, the sequencing further comprises step (3) removing the first plurality of sequencing read products from the concatemers and retaining the plurality of concatemers inside the cellular sample. In some embodiments, the sequencing further comprises step (4) repeating steps (1) -(3) at least once (e.g., FIG. 21). In some embodiments, step (4) comprises repeating steps (1) -(3) at least 2 times, at least 3 times, at least 4 times, at least 5 times, at least 6 times, at least 7 times, at least 8 times, at least 9 times, or at least 10 times. In some embodiments, step (4) comprises repeating steps (1) -(3) up to 10 times, up to 20 times, up to 30 time, up to 40 times, or up to 50 times.

In some embodiments, the reiterative sequencing of the first concatemers of step (g) can be conducting using a sequencing-by-binding procedure, labeled and/or non-labeled chain-terminating nucleotides, or multivalent molecules. Descriptions of these three sequencing methods is described below.

In some embodiments, the sequencing the third concatemers of step (g) comprises step (1) contacting the third plurality of concatemer molecules inside the cellular sample with (i) a plurality of third batch-specific sequencing primers, (ii) a plurality of sequencing polymerases, and (iii) a plurality of nucleotide reagents, under a condition suitable for hybridizing the plurality of third batch-specific sequencing primers to their respective third batch-specific sequencing primer binding sites on the third concatemers. In some embodiments, the sequencing further comprises step (2) conducting no more than 2-30 sequencing cycles to generate a second plurality of sequencing read products. In some embodiments, the sequencing further comprises step (3) removing the second plurality of sequencing read products from the concatemers and retaining the plurality of concatemers inside the cellular sample. In some embodiments, the sequencing further comprises step (4) repeating steps (1) -(3) at least once. In some embodiments, step (4) comprises repeating steps (1) -(3) at least 2 times, at least 3 times, at least 4 times, at least 5 times, at least 6 times, at least 7 times, at least 8 times, at least 9 times, or at least 10 times. In some embodiments, step (4) comprises repeating steps (1) -(3) up to 10 times, up to 20 times, up to 30 time, up to 40 times, or up to 50 times.

In some embodiments, the reiterative sequencing of the third concatemers of step (g) can be conducting using a sequencing-by-binding procedure, labeled and/or non-labeled chain-terminating nucleotides, or multivalent molecules. Descriptions of these three sequencing methods is described below.

In some embodiments, the plurality of universal sequencing primers can be hybridized to concatemer template molecules with a hybridization reagent comprising an SSC buffer (e.g., 2× saline-sodium citrate) buffer with formamide (e.g., 10-20% formamide). The hybridization conditions comprise a temperature of about 20-30° C., for about 10-60 minutes.

In some embodiments, the plurality of sequencing read products can be removed from the concatemers and the plurality of concatemers can be retained inside the cellular sample using a de-hybridization reagent comprising an SSC buffer (e.g., saline-sodium citrate) buffer, with or without formamide, at a temperature that promotes nucleic acid denaturation such as for example 30-90° C.

In some embodiments, the methods further comprise step (h): sequencing the second and fourth plurality of concatemer molecules inside the cellular sample under a condition that inhibits sequencing the first and third plurality of concatemers (e.g., FIG. 25). In some embodiments, step (h) comprises sequencing the second plurality of concatemers inside the cellular sample comprises conducting no more than 2-30 sequencing cycles to generate a plurality of third sequencing read products, wherein the sequences of the third sequencing read products are aligned with a third target reference sequence to confirm the presence of the second target RNA in the cellular sample. In some embodiments, step (h) comprises sequencing the second plurality of concatemers inside the cellular sample comprises conducting 1-250 sequencing cycles to generate a plurality of third sequencing read products, wherein the sequences of the third sequencing read products are aligned with a third target reference sequence to confirm the presence of the second target RNA in the cellular sample. In some embodiments, step (h) comprises sequencing the fourth plurality of concatemers inside the cellular sample comprises conducting no more than 2-30 sequencing cycles to generate a plurality of fourth sequencing read products, wherein the sequences of the fourth sequencing read products are aligned with a fourth target reference sequence to confirm the presence of the second target polypeptides in the cellular sample. In some embodiments, step (h) comprises sequencing the fourth plurality of concatemers inside the cellular sample comprises conducting 1-250 sequencing cycles to generate a plurality of fourth sequencing read products, wherein the sequences of the fourth sequencing read products are aligned with a fourth target reference sequence to confirm the presence of the second target polypeptides in the cellular sample.

In some embodiments in step (h), in the second concatemer molecules, only the second target barcode region (target BC-2) is sequenced. In some embodiments, in the second concatemer molecules, at least a portion or the full length of the second target barcode (target BC-2) is sequenced. In some embodiments, in the second concatemer molecules, the second target barcode (target BC-2) is sequenced and a portion of the second cDNA region is sequenced.

In some embodiments in step (h), in the fourth concatemer molecules, only the fourth target barcode region (target BC-4) is sequenced. In some embodiments, in the fourth concatemer molecules, at least a portion or the full length of the fourth target barcode (target BC-4) is sequenced. In some embodiments, in the fourth concatemer molecules, the fourth target barcode (target BC-4) is sequenced and a portion of the second oligonucleotide is sequenced.

In some embodiments, the sequencing of step (h) comprises sequencing at least a portion of the second and fourth nucleic acid concatemers using an optical imaging system comprising a field-of-view (FOV) greater than 1.0 mm².

In some embodiments, in the sequencing of step (h), the plurality of third and fourth sequencing read products are detectable by imaging, and wherein the sequencing comprises decoding the plurality of third and fourth sequencing read products from the images obtained during the no more than 2-30 sequencing cycles, or from the images obtained during the 1-250 sequencing cycles.

In some embodiments, in the sequencing of step (h), the plurality of the third and fourth sequencing read products are detectable by imaging, and wherein the sequencing comprises simultaneously imaging the plurality of third and fourth detectable sequencing read products in the cellular sample (co-localization of the third and fourth sequencing read products).

In some embodiments, the methods further comprise step (i): removing the plurality of third sequencing read products from the second concatemer molecules and retaining the second concatemer molecules inside the cellular sample, and removing the plurality of fourth sequencing read products from the fourth concatemer molecules and retaining the fourth concatemer molecules inside the cellular sample. In some embodiments, a 3′ blocking moiety can be added to the third and fourth sequencing read products to inhibit further sequencing reactions. For example, a nucleotide analog can be incorporated where the nucleotide analog inhibits incorporation of a subsequent nucleotide. Non-limiting example blocking nucleotide analogs include dideoxynucleotide or a nucleotide having a 2′ or 3′ chain terminating moiety.

In some embodiments, the methods further comprise step (j): reiteratively sequencing the plurality of second and fourth concatemers by repeating steps (h) and (i) at least once. In some embodiments, the repeating comprises repeating at least 2 times, 5 times, 10 times, 15 times, 20 times, 25 times, 30 times, 35 times, 40 times, 45 times, or at least 50 times.

In some embodiments, the plurality of nucleotide reagents comprises a plurality of nucleotides that are detectably labeled or non-labeled. In some embodiments, individual nucleotides are linked to a detectable reporter moiety. In some embodiments, the detectable reporter moiety comprises a fluorophore. In some embodiments, the plurality of detectably labeled nucleotide analogs comprise a plurality of chain terminating nucleotides, where the chain terminating moiety is linked to the 3′ nucleotide sugar position to form a 3′ blocked nucleotide analog. In some embodiments, the chain terminating moiety can be removed to convert the 3′ blocked nucleotide analog to an extendible nucleotide having a 3′ OH group on the sugar. In some embodiments, the labeled nucleotide analogs are linked to a different fluorophore that corresponds to the nucleobases adenine, cytosine, guanine, thymine or uracil, where the different fluorophores emit a fluorescent signal during sequencing. In some embodiments, a sequencing cycle comprises (1) contacting the concatemer/sequencing primer duplex with a sequencing polymerase and a detectably labeled chain terminating nucleotide under a condition suitable for polymerase-catalyzed incorporation of the detectably labeled chain terminating nucleotide into the terminal end of the sequencing primer, (2) detecting and imaging the fluorescent signal and color emitted by the incorporated chain terminating nucleotide, and (3) removing the chain terminating moiety (e.g., unblocking) and retaining the concatemer/sequencing primer duplex. In some embodiments, the sequencing cycles are conducted on the plurality of concatemers inside the cellular sample to generate a plurality of sequencing read products. In some embodiments, the sequence of the sequencing read product can be determined and aligned with a reference sequence to confirm the presence of target RNA molecules inside the cellular sample. In some embodiments, the sequence of the sequencing read product can be determined and aligned with a reference sequence to confirm the presence of target polypeptides inside the cellular sample. In some embodiments, the sequences of the sequencing read products can be aligned after each round of generating the sequencing read products, or after generating a set of reiterative sequencing read products. In some embodiments, the sequencing reactions are conducted on a sequencing apparatus having a detector that captures fluorescent signals from the sequencing reactions inside the cellular sample. The sequencing apparatus can be configured to relay the fluorescent signal data captured by the detector to a computer system that is programmed to display images of different fluorescent spots which are co-located in the cellular sample, where individual fluorescent spots correspond to different target RNA molecules or different target polypeptides. In some embodiments, when the sequencing is conducted using different fluorescently-labeled nucleotide reagents that correspond to different nucleobases (e.g., A, G, C, T/U), then the images can have different color fluorescent spots co-located in the same cellular sample at different sequencing cycles.

In some embodiments, out-of-sync phasing and/or pre-phasing events can occur during synchronized sequencing reactions on clonally amplified template amplicons, where the sequencing reactions comprise polymerase-catalyzed sequencing reactions employing detectably labeled chain terminator nucleotides. In some embodiments, a sequencing reaction on one template molecule in the clonally-amplified template molecules moves ahead (e.g., pre-phasing) or fall behind (e.g., phasing) of the sequencing of the other template molecules within the clonally-amplified template molecules. During sequencing, a fluorescent signal is typically detected which corresponds to incorporation of a labeled chain terminator nucleotide. Thus, phasing and pre-phasing events can be detected and monitored using incorporation of a labeled chain terminator nucleotide.

In some embodiments, the plurality of nucleotide reagents comprises a plurality of multivalent molecules each comprising a core attached to a plurality of nucleotide-arms, wherein the nucleotide-arms are attached to a nucleotide unit. In some embodiments, individual multivalent molecules are labeled with a detectably reporter moiety. In some embodiments, the detectable reporter moiety comprises a fluorophore. In some embodiments, the core of the multivalent molecule is labeled with a fluorophore, and wherein the fluorophore which is attached to a given core of the multivalent molecule corresponds to the nucleotide base (e.g., adenine, guanine, cytosine, thymine or uracil) of the nucleotide arm. In some embodiments, at least one of the nucleotide arms of the multivalent molecule comprises a linker and/or nucleotide base that is attached to a fluorophore, and wherein the fluorophore which is attached to a given nucleotide base corresponds to the nucleotide base (e.g., adenine, guanine, cytosine, thymine or uracil) of the nucleotide arm. In some embodiments, a sequencing cycle comprises (1) contacting the concatemer/sequencing primer duplex with a first sequencing polymerase to form a complexed polymerase, (2) contacting the complexed polymerase with a detectably labeled multivalent molecule under a condition suitable for binding a complementary nucleotide unit of the multivalent molecule to the complexed polymerase thereby forming a multivalent-binding complex, and the condition is suitable for inhibiting incorporation of the complementary nucleotide unit into the terminal end of the sequencing primer, (3) detecting and imaging the fluorescent signal and color emitted by the bound detectably labeled multivalent molecule, (4) removing the first sequencing polymerase and the bound detectably labeled multivalent molecule, and retaining the concatemer/sequencing primer duplex, (5) contacting the retained concatemer/sequencing primer duplex with a second sequencing polymerase and a non-labeled chain terminating nucleotide under a condition suitable for polymerase-catalyzed incorporation of the non-labeled chain terminating nucleotide into the terminal end of the sequencing primer, and (6) removing the chain terminating moiety (e.g., unblocking) and retaining the concatemer/sequencing primer duplex. In some embodiments, individual cycle times can be achieved in less than 30 minutes. In some embodiments, the field of view (FOV) can exceed 1 mm² and the cycle time for scanning large area (>10 mm²) can be less than 5 minutes. In some embodiments, no more than 2-30 sequencing cycles or 1-250 sequencing cycles are conducted on the plurality of concatemers inside the cellular sample to generate a plurality of sequencing read products. In some embodiments, the sequence of the sequencing read product can be determined and aligned with a reference sequence to confirm the presence of the target RNA molecules inside the cellular sample. In some embodiments, the sequence of the sequencing read product can be determined and aligned with a reference sequence to confirm the presence of the target polypeptides inside the cellular sample. In some embodiments, the sequences of the sequencing read products can be aligned after each round of generating the sequencing read products, or after generating a set of reiterative sequencing read products. In some embodiments, the sequencing reactions are conducted on a sequencing apparatus having a detector that captures fluorescent signals from the sequencing reactions inside the cellular sample. The sequencing apparatus can be configured to relay the fluorescent signal data captured by the detector to a computer system that is programmed to display images of different fluorescent spots which are co-located in the cellular sample, where individual fluorescent spots correspond to different target RNA molecules or different target polypeptides.

In some embodiments, when sequencing with detectably labeled multivalent molecules, step (2) in which multivalent-binding complexes are formed and step (3) in which the bound detectably labeled multivalent molecules are imaged and detected, the conditions are gentle compared to sequencing workflows that employ detectable labeled chain terminating nucleotides. For example, steps (2) and (3) can be conducted at a gentle temperature of about 35-45° C., or about 39-42° C. Steps (2) and (3) can be conducted at a gentle temperature which can help retain the compact size and shape of a DNA nanoball during multiple sequencing cycles (e.g., up to 30 cycles) which can improve FWHM (full width half maximum) of a spot image of the DNA nanoball inside a cellular sample. In some embodiments, the DNA nanoball does not unravel during multiple sequencing cycles. In some embodiments, the spot image of the DNA nanoball does not enlarge during multiple sequencing cycles. In some embodiments, the spot image of the DNA nanoball remains a discrete spot during multiple sequencing cycles. The spot image can be represented as a Gaussian spot and the size can be measured as a FWHM. A smaller spot size as indicated by a smaller FWHM typically correlates with an improved image of the spot. In some embodiments, the FWHM of a nanoball spot can be about 10 μm or smaller.

In some embodiments, out-of-sync phasing and/or pre-phasing events can occur during synchronized polymerase-catalyzed sequencing reactions employing detectably labeled multivalent molecules. During sequencing, a fluorescent signal can be detected which corresponds to binding of complementary nucleotide unit of a multivalent molecule to the complexed polymerase thereby forming a multivalent-binding complex. Thus, phasing and pre-phasing events can be detected and monitored using binding of labeled multivalent molecules. In some embodiments, when conducting up to 30 sequencing cycles with detectably labeled multivalent molecules, the phasing and/or pre-phasing rate can be less than about 5%, or less than about 1%, or less than about 0.01%, or less than about 0.001%. By contrast, the phasing and/or pre-phasing rates for conducting up to 30 sequencing cycles using labeled chain terminator nucleotides can be about 5%.

In any of the methods described herein, the biological sample comprises a cellular organelle, a cell, a whole cell, a group of whole cells, a tissue, an intact tissue, a tumor, an intact tumor, an organ, an organism, or any combination thereof. In any of the methods described herein, the biological sample may be immobilized on a surface.

In any of the methods described herein, the cellular sample comprises a whole cell, a plurality of whole cells, an intact tissue or an intact tumor. In some embodiments, the cellular sample comprises a fresh cellular sample, a freshly-frozen cellular sample, a sectioned cellular sample, or an FFPE cellular sample. In some embodiments, the cellular sample comprise one or more living cells or non-living cells.

In some embodiments, the cellular sample can be obtained from a virus, fungus, prokaryote or eukaryote. In some embodiments, the cellular sample can be obtained from an animal, insect or plant. In some embodiments, the cellular sample comprises one or more virally-infected cells.

In some embodiments, the cellular sample can be obtained from any organism including human, simian, ape, canine, feline, bovine, equine, murine, porcine, caprine, lupine, ranine, piscine, plant, insect bacteria, algae sample, viral sample, protozoa, or fungal.

In some embodiments, the cellular sample can be obtained from any organ including head, neck, brain, breast, ovary, cervix, colon, rectum, endometrium, gallbladder, intestines, bladder, prostate, testicles, liver, lung, kidney, esophagus, pancreas, thyroid, pituitary, thymus, skin, heart, larynx, or other organs.

In any of the methods described herein, the cellular sample harbors a plurality of RNA which include target RNA and non-target RNA. In some embodiments, cells typically produce RNA by gene expression which includes transcription of DNA (e.g., genomic DNA) into RNA molecules. The transcribed RNA can undergo splicing or may not be spliced. The transcribed RNA can be translated into a polypeptide (e.g., coding RNA), or do not undergo translation but can be processed into tRNA or rRNA (e.g., non-coding RNA).

In some embodiments, the plurality of RNA harbored by the cellular sample includes target and non-target RNA. In some embodiments, the plurality of RNA harbored by the cellular sample comprises wild type RNA, mutant RNA or splice variant RNA. In some embodiments, the plurality of RNA harbored by the cellular sample comprises pre-spliced RNA, partially spliced RNA, or fully spliced RNA. In some embodiments, the plurality of RNA harbored by the cellular sample comprises coding RNA, non-coding RNA, mRNA, tRNA, rRNA, microRNA (miRNA), mature microRNA, or immature microRNA. In some embodiments, the plurality of RNA harbored by the cellular sample comprises housekeeping RNA, cell-specific RNA, tissue-specific RNA or disease-specific RNA. In some embodiments, the plurality of RNA harbored by the cellular sample comprises RNA expressed by one or more cells in response to a stimulus such as heat, light, a chemical or a drug. In some embodiments, the plurality of RNA harbored by the cellular sample comprises RNA found in healthy cells or diseased cells. In some embodiments, the plurality of RNA harbored by the cellular sample comprises RNA transcribed from transgenic DNA sequences that are introduced into the cellular sample using recombinant DNA procedures. For example, the RNA can be transcribed from a transgenic DNA sequence that is controlled by an inducible or constitutive promoter sequence. In some embodiments, the plurality of RNA harbored by the cellular sample comprises RNA that is transcribed from DNA sequences that are not transgenic.

In any of the methods described herein, the cellular sample can be cultured on the support. In some embodiments, the methods comprise culturing the cellular sample on the support under a condition suitable for expanding the cellular sample for 2-10 generations or more. The cultured cellular sample can generate a colony of cells. In some embodiments, the methods comprise culturing the cellular sample to confluence or non-confluence. In some embodiments, the methods comprise culturing the cellular sample on the support in a simple or complex cell culture media. For example, the cell culture media comprises D-MEM high glucose (e.g., from Thermo Fisher Scientific, catalog No. 11965118), fetal bovine serum (e.g., 10% FBS; for example from Thermo Fisher Scientific, catalog No. A3160402), MEM non-essential amino acids (e.g., 0.1 mM MEM, for example from Thermo Fisher Scientific, catalog No. 11140050), L-glutamine (e.g., 6 mM L-glutamine, for example from Thermo Fisher Scientific, catalog No. A2916801), MEM sodium pyruvate (e.g., 1 mM sodium pyruvate, for example from Thermo Fisher Scientific, catalog No. 11360070), and an antibiotic (e.g., 1% penicillin-streptomycin-glutamine, for example from Thermo Fisher, catalog No. 10378016). In some embodiments, the methods comprise culturing the cellular sample at a humidity and temperature that is suitable for culturing the cell(s) on the support. Non-limiting example suitable conditions comprise approximately 37° C. with a humidified atmosphere of approximately 5-10% carbon dioxide in air. The cellular sample can be cultured with suitable aeration with oxygen and/or nitrogen.

In any of the methods described herein, the term “simple cell media” or related terms refers to a cell media that typically lacks ingredients to support cell growth and/or proliferation in culture. Simple cell media can be used for example to wash, suspend, or dilute the cellular sample. Simple cell media can be mixed with certain ingredients to prepare a cell media that can support cell growth and/or proliferation in culture. A simple cell media comprises any one or any combination of two or more of a buffer, a phosphate compound, a sodium compound, a potassium compound, a calcium compound, a magnesium compound and/or glucose. In some embodiments, the simple cell media comprises PBS (phosphate buffered saline), DPBS (Dulbecco's phosphate-buffered saline), HBSS (Hank's balanced salt solution), DMEM (Dulbecco's Modified Eagle's Medium), EMEM (Eagle's Minimum Essential Medium), and/or EBSS. In some embodiments, the cellular sample can be placed in a simple cell media prior to or during the step of conducting any of the nucleic acid methods described herein.

In any of the methods described herein, the term “complex cell media” or related terms refers to a cell media that can be used to support cell growth and/or proliferation in culture without supplementation or additives. Complex cell media can include any combination of two or more of a buffering system (e.g., HEPES), inorganic salt(s), amino acid(s), protein(s), polypeptide(s), carbohydrate(s), fatty acid(s), lipid(s), purine(s) and their derivatives (e.g., hypoxanthine), pyrimidine(s) and their derivatives, and/or trace element(s). Complex cell media includes fluids obtained from a fluid or tissue extract. Complex cell media includes artificial cell media. In some embodiments, complex cell media can be a serum-containing media, for example complex cell media includes fluids such as fetal bovine serum, blood plasma, blood serum, lymph fluid, human placental cord serum and amniotic fluid. In some embodiments, complex cell media can be a serum-free media, which are typically (but not necessarily) defined cell culture media. In some embodiments, complex cell media can be a chemically-defined media which typically (but not necessarily) include recombinant polypeptides, and ultra-pure inorganic and/or organic compounds. In some embodiments, complex cell media can be a protein-free media which include for example MEM (minimal essential media) and RPMI-1640 (Roswell Park Memorial Institute). In some embodiments, the complex cell media comprises IMDM (Iscove's Modified Dulbecco's Medium. In some embodiments, the complex cell media comprises DMEM (Dulbecco's Modified Eagle's Medium). In some embodiments, the cellular sample can be placed in a complex cell media prior to or during the step of conducting any of the nucleic acid methods described herein.

In any of the methods described herein, the cellular sample comprises a fixed cellular sample. In some embodiments, the cellular sample can be treated with a fixation reagent (e.g., a fixing reagent) that preserves the cell and its contents to inhibit degradation and can inhibit cell lysis. For example, the fixation reagent can preserve RNA harbored by the cellular sample. In some embodiments, the fixation reagent inhibits loss of nucleic acids from the cellular sample.

In some embodiments, the fixation reagent can cross-link the RNA to prevent the RNA from escaping the cellular sample. In some embodiments, a cross-linking fixation reagent comprises any combination of an aldehyde, formaldehyde, paraformaldehyde, formalin, glutaraldehyde, imidoesters, N-hydroxysuccinimide esters (NHS) and/or glyoxal (a bifunctional aldehyde).

In some embodiments, the fixation reagent comprises at least one alcohol, including methanol or ethanol. In some embodiments, the fixation reagent comprises at least one ketone, including acetone. In some embodiments, the fixation reagent comprises acetic acid, glacial acetic acid and/or picric acid. In some embodiments, the fixation reagent comprises mercuric chloride. In some embodiments, the fixation reagent comprises a zinc salt comprising zinc sulphate or zinc chloride. In some embodiments, the fixation reagent can denature polypeptides.

In some embodiments, the fixation reagent comprises 4% w/v of paraformaldehyde to water/PBS. In some embodiments, the fixation reagent comprises 10% of 35% formaldehyde at a neutral pH. In some embodiments, the fixation reagent comprises 2% v/v of glutaraldehyde to water/PBS. In some embodiments, the fixation reagent comprises 25% of 37% formaldehyde solution, 70% picric acid and 5% acetic acid.

In some embodiments, the cellular sample can be fixed on the support with 4% paraformaldehyde for about 30-60 minutes and washed with PBS.

In some embodiments, the cellular sample can be stained, de-stained or un-stained.

In any of the methods described herein, the cellular sample comprises a permeabilized cellular sample. In some embodiments, the methods comprise treating the cellular sample with a permeabilization reagent that alters the cell membrane to permit penetration of experimental reagents into the cells. For example, the permeabilization reagent removes membrane lipids from the cell membrane. In some embodiments, the cellular sample can be treated with a permeabilization reagent which comprises any combination of an organic solvent, detergent, chemical compound, cross-linking agent and/or enzyme. In some embodiments, the organic solvents comprise acetone, ethanol, and methanol. In some embodiments, the detergents comprise saponin, Triton X-100, Tween-20, sodium dodecyl sulfate (SDS), an N-lauroylsarcosine sodium salt solution, or a nonionic polyoxyethylene surfactant (e.g., NP40). In some embodiments, the cross-linking agent comprises paraformaldehyde. In some embodiments, the enzyme comprises trypsin, pepsin or protease (e.g. proteinase K). In some embodiments, the cells can be permeabilized using an alkaline condition, or an acidic condition with a protease enzyme. In some embodiments, the permeabilization reagent comprises water and/or PBS.

For example, the fixed cells can be permeabilized with 70% ethanol for about 30-60 minutes, and the permeabilizing reagent can be exchanged with PBS-T (e.g., PBS with 0.05% Tween-20). In some embodiments, the cells can be post-fixed with 3% paraformaldehyde and 0.1% glutaraldehyde for about 30-60 minutes, and washed with PBS-T multiple times.

In any of the methods described herein, the cellular sample is infused with a swellable polyelectrolyte hydrogel (U.S. Pat. No. 10,309,879 and Chen 2015 Science 347:543, the contents of these documents are incorporated by reference in their entireties). In some embodiments, a fixed and permeabilized cellular sample can be infused with sodium acrylate, acrylamide and a cross-linker N-N′-methylenebisacrylamide. In some embodiments, ammonium persulfate (APS) initiator and tetramethylethylenediamine (TEMED) accelerator were infused to achieve polymerization. In some embodiments, the cellular sample can be infused with proteinase K for proteolysis and incubated in a digestion buffer. In some embodiments, the gel inside the cellular sample can be swelled by addition of water.

The present disclosure provides methods for detecting in situ at least two different target RNA molecules in a cellular sample, which can include converting the plurality of RNAs inside cellular sample to cDNA. In some embodiments, the methods can comprise contacting the plurality of RNA inside the fixed and permeabilized cellular sample with (i) a plurality of reverse transcription primers, (ii) a plurality of reverse transcriptase enzymes, and (iii) a plurality of nucleotides, under a condition suitable for conducting a reverse transcription reaction to generate a plurality of cDNA molecules (e.g., a plurality of first strand cDNA molecules) in the cellular sample. In some embodiments, the method can comprise contacting the plurality of RNA inside the fixed and permeabilized cellular sampled with a plurality of reverse transcription primers. In some embodiments, the method can comprise contacting the plurality of RNA inside the fixed and permeabilized cellular sampled with a plurality of reverse transcriptase enzymes. In some embodiments, the method can comprise contacting the plurality of RNA inside the fixed and permeabilized cellular sampled with a plurality of nucleotides. In some embodiments, synthesis of second strand cDNA molecules may be omitted.

### Reverse Transcriptase

The present disclosure provides methods for conducting in situ analysis of a nucleic acid. The analysis may comprise a sequencing of the nucleic acid. In some embodiments, the nucleic acid may be an RNA. The analysis of the RNA may comprise reverse transcribing the RNA into a complementary DNA (cDNA). A reverse transcriptase may be used to transcribe the RNA into a cDNA.

Methods as described herein may comprise use of a reverse transcriptase. In some embodiments, the reverse transcriptase enzyme can exhibit RNA-dependent DNA polymerase activity. In some embodiments, the reverse transcriptase enzyme can comprise a reverse transcriptase enzyme from AMV (avian myeloblastosis virus), M-MMLV (moloney murine leukemia virus), or HIV (human immunodeficiency virus). In some embodiment, the reverse transcriptase enzyme can comprise a recombinant enzyme that exhibits reduced RNase H activity, for example REVERTAID (e.g., from Thermo Fisher Scientific, catalog No. EP0441). In some embodiments, the reverse transcriptase can be a commercially-available enzyme, including MULTISCRIBE (e.g., from Thermo Fisher Scientific, catalog #4311235), THERMOSCRIPT (e.g., from Thermo Fisher Scientific, catalog #12236-014), or ARRAYSCRIPT (e.g., from Ambion, catalog No. AM2048). In some embodiments, the reverse transcriptase enzyme can comprise SUPERSCRIPT II (e.g., catalog No. 18064014), SUPERSCRIPT III (e.g., catalog No. 18080044), or SUPERSCRIPT IV enzymes (e.g., catalog No. 18090010) (all SUPERSCRIPT enzymes from Invitrogen). In some embodiments, the reverse transcription reaction can include an RNase inhibitor. Further examples of reverse transcriptases can also be found in US Publication No. US20210139884, which is incorporated herein by reference in its entirety.

In some embodiments, the reverse transcription primers comprise a single-stranded oligonucleotide comprising DNA, RNA, or chimeric DNA/RNA. In some embodiments, the reverse transcription primers Any combination of adenine (A), thymine (T), guanine (G), cytosine (C), uracil (U) and/or inosine (I). In some embodiments, the reverse transcription primers can be any length, for example 5-25 bases, or 25-50 bases, or 50-75 bases, or 75-100 bases in length or longer. The reverse transcription primers each comprise a 5′ end and 3′ end. In some embodiments, the 3′ end of the reverse transcription primers can include a 3′ OH moiety which serves as a nucleotide polymerization initiation site in a polymerase-catalyzed primer extension reaction. In some embodiments, the 3′ end of the reverse transcription primers have a chain terminating moiety which blocks a polymerase-catalyzed primer extension reaction. The chain terminating moiety can be removed to convert the 3′ sugar position to an extendible 3′OH.

In some embodiments, the reverse transcription primers are modified to confer resistance to nuclease degradation (e.g., ribonuclease degradation). For example, the reverse transcription primers comprise at least one phosphorothioate diester bond at their 5′ ends which can render the reverse transcription primers resistant to nuclease degradation. In some embodiments, the reverse transcription primers comprise 2-5 or more consecutive phosphorothioate diester bonds at their 5′ ends. In some embodiments, the plurality of reverse transcription primers comprise at least one ribonucleotide and/or at least one 2′-O-methyl, 2′-O-methoxyethyl (MOE), 2′ fluoro-base nucleotide. In some embodiments, the reverse transcription primers comprise phosphorylated 3′ ends. In some embodiments, the reverse transcription primers comprise locked nucleic acid (LNA) bases. In some embodiments, the reverse transcription primers comprise a phosphorylated 5′ end (e.g., using a polynucleotide kinase).

In some embodiments, the entire length of a reverse transcription primer can hybridize to a portion of an RNA molecule. In some embodiments, individual reverse transcription primers comprise a 3′ region having a sequence that hybridizes to a portion of an RNA molecule and a 5′ region that carries a tail that does not hybridize to an RNA molecule. In some embodiments, the 5′ tail comprises a universal adaptor sequence including any one or any combination of two or more of a sample barcode sequence, an amplification primer binding site, a sequencing primer binding site, a compaction oligonucleotide binding site and/or a surface capture primer binding site. In some embodiments, the 5′ tail comprises a unique identification sequence (e.g., unique molecular index (UMI). In some embodiments, the 5′ tail comprises a restriction enzyme recognition sequence. In some embodiments, individual reverse transcription primers comprise at least a portion of the 3′ region having a homopolymer sequence, for example poly-A, poly-T, poly-C, poly-G or poly-U. In some embodiments, the reverse transcription primers can hybridize to any portion of an RNA molecule, including the 5′ or the 3′ end of the RNA molecule, or an internal portion of the RNA molecule.

In some embodiments, the plurality of reverse transcription primers comprises a first sub-population of target-specific reverse transcription primers that hybridize selectively to the first target RNA (e.g., targeted transcriptomics). In some embodiments, the plurality of reverse transcription primers further comprise a second sub-population of target-specific reverse transcription primers that hybridize selectively to the second target RNA. In some embodiments, the target-specific reverse transcription primers comprise a pre-determined sequence at the 3′ region which hybridizes to a target RNA molecule. In some embodiments, the pre-determined sequence portion of the reverse transcription primers can be 4-20 bases, or 20-40 bases, or 40-50 bases in length.

In some embodiments, the first sub-population of target-specific reverse transcription primers can selectively hybridize to an RNA transcribed in the cellular sample by a housekeeping gene. In some embodiments, selection of the housekeeping gene may be dependent upon the type of cellular sample to be used for the in situ methods described herein. Non-limiting example housekeeping genes include glyceraldehyde-3-phosphate dehydrogenase (GAPDH), beta-actins (ACTB), tubulins, PPIA (peptidyl-prolyl cis-trans isomerase), NME4 (NME/NM23 nucleoside diphosphate kinase 4), SMARCAL1 (SWI/SNF related matrix associated actin dependent regulator of chromatin, subfamily A like 1), and POMK (protein-O-mannose kinase). The skilled artisan can design the first sub-population of target-specific reverse transcription primers to hybridize to RNA transcripts from any of the numerous housekeeping genes.

In some embodiments, the second sub-population of target-specific reverse transcription primers can selectively hybridize to an RNA transcribed from a gene that is expressed in the cellular sample being examined (e.g., a cell-specific or tissue-specific RNA).

In some embodiments, the plurality of reverse transcription primers comprises a first sub-population of random-sequence reverse transcription primers that hybridize to the first target RNA (e.g., whole transcriptomics). In some embodiments, the plurality of reverse transcription primers further comprises a second sub-population of random-sequence reverse transcription primers that hybridize to the second target RNA. In some embodiments, the reverse transcription primers comprise a random and/or degenerate sequence at the 3′ region which hybridizes to an RNA molecule. In some embodiments, the random-sequence or the degenerate-sequence portion of the reverse transcription primers can be 4-20 bases, or 20-40 bases, or 40-50 bases in length.

### Padlock Probes

The present disclosure provides a padlock probe for use in any of the methods as described herein. A padlock probe may be designed to selectively detect a target molecule. For example, the padlock probe may be designed to selectively detect a target nucleic acid or polypeptide. The target nucleic acid may be a target DNA or a target RNA. By binding the padlock probe to the target nucleic acid or protein/polypeptide, one can learn information about one or more properties of the target nucleic acid or protein/polypeptide. For example, a location of the target nucleic acid or protein/polypeptide can be ascertained. The padlock probe may comprise one or more batch-specific primer-binding sites, which are specific to a primer. The primer may be spatially localized, and the padlock probe may therefore not require a barcode sequence. The location of the target can give information about the sample, e.g., cellular origin. In such instances, it may not be necessary for the padlock probe to carry a barcode. However, in other non-limiting instances, the padlock probe may carry a barcode.

The present disclosure provides a padlock probe that is specific to a target RNA. The RNA-specific padlock probe may selectively hybridize to a cDNA that corresponds to the target RNA. The RNA-specific probe may lack a barcode that uniquely identifies the cDNA. In such instances, a spatial information may be obtained with respect to the RNA-specific probe. The RNA-specific probe may carry a barcode that uniquely identifies the cDNA. The RNA-specific padlock probes may also carry a sequencing primer binding site. The sequencing primer binding site may be a batch-specific primer binding site—in other words, a primer may bind to the primer binding site that is unique to the probe. One or more padlock probes may be used. Each probe may have a unique batch-specific primer binding site. The use of a batch-specific primer binding site may enable the spatial localization of the padlock probe, thereby obviating the need for a barcode sequence on the padlock probe.

In any of the methods described herein, the plurality of cDNA inside the cellular sample can be amplified to generate cDNA amplicons (e.g., concatemers). In some embodiments, the plurality of cDNA molecules can be amplified by conducting a padlock probe circularization and rolling circle amplification workflow. In some embodiments, the methods comprise contacting the plurality of cDNA molecules with a plurality of padlock probes, including a first plurality of target-specific padlock probes that hybridize with first target cDNA molecules, and a second plurality of target-specific padlock probes that hybridize with second target cDNA molecules.

In some embodiments, the padlock probes comprise single-stranded oligonucleotides. In some embodiments, the padlock probes comprise DNA, RNA, DNA and RNA. In some embodiments, the padlock probes comprise canonical nucleotides and/or nucleotide analogs. In some embodiments, the padlock probes are modified to confer resistance to nuclease degradation (e.g., ribonuclease degradation). For example, the padlock probes comprise at least one phosphorothioate diester bond at their 5′ ends which can render the padlock probes resistant to nuclease degradation. In some embodiments, the padlock probes comprise 2-5 or more consecutive phosphorothioate diester bonds at their 5′ ends. In some embodiments, the padlock probes comprise at least one ribonucleotide and/or at least one 2′-O-methyl, 2′-O-methoxyethyl (MOE), 2′ fluoro-base nucleotide. In some embodiments, the padlock probes comprise phosphorylated 3′ ends. In some embodiments, the padlock probes comprise at least one locked nucleic acid (LNA) base. In some embodiments, the padlock probes comprise a phosphorylated 5′ end (e.g., using a polynucleotide kinase).

In some embodiments, the methods can comprise contacting the plurality of cDNA molecules with a plurality of padlock probes. In some embodiments, the plurality of padlock probes can include a first plurality of target-specific padlock probes. In some embodiments, the first plurality of target-specific padlock probes can hybridize with the first target cDNA molecules. In some embodiments, the plurality of padlock probes can include a second plurality of target-specific padlock probes. In some embodiments, the second plurality of target-specific padlock probes can hybridize with the second target cDNA molecules.

In some embodiments, individual padlock probes comprise first and second terminal regions that hybridize to portions of cDNA molecules to form a plurality of cDNA-padlock probe complexes, wherein individual complexes have the first and second terminal probe regions hybridized to proximal regions of a cDNA molecule to form a nick or gap between the first and second terminal probe ends. In some embodiments, the first terminal region of an individual padlock probe has a first target-specific sequence that selectively hybridizes to a first region of a target cDNA molecule, and the second terminal region of the individual padlock probe has a second target-specific sequence that selectively hybridizes to a second region of the same target cDNA molecule, where a nick or gap is formed between the hybridized first and second terminal regions, thereby circularizing the padlock probe. In some embodiments, the first terminal region of an individual padlock probe can have a first target-specific sequence that can selectively hybridize to a first region of a target cDNA molecule. In some embodiments, the second terminal region of an individual padlock probe can have a second target-specific sequence that can selectively hybridize to a second region of a target cDNA molecule. In some embodiments, a nick or gap can be formed between the hybridized first and second terminal regions. In some embodiments, the hybridization of the first terminal region and the second terminal region can circularize the padlock probe.

In some embodiments, individual padlock probes in a set of padlock probes (e.g., a plurality of padlock probes) comprise first and second terminal regions that hybridize to the same target regions of the target cDNA molecules to form a plurality of cDNA-padlock probe complexes having the same cDNA sequence.

In some embodiments, a set of padlock probes (e.g., a plurality of padlock probes) comprise at least two sub-sets of padlock probes. In some embodiments, individual padlock probes in a first sub-set of padlock probes comprise first and second terminal regions that hybridize to the same target regions (e.g., a first target region) of the target cDNA molecules to form a first plurality of cDNA-padlock probe complexes having the same cDNA sequence. In some embodiments, individual padlock probes in a second sub-set of padlock probes comprise first and second terminal regions that hybridize to the same target regions (e.g., a second target region) of the target cDNA molecules to form a second plurality of cDNA-padlock probe complexes having the same cDNA sequence. In some embodiments, the first and second sub-sets of padlock probes hybridize to different target regions of the same target cDNA molecules. In some embodiments, the first and second sub-sets of padlock probes hybridize to different target regions of different target cDNA molecules. In some embodiments, the set of padlock probes comprise 2-10 sub-sets of padlock probes, or 10-25 sub-sets of padlock probes, or 25-50 sub-sets of padlock probes, or up to 100 sub-sets of padlock probes. In some embodiments, the set of padlock probes comprise at least 100 sub-sets of padlock probes, at least 500 sub-sets of padlock probes, at least 1000 sub-sets of padlock probes, at least 10,000 sub-sets of padlock probes, or more sub-sets of padlock probes.

In some embodiments, the nicks can be enzymatically ligated to generate covalently closed circular padlock probes. In some embodiments, the ligase enzyme can discriminate between matched and mis-matched hybridized ends to ensure target-specific hybridization. In some embodiments, the ligation reaction comprises use of a ligase enzyme, including a T3, T4, T7 or Taq DNA ligase enzyme.

In some embodiments, the size of the gap between the hybridized first and second terminal regions is 1-25 bases. The 3′OH end of hybridized padlock probe can serve as an initiation site for a polymerase-catalyzed fill-in reaction (e.g., gap fill-in reaction) using the target cDNA molecule as a template. After the fill-in reaction, the remaining nick can be enzymatically ligated to generate covalently closed circular padlock probes.

In some embodiments, the gap-filling reaction comprises contacting the circularized padlock probe with a DNA polymerase and a plurality of nucleotides. In some embodiments, the DNA polymerase comprises E. coli DNA polymerase I, Klenow fragment of E. coli DNA polymerase I, T7 DNA polymerase, or T4 DNA polymerase. In some embodiments, the ligase enzyme can discriminate between matched and mis-matched hybridized ends to ensure target-specific hybridization. In some embodiments, the ligation reaction comprises use of a ligase enzyme, including a T3, T4, T7 or Taq DNA ligase enzyme.

In some embodiments, the padlock probes comprise at least one universal adaptor sequence including a sample barcode sequence, an amplification primer binding site, a sequencing primer binding site, a compaction oligonucleotide binding site and/or a surface capture primer binding site. In some embodiments, the padlock probes comprise at least one unique identification sequence (e.g., unique molecular index (UMI). In some embodiments, the padlock probes comprise at least one restriction enzyme recognition sequence.

### Rolling Circle Amplification (RCA)

In any of the methods described herein, the plurality of covalently closed circular padlock probes can be subjected to a rolling circle amplification reaction to generate a plurality of concatemer molecules each having two or more tandem copies of a unit wherein the unit comprises a target sequence that corresponds to a target RNA molecules and any additional sequence(s) carried by the padlock probes including universal adaptor sequence(s), unique molecular index sequence(s) and/or restriction enzyme recognition sequence(s).

In some embodiments, the rolling circle amplification reaction can generate a plurality of concatemer molecules. In some embodiments, each concatemer molecule of the plurality of concatemer molecule can have two or more tandem copies of a unit. In some embodiments, a unit can comprise a target sequence that can correspond to a target RNA molecule. In some embodiments, a unit can comprise additional sequence(s) carried by the padlock probes. In some embodiments, the additional sequences can include universal adaptor sequence(s), unique molecular index sequence(s), restriction enzyme recognition sequence(s), or any combination thereof.

In some embodiments, the rolling circle amplification reaction comprises contacting the covalently closed circularized padlock probes with an amplification primer (e.g., a universal rolling circle amplification primer), a strand-displacing DNA polymerase, and a plurality of nucleotides, under a condition suitable for hybridizing individual amplification primers to a covalently closed padlock probe, and under a condition suitable for conducting primer extension using the covalently closed padlock probe as a template molecule to generate a nucleic acid concatemer. In some embodiments, the rolling circle amplification reaction can comprise contacting the covalently closed circularized padlock probes with an amplification primer. In some embodiments, the rolling circle amplification reaction can comprise contacting the covalently closed circularized padlock probes with a strand-displacing DNA polymerase. In some embodiments, the rolling circle amplification reaction can comprise contacting the covalently closed circularized padlock probes with a plurality of nucleotides. In some embodiments, the rolling circle amplification can occur under a condition suitable for hybridizing individual amplification primers to a covalently closed padlock probe. In some embodiments, the rolling circle amplification can occur under a condition suitable for conducting primer extension. In some embodiments, primer extension can occur using the covalently closed padlock probe as a template molecule. In some embodiments, the primer extension can generate a nucleic acid concatemer. In some embodiments, the plurality of nucleotides in the rolling circle amplification reaction comprise any mixture of two or more of dATP, dGTP, dCTP, dTTP and/or dUTP. In some embodiments, any of the rolling circle amplification reactions described herein can be conducted in the presence or in the absence of a plurality of compaction oligonucleotides.

In some embodiments, when the rolling circle amplification reaction includes a plurality of nucleotide which includes dUTP, the resulting concatemer can be cross-linked to a cross-linking reactive group by treating the cellular sample with a succininuide ester (NHS), maleimide (Sulfo-SMCC), imidoester (DMP), carbodiimide (DCC, EDC) or phenyl azide. in some embodiments, polymerization of the cross-linking reactive group can be initiated with light or UV light. in some embodiments, the resulting concatemer can be cross-linked to a matrix by treating the cellular sample with a cross-linked agarose, cross-linked dextran or cross-linked polyethylene glycol (PEG), polyacrylamide, cellulose alginate or polyamide. In some embodiments, the PEG comprises a sulfo-NHS ester moiety at one or both ends, for example a PEGylated bis(sulfosuccinimidyl)suberate) (e.g., BS(PEG)9 from Thermo Fisher Scientific, catalog No. 21582).

In some embodiments, the rolling circle amplification reaction can be conducted at a constant temperature (e.g., isothermal) wherein the constant temperature is at room temperature to about 30° C., or about 30-40° C., or about 40-50° C., or about 50-65° C.

In some embodiments, the DNA polymerase having a strand displacing activity can be selected from a group consisting of phi29 DNA polymerase, large fragment of Bst DNA polymerase, large fragment of Bsu DNA polymerase, and Bca (exo-) DNA polymerase, Klenow fragment of E. coli DNA polymerase, T5 polymerase, M-MuLV reverse transcriptase, HIV viral reverse transcriptase, or Deep Vent DNA polymerase. In some embodiments, the phi29 DNA polymerase can be wild type phi29 DNA polymerase (e.g., MagniPhi from Expedeon), or variant EquiPhi29 DNA polymerase (e.g., from Thermo Fisher Scientific), and chimeric QualiPhi DNA polymerase (e.g., from 4basebio).

In some embodiments, the rolling circle amplification primers can be modified to increase resistance to nuclease degradation. In some embodiments, the rolling circle amplification primers comprise at least one phosphorothioate diester bond at their 5′ ends which can render the amplification primers resistant to exonuclease degradation. In some embodiments, the rolling circle amplification primers comprise 2-5 or more consecutive phosphorothioate diester bonds at their 5′ ends. In some embodiments, the rolling circle amplification primers comprise at least one ribonucleotide and/or at least one 2′-O-methyl or 2′-O-methoxyethyl (MOE) nucleotide.

In some embodiments, the rolling circle amplification reaction can be conducted in the presence of a plurality of compaction oligonucleotides which, when hybridized to a concatemer molecule, can compact the size, the shape, or a combination of the size and the shape of the concatemer to form a compact nanoball. In some embodiments, the rolling circle amplification reaction can be conducted in the presence of a plurality of compaction oligonucleotides. In some embodiments, the compaction oligonucleotides can hybridize to a concatemer molecule. In some embodiments, the compaction oligonucleotide hybridized to the concatemer molecule can compact the size, the shape, or a combination of the size and the shape of the concatemer molecule. In some embodiments, the compacted concatemer molecule can form a compact nanoball. In some embodiments, the compaction oligonucleotides can comprise single stranded oligonucleotides having a first region at one end that can hybridize to a portion of a concatemer molecule and a second region at the other end that can hybridize to another portion of the same concatemer molecule, where hybridization of the compaction oligonucleotide to a given concatemer can compact the size, the shape, or a combination of the size or the shape of the concatemer. In some embodiments, the compaction oligonucleotides can comprise single stranded oligonucleotides having a first region at one end that can hybridize to a portion of a concatemer molecule and a second region at the other end that can hybridize to another portion of the same concatemer molecule. In some embodiments, the hybridization of the compaction oligonucleotide to a concatemer molecule can compact the size, the shape, or a combination of the size and the shape of the concatemer molecule.

The compaction oligonucleotides include a 5′ region, an optional internal region (intervening region), a 3′ region, or any combination thereof. A 5′ and 3′ regions of the compaction oligonucleotide can hybridize to different portions of the concatemer to pull together distal portions of the concatemer causing compaction of the concatemer to form a DNA nanoball. For example, the 5′ region of the compaction oligonucleotide is designed to hybridize to a first portion of the concatemer molecule (e.g., a universal compaction oligonucleotide binding site), and the 3′ region of the compaction oligonucleotide is designed to hybridized to a second portion of the concatemer molecule (e.g., a universal compaction oligonucleotide binding site). Inclusion of compaction oligonucleotides during RCA can promote formation of DNA nanoballs having tighter size and shape compared to concatemers generated in the absence of the compaction oligonucleotides. The compact and stable characteristics of the DNA nanoballs improves in situ sequencing accuracy by increasing signal intensity and the nanoballs retain their shape and size during multiple sequencing cycles.

In some embodiments, the compaction oligonucleotides comprise single stranded oligonucleotides comprising DNA, RNA, or a combination of DNA and RNA. The compaction oligonucleotides can be any length, including 20-150 nucleotides, or 30-100 nucleotides, or 40-80 nucleotides in length.

In some embodiments, the compaction oligonucleotides comprises a 5′ region and a 3′ region, and optionally an intervening region between the 5′ and 3′ regions. The intervening region can be any length, for example about 2-20 nucleotides in length. The intervening region comprises a homopolymer having consecutive identical bases (e.g., AAA, GGG, CCC, TTT or UUU). The intervening region comprises a non-homopolymer sequence.

The 5′ region of the compaction oligonucleotides can be wholly complementary or partially complementary along its length to a first portion of a concatemer molecule. The 5′ region of the compaction oligonucleotides can be wholly complementary along its length to a first portion of a concatemer molecule. The 5′ region of the compaction oligonucleotides can be partially complementary along its length to a first portion of a concatemer molecule. The 3′ region of the compaction oligonucleotides can be wholly complementary or partially complementary along its length to a second portion of a concatemer molecule. The 3′ region of the compaction oligonucleotides can be wholly complementary along its length to a second portion of a concatemer molecule. The 3′ region of the compaction oligonucleotides can be partially complementary along its length to a second portion of a concatemer molecule. The 5′ region of the compaction oligonucleotides can hybridize to a first universal sequence portion of a concatemer molecule. The 3′ region of the compaction oligonucleotides can hybridize to a second universal sequence portion of a concatemer molecule . . . .

In some embodiments, the 5′ region of the compaction oligonucleotide can have the same sequence as the 3′ region. The 5′ region of the compaction oligonucleotide can have a sequence that is different from the 3′ region. In some embodiments, the 3′ region of the compaction oligonucleotide can have a sequence that is a reverse sequence of the 5′ region. In some embodiments, the 5′ region of the compaction oligonucleotide can have a sequence that is a reverse sequence of the 3′ region.

In some embodiments, the 3′ region of any of the compaction oligonucleotides can include an additional three bases at the terminal 3′ end which comprises 2′-O-methyl RNA bases (e.g., designated mUmUmU) or the terminal 3′ end lacks additional 2′-O-methyl RNA bases.

In some embodiments, the compaction oligonucleotides comprise one or more modified bases or linkages at their 5′ or 3′ ends to confer certain functionalities. In some embodiments, the compaction oligonucleotides comprise at least one phosphorothioate linkages at their 5′ and/or 3′ ends to confer exonuclease resistance. In some embodiments, at least one nucleotide at or near the 3′ end comprises a 2′ fluoro base which confers exonuclease resistance. In some embodiments, the 3′ end of the compaction oligonucleotides comprise at least one 2′-O-methyl RNA base which blocks polymerase-catalyzed extension. For example, the 3′ end of the compaction oligonucleotide comprises three bases comprising 2′-O-methyl RNA base (e.g., designated mUmUmU). In some embodiments, the compaction oligonucleotides comprise a 3′ inverted dT at their 3′ ends which blocks polymerase-catalyzed extension. In some embodiments, the compaction oligonucleotides comprise 3′ phosphorylation which blocks polymerase-catalyzed extension. In some embodiments, the internal region of the compaction oligonucleotides comprise at least one locked nucleic acid (LNA) which increases the thermal stability of duplexes formed by hybridizing a compaction oligonucleotide to a concatemer molecule. In some embodiments, the compaction oligonucleotides comprise a phosphorylated 5′ end (e.g., using a polynucleotide kinase).

In some embodiments, the compaction oligonucleotide comprises the sequence 5′-CATGTAATGCACGTACTTTCAGGGTAAACATGTAATGCACGTACTTTCAGGGT-3′ (SEQ ID NO: 14). In some embodiments, the compaction oligonucleotides includes an additional three bases at the terminal 3′ end which comprises 2′-O-methyl RNA bases (e.g., designated mUmUmU) or the terminal 3′ end lacks additional 2′-O-methyl RNA bases.

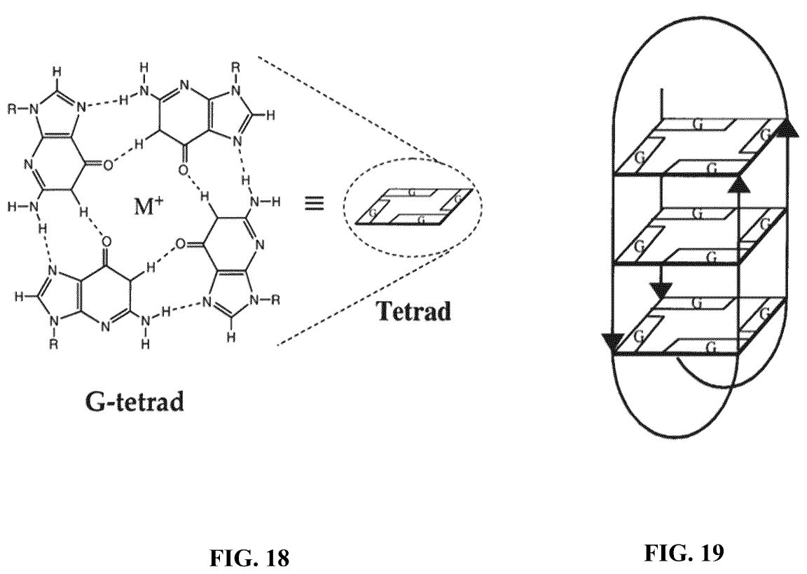

In some embodiments, the compaction oligonucleotides can include at least one region having consecutive guanines. For example, the compaction oligonucleotides can include at least one region having 2, 3, 4, 5, 6 or more consecutive guanines. In some embodiments, the compaction oligonucleotides comprise four consecutive guanines which can form a guanine tetrad structure (see FIG. 18). The guanine tetrad structure can be stabilized via Hoogsteen hydrogen bonding. The guanine tetrad structure can be stabilized by a central cation including potassium, sodium, lithium, rubidium or cesium.

At least one compaction oligonucleotide can form a guanine tetrad (FIG. 18) and hybridize to the universal binding sequences in a concatemer which can cause the concatemer to fold to form an intramolecular G-quadruplex structure (FIG. 19). The concatemers can self-collapse to form compact nanoballs. Formation of the guanine tetrads and G-quadruplexes in the nanoballs may increase the stability of the nanoballs to retain their compact size and shape which can withstand changes in pH, temperature and/or repeated flows of reagents during sequencing inside the cellular sample.

In some embodiments, the plurality of compaction oligonucleotides in the rolling circle amplification reaction have the same sequence. Alternatively, the plurality of compaction oligonucleotides in the rolling circle amplification reaction comprise a mixture of two or more different populations of compaction oligonucleotides having different sequences.

In some embodiment, the immobilized concatemer template molecule can self-collapse into a compact nucleic acid nanoball. The nanoballs can be imaged and a FWHM measurement can be obtained to give the shape/size of the nanoballs.

In some embodiments, inclusion of compaction oligonucleotides in the rolling circle amplification reaction can promote collapsing of a concatemer into a DNA nanoball. Conducting RCA with compaction oligonucleotides helps retain the compact size and shape of a DNA nanoball during multiple sequencing cycles which can improve FWHM (full width half maximum) of a spot image of the DNA nanoball inside a cellular sample. In some embodiments, the DNA nanoball does not unravel during multiple sequencing cycles. In some embodiments, the spot image of the DNA nanoball does not enlarge during multiple sequencing cycles. In some embodiments, the spot image of the DNA nanoball remains a discrete spot during multiple sequencing cycles. The spot image can be represented as a Gaussian spot and the size can be measured as a FWHM. A smaller spot size as indicated by a smaller FWHM typically correlates with an improved image of the spot. In some embodiments, the FWHM of a nanoball spot can be about 10 μm or smaller.

### Protein-Binding Composition

The present disclosure provides a protein-binding composition. The protein-binding composition may be specific to a target polypeptide or protein corresponding to a target RNA. The target RNA may then be sequenced. In this manner, the protein-binding composition may increase the specificity of the methods of sequencing as disclosed herein.

In any of the methods described herein, the protein-binding composition can be used to selectively bind a target polypeptide or protein. In some embodiments, the protein-binding composition is linked to an oligonucleotide carrying at least one tag sequence which uniquely identifies the target polypeptide or protein to which the protein-binding composition selectively binds. In some embodiments, the protein-binding composition comprises an antibody.

In some embodiments, the protein-binding composition comprises a protein-binding moiety. The protein-binding moiety may comprise one or more antigen recognition domains. The one or more antigen recognition domains of the protein-binding moiety may recognize the target polypeptide or protein. The multivalent molecule may be or comprise an antibody or portion thereof, a chimeric antigen receptor (CAR) or portion thereof, or a T cell receptor (TCR) or portion thereof.

### CAR/TCR

Generally, chimeric antigen receptors (CARs) are engineered fusion proteins constructed from antigen recognition, signaling, and costimulatory domains that may be expressed in T cells to reprogram the T cells to specifically target tumor cells. In some embodiments, a CAR is a recombinant polypeptide construct comprising at least an extracellular antigen binding domain, a transmembrane domain, and a cytoplasmic signaling domain comprising a functional signaling domain derived from a stimulatory molecule. Exogenous T cell receptors (TCRs) are similar to CARs in that they may be engineered to recognize an antigen (e.g., tumor antigen). In some embodiments the TCR is a recombinant polypeptide.

Protein-binding compositions as disclosed herein may comprise a CAR or a TCR. A antigen recognition domain of the CAR or TCR may be configured to recognize or bind to the target protein encoded by the target RNA. The protein-binding composition may further comprise an oligonucleotide tag linked to the CAR or the TCR. Upon the binding of the CAR or the TCR to the target protein, the oligonucleotide tag may hybridize to a unique padlock probe. The binding of the oligonucleotide tag to the padlock probe may topologically lock the padlock probe. The oligonucleotide tag may then be amplified. For example, it may be amplified via rolling circle amplification (RCA), bridge amplification, polymerase chain reaction (PCR), or any combination thereof.

In some embodiments, the extracellular antigen binding domain is an antigen binding fragment of an antibody, or a functional derivative thereof (e.g., an scFv). The specificity of the antigen binding domain may be modified to treat a variety of different disorders, and may be mono-valent or multi-valent (e.g. di-valent, tri-valent). In some embodiments, the antigen binding domain comprises an scFv, and multivalent binding is provided by tandem addition of multiple scFvs bearing different antigen specificities. In some embodiments, the specificity and intended indication of the antigen binding matches that of any of the CAR-T constructs in contemporary clinical trials. For example, the specificity may include anti-CD19 (e.g. axicabtagene ciloleucel for R/R diffuse large cell lymphoma; or Tisagenlecleucel, for R/R B cell ALL and non-Hodgkin lymphoma), anti-CD22 (e.g. for R/RB-ALL), anti-CD19/CD22 dual targeted (e.g. for R/R ALL), anti-CAIX (carbonic anhydrase 9), anti-PSMA (a.k.a FOLH1, e.g. for renal cell carcinoma), anti-MUC1 (e.g. for seminal vesicle carcinoma), anti-CD33(e.g. for acute myeloid leukemia), anti-mesothelin mRNA (e.g. for adenocarcinoma and pleural mesothelioma), anti-FOLR1 (e.g. for metastatic ovarian cancer), anti-carcinoembryonic antigen (a.k.a. CEA, e.g. for CEA-expressing adenocarcinoma liver metastases), anti-IL13RA2 (e.g. for glioblastoma), anti-HER2 (e.g. for sarcoma), or any combination thereof. In some embodiments, one or more of the following antigens may be bound by the CAR-T construct: 1-40-β-amyloid, 4-1BB, 5AC, 5T4, 707-AP, A kinase anchor protein 4 (AKAP-4), activin receptor type-2B (ACVR2B), activin receptor-like kinase 1 (ALK1), adenocarcinoma antigen, adipophilin, adrenoceptor β 3 (ADRB3), AGS-22M6, α folate receptor, α-fetoprotein (AFP), AIM-2, anaplastic lymphoma kinase (ALK), androgen receptor, angiopoietin 2, angiopoietin 3, angiopoietin-binding cell surface receptor 2 (Tie 2), anthrax toxin, AOC3 (VAP-1), B cell maturation antigen (BCMA), B7-H3 (CD276), Bacillus anthracis anthrax, B-cell activating factor (BAFF), B-lymphoma cell, bone marrow stromal cell antigen 2 (BST2), Brother of the Regulator of Imprinted Sites (BORIS), C242 antigen, C5, CA-125, cancer antigen 125 (CA-125 or MUC16), Cancer/testis antigen 1 (NY-ESO-1), Cancer/testis antigen 2 (LAGE-la), carbonic anhydrase 9 (CA-IX), Carcinoembryonic antigen (CEA), cardiac myosin, CCCTC-Binding Factor (CTCF), CCL11 (eotaxin-1), CCR4, CCR5, CD 11, CD123, CD125, CD140a, CD147 (basigin), CD15, CD152, CD154 (CD40L), CD171, CD179a, CD18, CD19, CD2, CD20, CD200, CD22, CD221, CD23 (IgE receptor), CD24, CD25 (α chain of IL-2receptor), CD27, CD274, CD28, CD3, CD3 E, CD30, CD300 molecule-like family member f (CD300LF), CD319 (SLAMF7), CD33, CD37, CD38, CD4, CD40, CD40 ligand, CD41, CD44 v7, CD44 v8, CD44 v6, CD5, CD51, CD52, CD56, CD6, CD70, CD72, CD74, CD79A, CD79B, CD80, CD97, CEA-related antigen, CFD, ch4D5, chromosome X open reading frame 61 (CXORF61), claudin 18.2 (CLDN18.2), claudin 6 (CLDN6), Clostridium difficile, clumping factor A, CLCA2, colony stimulating factor 1 receptor (CSF1R), CSF2, CTLA-4, C-type lectin domain family 12 member A (CLEC12A), C-type lectin-like molecule-1 (CLL-1 or CLECL1), C-X-C chemokine receptor type 4, cyclin B1, cytochrome P4501B1 (CYP1B1), cyp-B, cytomegalovirus, cytomegalovirus glycoprotein B, dabigatran, DLL4, DPP4, DR5, E. coli shiga toxin type-1, E. coli shiga toxin type-2, ecto-ADP-ribosyltransferase 4 (ART4), EGF-like module-containing mucin-like hormone receptor-like 2 (EMR2), EGF-like-domain multiple 7 (EGFL7), elongation factor 2 mutated (ELF2M), endotoxin, Ephrin A2, Ephrin B2, ephrin type-A receptor 2, epidermal growth factor receptor (EGFR), epidermal growth factor receptor variant III (EGFRvIII), episialin, epithelial cell adhesion molecule (EpCAM), epithelial glycoprotein 2 (EGP-2), epithelial glycoprotein 40 (EGP-40), ERBB2, ERBB3, ERBB4, ERG (transmembrane protease, serine 2 (TMPRSS2) ETS fusion gene), Escherichia coli, ETS translocation-variant gene 6, located on chromosome 12p (ETV6-AML), F protein of respiratory syncytial virus, FAP, Fc fragment of IgA receptor (FCAR or CD89), Fc receptor-like 5 (FCRL5), fetal acetylcholine receptor, fibrin II β chain, fibroblast activation protein u (FAP), fibronectin extra domain-B, FGF-5, Fms-Like Tyrosine Kinase 3 (FLT3), folate binding protein (FBP), folate hydrolase, folate receptor 1, folate receptor α, folate receptor β, Fos-related antigen 1, Frizzled receptor, Fucosyl GM1, G250, G protein-coupled receptor 20 (GPR20), G protein-coupled receptor class C group 5, member D (GPRC5D), ganglioside G2 (GD2), GD3 ganglioside, glycoprotein 100 (gp100), glypican-3 (GPC3), GMCSF receptor α-chain, GPNMB, GnT-V, growth differentiation factor 8, GUCY2C, heat shock protein 70-2 mutated (mut hsp70-2), hemagglutinin, Hepatitis A virus cellular receptor 1 (HAVCR1), hepatitis B surface antigen, hepatitis B virus, HER1, HER2/neu, HER3, hexasaccharide portion of globoH glycoceramide (GloboH), HGF, HHGFR, high molecular weight-melanoma-associated antigen (HMW-MAA), histone complex, HIV-1, HLA-DR, HNGF, Hsp90, HST-2 (FGF6), human papilloma virus E6 (HPV E6), human papilloma virus E7 (HPV E7), human scatter factor receptor kinase, human Telomerase reverse transcriptase (hTERT), human TNF, ICAM-1 (CD54), iCE, IFN-α, IFN-β, IFN-γ, IgE, IgE Fc region, IGF-1, IGF-1 receptor, IGHE, IL-12, IL-13, IL-17, IL-17A, IL-17F, IL-10, IL-20, IL-22, IL-23, IL-31, IL-31RA, IL-4, IL-5, IL-6, IL-6 receptor, IL-9, immunoglobulin lambda-like polypeptide 1 (IGLL1), influenza A hemagglutinin, insulin-like growth factor 1 receptor (IGF-1 receptor), insulin-like growth factor 2 (ILGF2), integrin α4β7, integrin β2, integrin α2, integrin α4, integrin α5β1, integrin α707, integrin αIbP3, integrin αvP3, interferon α/β receptor, interferon γ-induced protein, Interleukin 11 receptor α (IL-1IRα), Interleukin-13 receptor subunit α-2 (IL-13Ra2 or CD213A2), intestinal carboxyl esterase, kinase domain region (KDR), KIR2D, KIT (CD117), L1-cell adhesion molecule (L1-CAM), legumain, leukocyte immunoglobulin-like receptor subfamily A member 2 (LILRA2), leukocyte-associated immunoglobulin-like receptor 1 (LAIRI), Lewis-Y antigen, LFA-1 (CD11a), LINGO-1, lipoteichoic acid, LOXL2, L-selectin (CD62L), lymphocyte antigen 6 complex, locus K 9 (LY6K), lymphocyte antigen 75 (LY75), lymphocyte-specific protein tyrosine kinase (LCK), lymphotoxin-α (LT-α) or Tumor necrosis factor-β (TNF-β), macrophage migration inhibitory factor (MIF or MMIF), M-CSF, mammary gland differentiation antigen (NY-BR-1), MCP-1, melanoma cancer testis antigen-1 (MAD-CT-1), melanoma cancer testis antigen-2 (MAD-CT-2), melanoma inhibitor of apoptosis (ML-IAP), melanoma-associated antigen 1 (MAGE-A1), mesothelin, mucin 1, cell surface associated (MUC1), MUC-2, mucin CanAg, myelin-associated glycoprotein, myostatin, N-Acetyl glucosaminyl-transferase V (NA17), NCA-90 (granulocyte antigen), nerve growth factor (NGF), neural apoptosis-regulated proteinase 1, neural cell adhesion molecule (NCAM), neurite outgrowth inhibitor (e.g., NOGO-A, NOGO-B, NOGO-C), neuropilin-1 (NRP1), N-glycolylneuraminic acid, NKG2D, Notch receptor, o-acetyl-GD2 ganglioside (OAcGD2), olfactory receptor 51E2 (OR51E2), oncofetal antigen (h5T4), oncogene fusion protein consisting of breakpoint cluster region (BCR) and Abelson murine leukemia viral oncogene homolog 1 (Abl) (bcr-abl), Oryctolagus cuniculus, OX-40, oxLDL, p53 mutant, paired box protein Pax-3 (PAX3), paired box protein Pax-5 (PAX5), pannexin 3 (PANX3), phosphate-sodium co-transporter, phosphatidylserine, placenta-specific 1 (PLAC1), platelet-derived growth factor receptor α (PDGF-R α), platelet-derived growth factor receptor β (PDGFR-0), polysialic acid, proacrosin binding protein sp32 (OY-TES1), programmed cell death protein 1 (PD-1), proprotein convertase subtilisin/kexin type 9 (PCSK9), prostase, prostate carcinoma tumor antigen-1 (PCTA-1 or Galectin 8), melanoma antigen recognized by T cells 1 (MelanA or MART1), P15, P53, PRAME, prostate stem cell antigen (PSCA), prostate-specific membrane antigen (PSMA), prostatic acid phosphatase (PAP), prostatic carcinoma cells, prostein, Protease Serine 21 (Testisin or PRSS21), Proteasome (Prosome, Macropain) Subunit, R Type, 9 (LMP2), Pseudomonas aeruginosa, rabies virus glycoprotein, RAGE, Ras Homolog Family Member C (RhoC), receptor activator of nuclear factor kappa-B ligand (RANKL), Receptor for Advanced Glycation Endproducts (RAGE-1), receptor tyrosine kinase-like orphan receptor 1 (ROR1), renal ubiquitous 1 (RU1), renal ubiquitous 2 (RU2), respiratory syncytial virus, Rh blood group D antigen, Rhesus factor, sarcoma translocation breakpoints, sclerostin (SOST), selectin P, sialyl Lewis adhesion molecule (sLe), sperm protein 17 (SPA17), sphingosine-1-phosphate, squamous cell carcinoma antigen recognized by T Cells 1, 2, and 3 (SART1, SART2, and SART3), stage-specific embryonic antigen-4 (SSEA-4), Staphylococcus aureus, STEAP1, surviving, syndecan 1 (SDC1)+A314, SOX10, survivin, surviving-2B, synovial sarcoma, X breakpoint 2 (SSX2), T-cell receptor, TCR Γ Alternate Reading Frame Protein (TARP), telomerase, TEM1, tenascin C, TGF-β (e.g., TGF-β 1, TGF-β 2, TGF-β 3), thyroid stimulating hormone receptor (TSHR), tissue factor pathway inhibitor (TFPI), Tn antigen ((Tn Ag) or (GalNAcα-Ser/Thr)), TNF receptor family member B cell maturation (BCMA), TNF-α, TRAIL-R1, TRAIL-R2, TRG, transglutaminase 5 (TGS5), tumor antigen CTAA16.88, tumor endothelial marker 1 (TEM1/CD248), tumor endothelial marker 7-related (TEM7R), tumor protein p53 (p53), tumor specific glycosylation of MUC1, tumor-associated calcium signal transducer 2, tumor-associated glycoprotein 72 (TAG72), tumor-associated glycoprotein 72 (TAG-72)+A327, TWEAK receptor, tyrosinase, tyrosinase-related protein 1 (TYRP1 or glycoprotein 75), tyrosinase-related protein 2 (TYRP2), uroplakin 2 (UPK2), vascular endothelial growth factor (e.g., VEGF-A, VEGF-B, VEGF-C, VEGF-D, PIGF), vascular endothelial growth factor receptor 1 (VEGFR1), vascular endothelial growth factor receptor 2 (VEGFR2), vimentin, v-myc avian myelocytomatosis viral oncogene neuroblastoma derived homolog (MYCN), von Willebrand factor (VWF), Wilms tumor protein (WT1), X Antigen Family, Member TA (XAGE1), β-amyloid, and κ-light chain.

In some embodiments, the transmembrane domain of the CAR is a domain that localizes the CAR to the correct membrane location and stabilizes its structure. In some embodiments, the transmembrane domain comprises a transmembrane domain from CD28. In some embodiments, the CAR comprises a cytoplasmic signaling domain comprising a functional signaling domain derived from a stimulatory molecule. In some embodiments, the stimulatory molecule is a stimulatory receptor molecule. In some embodiments, the stimulatory receptor molecule is a stimulatory receptor molecule of an adaptive immune cell. In some embodiments, the stimulatory molecule is the zeta chain associated with the T cell receptor complex. In some embodiments, the stimulatory molecule is e.g. FCER1G, Fc gamma RIIa, FcR beta (Fc Epsilon Rib), CD3 gamma, CD3 delta, CD3 epsilon, CD79a, CD79b, DAP10, or DAP1. In some embodiments, the intracellular signaling domain comprises one or more functional signaling domains derived from at least one costimulatory molecule. In some embodiments, the costimulatory molecule comprises 4-1BB (i.e., CD137), CD27, CD28 CD30, CD40, PD-1, CD2, CD7, CD258, NKG2C, B7-H3, a ligand that binds to CD83, ICAM-1, LFA-1 (CD1 Ia/CD18), ICOS, or a combination thereof. In some embodiments, the CAR comprises a leader sequence at the amino-terminus (N-terminus) of the CAR fusion protein. In some embodiments, the CAR comprises a signal peptide sequence at the N-terminus of the extracellular antigen recognition domain, wherein the signal peptide sequence is optionally cleaved from the antigen recognition domain (e.g., a scFv) during cellular processing and localization of the CAR to the cellular membrane

In some embodiments, a CAR disclosed herein is a first-, second-, third-, or fourth-generation CAR system, a functional variant thereof, or a combination thereof. In some embodiments, a first-generation CAR comprises an antigen binding domain with specificity for a particular antigen (e.g. an antibody or antigen-binding fragment thereof, such as an scFv, a Fab fragment, a VHH domain, or a VH domain of a heavy-chain only antibody), a transmembrane domain derived from an adaptive immune receptor (e.g. the transmembrane domain from the CD28 receptor), and a signaling domain derived from an adaptive immune receptor (e.g. the three ITAM domains derived from the intracellular region of the CD3 ζ receptor or FcεRIγ). In some embodiments, a second-generation CAR construct comprises the elements of the first-generation CAR and an addition of a co-stimulatory domain to the intracellular signaling domain portion of the CAR (e.g., derived from co-stimulatory receptors that act alongside T-cell receptors such as CD28, CD137/4-1BB, and CD134/OX40). In some embodiments, the co-stimulatory domain abrogates the need for administration of IL-2 alongside a first-generation CAR. In some embodiments, a third-generation CAR comprises the elements of a first generation CAR with the addition of multiple co-stimulatory domains to the intracellular signaling domain portion of the CAR (e.g. CD3ζ-CD28-OX40, or CD3ζ-CD28-41BB). In some embodiments, fourth-generation CAR comprises the elements of a second- or third-generation CARs with the addition of an activating cytokine (e.g. IL-12, IL-23, or IL-27) to the intracellular signaling portion of the CAR (typically between one or more of the costimulatory domains and the CD3ζITAM domain) or under the control of a CAR-induced promoter (e.g. the NFAT/IL-2 minimal promoter).

### Antibodies

Protein-binding compositions as disclosed herein may comprise an antibody or fragment thereof. A “fragment thereof” refers to a moiety, other than a full-length intact antibody, that (1) comprises a portion of an antibody and (2) binds to the corresponding antigen (e.g., target protein). This includes, but is not limited to, Fv, Fab, Fab′, Fab′-SH, F(ab′)₂, diabodies, linear antibodies, single domain antibodies, single domain camelid antibodies, and single-chain antibody (scFv) molecules. A Fab region of the antibody may be configured to recognize or bind to the target protein encoded by the target RNA. The protein-binding composition may further comprise an oligonucleotide tag linked to the antibody or fragment thereof. Upon the binding of the antibody or fragment thereof to the target protein, the oligonucleotide tag may hybridize to a unique padlock probe. The binding of the oligonucleotide tag to the padlock probe may topologically lock the padlock probe. The oligonucleotide tag may then be amplified. For example, it may be amplified via rolling circle amplification (RCA), bridge amplification, polymerase chain reaction (PCR), or any combination thereof.

In some embodiments, an antibody and oligonucleotide tag may be joined via a linker moiety. The antibody may comprise a plurality of oligonucleotide tags linked via a plurality of linker moieties. The antibody may be specific to a target protein or polypeptide corresponding to a target RNA. The oligonucleotide tag may correspond to a sequence of a padlock probe as disclosed herein. The successful binding of the oligonucleotide tag to a padlock probe as disclosed herein may lock the topological configuration of the padlock probe. In some embodiments, the oligonucleotide tag sequence is designed to provide a sequencing read-out that is associated with binding between the antibody-oligonucleotide conjugate and the target polypeptide. In some embodiments, the sequence of the oligonucleotide tag is designed to exhibit minimal hybridization to RNA (e.g., target RNA) in the cellular sample.

The present disclosure provides one or more sets of antibody-oligonucleotide conjugates, comprising at least a first and a second antibody-oligonucleotide conjugate. A set of antibody-oligonucleotide conjugates can be used to conduct any of the detecting and/or sequencing methods described herein.

In some embodiments, a set comprises at least a first antibody-oligonucleotide conjugate comprising a first antibody which selectively binds a first target polypeptide. The first antibody is linked to a first oligonucleotide carrying an oligonucleotide tag sequence which uniquely identifies the first antibody which selectively binds the first target polypeptide.

In some embodiments, the set comprises at least a second antibody-oligonucleotide conjugate comprising a second antibody which selectively binds a second target polypeptide. The second antibody is linked to a second oligonucleotide carrying an oligonucleotide tag sequence which uniquely identifies the second antibody which selectively binds the second target polypeptide.

In some embodiments, the first antibody-oligonucleotide conjugate and the second antibody-oligonucleotide conjugate are the same (e.g., FIG. 28). In some embodiments, the first antibody-oligonucleotide conjugate and the second antibody-oligonucleotide conjugate are different (e.g., FIG. 29).

In some embodiments, the first and second oligonucleotide tag sequences are designed to selectively bind to left and right binding arms of a padlock probe. In some embodiments, the first and second oligonucleotide tag sequences differ from each other.

In some embodiments, a set of antibody-oligonucleotide conjugates comprises 2-20 antibody-oligonucleotide conjugates, wherein each antibody-oligonucleotide conjugate binds its respective target polypeptide. In some embodiments, the set comprises 20-100 antibody-oligonucleotide conjugates, or 100-500 antibody-oligonucleotide conjugates, or 500-1000 antibody-oligonucleotide conjugate, or more antibody-oligonucleotide conjugates.

The present disclosure provides antibody-oligonucleotide conjugates each comprising an antibody linked to an oligonucleotide. In some embodiments, the antibody comprises an intact immunoglobulin, antibody fragment, an antigen binding portion of an antibody, or single-chain antibody. The antibodies can be monoclonal or polyclonal antibodies. The antibodies are capable of binding specifically to a target analyte. The target analyte includes polypeptides, polynucleotides, carbohydrates, saccharides and lipids. In some embodiments, target analytes comprise intact polypeptides or peptide fragments. The antibodies comprises an antigen-binding region (e.g., paratope) that binds specifically to a target analyte.

An immunoglobulin is typically a tetrameric molecule comprising two identical pairs of polypeptide chains where each pair includes a light chain and a heavy chain. The amino portion of the heavy and light chains each comprise a variable region which associate with each other to form an antigen binding region (e.g., paratope). Thus, a typical immunoglobulin can bind two antigens or can bind two target analytes. The carboxyl portion of the heavy chain comprise a constant region which associate with each other to form an Fc region for effector function. The Fc portion of the heavy chains can define the class of antibody which includes IgG, IgM, IgD, IgA or IgE isotype. The heavy and/or light chains can be prepared using recombinant techniques or by immunizing an animal with an antigen of interest.

The antibody fragment generally comprises a portion of an intact immunoglobulin that can bind an antigen. Examples of antibody fragments include but are not limited to Fv, Fab, Fab′, Fab′-SH, F(ab′)₂, and Fd.

In some embodiments, an Fv fragment comprises a variable light chain region (VL) and variable heavy chain region (VH).

In some embodiments, an Fab fragment comprises a monovalent antibody fragment having a variable light chain region (VL), constant light chain region (CL), variable heavy chain region (VH), and first constant region (CH1).

In some embodiments, an Fab′ fragment comprises a monovalent antibody fragment having a variable light chain region (VL), constant light chain region (CL), variable heavy chain region (VH), first constant region (CH1), hinge region, and at least a portion of a second constant region (CH2).

In some embodiments, an F(ab′)₂ fragment comprises a bivalent antibody fragment having two Fab fragments linked via a disulfide bridge at the hinge region.

A single-chain antibody (scFv) typically comprises a single polypeptide chain (e.g., a monovalent antibody molecule) having a variable light chain region (VL) and variable heavy chain region (VH) joined by a polypeptide linker (see, e.g., Bird et al., 1988, Science 242:423-26 and Huston et al., 1988, Proc. Natl. Acad. Sci. USA 85:5879-83). The amino-terminal end of the single-chain antibody comprises either the variable light chain region (VL) or the variable heavy chain region (VH). In some embodiments, the single-chain antibody comprises an scFv-Fc antibody which further comprises an antibody hinge region, and at least a portion of the Fc region including the CH2 and/or the CH3 region. In some embodiments, the single-chain antibody comprises an scFv-CH antibody which further comprises an antibody hinge region, and at least a portion of the CH3 region.

In some embodiments, the antibody-oligonucleotide conjugates comprise an antibody linked to an oligonucleotide by a linker moiety. In some embodiments, the linker moiety comprises streptavidin (or an avidin-like moiety), biotin, an amine group, or a disulfide group. In some embodiments, the linker moiety is not cleavable or not removable. In some embodiments, the linker moiety is cleavable or removable. For example, the linker moiety can be cleavable with light (e.g., UV light), chemically-cleavable (e.g., dithiothreitol), heat-cleavable, or enzymatically cleavable.

In some embodiments, antibody-oligonucleotide conjugates can be prepared by cross-linking amino groups on the antibody and oligonucleotide using glutaraldehyde. The lysine side chain epsilon-amide is commonly targeted to conjugate to oligonucleotides. In some embodiments, maleimide-modified antibodies can be reacted with sulfhydryl-modified oligonucleotides. In some embodiments, heterobifunctional cross-linkers are introduced as bridges to link together the antibody and oligonucleotide.

In some embodiments, the antibody-oligonucleotide conjugates comprise an antibody linked to an oligonucleotide. In some embodiments, the oligonucleotide comprises an nucleic acid comprising DNA, RNA, or chimeric DNA and RNA. In some embodiments, the oligonucleotide comprises canonical nucleotides or nucleotide analogs such as locked nucleic acids (LNA). In some embodiments, the length of the oligonucleotide can be 10-50 nucleotides in length, or 50-100 nucleotides in length, or 100-200 nucleotides in length.

In some embodiments, the oligonucleotide is designed to carry at least one tag sequence, where the tag sequence selectively binds to left and right binding arms of a padlock probe. In some embodiments, the oligonucleotide tag sequence also uniquely identifies the antibody to which it is conjugated, where the antibody selectively binds a target polypeptide. In some embodiments, the oligonucleotide tag sequence is designed to provide a sequencing read-out that is associated with binding between the antibody-oligonucleotide conjugate and the target polypeptide. In some embodiments, the sequence of the oligonucleotide tag is designed to exhibit minimal hybridization to RNA (e.g., target RNA) in the cellular sample.

In some embodiments, a set of antibody-oligonucleotide conjugates comprises at least a first and a second antibody-oligonucleotide conjugate. In some embodiments, a set of oligonucleotide tags comprises at least a first and a second oligonucleotide tag. In some embodiments, the set of oligonucleotide tags comprises 2-20 oligonucleotide tags wherein each oligonucleotide tag in the set uniquely identifies its respective conjugated antibody where the antibody selectively binds a target polypeptide. The set of oligonucleotide tags comprises 20-100 oligonucleotide tags, or 100-500 oligonucleotide tags, or 500-1000 oligonucleotide tags, or more oligonucleotide tags.

As depicted in the non-limiting embodiment of FIG. 28, a first antibody-nucleotide conjugate and a second antibody-nucleotide conjugate may be used against a protein (in FIG. 28, “Protein-1 encoded by RNA-1”). The first antibody-nucleotide conjugate, the second antibody-nucleotide conjugate, or both antibody-nucleotide conjugates may comprise a linker moiety. A first oligonucleotide tag may be linked to the first antibody-oligonucleotide conjugate via a first linker moiety. The first oligonucleotide tag may conjugate to a padlock probe. The first oligonucleotide tag of the first antibody-oligonucleotide conjugate may, e.g., ligate, chemically conjugate, or have proximity crosslinking to the padlock probe. A second oligonucleotide tag of the second antibody-oligonucleotide conjugate may be linked to the second antibody via a second linker moiety. The second oligonucleotide tag may conjugate to a padlock probe. The second oligonucleotide tag of the second antibody-oligonucleotide conjugate may, e.g., ligate, chemically conjugate, or have proximity crosslinking to the padlock probe. A dehybridization step may be completed to eliminate circulate probes from a region of the padlock probe that is not ligated or not crosslinked to an oligonucleotide tag. Resultantly, successful ligation or chemical crosslinking of one or more oligonucleotide tag to the padlock probe may topologically lock the padlock probe in place. In the example of FIG. 28, the hybridization of both the first oligonucleotide tag and the second oligonucleotide tag to the padlock probe is required to topologically lock the padlock probe. Next, a rolling circle amplification (RCA) step may be completed. The RCA step may comprise hybridizing an RCA primer to a sequence of the padlock probe, and then completing one or more cycles of RCA. The RCA step may comprise cleaving a phosphate group of the first oligonucleotide tag or the second oligonucleotide tag (in FIG. 28, cleaving of a phosphate group of second oligonucleotide is denoted by a star), thereby generating a 3′ end, and then completing one or more cycles of RCA.

As depicted in the non-limiting embodiment of FIG. 29, a first antibody-oligonucleotide conjugate and a second antibody-oligonucleotide conjugate may be used against a target protein (in FIG. 29, “Protein-1 encoded by RNA-1”). The first antibody-oligonucleotide conjugate may comprise a first antibody and a first oligonucleotide tag. The first oligonucleotide tag may be linked to the first antibody via a first linker moiety. The second antibody-oligonucleotide tag may comprise a second antibody and a second oligonucleotide tag. Thee second oligonucleotide tag may be linked to the second antibody via a second linker moiety. The first antibody-oligonucleotide conjugate and the second antibody-oligonucleotide conjugate may be specific to the same target protein. A first oligonucleotide tag (denoted in FIG. 29 by the dashed line) of the first antibody-oligonucleotide conjugate may conjugate to a padlock probe. The first oligonucleotide tag of the first antibody-oligonucleotide conjugate may, e.g., ligate, chemically conjugate, or have proximity crosslinking to the padlock probe. A second oligonucleotide tag (denoted in FIG. 29 by the dotted line) of the second antibody-oligonucleotide conjugate may conjugate to a padlock probe. The second oligonucleotide tag of the second antibody-oligonucleotide conjugate may, e.g., ligate, chemically conjugate, or have proximity crosslinking to the padlock probe. A dehybridization step may be completed to eliminate the first oligonucleotide tag (dashed) or the second oligonucleotide tag (dotted). Resultantly, successful ligation or chemical crosslinking of the first oligonucleotide tag or the second oligonucleotide tag to the padlock probe may topologically lock the padlock probe in place. In the example of FIG. 29, the hybridization of the first oligonucleotide tag and second oligonucleotide tag is required to successfully ligate to the padlock probe. This step is intended to prevent false positives, wherein false positives would lack the topological locking of the padlock probe. Next, a rolling circle amplification (RCA) step may be completed. The RCA step may comprise hybridizing an RCA primer to a sequence of the padlock probe, and then completing one or more cycles of RCA. The RCA step may comprise cleaving a phosphate group of the first oligonucleotide tag or the second oligonucleotide tag (in FIG. 29, cleaving of a phosphate group of the second oligonucleotide is denoted by a star), thereby generating a 3′ end, and then completing one or more cycles of RCA. The RCA step may comprise ligation of the first (dashed) oligonucleotide tag, and no ligation of the second (dotted) oligonucleotide tag, as the second oligonucleotide tag is missing a 5′ phosphate group. Thus, the 3′ end of the first oligonucleotide tag is hybridized to the padlock probe, as is used as an RCA primer. One or more cycles of RCA may then be conducted.

In some embodiments, the target analytes comprise polypeptides or peptide fragments. In some embodiments, a target polypeptide is encoded by a target RNA in the cellular sample. Target polypeptides include polypeptides having post-translationally modified forms, including methylation, phosphorylation, glycosylation, hydroxylation, ubiquitination, nitrosylation, acetylation, lipidation, ADP-ribosylation, carbonylation, SUMOylation and/or disulfide bond formation. The target polypeptides can be subjected to proteolysis for example by a protease or cleavage due to ribosomal skipping. Target polypeptides also include precursor molecules that have not yet been subjected to post-translation modification. Target polypeptides include muteins, variants, chimeric proteins and fusion proteins. Target polypeptides can be labeled with a binding partner molecule having an affinity moiety, such as for example biotin (or its derivatives), digoxigenin, fluorescein, cholesterol, maltose, or any of the affinity molecules described below.

In some embodiments, polysaccharides inside the cellular sample can be detected by contacting the cellular sample with lectin conjugated to an oligonucleotide carrying a tag sequence that is designed to bind the left and right arms of a padlock probe. Lectin-oligonucleotide conjugates can permit detection of polysaccharides using a sequencing readout.

In some embodiments, lipids inside the cellular sample can be detected by contacting the cellular sample with a lipid-specific binding protein or an amphipathic polypeptide which are conjugated to an oligonucleotide carrying a tag sequence that is designed to bind the left and right arms of a padlock probe. Lipid-specific binding proteins and amphipathic polypeptides that are conjugated to tagged oligonucleotides can permit detection of lipids using a sequencing readout.

### Sequencing Polymerases

In any of the methods described herein, sequencing polymerases can be used for conducting sequencing reactions. In some embodiments, the sequencing polymerase(s) is/are capable of binding and incorporating a complementary nucleotide opposite a nucleotide in a concatemer template molecule. In some embodiments, the sequencing polymerase(s) is/are capable of binding a complementary nucleotide unit of a multivalent molecule opposite a nucleotide in a concatemer template molecule. In some embodiments, the plurality of sequencing polymerases comprise recombinant mutant polymerases.

Examples of suitable polymerases for use in sequencing with nucleotides and/or multivalent molecules include but are not limited to: Klenow DNA polymerase; Thermus aquaticus DNA polymerase I (Taq polymerase); KlenTaq polymerase; Candidatus altiarchaeales archaeon; Candidatus Hadarchaeum Yellowstonense; Hadesarchaea archaeon; Euryarchaeota archaeon; Thermoplasmata archaeon; Thermococcus polymerases such as Thermococcus litoralis, bacteriophage T7 DNA polymerase; human alpha, delta and epsilon DNA polymerases; bacteriophage polymerases such as T4, RB69 and phi29 bacteriophage DNA polymerases; Pyrococcus furiosus DNA polymerase (Pfu polymerase); Bacillus subtilis DNA polymerase III; E. coli DNA polymerase III alpha and epsilon; 9 degree N polymerase; reverse transcriptases such as HIV type M or 0 reverse transcriptases; avian myeloblastosis virus reverse transcriptase; Moloney Murine Leukemia Virus (MMLV) reverse transcriptase; or telomerase. Further non-limiting examples of DNA polymerases include those from various Archaea genera, such as, Aeropyrum, Archaeglobus, Desulfurococcus, Pyrobaculum, Pyrococcus, Pyrolobus, Pyrodictium, Staphylothermus, Stetteria, Sulfolobus, Thermococcus, and Vulcanisaeta and the like or variants thereof, including such polymerases as are known in the art such as 9 degrees N, VENT, DEEP VENT, THERMINATOR, Pfu, KOD, Pfx, Tgo and RB69 polymerases.

Sequencing cDNA Amplicons

In any of the methods described herein, the sequencing comprises conducting sequencing reactions inside a cellular sample, where the cDNA amplicons are the concatemer molecules. In some embodiments, the sequencing employs non-labeled chain-terminating nucleotides. In some embodiments, a cycle of sequencing comprises the steps of (a) sequentially contacting a primed concatemer (e.g., a concatemer annealed to a plurality of sequencing primers) with at least two separate mixtures under ternary complex stabilizing conditions, wherein the at least two separate mixtures each include a polymerase and a nucleotide, whereby the sequentially contacting results in the primed concatemer being contacted, under the ternary complex stabilizing conditions, with nucleotide cognates for first, second and third base type base types in the template; (b) examining the at least two separate mixtures to determine whether a ternary complex formed; and (c) identifying the next correct nucleotide for the primed concatemer, wherein the next correct nucleotide is identified as a cognate of the first, second or third base type if ternary complex is detected in step (b), and wherein the next correct nucleotide is imputed to be a nucleotide cognate of a fourth base type based on the absence of a ternary complex in step (b); (d) adding a next correct nucleotide to the primer of the primed concatemer after step (b), thereby producing an extended primer; and (e) repeating steps (a) through (d) at least once on the primed concatemer that comprises the extended primer. In some embodiments, the repeating comprises repeating at least 2 times, 5 times, 10 times, 15 times, 20 times, 25 times, 30 times, 35 times, 40 times, 45 times, or at least 50 times. In some embodiments, the sequencing is sequencing-by-binding (SBB). In some embodiments, a cycle of sequencing-by-binding (SBB) can comprise sequentially contacting a primed concatemer (e.g., a concatemer annealed to a plurality of sequencing primers) with at least two separate mixtures under ternary complex stabilizing conditions, wherein the at least two separate mixtures can each include a polymerase and a nucleotide, whereby the sequentially contacting can result in the primed concatemer being contacted, under the ternary complex stabilizing conditions, with nucleotide cognates for first, second and third base type base types in the template. In some embodiments, a cycle of sequencing-by-binding (SBB) can comprise examining the at least two separate mixtures to determine whether a ternary complex formed. In some embodiments, a cycle of sequencing-by-binding (SBB) can comprise identifying the next correct nucleotide for the primed concatemer, wherein the next correct nucleotide can be identified as a cognate of the first, second or third base type if ternary complex was detected and wherein the next correct nucleotide can be imputed to be a nucleotide cognate of a fourth base type based on the absence of a ternary complex. In some embodiments, a cycle of sequencing-by-binding (SBB) can comprise adding a next correct nucleotide to the primer of the primed concatemer, thereby producing an extended primer. Sequencing-by-binding methods are described in U.S. Pat. Nos. 10,246,744 and 10,731,141 (where the contents of both patents are hereby incorporated by reference in their entireties). In some embodiments, no more than 2-30 sequencing-by-binding (SBB) cycles can be conducted.

### Nucleotides and Chain-Terminating Nucleotides

The present disclosure provides methods for detecting the sequence of a nucleic acid in a biological sample. The nucleic acid may be, e.g., DNA or RNA. The biological sample may be a cellular sample.

The present disclosure provides methods for detecting in situ at least two different target RNA molecules in a cellular sample, which can include conducting sequencing reactions inside the cellular sample, where the cDNA amplicons can be the concatemer molecules.

In any of the methods described herein, any of the sequencing methods described herein can employ at least one nucleotide. The nucleotides comprise a base, sugar and at least one phosphate group. In some embodiments, at least one nucleotide in the plurality comprises an aromatic base, a five carbon sugar (e.g., ribose or deoxyribose), and one or more phosphate groups (e.g., 1-10 phosphate groups). The plurality of nucleotides can comprise at least one type of nucleotide selected from a group consisting of dATP, dGTP, dCTP, dTTP and dUTP. The plurality of nucleotides can comprise at a mixture of any combination of two or more types of nucleotides selected from a group consisting of dATP, dGTP, dCTP, dTTP and/or dUTP. In some embodiments, at least one nucleotide in the plurality is not a nucleotide analog. In some embodiments, at least one nucleotide in the plurality comprises a nucleotide analog.

In some embodiments, in any of the methods for sequencing described herein, at least one nucleotide in the plurality of nucleotides comprise a chain of one, two or three phosphorus atoms where the chain is typically attached to the 5′ carbon of the sugar moiety via an ester or phosphoramide linkage. In some embodiments, at least one nucleotide in the plurality is an analog having a phosphorus chain in which the phosphorus atoms are linked together with intervening O, S, NH, methylene or ethylene. In some embodiments, the phosphorus atoms in the chain include substituted side groups including 0, S or BH3. In some embodiments, the chain includes phosphate groups substituted with analogs including phosphoramidate, phosphorothioate, phosphordithioate, and O-methyl phosphoramidite groups.

In some embodiments, in any of the methods for sequencing described herein, at least one nucleotide in the plurality of nucleotides comprises a terminator nucleotide analog having a chain terminating moiety (e.g., blocking moiety) at the sugar 2′ position, at the sugar 3′ position, or at the sugar 2′ and 3′ position. In some embodiments, the chain terminating moiety can inhibit polymerase-catalyzed incorporation of a subsequent nucleotide unit or free nucleotide in a nascent strand during a primer extension reaction. In some embodiments, the chain terminating moiety is attached to the 3′ sugar hydroxyl position where the sugar comprises a ribose or deoxyribose sugar moiety. In some embodiments, the chain terminating moiety is removable/cleavable from the 3′ sugar hydroxyl position to generate a nucleotide having a 3′OH sugar group which is extendible with a subsequent nucleotide in a polymerase-catalyzed nucleotide incorporation reaction. In some embodiments, the chain terminating moiety comprises an alkyl group, alkenyl group, alkynyl group, allyl group, aryl group, benzyl group, azide group, amine group, amide group, keto group, isocyanate group, phosphate group, thiol group, disulfide group, carbonate group, urea group, or silyl group. In some embodiments, the chain terminating moiety is cleavable/removable from the nucleotide, for example by reacting the chain terminating moiety with a chemical agent, pH change, light or heat. In some embodiments, the chain terminating moieties alkyl, alkenyl, alkynyl and allyl are cleavable with tetrakis(triphenylphosphine)palladium(0) (Pd(PPh3)4) with piperidine, or with 2,3-Dichloro-5,6-dicyano-1,4-benzo-quinone (DDQ). In some embodiments, the chain terminating moieties aryl and benzyl are cleavable with H2 Pd/C. In some embodiments, the chain terminating moieties amine, amide, keto, isocyanate, phosphate, thiol, disulfide are cleavable with phosphine or with a thiol group including beta-mercaptoethanol or dithiothritol (DTT). In some embodiments, the chain terminating moiety carbonate is cleavable with potassium carbonate (K₂CO₃) in MeOH, with triethylamine in pyridine, or with Zn in acetic acid (AcOH). In some embodiments, the chain terminating moieties urea and silyl are cleavable with tetrabutylammonium fluoride, pyridine-HF, with ammonium fluoride, or with triethylamine trihydrofluoride.

In some embodiments, in any of the methods for sequencing described herein, at least one nucleotide in the plurality of nucleotides comprises a terminator nucleotide analog having a chain terminating moiety (e.g., blocking moiety) at the sugar 2′ position, at the sugar 3′ position, or at the sugar 2′ and 3′ position. In some embodiments, the chain terminating moiety comprises an azide, azido or azidomethyl group. In some embodiments, the chain terminating moiety comprises a 3′-O-azido or 3′-O-azidomethyl group. In some embodiments, the chain terminating moieties azide, azido and azidomethyl group are cleavable/removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (BS-TPP) or Tri(hydroxyproyl)phosphine (THPP). In some embodiments, the cleaving agent comprises 4-dimethylaminopyridine (4-DMAP).

In some embodiments, in any of the methods for sequencing described herein, the nucleotide comprises a chain terminating moiety which is selected from a group consisting of 3′-deoxy nucleotides, 2′,3′-dideoxynucleotides, 3′-methyl, 3′-azido, 3′-azidomethyl, 3′-O-azidoalkyl, 3′-O-ethynyl, 3′-O-aminoalkyl, 3′-O-fluoroalkyl, 3′-fluoromethyl, 3′-difluoromethyl, 3′-trifluoromethyl, 3′-sulfonyl, 3′-malonyl, 3′-amino, 3′-O-amino, 3′-sulfhydral, 3′-aminomethyl, 3′-ethyl, 3′butyl, 3′-tert butyl, 3′-Fluorenylmethyloxycarbonyl, 3′ tert-Butyloxycarbonyl, 3′-O-alkyl hydroxylamino group, 3′-phosphorothioate, and 3-O-benzyl, or derivatives thereof.

In some embodiments, in any of the methods for sequencing described herein, the plurality of nucleotides comprises a plurality of nucleotides labeled with detectable reporter moiety. The detectable reporter moiety comprises a fluorophore. In some embodiments, the fluorophore is attached to the nucleotide base. In some embodiments, the fluorophore is attached to the nucleotide base with a linker which is cleavable/removable from the base. In some embodiments, at least one of the nucleotides in the plurality is not labeled with a detectable reporter moiety. In some embodiments, a particular detectable reporter moiety (e.g., fluorophore) that is attached to the nucleotide can correspond to the nucleotide base (e.g., dATP, dGTP, dCTP, dTTP or dUTP) to permit detection and identification of the nucleotide base.

In some embodiments, in any of the methods for sequencing nucleic acid molecules described herein, the cleavable linker on the nucleotide base comprises a cleavable moiety comprising an alkyl group, alkenyl group, alkynyl group, allyl group, aryl group, benzyl group, azide group, amine group, amide group, keto group, isocyanate group, phosphate group, thio group, disulfide group, carbonate group, urea group, or silyl group. In some embodiments, the cleavable linker on the base is cleavable/removable from the base by reacting the cleavable moiety with a chemical agent, pH change, light or heat. In some embodiments, the cleavable moieties alkyl, alkenyl, alkynyl and allyl are cleavable with tetrakis(triphenylphosphine)palladium(0) (Pd(PPh3)4) with piperidine, or with 2,3-Dichloro-5,6-dicyano-1,4-benzo-quinone (DDQ). In some embodiments, the cleavable moieties aryl and benzyl are cleavable with H2 Pd/C. In some embodiments, the cleavable moieties amine, amide, keto, isocyanate, phosphate, thiol, disulfide are cleavable with phosphine or with a thiol group including beta-mercaptoethanol or dithiothritol (DTT). In some embodiments, the cleavable moiety carbonate is cleavable with potassium carbonate (K2CO3) in MeOH, with triethylamine in pyridine, or with Zn in acetic acid (AcOH). In some embodiments, the cleavable moieties urea and silyl are cleavable with tetrabutylammonium fluoride, pyridine-HF, with ammonium fluoride, or with triethylamine trihydrofluoride.

In some embodiments, in any of the methods for sequencing described herein, the cleavable linker on the nucleotide base comprises cleavable moiety including an azide, azido or azidomethyl group. In some embodiments, the cleavable moieties azide, azido and azidomethyl group are cleavable/removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (BS-TPP) or Tri(hydroxyproyl)phosphine (THPP). In some embodiments, the cleaving agent comprises 4-dimethylaminopyridine (4-DMAP).

In some embodiments, in any of the methods for sequencing described herein, the chain terminating moiety (e.g., at the sugar 2′ and/or sugar 3′ position) and the cleavable linker on the nucleotide base have the same or different cleavable moieties. In some embodiments, the chain terminating moiety (e.g., at the sugar 2′ and/or sugar 3′ position) and the detectable reporter moiety linked to the base are chemically cleavable/removable with the same chemical agent. In some embodiments, the chain terminating moiety (e.g., at the sugar 2′ and/or sugar 3′ position) and the detectable reporter moiety linked to the base are chemically cleavable/removable with different chemical agents.

### Multivalent Molecules

Methods as described herein may employ at least one multivalent molecule. A multivalent binding complex may be formed between one or more polymer-nucleotide conjugates, one or more polymerases, one or more plurality of primed target nucleic acid molecules, or any combination thereof. The multivalent binding complex may be stable, and may be used in any sequencing method as described herein. For example, the multivalent binding complex may allow a detection or base-calling step in a sequencing cycle to be separated from the nucleotide incorporation step.

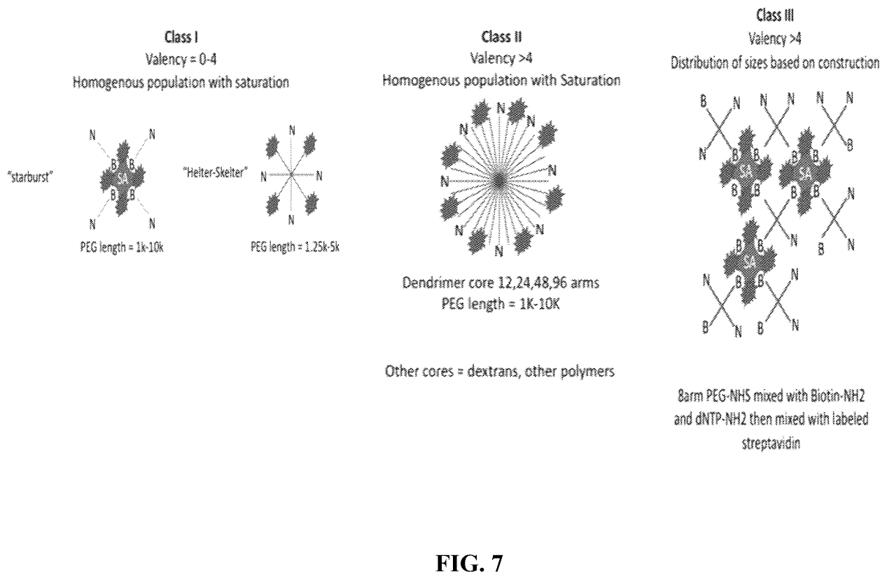

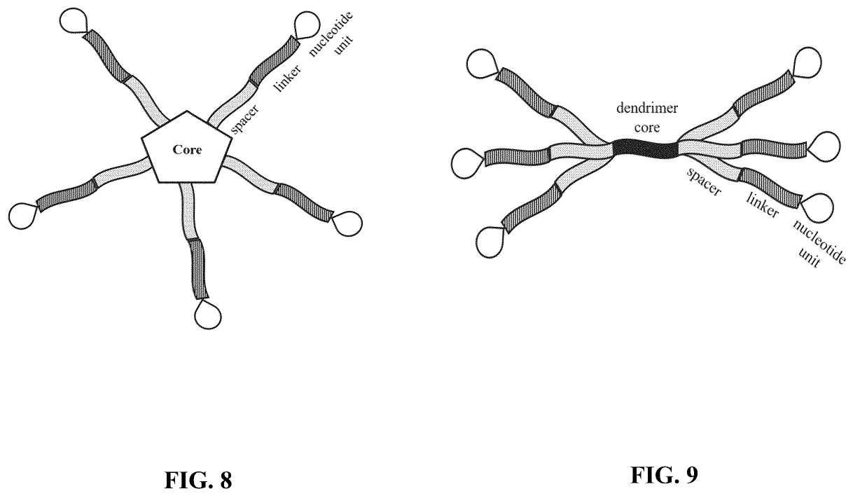

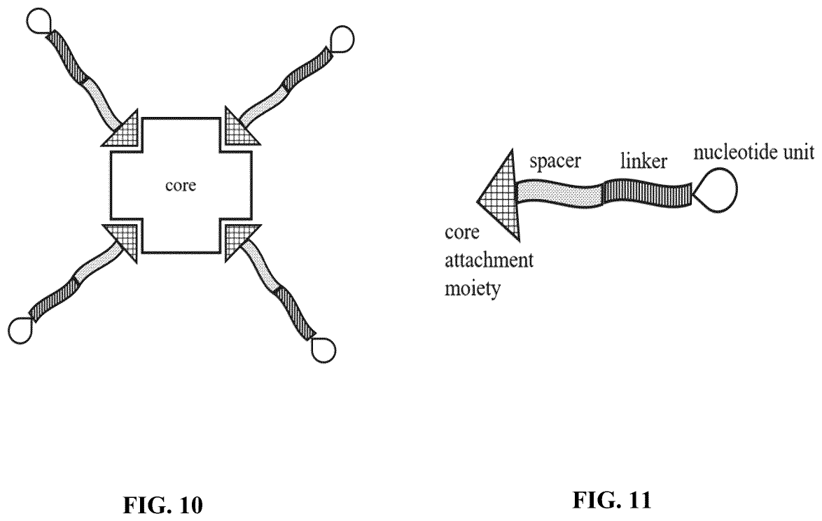

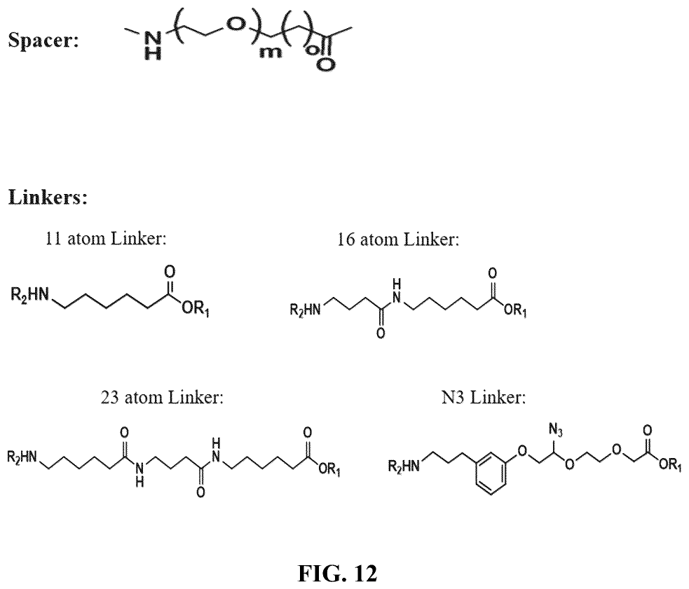

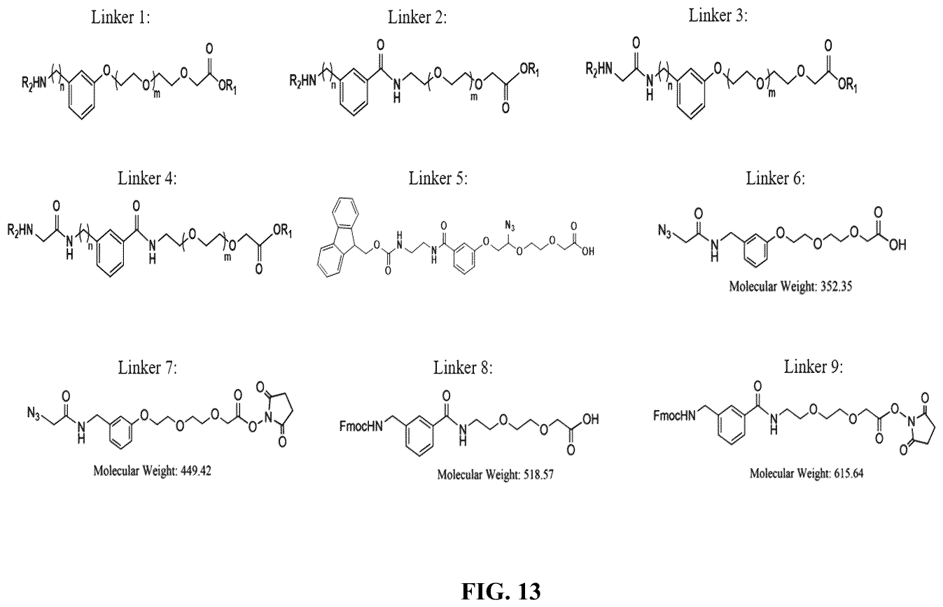

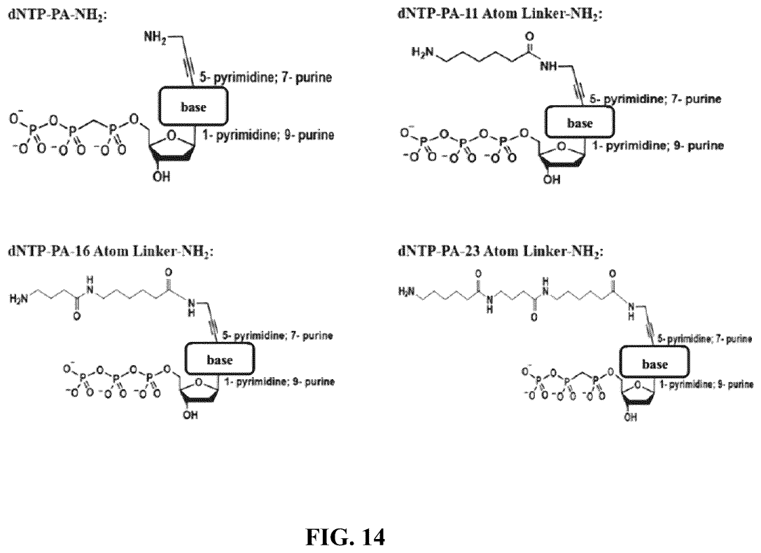

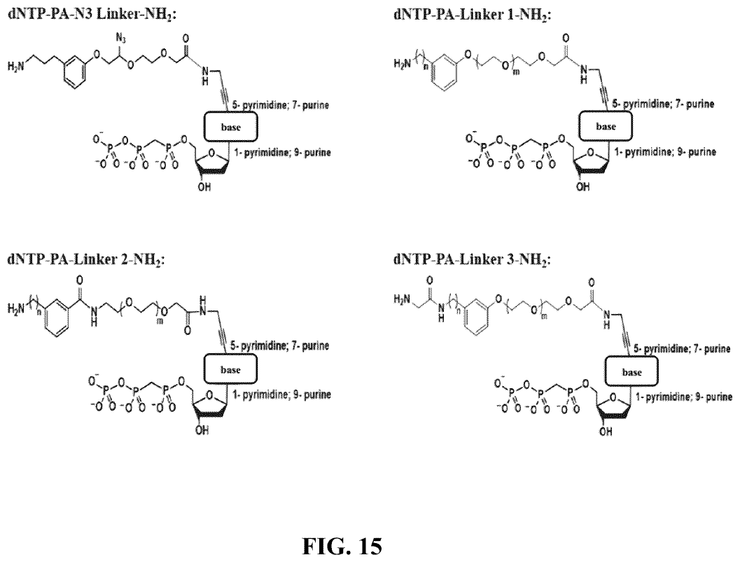

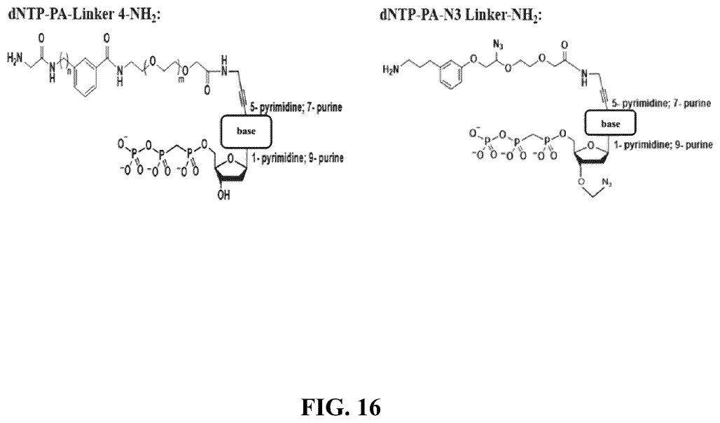

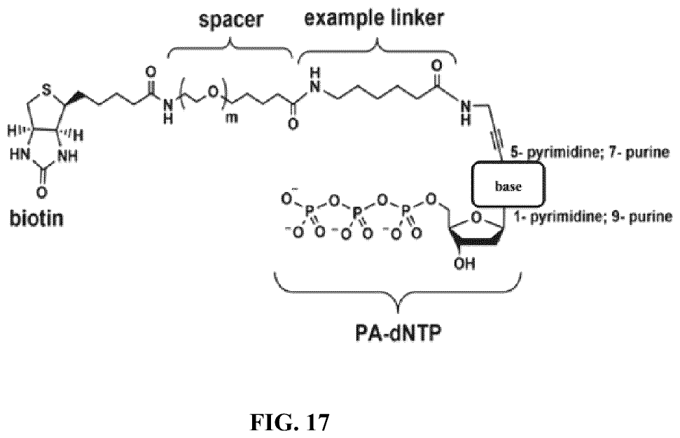

Multivalent molecules as disclosed herein may comprise a plurality of nucleotide arms attached to a core and having any configuration including a starburst, helter skelter, or bottle brush configuration (e.g., FIG. 7). The multivalent molecule comprises: (1) a core; and (2) a plurality of nucleotide arms which comprise (i) a core attachment moiety, (ii) a spacer comprising a PEG moiety, (iii) a linker, and (iv) a nucleotide unit, wherein the core is attached to the plurality of nucleotide arms, wherein the spacer is attached to the linker, wherein the linker is attached to the nucleotide unit. In some embodiments, the multivalent molecule can comprise a core. In some embodiments, the multivalent molecule can comprise one or more linker moieties (e.g., FIG. 26). In some embodiments, the multivalent molecule can comprise a plurality of nucleotide arms. In some embodiments, a nucleotide arm from the plurality of nucleotide arms can comprise a core In some embodiments, a nucleotide arm can comprise a spacer. In some embodiments, the spacer can comprise a PEG moiety. In some embodiments, a nucleotide arm can comprise a linker. In some embodiments, a nucleotide arm can comprise a nucleotide unit. In some embodiments, the spacer can be attached to the linker. In some embodiments, the linker can be attached to the nucleotide unit. In some embodiments, the core can be attached to the plurality of nucleotide arms. In some embodiments, the nucleotide unit can comprise a base, sugar and at least one phosphate group, and the linker can be attached to the nucleotide unit through the base. In some embodiments, the linker can comprise an aliphatic chain or an oligo ethylene glycol chain where both linker chains can have 2-6 subunits. In some embodiments, the linker can also include an aromatic moiety. A non-limiting example nucleotide arm is shown in FIG. 11. Non-limiting example multivalent molecules are shown in FIGS. 7-10. A non-limiting example spacer is shown in FIG. 12 (top) and non-limiting example linkers are shown in FIG. 12 (bottom) and FIG. 13. Non-limiting example nucleotides attached to a linker are shown in FIGS. 14-16. A non-limiting example biotinylated nucleotide arm is shown in FIG. 17.

In some embodiments, a multivalent molecule comprises a core attached to multiple nucleotide arms, and wherein the multiple nucleotide arms have the same type of nucleotide unit which is selected from a group consisting of dATP, dGTP, dCTP, dTTP and dUTP.

In some embodiments, a multivalent molecule comprises a core attached to multiple nucleotide arms, where each arm includes a nucleotide unit. The nucleotide unit comprises an aromatic base, a five carbon sugar (e.g., ribose or deoxyribose), and one or more phosphate groups (e.g., 1-10 phosphate groups). The plurality of multivalent molecules can comprise one type multivalent molecule having one type of nucleotide unit selected from a group consisting of dATP, dGTP, dCTP, dTTP and dUTP. The plurality of multivalent molecules can comprise at a mixture of any combination of two or more types of multivalent molecules, where individual multivalent molecules in the mixture comprise nucleotide units selected from a group consisting of dATP, dGTP, dCTP, dTTP and/or dUTP.

In some embodiments, the nucleotide unit comprises a chain of one, two or three phosphorus atoms where the chain is typically attached to the 5′ carbon of the sugar moiety via an ester or phosphoramide linkage. In some embodiments, at least one nucleotide unit is a nucleotide analog having a phosphorus chain in which the phosphorus atoms are linked together with intervening O, S, NH, methylene or ethylene. In some embodiments, the phosphorus atoms in the chain include substituted side groups including 0, S or BH3. In some embodiments, the chain includes phosphate groups substituted with analogs including phosphoramidate, phosphorothioate, phosphordithioate, and O-methyl phosphoramidite groups.

In some embodiments, the multivalent molecule comprises a core attached to multiple nucleotide arms, and wherein individual nucleotide arms comprise a nucleotide unit which is a nucleotide analog having a chain terminating moiety (e.g., blocking moiety) at the sugar 2′ position, at the sugar 3′ position, or at the sugar 2′ and 3′ position. In some embodiments, the nucleotide unit comprises a chain terminating moiety (e.g., blocking moiety) at the sugar 2′ position, at the sugar 3′ position, or at the sugar 2′ and 3′ position. In some embodiments, the chain terminating moiety can inhibit polymerase-catalyzed incorporation of a subsequent nucleotide unit or free nucleotide in a nascent strand during a primer extension reaction. In some embodiments, the chain terminating moiety is attached to the 3′ sugar hydroxyl position where the sugar comprises a ribose or deoxyribose sugar moiety. In some embodiments, the chain terminating moiety is removable/cleavable from the 3′ sugar hydroxyl position to generate a nucleotide having a 3′OH sugar group which is extendible with a subsequent nucleotide in a polymerase-catalyzed nucleotide incorporation reaction. In some embodiments, the chain terminating moiety comprises an alkyl group, alkenyl group, alkynyl group, allyl group, aryl group, benzyl group, azide group, amine group, amide group, keto group, isocyanate group, phosphate group, thiol group, disulfide group, carbonate group, urea group, or silyl group. In some embodiments, the chain terminating moiety is cleavable/removable from the nucleotide unit, for example by reacting the chain terminating moiety with a chemical agent, pH change, light or heat. In some embodiments, the chain terminating moieties alkyl, alkenyl, alkynyl and allyl are cleavable with tetrakis(triphenylphosphine)palladium(0) (Pd(PPh3)4) with piperidine, or with 2,3-Dichloro-5,6-dicyano-1,4-benzo-quinone (DDQ). In some embodiments, the chain terminating moieties aryl and benzyl are cleavable with H2 Pd/C. In some embodiments, the chain terminating moieties amine, amide, keto, isocyanate, phosphate, thiol, disulfide are cleavable with phosphine or with a thiol group including beta-mercaptoethanol or dithiothritol (DTT). In some embodiments, the chain terminating moiety carbonate is cleavable with potassium carbonate (K2CO3) in MeOH, with triethylamine in pyridine, or with Zn in acetic acid (AcOH). In some embodiments, the chain terminating moieties urea and silyl are cleavable with tetrabutylammonium fluoride, pyridine-HF, with ammonium fluoride, or with triethylamine trihydrofluoride.

In some embodiments, the nucleotide unit comprises a chain terminating moiety (e.g., blocking moiety) at the sugar 2′ position, at the sugar 3′ position, or at the sugar 2′ and 3′ position. In some embodiments, the chain terminating moiety comprises an azide, azido or azidomethyl group. In some embodiments, the chain terminating moiety comprises a 3′-O-azido or 3′-O-azidomethyl group. In some embodiments, the chain terminating moieties azide, azido and azidomethyl group are cleavable/removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (B S-TPP) or Tri(hydroxyproyl)phosphine (THPP). In some embodiments, the cleaving agent comprises 4-dimethylaminopyridine (4-DMAP).

In some embodiments, the nucleotide unit comprising a chain terminating moiety which is selected from a group consisting of 3′-deoxy nucleotides, 2′,3′-dideoxynucleotides, 3′-methyl, 3′-azido, 3′-azidomethyl, 3′-O-azidoalkyl, 3′-O-ethynyl, 3′-O-aminoalkyl, 3′-O-fluoroalkyl, 3′-fluoromethyl, 3′-difluoromethyl, 3′-trifluoromethyl, 3′-sulfonyl, 3′-malonyl, 3′-amino, 3′-O-amino, 3′-sulfhydral, 3′-aminomethyl, 3′-ethyl, 3′butyl, 3′-tert butyl, 3′-Fluorenylmethyloxycarbonyl, 3′ tert-Butyloxycarbonyl, 3′-O-alkyl hydroxylamino group, 3′-phosphorothioate, and 3-O-benzyl, or derivatives thereof.

In some embodiments, the multivalent molecule comprises a core attached to multiple nucleotide arms, wherein the nucleotide arms comprise a spacer, linker and nucleotide unit, and wherein the core, linker and/or nucleotide unit is labeled with detectable reporter moiety. In some embodiments, the detectable reporter moiety comprises a fluorophore. In some embodiments, a particular detectable reporter moiety (e.g., fluorophore) that is attached to the multivalent molecule can correspond to the base (e.g., dATP, dGTP, dCTP, dTTP or dUTP) of the nucleotide unit to permit detection and identification of the nucleotide base.

In some embodiments, at least one nucleotide arm of a multivalent molecule has a nucleotide unit that is attached to a detectable reporter moiety. In some embodiments, the detectable reporter moiety is attached to the nucleotide base. In some embodiments, the detectable reporter moiety comprises a fluorophore. In some embodiments, a particular detectable reporter moiety (e.g., fluorophore) that is attached to the multivalent molecule can correspond to the base (e.g., dATP, dGTP, dCTP, dTTP or dUTP) of the nucleotide unit to permit detection and identification of the nucleotide base.

In some embodiments, the core of a multivalent molecule comprises an avidin-like or streptavidin-like moiety and the core attachment moiety comprises biotin. In some embodiments, the core of a multivalent molecule can comprise an avidin-like moiety. In some embodiments, the core of a multivalent molecule can comprise a streptavidin-like moiety. In some embodiments, the core of a multivalent molecule can comprise the core attachment moiety can comprise biotin. In some embodiments, the core comprises an streptavidin-type or avidin-type moiety which includes an avidin protein, as well as any derivatives, analogs and other non-native forms of avidin that can bind to at least one biotin moiety. Other forms of avidin moieties include native and recombinant avidin and streptavidin as well as derivatized molecules, e.g. non-glycosylated avidin and truncated streptavidins. For example, avidin moiety includes de-glycosylated forms of avidin, bacterial streptavidin produced by Streptomyces (e.g., Streptomyces avidinii), as well as derivatized forms, for example, N-acyl avidins, e.g., N-acetyl, N-phthalyl and N-succinyl avidin, and the commercially-available products EXTRAVIDIN, CAPTAVIDIN, NEUTRAVIDIN and NEUTRALITE AVIDIN.

In some embodiments, any of the methods for sequencing nucleic acid molecules described herein can include forming a binding complex, where the binding complex comprises (i) a polymerase, a nucleic acid concatemer molecule duplexed with a primer, and a nucleotide, or the binding complex comprises (ii) a polymerase, a nucleic acid concatemer molecule duplexed with a primer, and a nucleotide unit of a multivalent molecule. In some embodiments, the binding complex can comprise (i) a polymerase, a nucleic acid concatemer molecule duplexed with a primer, and a nucleotide. In some embodiments, the binding complex can comprise (ii) a polymerase, a nucleic acid concatemer molecule duplexed with a primer, and a nucleotide unit of a multivalent molecule. In some embodiments, the binding complex has a persistence time of greater than about 0.1, 0.2, 0.3, 0.4, 0.5, 0.6, 0.7, 0.8, 0.9 or 1 second. The binding complex has a persistence time of greater than about 0.1-0.25 seconds, or about 0.25-0.5 seconds, or about 0.5-0.75 seconds, or about 0.75-1 second, or about 1-2 seconds, or about 2-3 seconds, or about 3-4 second, or about 4-5 seconds, and/or wherein the method is or may be carried out at a temperature of at or above 15° C., at or above 20° C., at or above 25° C., at or above 35° C., at or above 37° C., at or above 42° C. at or above 55° C. at or above 60° C., or at or above 72° C., or at or above 80° C., or within a range defined by any of the foregoing, or a combination of the binding complex can have a persistence time of greater that about 0.1-0.25 seconds, or about 0.25-0.5 seconds, or about 0.5-0.75 seconds, or about 0.75-1 second, or about 1-2 seconds, or about 2-3 seconds, or about 3-4 second, or about 4-5 seconds and wherein the methodcan beor may be carried out at a temperature of at or above 15° C., at or above 20° C., at or above 25° C., at or above 35° C., at or above 37° C., at or above 42° C. at or above 55° C. at or above 60° C., or at or above 72° C., or at or above 80° C., or within a range defined by any of the foregoing . . . . The binding complex (e.g., ternary complex) remains stable until subjected to a condition that causes dissociation of interactions between any of the polymerase, template molecule, primer and/or the nucleotide unit or the nucleotide. For example, a dissociating condition comprises contacting the binding complex with any one or any combination of a detergent, EDTA and/or water. In some embodiments, the present disclosure provides said method wherein the binding complex is deposited on, attached to, or hybridized to, a surface showing a contrast to noise ratio in the detecting step of greater than 20. In some embodiments, the present disclosure provides said method wherein the contacting is performed under a condition that stabilizes the binding complex when the nucleotide or nucleotide unit is complementary to a next base of the template nucleic acid, and destabilizes the binding complex when the nucleotide or nucleotide unit is not complementary to the next base of the template nucleic acid.

### Avidity Complexes

In any of the sequencing methods as disclosed herein, a multivalent molecule may be used. Multivalent binding compositions as disclosed herein may comprise a particle-nucleotide conjugate having a plurality of copies of a nucleotide attached to the particle. The multivalent binding composition may allow one to localize detectable signals, and can be used to identify sites of base incorporation in elongating nucleic acid chains during polymerase reactions. Multivalent binding compositions may be used to provide improved base discrimination for sequencing and array based applications. In some embodiments, in any of the sequencing methods that employ multivalent molecules, the binding of the plurality of first complexed polymerases with the plurality of multivalent molecules forms at least one avidity complex.

The method can comprise: (a) binding a first nucleic acid primer, a first sequencing polymerase, and a first multivalent molecule to a first portion of a concatemer template molecule thereby forming a first binding complex, wherein a first nucleotide unit of the first multivalent molecule can bind to the first sequencing polymerase; and (b) binding a second nucleic acid primer, a second sequencing polymerase, and the first multivalent molecule to a second portion of the same concatemer template molecule thereby forming a second binding complex, wherein a second nucleotide unit of the first multivalent molecule can bind to the second sequencing polymerase, wherein the first and second binding complexes which can include the same multivalent molecule forms an avidity complex. In some embodiments, the method can comprise binding a first nucleic acid primer, a first sequencing polymerase, and a first multivalent molecule to a first portion of a concatemer template molecule thereby forming a first binding complex.

In some embodiments, the first multivalent molecule can be detectably-labeled multivalent molecule. In some embodiments, a first nucleotide unit of the first multivalent molecule can bind to the first sequencing polymerase. In some embodiments, the method can comprise binding a second nucleic acid primer, a second sequencing polymerase, and the first multivalent molecule to a second portion of the same concatemer template molecule thereby forming a second binding complex. In some embodiments, a second nucleotide unit of the first multivalent molecule can bind to the second sequencing polymerase, wherein the first and second binding complexes which can include the same multivalent molecule forms an avidity complex. In some embodiments, the first multivalent molecule can comprise a core attached to a plurality of nucleotide arms. In some embodiments, each nucleotide arm can be attached to a nucleotide unit. In some embodiments, the binding can be conducted under a condition suitable to inhibit polymerase-catalyzed incorporation of the first nucleotide unit and the second nucleotide unity in the first binding complex and the second binding complex. In some embodiments, the method can further comprise detecting the first binding complex and the second binding complex on the concatemer molecule. In some embodiments, the method can comprise detecting the first binding complex on the concatemer molecule. In some embodiments, the method can comprise detecting the second binding complex on the concatemer molecule. In some embodiments, the method can further comprise identifying the first nucleotide unit in the first binding complex thereby determining the sequence of the first portion of the concatemer molecule and identifying the second nucleotide unit in the second binding complex thereby determining the sequence of the second portion of the concatemer molecule. In some embodiments, the method can comprise identifying the first nucleotide unit in the first binding complex thereby determining the sequence of the first portion of the concatemer molecule. In some embodiments, the method can comprise identifying the second nucleotide unit in the second binding complex thereby determining the sequence of the second portion of the concatemer molecule. In some embodiments, the first sequencing polymerase can comprise any wild type or mutant polymerase described herein. In some embodiments, the second sequencing polymerase can comprise any wild type or mutant polymerase described herein. The concatemer template molecule can comprise tandem repeat sequences of a sequence of interest and at least one universal sequencing primer binding site. The first and second nucleic acid primers can bind to a sequencing primer binding site along the concatemer template molecule. Example multivalent molecules are shown in FIGS. 7-10.

In some embodiments, in any of the sequencing methods that employ multivalent molecules, the method can include binding the plurality of first complexed polymerases with the plurality of multivalent molecules to form at least one avidity complex, the method can comprise: (a) contacting the plurality of sequencing polymerases and the plurality of nucleic acid primers with different portions of a concatemer nucleic acid concatemer molecule to form at least first and second complexed polymerases on the same concatemer template molecule; (b) contacting a plurality of multivalent molecules to the at least first and second complexed polymerases on the same concatemer template molecule, under conditions suitable to bind a single multivalent molecule from the plurality to the first and second complexed polymerases, wherein at least a first nucleotide unit of the single multivalent molecule can be bound to the first complexed polymerase which can include a first primer hybridized to a first portion of the concatemer template molecule thereby forming a first binding complex (e.g., first ternary complex), and wherein at least a second nucleotide unit of the single multivalent molecule can be bound to the second complexed polymerase which can include a second primer hybridized to a second portion of the concatemer template molecule thereby forming a second binding complex (e.g., second ternary complex), wherein the contacting can be conducted under a condition suitable to inhibit polymerase-catalyzed incorporation of the bound first and second nucleotide units in the first and second binding complexes, and wherein the first and second binding complexes which can be bound to the same multivalent molecule forms an avidity complex; (c) detecting the first and second binding complexes on the same concatemer template molecule, and (d) identifying the first nucleotide unit in the first binding complex thereby determining the sequence of the first portion of the concatemer template molecule, and identifying the second nucleotide unit in the second binding complex thereby determining the sequence of the second portion of the concatemer template molecule.

In some embodiments, the method can comprise contacting the plurality of sequencing polymerases and the plurality of nucleic acid primers with different portions of a concatemer nucleic acid concatemer molecule to form at least first and second complexed polymerases on the same concatemer template molecule. In some embodiments, the method can comprise contacting a plurality of multivalent molecules to the at least first and second complexed polymerases on the same concatemer template molecule. In some embodiments, the contacting of the plurality of multivalent molecules to the at least first and second complexed polymerases can occur under conditions suitable to bind a single multivalent molecule from the plurality to the first and second complexed polymerases. In some embodiments, at least a first nucleotide unit of the single multivalent molecule can be bound to the first complexed polymerase. In some embodiments, the first complexed polymerase can include a first primer hybridized to a first portion of the concatemer template molecule thereby forming a first binding complex (e.g., first ternary complex). In some embodiments, at least a second nucleotide unit of the single multivalent molecule can be bound to the second complexed polymerase. In some embodiments, the second complexed polymerase can include a second primer hybridized to a second portion of the concatemer template molecule thereby forming a second binding complex (e.g., second ternary complex). In some embodiments, the contacting can be conducted under a condition suitable to inhibit polymerase-catalyzed incorporation of the bound first and second nucleotide units in the first and second binding complexes. In some embodiments, the first and second binding complexes which can be bound to the same multivalent molecule forms an avidity complex. In some embodiments, the method can comprise detecting the first and second binding complexes on the same concatemer template molecule. In some embodiments, the method can comprise identifying the first nucleotide unit in the first binding complex thereby determining the sequence of the first portion of the concatemer template molecule, and identifying the second nucleotide unit in the second binding complex thereby determining the sequence of the second portion of the concatemer template molecule. In some embodiments, the method can comprise identifying the first nucleotide unit in the first binding complex thereby determining the sequence of the first portion of the concatemer template molecule. In some embodiments, the method can comprise identifying the second nucleotide unit in the second binding complex thereby determining the sequence of the second portion of the concatemer template molecule. In some embodiments, the method can comprise contacting the plurality of concatemer molecules with(i) a plurality of sequencing polymerases and (ii) a plurality of soluble sequencing primers. In some embodiments, the method can comprise contacting the plurality of concatemer molecules with a plurality of sequencing polymerases. In some embodiments, the plurality of sequencing polymerases can comprise a plurality of first sequencing polymerases. In some embodiments, the plurality of sequencing polymerases can comprise a plurality of second sequencing polymerases. In some embodiments, the method can comprise contacting the plurality of concatemer molecules with a plurality of soluble sequencing primers. In some embodiments, the contacting can be conducted under a condition suitable to form a plurality of complexed sequencing polymerases. In some embodiments, each complexed sequencing polymerase can comprise a sequencing polymerase bounds to a nucleic acid duplex. In some embodiments, the nucleic acid duplex can comprise a concatemer molecule hybridized to a soluble sequencing primer. In some embodiments, the method can further comprise contacting the plurality of complexed sequencing polymerases with a plurality of nucleotides. In some embodiments, the contacting can be conducted under a condition suitable for binding at least one nucleotide to a complexed sequencing polymerase. In some embodiments, the plurality of nucleotides can comprise at least one nucleotide analog. In some embodiments, the at least one nucleotide analog can be labeled with a fluorophore. In some embodiments, the at least one nucleotide analog can have a removable chain terminating moiety at the sugar 3′position. In some embodiments, the at least one nucleotide analog can be labeled with a fluorophore and can have a removable chain terminating moiety at the sugar 3′ position. In some embodiments, the method can further comprise incorporating the at least nucleotide analog into the 3′ end of the plurality of soluble sequencing primers, thereby generating a plurality of nascent extended sequencing primers. In some embodiments, the method can further comprise detecting the incorporated at least one nucleotide analog. In some embodiments, the method can further comprise identifying the nucleobase of the incorporated at least one nucleotide analog.

In some embodiments, the method can comprise contacting the plurality of concatemer molecules with (i) a plurality of sequencing polymerases and (ii) a plurality of soluble sequencing primers. In some embodiments, the method can comprise contacting the plurality of concatemer molecules with a plurality of sequencing polymerases. In some embodiments, the plurality of sequencing polymerases can comprise a plurality of first sequencing polymerases. In some embodiments, the plurality of sequencing polymerases can comprise a plurality of second sequencing polymerases. In some embodiments, the method can comprise contacting the plurality of concatemer molecules with a plurality of soluble sequencing primers. In some embodiments, the contacting can be conducted under a condition suitable to form a plurality of complexed sequencing polymerases. In some embodiments, each complexed sequencing polymerase can comprise a sequencing polymerase bounds to a nucleic acid duplex. In some embodiments, the nucleic acid duplex can comprise a concatemer molecule hybridized to a soluble sequencing primer. In some embodiments, the method can further comprise contacting the plurality of first complexed sequencing polymerases with a plurality of detectably labeled multivalent molecules to form a plurality of multivalent-complexed polymerases. In some embodiments, the contacting can be conducted under a condition suitable for binding complementary nucleotide units of the plurality of detectably labeled multivalent molecules to at least two of the plurality of first complexed sequencing polymerases, thereby forming the plurality of multivalent-complexed polymerases. In some embodiments, the contacting can be conducted under the condition that can inhibit incorporation of the complementary nucleotide units into the plurality of soluble universal sequencing primers of the plurality of multivalent-complexed polymerases. In some embodiments, individual multivalent molecules in the plurality of detectably labeled multivalent molecules can comprise a core attached to a nucleotide unit. In some embodiments, the method can further comprise detecting the plurality of multivalent-complexed polymerases. In some embodiments, the method can further comprise identifying the nucleobase of the complementary nucleotide units that are bound to the plurality of first complexed sequencing polymerases in the plurality of multivalent-complexed polymerases, thereby determining the sequence of a nucleic acid template. In some embodiments, the method can further comprise dissociating the plurality of multivalent-complexed polymerases. In some embodiments, the method can further comprise removing the plurality of first sequencing polymerases and the plurality of detectably labeled multivalent molecules. In some embodiments, the plurality of nucleic acid duplexes can be retained. In some embodiments, the method can further comprise contacting the plurality of nucleic acid duplexes with a plurality of second sequencing polymerases. In some embodiments, the contacting can be conducted under a condition suitable for binding the plurality of second sequencing polymerases to the plurality of nucleic acid duplexes, thereby forming a plurality of second complexed sequencing polymerases. In some embodiments, each second complexed sequencing polymerase can comprise a second sequencing polymerase bound to the nucleic acid duplex. In some embodiments, the method can further comprise contacting the plurality of second complexed sequencing polymerases with a plurality of non-labeled nucleotides. In some embodiments, the plurality of non-labeled nucleotides can comprise at least one nucleotide analog that can have a removable chain terminating moiety at the sugar 3′ position. In some embodiments, the contacting can be conducted under a condition suitable for binding complementary nucleotides from the plurality of non-labeled nucleotides to at least two of the plurality of second complexed sequence polymerases, thereby forming a plurality of nucleotide-complexed polymerases. In some embodiments, the contacting can be conducted under the condition suitable for promoting incorporation of the complementary nucleotides into the plurality of soluble universal sequencing primers of the plurality of nucleotide-complexed polymerases. In some embodiments, the plurality of sequencing polymerases comprise any wild type or mutant sequencing polymerase described herein. The concatemer template molecule comprises tandem repeat sequences of a sequence of interest and at least one universal sequencing primer binding site. The plurality of nucleic acid primers can bind to a sequencing primer binding site along the concatemer template molecule. Non-limiting example multivalent molecules are shown in FIGS. 7-10.

### Sample Analysis

In any of the methods as described herein a biological sample may be processed. For example, a cellular sample may be extracted from a biological sample. The cellular sample may be extracted from, e.g., blood or tissue. The cellular sample may be separated from other components of the biological sample via immunomagnetic cell separations, fluorescence-activated cell sorting (FACS), density gradient centrifugation, immunodensity cell isolation, microfluidics, or any combination thereof. Further examples of cell isolation techniques may be found at Hu, Ping, et al. “Single cell isolation and analysis.” Frontiers in cell and developmental biology 4 (2016): 116, which is incorporated herein by reference in its entirety. A cellular sample may be subjected to any assay, e.g., a viability assay, cell proliferation assay, reporter gene assay, cell signaling assays, functional assay, live cell analysis, protein assay, reactive oxygen species assay, or any combination thereof.

### Flow Cells

In any of the methods described herein, the cellular sample can be deposited onto a solid support (e.g., a flow cell). In some embodiments, the cellular sample is deposited onto a flow cell having walls (e.g., top or first wall, and bottom or second wall) and a gap in-between, where the gap can be filled with a fluid, where the flow cell is positioned in a fluorescence optical imaging system. The cellular sample has a thickness that may require using the imaging system to focus separately on the first and second surfaces of the flow cell, when using a traditional imaging system. For improved imaging of the sequencing reaction of the concatemers in the cellular sample, the flow cell can be positioned in a high performance fluorescence imaging system, which comprises two or more tube lenses which are designed to provide optimal imaging performance for the first and second surfaces of the flow cell at two or more fluorescence wavelengths. In some embodiments, the high-performance imaging system further comprises a focusing mechanism configured to refocus the optical system between acquiring images of the first and second surfaces of the flow cell. In some embodiments, the high performance imaging system is configured to image two or more fields-of-view on at least one of the first flow cell surface or the second flow cell surface.

### Supports and Coatings

In any of the methods described herein, the solid support comprises a flow cell having a coating that promotes cell adhesion. In some embodiments, the flow cell comprises a support which can be a planar or non-planar support. The support can be solid or semi-solid. In some embodiments, the support can be porous, semi-porous or non-porous. The support can be made of any material such as glass, plastic or a polymer material. In some embodiments, the surface of the support can be coated with one or more compounds to produce a passivated layer on the support. In some embodiments, the passivated layer forms a porous or semi-porous layer. In some embodiments, the support is coated with a lysine compound, poly-lysine compound, arginine compound or an amino-terminated compound. The support can be coated with an unbranched compound, a branched compound, or a mixture of unbranched and branched compounds. In some embodiments, the support is coated with surface primers for capturing nucleic acids from the cellular sample. Alternatively, the support lacks surface primers.

### Automated Mode

In any of the methods described herein, any combination of the steps for conducting in situ reiterative short read sequencing can be performed in an automated mode using the fluid dispensing system, including cell seeding, cell fixation, cell permeabilization, reverse transcription reactions, padlock probe hybridization, padlock probe ligation reaction, rolling circle amplification, and sequencing.

The present disclosure provides apparatus and methods for growing/culturing a cellular sample on a flow cell and conducting nucleic acid workflows of the cultured cellular sample on the flow cell.

In some embodiments, the cellular sample is deposited on the flow cell. The flow cell can be coated with a reagent that promotes cell adhesion to the flow cell. The flow cell, having a cell sample adhered thereon, can be placed onto a sequencing apparatus having a flow cell holder/cradle which is fluidically connected to an automated fluid dispensing system and configured on a fluorescent microscope. In some embodiments, the sequencing apparatus can be configured with at least one fluidic delivery device, at least one fluidics device (e.g., microfluidics device), at least one imaging device and/or at least one sensor to detect signals from the sequencing reactions.

In some embodiments, the automated fluid dispensing system can be used to deliver simple or complex cell culture media to the cellular sample on the flow cell. In some embodiments, the cellular sample can be cultured/expanded on the flow cell for 2-10 generations or more. In some embodiments, the cellular sample can be expanded to confluence or non-confluence.

In some embodiments, the automated fluid dispensing system can be used to deliver fixation reagents to the expanded cellular sample on the flow cell, and the cellular sample can be incubated under conditions suitable for cell fixation.

In some embodiments, the automated fluid dispensing system can be used to deliver permeabilization reagents to the fixed cellular sample on the flow cell, and the cellular sample can be incubated under conditions suitable for cell permeabilization.

In some embodiments, the automated fluid dispensing system can be used to deliver reagents for conducting reverse transcription of RNA inside the fixed and permeabilized cellular sample under a condition suitable for generating a plurality of cDNA inside the cellular sample.

In some embodiments, the automated fluid dispensing system can be used to deliver reagents for conducting padlock probe hybridization, circularization and ligation under a condition suitable for generating a plurality of covalently closed circular padlock probes inside the cellular sample.

In some embodiments, the automated fluid dispensing system can be used to deliver reagents for conducting rolling circle amplification under a condition suitable for generating a plurality of concatemer molecules inside the cellular sample.

In some embodiments, the automated fluid dispensing system can be used to deliver sequencing reagents for conducting sequencing cycles of the concatemer molecules under a condition suitable for generating a plurality of sequencing read products inside the cellular sample. In some embodiments, individual cycle times can be achieved in less than 30 minutes. In some embodiments, the field of view (FOV) can exceed 1 mm² and the cycle time for scanning large area (>10 mm²) can be less than 5 minutes.

In some embodiments, the automated fluid dispensing system can be used to deliver reagents for removing the plurality of sequencing read products from the concatemer molecules and retaining the concatemer molecules inside the cellular sample.

In some embodiments, the automated fluid dispensing system can be used to deliver sequencing reagents for conducting sequencing cycles of the concatemer molecules under a condition suitable for generating another plurality of sequencing read products inside the cellular sample. In some embodiments, individual cycle times can be achieved in less than 30 minutes. In some embodiments, the field of view (FOV) can exceed 1 mm² and the cycle time for scanning large area (>10 mm²) can be less than 5 minutes.

### Kits

The present disclosure provides kits for carrying out the methods disclosed herein using the systems and compositions disclosed herein. A kit may comprise a detectable polymer-nucleotide conjugate. The polymer-nucleotide conjugate may comprise a polymer core. The polymer-nucleotide conjugate may comprise two or more nucleotide moieties attached to the polymer core. The kits described herein may have at least one, two, three, or four different types of detectable polymer-nucleotide conjugate, for example, in which each type of detectable polymer-nucleotide conjugate has a different nucleotide moiety. The kit may have a substrate comprising a surface having coupled thereto a polymer layer suitable to immobilize a biological sample or derivative thereof to said surface. In some kits, the biological sample (e.g., cell or tissue) is included in the kit. In some kits, the biological sample is not included in the kit. The kit may comprise a hybridization buffer disclosed herein, for example, comprising (i) a first polar aprotic solvent having a dielectric constant that is no greater than 40 and having a polarity index of 4-9; and/or (ii) a second polar aprotic solvent having a dielectric constant that is less than or equal to 115. Optionally, capture oligonucleotides or components thereof, in situ amplification reagents (e.g., buffers, primers, detectable labels), or combinations thereof are included in the kit.

Instructions may be provided in the kits described herein, including instructions for hybridizing at least a portion of said target nucleic acid sequence to at least a portion of a capture oligonucleotide coupled to said surface. The kit may also comprise instructions for identifying at least a portion of the target nucleic acid sequence within the biological sample or derivative thereof by contacting said detectable polymer-nucleotide conjugate with said biological sample or derivative thereof (e.g., containing the target nucleic acid molecule) under conditions sufficient to form a multivalent binding complex between said two or more nucleotide moieties and said target nucleic acid sequence.

The kit may also comprise instructions for identifying at least a portion of a sub-cellular component within a cell or tissue in situ by contacting said detectable polymer-nucleotide conjugate with said sub-cellular component under conditions sufficient to form a multivalent binding complex between said two or more nucleotide moieties and said sub-cellular component.

Optionally, the kit also contains other useful components, such as, diluents, buffers, pharmaceutically acceptable carriers, syringes, catheters, applicators, pipetting or measuring tools, bandaging materials or other useful paraphernalia. The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as compositions and the like. The packaging material is constructed by well-known methods, preferably to provide a sterile, contaminant-free environment. The packaging materials employed in the kit are those customarily utilized in gene expression assays and in the administration of treatments. As used herein, the term “package” refers to a suitable solid matrix or material such as glass, plastic, paper, foil, and the like, capable of holding the individual kit components. Thus, for example, a package can be a glass vial or prefilled syringes used to contain suitable quantities of the pharmaceutical composition. The packaging material has an external label which indicates the contents and/or purpose of the kit and its components.

In some embodiments, a kit comprises one or more containers comprising: a synthetic polypeptide disclosed herein. In some embodiments, the kit comprises a nucleotide unit. In some embodiments, the nucleotide unit is detectable. In some embodiments, the kit comprises instructions for introducing the synthetic polypeptide and the nucleotide unit to a primed nucleic acid sequence. In some embodiments, the primed nucleic acid sequence comprises a nucleotide complementary to the nucleotide unit under conditions sufficient to form a binding complex. In some embodiments, the binding complex comprises the synthetic polypeptide, the nucleotide unit, and the primed nucleic acid sequence. In some embodiments, the kit further comprises a composition, such as a nucleotide conjugate or a nucleotide conjugate disclosed herein. In some embodiments, the composition comprises a core and at least two nucleotide arms. In some embodiments, a nucleotide arm of the at least two nucleotide arms comprises: a core attachment moiety coupled to the core; a spacer coupled to the core attachment moiety; a linker coupled to the spacer; and the nucleotide unit coupled to the linker.

In some embodiments, the kit comprises one or more padlock probes for batch-specific sequencing as disclosed herein. Kits as disclosed herein may comprise one or more antibodies for use in any of the methods as described herein. Kits as disclosed herein may comprise one or more antibody-nucleotide conjugates for use in any of the methods or compositions as described herein. Kits as disclosed herein may comprise one or more sequencing polymerases. Kits as disclosed herein may comprise one or more reverse transcriptase. Kits as disclosed herein may comprise one or more reagents for performing rolling circle amplification (RCA).

Disclosed herein, in some embodiments, are kits for nucleic acid molecule processing. In some embodiments, the kit comprises a composition disclosed herein, or a formulation disclosed herein. In some embodiments, the kit comprises an instruction for use of the composition in a nucleotide identification reaction. In some embodiments, the instructions for use of the composition or the formulation comprise introducing the nucleotide conjugate and/or the synthetic polypeptide to a nucleic acid sequence (e.g., primed nucleic acid sequence) under conditions sufficient to form a binding complex between a nucleotide of the nucleic acid sequence and a nucleotide unit of the nucleotide conjugate or the synthetic polypeptide or a combination thereof. In some embodiments, instructions further comprise use of the composition for performing a nucleotide binding, nucleotide incorporation, or a nucleotide identification reaction therewith.

In some embodiments, the kit further comprises: an agent that reacts with the reactive group in the linker of the composition. In some embodiments, the kit further comprises: an agent that reacts with the reactive group at the 3′ carbon of the sugar moiety in the nucleotide unit of the composition. In some embodiments, the kit further comprises a reagent for use in the nucleotide binding reaction. In some embodiments, the reagent comprises a cation.

In some embodiments, the kit further comprises: (i) a solution comprising a cation; (ii) one or more polymerizing enzymes; (iii) one or more primer sequences; (iv) one or more unlabeled nucleotides; or any combination of (i) to (iv).

The present disclosure provides a kit comprising any one or any combination of two or more of any of the nucleotide conjugates described herein. The kit can comprise for example a plurality of one type of a nucleotide conjugate, a mixture of different types (sub-populations) of the nucleotide conjugates, or a combination of a plurality of one type of a nucleotide conjugate and a mixture of different types of the nucleotide conjugates. The nucleotide conjugates in the kit can be labeled (e.g., fluorescently labeled), non-labeled, or a mixture of labeled and non-labeled forms. The nucleotide conjugates in the kit can include wild type or mutant forms of a streptavidin or avidin core. The nucleotide conjugates in the kit can include core moieties that are labeled with the same type of detectable reporter moiety (e.g., fluorophore) or different types of detectable reporter moieties (e.g., different fluorophores). The nucleotide conjugates in the kit can include nucleotide arms having the same type of spacer or different types of spacers. The nucleotide conjugates in the kit can include nucleotide arms having the same type of linker or different types of linkers. The nucleotide conjugates in the kit can include nucleotide arms comprising linkers with the same type of reactive group or different types of reactive groups. The nucleotide conjugates in the kit can include nucleotide arms having the same type of nucleotide units or different types of nucleotide units. The nucleotide conjugates in the kit can include nucleotide arms having nucleotide units having the same type of reactive groups at the sugar 3′ position or different types of reactive groups.

The kit can further include one or more chemical agents that react with a reactive group in the linker of the nucleotide conjugates. The kit can further include one or more chemical agents that react with a reactive group at the sugar 3′ group in the nucleotide unit of the nucleotide conjugates.

In some embodiments, the kit can further comprise at least one reagent suitable for use in conducting a nucleotide unit binding reaction, a nucleotide unit incorporation reaction, or a combination of a nucleotide unit binding reaction and a nucleotide unit incorporation reaction. In some embodiments, the reagent can comprise cations including any one or any combination of two or more of sodium, magnesium, strontium, barium, potassium, manganese, calcium, lithium, nickel, cobalt, or any combination thereof or other cations suitable for conducting a nucleotide unit binding reaction, a nucleotide incorporation reaction, or a combination of a nucleotide unit binding reaction and a nucleotide unit incorporation reaction. For example, the kit can comprise a reagent comprising a non-catalytic divalent cation including strontium, barium, calcium, or a combination thereof. The kits comprise a reagent comprising a catalytic divalent cation including magnesium, manganese, or a combination of magnesium and manganese.

The kits can comprise one or more containers that contain any one or any combination of two or more of any of the nucleotide conjugates described herein. In some embodiments, the kit can further comprise one or more containers that contain at least one cation, at least one polymerase, primers, a plurality of nucleotides, or a combination thereof. The cation, polymerase and/or nucleotides can be combined in any combination and can be contained in a single container, or can be contained in separate containers, or any combination thereof.

The kit can include instructions for use of the kit for conducting a nucleotide binding reaction, a nucleotide incorporation reaction, a nucleic acid sequencing reaction, or a combination thereof using nucleotide conjugates.

In some embodiments, the kit is configured for detecting in situ at least two target RNA sequences and at least two polypeptides in a biological sample. In some embodiments, the kit comprises the first target polypeptide encoded by the first target nucleic acid molecule or a reverse complement thereof. In some embodiments, the second target polypeptide is encoded by the second target nucleic acid molecule or a reverse complement thereof. In some embodiments, the full sequence of the first target RNA sequence and the full sequence of the second target RNA sequence have at least one nucleotide of difference. In some embodiments, the full sequence of the first target polypeptide and the full sequence of the second target polypeptide have at least one amino acid of difference. In some embodiments, the first, second, or third nucleic acid enzyme comprises a nucleic acid ligase, a nucleic acid ligation enzyme, a nucleic acid polymerase, a nucleic acid polymerization enzyme, or combinations thereof. In some embodiments, the nucleotide of the plurality of nucleotides comprises a fluorescent label. In some embodiments, the nucleotide of the plurality of nucleotides comprises a removable blocking group at the 3′ carbon position of the sugar moiety. In some embodiments, the plurality of nucleotides consist of at least two of the same type of nucleotide comprising a fluorescent label, wherein the same type of nucleotide is selected from the group comprising dATP, dGTP, dCTP, dTTP, and dUTP, wherein the fluorescent label of one type of nucleotide of the group emits light at a wavelength that is different from the wavelength of light emitted from the fluorescent label of another type of nucleotide of the group. In some embodiments, the plurality of nucleotides comprise at least two types of nucleotides, wherein a type of the at least two types of nucleotides comprises a fluorescent label, wherein the at least two types of nucleotides are selected from the group comprising dATP, dGTP, dCTP, dTTP, and dUTP, wherein the fluorescent label of one type of nucleotide of the group emits light at a wavelength that is different from the wavelength of light emitted from the fluorescent label of another type of nucleotide of the group. In some embodiments, the nucleotide conjugate comprises a detectable label. In some embodiments, the nucleotide conjugate comprises a fluorescent label. In some embodiments, the biological sample comprises a human sample, a simian sample, an ape sample, a canine sample, a feline sample, a bovine sample, an equine sample, a murine sample, a porcine sample, a caprine sample, a lupine sample, a ranine sample, a piscine sample, a plant sample, an insect sample, a bacteria sample, an algae sample, a viral sample, a protozoa sample, or a fungus sample. In some embodiments, the biological sample comprises a cellular organelle, a cell, a whole cell, a group of whole cells, a tissue, an intact tissue, a tumor, an intact tumor, an organ, or an organism. In some embodiments, the biological sample comprises a cellular organelle, a cell, a whole cell, a group of whole cells, a tissue, an intact tissue, a tumor, an intact tumor, an organ, an organism, a protozoa, an algae, a bacteria, a virus, a plant, a fungus, an insect, or an animal. In some embodiments, the biological sample comprises a fresh sample, a processed sample, a freshly-frozen sample, a sectioned sample, or a formalin-fixed and paraffin-embedded (FFPE) sample. In some embodiments, the first target nucleic acid sequence comprises DNA, cDNA, RNA, coding RNA, non-coding RNA, mRNA, tRNA, rRNA, miRNA, gRNA, snRNA, siRNA, anti-sense RNA, mature microRNA, or immature microRNA, and/or, wherein the first target RNA sequence comprises coding RNA, non-coding RNA, mRNA, tRNA, rRNA, miRNA, gRNA, snRNA, siRNA, anti-sense RNA, mature microRNA, or immature microRNA. In some embodiments, the second target nucleic acid sequence comprises DNA, cDNA, RNA, coding RNA, non-coding RNA, mRNA, tRNA, rRNA, miRNA, gRNA, snRNA, siRNA, anti-sense RNA, mature microRNA, or immature microRNA, and/or wherein the second target RNA sequence comprises coding RNA, non-coding RNA, mRNA, tRNA, rRNA, miRNA, gRNA, snRNA, siRNA, anti-sense RNA, mature microRNA, or immature microRNA. In some embodiments, the blocking group comprises an alkyl group, an alkenyl group, an alkynyl group, an allyl group, an aryl group, a benzyl group, an azide group, an azido group, an O-azidomethyl group, an amine group, an amide group, a keto group, an isocyanate group, a phosphate group, a thiol group, a disulfide group, a carbonate group, a urea group, or a silyl group. In some embodiments, the kit further comprises a first oligonucleotide conjugate comprising a first short nucleic acid and a first binding moiety that binds specifically to the first target polypeptide. In some embodiments, the first short nucleic acid comprises a first tag sequence, a second tag sequence. In some embodiments, the first oligonucleotide conjugate binds specifically to the first target polypeptide through the first binding moiety to form a first binding complex. In some embodiments, the first and second tag sequences identify the first binding moiety. In some embodiments, the kit comprises a second oligonucleotide conjugate comprising a second short nucleic acid and a second binding moiety that binds specifically to the second target polypeptide. In some embodiments, the second short nucleic acid comprises a third tag sequence, a fourth tag sequence, wherein the second oligonucleotide conjugate binds specifically to the second target polypeptide through the second binding moiety to form a second binding complex, wherein the third and fourth tag sequences identify the second binding moiety. In some embodiments, the kit further comprises a third oligonucleotide comprising a first end portion and a second end portion. In some embodiments, the first end portion and second end portion of the third oligonucleotide are complementary and bind to the first tag sequence and the second tag sequence of the first oligonucleotide conjugate so that the third oligonucleotide forms a circular structure with a gap between the first end portion and the second end portion. In some embodiments, the kit further comprises a fourth oligonucleotide comprising a first end portion and a second end portion. In some embodiments, the first end portion and second end portion of the fourth oligonucleotide are complementary and bind to the third tag sequence and the fourth tag sequence so that the fourth oligonucleotide forms a circular structure with a gap between the first end portion and the second end portion. In some embodiments, the third oligonucleotide comprises a third identification sequence that identifies the first binding moiety, and wherein the fourth oligonucleotide comprises a fourth identification sequence that identifies the second binding moiety. In some embodiments, the first oligonucleotide and third oligonucleotide comprise the same first sequencing primer or the reverse complement thereof, wherein the second oligonucleotide and the fourth oligonucleotide comprise the same second sequencing primer or the reverse complement thereof. In some embodiments, the kit further comprises an agent that reacts with the reactive group at the 3′ carbon of the sugar moiety in the nucleotide moiety of the nucleotide conjugate. In some embodiments, the kit further comprises a reagent for use in the nucleotide binding reaction. In some embodiments, the reagent comprises a cation. In some embodiments, the kit further comprises a reverse transcriptase, a primer for reverse transcription, a sequencing primer, a reagent configured to permeabilize the biological sample, a reagent configured to fix the biological sample, an unblocked nucleotide, a blocked nucleotide. a reagent for use in the nucleotide incorporation reaction, a solution comprising a cation, one or more unlabeled nucleotides, one or more buffers for reverse transcription, one or more buffers for nucleic acid binding, one or more buffers for nucleic acid amplification, or one or more buffers for nucleic acid dissociation. In some embodiments, the kit further comprises instructions for use of the kit to detect in situ the at least two target nucleic acid sequences in the biological sample. In some embodiments, the kit further comprises instructions for use of the kit to detect in situ the at least two target RNA sequences and the at least two polypeptides in the biological sample. In some embodiments, the instructions comprise performing a sequencing by synthesis reaction. In some embodiments, the instructions comprise performing a sequencing by binding reaction in which detection in situ is not contemporaneous with a nucleotide incorporation step. In some embodiments, the kit further comprises instructions for use of the kit using any of the methods provided in the present disclosure.

### Compositions

The present disclosure provides compositions for analyzing or processing a nucleic acid sequence. The compositions described herein may be used to employ in situ reiterative short read sequencing within a cellular sample including a single cell, multiple cells, a tissue or a tumor. By conducting reiterative short sequencing cycles, the RNA content of the cellular sample can be discovered. Compositions as described herein may be employed in conducting transcriptomics workflows which leverages massively parallel sequencing technologies. Compared to long read sequencing workflows, the reiterative short sequencing cycles described herein use a reduced amount of sequencing reagents which reduces cost and saves time. Compositions for conducting reiterative short sequencing cycles has many uses, including, but not limited to, detecting specific RNAs of interest, mutant RNA sequences, splice variants, and their abundance levels thereof.

One non-limiting example purpose of the compositions described herein is to detect and image the spatial localization of RNAs within a cellular sample using massively parallel sequencing. Any method as disclosed herein may be used for such a purpose.

The compositions described herein offer several advantages over other in situ transcriptomics workflows, including a simpler workflow, fewer reagents, lower cost, less time, gentler conditions on the cellular sample, and no requirement for specialized equipment.

Compositions for Conducting in situ Reiterative Short Read Sequencing

The present disclosure provides compositions for conducting in situ sequencing in a biological sample. One or more nucleic acids may be analyzed in a biological sample, e.g., a cell. The nucleic acid may comprise, e.g., deoxyribonucleic acid (DNA) or ribonucleic acid (RNA).

The present disclosure provides compositions comprising at least two target nucleic acid molecules. The at least two target nucleic acid sequences may comprise a first target nucleic acid sequence and a second target nucleic acid sequence. In some embodiments, the at least two target polypeptides comprise a first target polypeptide encoded by the first target nucleic acid sequence or a reverse complement thereof. In some embodiments, the at least two target polypeptides comprise a second target polypeptide encoded by the second target nucleic acid sequence or a reverse complement thereof.

In some embodiments, the composition further comprises a biological sample. In some embodiments, the biological sample comprises a first nucleic acid molecule, comprising the first target nucleic acid sequence or a portion thereof, or the reverse complement of the first target nucleic acid sequence or a portion thereof. In some embodiments, the biological sample comprises a second nucleic acid molecule, comprising the second target nucleic acid sequence or a portion thereof, or the reverse complement of the second target nucleic acid sequence or a portion thereof. In some embodiments, the biological sample comprises a third nucleic acid molecule, comprising a third target nucleic acid sequence or a portion thereof, or the reverse complement of the third target nucleic acid sequence or a portion thereof. In some embodiments, the biological sample comprises a fourth nucleic acid molecule, comprising a fourth target nucleic acid sequence or a portion thereof, or the reverse complement of the fourth target nucleic acid sequence or a portion thereof.

In some embodiments, the composition further comprises a first target complementary DNA (cDNA) sequence. In some embodiments, the first target cDNA sequence is generated through reverse transcription of the first target RNA sequence. In some embodiments, the composition further comprises a second target cDNA sequence. In some embodiments, the second target cDNA sequence is generated through reverse transcription of the second target RNA sequence. In some embodiments, the composition further comprises a first oligonucleotide comprising a first end portion and a second end portion. In some embodiments, the first end portion and second end portion of the first oligonucleotide are complementary and bind to two neighboring segments of the first target cDNA sequence so that the first oligonucleotide forms a circular structure with a gap between the first end portion and the second end portion. In some embodiments, the second oligonucleotide comprises a first end portion and a second end portion. In some embodiments, the first end portion and second end portion of the second oligonucleotide are complementary and bind to two neighboring segments of the second target cDNA sequence, so that the second oligonucleotide forms a circular structure with a gap between the first end portion and the second end portion. In some embodiments, the second oligonucleotide comprises a second identification sequence that identifies the second target RNA sequence, a second sequencing primer, and a nucleic acid amplification primer. In some embodiments, the first sequencing primer and the second sequencing primer have at least one nucleotide of difference.

In some embodiments, the composition further comprises a first oligonucleotide conjugate comprising a first short nucleic acid and a first binding moiety. In some embodiments, the first binding moiety binds specifically to the first target polypeptide, wherein the first short nucleic acid comprises a first tag sequence, a second tag sequence, wherein the first oligonucleotide conjugate binds specifically to the first target polypeptide through the first binding moiety to form a first binding complex, wherein the first and second tag sequences identify the first binding moiety. In some embodiments, the composition further comprises a second oligonucleotide conjugate comprising a second short nucleic acid and a second binding moiety that binds specifically to the second target polypeptide. In some embodiments, the second short nucleic acid comprises a third tag sequence, a fourth tag sequence, wherein the second oligonucleotide conjugate binds specifically to the second target polypeptide through the second binding moiety to form a second binding complex, wherein the third and fourth tag sequences identify the second binding moiety.

In some embodiments, the composition further comprises a third oligonucleotide comprising a first end portion and a second end portion. In some embodiments, the first end portion and second end portion of the third oligonucleotide are complementary. In some embodiments, the first end portion and the second end portion bind to the first tag sequence and the second tag sequence of the first oligonucleotide conjugate so that the third oligonucleotide forms a circular structure with a gap between the first end portion and the second end portion. In some embodiments, the composition further comprises a fourth oligonucleotide comprising a first end portion and a second end portion. In some embodiments, the first end portion and second end portion of the fourth oligonucleotide are complementary and bind to the third tag sequence and the fourth tag sequence so that the fourth oligonucleotide forms a circular structure with a gap between the first end portion and the second end portion. In some embodiments, the composition further comprises a first circular oligonucleotide. In some embodiments, the first circular oligonucleotide is formed by joining the first and second end portions of the first oligonucleotide. In some embodiments, the first and second end portions of the second oligonucleotide are joined to produce a second circular oligonucleotide. In some embodiments, the first and second end portions of the third oligonucleotide are joined to produce a third circular oligonucleotide. In some embodiments, the first and second end portions of the fourth oligonucleotide are joined to produce a fourth circular oligonucleotide. In some embodiments, the first circular oligonucleotide and the third circular oligonucleotide comprise a first sequencing primer or the reverse complement thereof. In some embodiments, the second circular oligonucleotide and the fourth circular oligonucleotide comprise a second sequencing primer or the reverse complement thereof. In some embodiments, the sequences of the first and second sequencing primers have at least one nucleotide of difference. In some embodiments, the first circular oligonucleotide is amplified through rolling circle amplification to produce a first concatemer comprising a plurality of the first circular oligonucleotides. In some embodiments, the second circular oligonucleotide is amplified through rolling circle amplification to produce a second concatemer comprising a plurality of the second circular oligonucleotides. In some embodiments, the third circular oligonucleotide is amplified through rolling circle amplification to produce a third concatemer comprising a plurality of the third circular oligonucleotides. In some embodiments, the fourth circular oligonucleotide is amplified through rolling circle amplification to produce a fourth concatemer comprising a plurality of the fourth circular oligonucleotides. In some embodiments, the composition further comprises a first sequencing product nucleic acid molecule that is complementary and binds to the first concatemer. In some embodiments, the sequence of the first concatemer or the portion thereof consists of 2-30 nucleotides. In some embodiments, the composition comprises a third sequencing product nucleic acid molecule that is complementary and binds to the third concatemer. In some embodiments, the sequence of the third concatemer or the portion thereof consists of 2-30 nucleotides. In some embodiments, the composition comprises a second sequencing product nucleic acid molecule that is complementary and binds to the second concatemer. In some embodiments, the sequence of the second concatemer or the portion thereof consists of 2-30 nucleotides. In some embodiments, the composition further comprises a fourth sequencing product nucleic acid molecule that is complementary and binds to the fourth concatemer. In some embodiments, the sequence of the fourth concatemer or the portion thereof consists of 2-30 nucleotides. In some embodiments, the full sequence of the first target RNA sequence and the full sequence of the second target RNA sequence have at least one nucleotide of difference. In some embodiments, the full sequence of the first target polypeptide and the full sequence of the second target polypeptide have at least one amino acid of difference.

In some embodiments, the biological sample comprises a cellular organelle, a cell, a whole cell, a group of whole cells, a tissue, an intact tissue, a tumor, an intact tumor, an organ, an organism, a protozoa, an algae, a bacteria, a virus, a plant, a fungus, an insect, or an animal. In some embodiments, the biological sample comprises a fresh sample, a processed sample, a freshly-frozen sample, a sectioned sample, or a formalin-fixed and paraffin-embedded (FFPE) sample. In some embodiments, the first target nucleic acid sequence comprises DNA, cDNA, RNA, coding RNA, non-coding RNA, mRNA, tRNA, rRNA, miRNA, gRNA, snRNA, siRNA, anti-sense RNA, mature microRNA, or immature microRNA, and/or wherein the first target RNA sequence comprises coding RNA, non-coding RNA, mRNA, tRNA, rRNA, miRNA, gRNA, snRNA, siRNA, anti-sense RNA, mature microRNA, or immature microRNA. In some embodiments, the second target nucleic acid sequence comprises DNA, cDNA, RNA, coding RNA, non-coding RNA, mRNA, tRNA, rRNA, miRNA, gRNA, snRNA, siRNA, anti-sense RNA, mature microRNA, or immature microRNA, and/or wherein the second target RNA sequence comprises coding RNA, non-coding RNA, mRNA, tRNA, rRNA, miRNA, gRNA, snRNA, siRNA, anti-sense RNA, mature microRNA, or immature microRNA. In some embodiments, the biological sample comprises a human sample, a simian sample, an ape sample, a canine sample, a feline sample, a bovine sample, an equine sample, a murine sample, a porcine sample, a caprine sample, a lupine sample, a ranine sample, a piscine sample, a plant sample, an insect sample, a bacteria sample, an algae sample, a viral sample, a protozoa sample, or a fungus sample.

In some embodiments, the first, second, third or fourth concatemer further comprises a compaction oligonucleotide, wherein: a first segment of the compaction oligonucleotide is complementary and binds to a first portion of the first, second, third, or fourth concatemer. In some embodiments, a second segment of the compaction oligonucleotide is complementary and binds to a second portion of the first, second, third, or fourth concatemer, to result in a reduction in the size or a change in the shape of the second concatemer. In some embodiments, the first sequencing product nucleic acid molecule comprises a first identification sequence that identifies the first target RNA sequence and the second sequencing product nucleic acid molecule comprises a second identification sequence that identifies the second target RNA sequence. In some embodiments, the first sequencing product nucleic acid molecule comprises a first identification sequence that identifies the first target RNA sequence and a portion of the first target RNA sequence, wherein the second sequencing product nucleic acid molecule comprises a second identification sequence that identifies the second target RNA sequence and a portion of the second target RNA sequence. In some embodiments, the nucleotide comprises a fluorescent label and a removable blocking group at the 3′ carbon position of the sugar moiety. In some embodiments, the determining further comprises incorporating the nucleotide into the 3′ end of the primer sequence. In some embodiments, the determining further comprises identifying the nucleobase of the incorporated nucleotide by imaging the fluorescent label of the incorporated nucleotide. In some embodiments, the determining further comprises contacting the nucleotide with an agent to remove a blocking group from the nucleotide and generate a 3′ OH group on the sugar moiety. In some embodiments, the blocking group comprises an alkyl group, an alkenyl group, an alkynyl group, an allyl group, an aryl group, a benzyl group, an azide group, an azido group, an O-azidomethyl group, an amine group, an amide group, a keto group, an isocyanate group, a phosphate group, a thiol group, a disulfide group, a carbonate group, a urea group, or a silyl group. In some embodiments, the plurality of nucleotides comprise one type of nucleotide selected from a group comprising dATP, dGTP, dCTP, dTTP and dUTP. In some embodiments, the plurality of nucleotides comprise a mixture of any combination of two or more types of nucleotides selected from a group consisting of dATP, dGTP, dCTP, dTTP and/or dUTP.

Compositions as disclosed herein may comprise two of the first, second, third, or fourth concatemer; and two of a polymerizing enzyme, a plurality of nucleotide conjugates, and two of a primer sequence that is complementary to a portion of the first, second, third, or fourth concatemer. In some embodiments, two of the first, second, third, or fourth concatemer are contacted with two of a polymerizing enzyme, a plurality of nucleotide conjugates, and two of a primer sequence that is complementary to a portion of the first, second, third, or fourth concatemer to form a multivalent binding complex comprising each of the two of the polymerizing enzyme, a nucleotide conjugate of the plurality of nucleotide conjugates, and each of the two of the first, second, third, or fourth concatemer hybridized to each of the two of the primer sequence. In some embodiments, the nucleotide conjugate comprises a label and at least two of a nucleotide moiety. In some embodiments, two of the at least two of the nucleotide moiety are each complementary and bind to a nucleotide of each of the two of the first concatemer. In some embodiments, the composition comprises one or more of a second polymerizing enzyme. In some embodiments, the composition comprises a plurality of unlabeled nucleotides under conditions suitable for forming a binding complex comprising each of the two of the second polymerizing enzyme, each of the two of the first, second, third, or fourth concatemer hybridized to each of the two of the primer sequence, and two of the plurality of the unlabeled nucleotides and incorporating each of the two of the plurality of the unlabeled nucleotides into each of the two of the primer sequence. In some embodiments, each of the two of the plurality of the unlabeled nucleotides is complementary and binds to a nucleotide of each of the two of the first, second, third, or fourth concatemer. In some embodiments, an unlabeled nucleotide of the plurality of unlabeled nucleotides comprises a removable blocking group at the 3′ carbon of the sugan moiety.

The present disclosure provides compositions for detecting in situ at least two different target RNA molecules in a cellular sample. The composition may be configured to interact with a cellular sample deposited on a solid support, wherein the cellular sample harbors at least a first plurality of DNA amplicons that correspond to a first target RNA molecule and the cellular sample harbors a second plurality of DNA amplicons that correspond to a second target RNA molecule. In some embodiments, the cellular sample can comprise a first target RNA molecule. In some embodiments, the cellular sample can comprise at least a first plurality of DNA amplicons that can correspond to a first target RNA molecule. In some embodiments, the cellular sample can comprise a second target RNA molecule. In some embodiments, the cellular sample can comprise at least a second plurality of DNA amplicons that can correspond to a second target RNA. In some embodiments, the cellular sample can be deposited on a solid support.

Compositions as disclosed herein may comprise one or more of (i) a plurality of reverse transcription primers, (ii) a plurality of reverse transcriptase enzymes, and (iii) a plurality of nucleotides, under a condition suitable for conducting a reverse transcription reaction to generate a plurality of cDNA molecules (e.g., a plurality of first strand cDNA molecules) in the cellular sample (e.g., FIG. 1). In some embodiments, the plurality of reverse transcription primers comprises a first sub-population of target-specific reverse transcription primers that hybridize selectively to the first target RNA, and comprises a second sub-population of target-specific reverse transcription primers that hybridize selectively to the second target RNA. In some embodiments, the plurality of reverse transcription primers comprises a first sub-population of random-sequence reverse transcription primers that hybridize to the first target RNA, and comprises a second sub-population of random-sequence reverse transcription primers that hybridize to the second target RNA.

In some embodiments, compositions as disclosed herein may comprise a plurality of cDNA molecules. The cDNA molecules can include at least a first target cDNA molecule that can corresponds to the first target RNA molecule, and the plurality of cDNA molecules can include a second target cDNA molecule that can correspond to the second target RNA molecule. In some embodiments, the plurality of cDNA molecules can include at least a first target cDNA molecule. In some embodiments, the first target cDNA molecule can correspond to the first target RNA molecule. In some embodiments, the plurality of cDNA molecules can include at least a second target cDNA molecule. In some embodiments, the second target cDNA molecule can correspond to the second target RNA molecule. In some embodiments, the plurality of cDNA molecules can be made by contacting the plurality of RNA inside the cellular sample with (i) a plurality of reverse transcription primers, (ii) a plurality of reverse transcriptase enzymes, and (iii) a plurality of nucleotides, under a condition suitable for conducting a reverse transcription reaction to generate the plurality of cDNA molecules (e.g., a plurality of first strand cDNA molecules) in the cellular sample (e.g., FIG. 1). In some embodiments, the generating of (b) can comprise contacting the plurality of RNA inside the cellular sample with a plurality of reverse transcription primers. In some embodiments, the generating of (b) can comprise contacting the plurality of RNA inside the cellular sample with a plurality of reverse transcription enzymes. In some embodiments, the generating of (b) can comprise contacting the plurality of RNA inside the cellular sample with a plurality of nucleotides. In some embodiments, the plurality of reverse transcription primers can comprises a first sub-population of target-specific reverse transcription primers that can hybridize selectively to the first target RNA, and can comprises a second sub-population of target-specific reverse transcription primers that can hybridize selectively to the second target RNA. In some embodiments, the plurality of reverse transcription primers can comprise a first sub-population of target-specific reverse transcription primers. In some embodiments, the first sub-population of target-specific reverse transcription primers can hybridize selectively to the first target RNA. In some embodiments, the plurality of reverse transcription primers can comprise a second sub-population of target-specific reverse transcription primers. In some embodiments, the second sub-population of target-specific reverse transcription primers can hybridize selectively to the second target RNA. In some embodiments, the plurality of reverse transcription primers can comprise a first sub-population of random-sequence reverse transcription primers that can hybridize to the first target RNA, and can comprise a second sub-population of random-sequence reverse transcription primers that can hybridize to the second target RNA. In some embodiments, the plurality of reverse transcription primers can comprise a first sub-population of random-sequence reverse transcription primers. In some embodiments, the first sub-population of random-sequence reverse transcription primers can hybridize to the first target RNA. In some embodiments, the plurality of reverse transcription primers can comprise a second sub-population of random-sequence reverse transcription primers. In some embodiments, the second sub-population of random-sequence reverse transcription primers can hybridize to the second target RNA.

Compositions as disclosed herein may further comprise a plurality of target-specific padlock probes. The plurality of target-specific padlock probes may comprise at least a first plurality of target-specific padlock probes and a second plurality of target-specific padlock probes. In some embodiments, the plurality of target-specific padlock probes can include at least a first plurality of target-specific padlock probes. In some embodiments, the plurality of target-specific padlock probes can include at least a second plurality of target-specific padlock probes. In some embodiments, the method comprises contacting the plurality of cDNA molecule in the cellular sample with at least 2-10,000 different target-specific padlock probes.

In some embodiments, individual padlock probes in the plurality of first target-specific padlock probes comprise a first and second end (e.g., first and second padlock binding arms), wherein the first end selectively hybridizes to a first region of the first target cDNA molecule and the second end selectively hybridizes to a second region of the first target cDNA molecule. In some embodiments, the first and second ends of the first target-specific padlock probes can be hybridized to proximal positions on the first target cDNA molecule to form a circularized first target-specific padlock probe having a nick or gap between the hybridized first and second ends (e.g., FIG. 1). In some embodiments, the first target-specific padlock probe comprises a first target barcode sequence that corresponds to the first target cDNA sequence. In some embodiments, the first target-specific padlock probe comprises a first target barcode sequence that is located adjacent to one of the regions of the first target-specific padlock probe that selectively hybridizes to the first target cDNA molecule. In some embodiments, the first target-specific padlock probe comprises at least one universal adaptor sequence, such as for example a universal sequencing primer binding site (or a complementary sequence thereof). In some embodiments, the first target-specific padlock probe comprises a universal primer binding site for a rolling circle amplification primer (or a complementary sequence thereof). In some embodiments, the first target-specific padlock probe comprises a universal compaction oligonucleotide binding site (or a complementary sequence thereof).

In some embodiments, individual padlock probes in the plurality of second target-specific padlock probes comprise a first and second end, wherein the first end selectively hybridizes to a first region of the second target cDNA molecule and the second end selectively hybridizes to a second region of the second target cDNA molecule. In some embodiments, the first and second ends of the second target-specific padlock probes can be hybridized to proximal positions on the second target cDNA molecule to form a circularized second target-specific padlock probe having a nick or gap between the hybridized first and second ends. In some embodiments, the second target-specific padlock probe comprises a second target barcode sequence that corresponds to the second target cDNA sequence. In some embodiments, the second target-specific padlock probe comprises a second target barcode sequence that is located adjacent to one of the regions of the second target-specific padlock probe that selectively hybridizes to the second target cDNA molecule. In some embodiments, the second target-specific padlock probe at least one universal adaptor sequence, such as for example a comprises at least one universal sequencing primer binding site (or a complementary sequence thereof). In some embodiments, the second target-specific padlock probe comprises a universal primer binding site for a rolling circle amplification primer (or a complementary sequence thereof). In some embodiments, the second target-specific padlock probe comprises a universal compaction oligonucleotide binding site (or a complementary sequence thereof).

In some embodiments, the target-specific padlock probes comprise a universal sequencing primer binding site and a target barcode sequence that are adjacent to each other so that the target barcode region of the concatemer is sequenced first. The target barcode sequence can be any length, for example 3-15 bases, or 15-25 bases, or 25-40 bases, or longer.

In some embodiments, compositions a disclosed herein may further comprise one or more components or reagents configured to close a nick or gap in a circularized target-specific padlock probe. The components or reagent may be configured to conduct an enzymatic reaction, thereby generating a covalently closed circular padlock probe. In some embodiments, the closing the nick can comprise conducting an enzymatic ligation reaction. In some embodiments, closing the gap can comprise conducting a polymerase-catalyzed fill-in reaction using the first or second target cDNA molecule as a template, and conducting an enzymatic ligation reaction. In some embodiments, closing the gap can comprise conducting a polymerase-catalyzed fill-in reaction using the first target cDNA molecule as a template. In some embodiments, closing the gap can comprise conducting a polymerase-catalyzed fill-in reaction using the second target cDNA molecule as a template. In some embodiments, the method can comprise closing the nick or gap in at least 2-10,000 circularized target-specific padlock probes by conducting an enzymatic reaction, thereby generating at least 2-10,000 covalently closed circular padlock probes inside the cellular sample.

In some embodiments, compositions as disclosed herein may further comprise one or more components or reagents for conducting a rolling circle amplification reaction inside a cellular sample. In some embodiments, the first and second covalently closed circular padlock probes can be used as template molecules, thereby generating a plurality of concatemer molecules including at least a first concatemer molecule that corresponds to a first target RNA molecule, and the plurality of concatemer molecules can include at least a second concatemer molecule that corresponds to a second target RNA molecule. In some embodiments, a rolling circle amplification reaction can be conducted inside the cellular sample using the first covalently closed circular padlock probe as a template molecule. In some embodiments, the rolling circle amplification of the first covalently closed circular padlock probe can generate a plurality of concatemer molecule. In some embodiments, the plurality of concatemer molecule can include a first concatemer molecule. In some embodiments, the first concatemer molecule can correspond to a first target RNA molecule. In some embodiments, a rolling circle amplification reaction can be conducted inside the cellular sample using the second covalently closed circular padlock probe as a template molecule. In some embodiments, the rolling circle amplification of the second covalently closed circular padlock probe can generate a plurality of concatemer molecules. In some embodiments, the plurality of concatemer molecule can include a second concatemer molecule. In some embodiments, the second concatemer molecule can correspond to a second target RNA molecule. In some embodiments, the first concatemer molecule can comprise tandem repeat units, wherein a unit can comprise the sequence of the first target cDNA and the universal sequencing primer binding site (or a complementary sequence thereof). In some embodiments, the second concatemer molecule can comprises tandem repeat units, wherein a unit can comprise the sequence of the second target cDNA and the universal sequencing primer binding site (or a complementary sequence thereof). In some embodiments, a rolling circle amplification reaction can be conducted inside the cellular sample using at least 2-10,000 covalently closed circular padlock probes as template molecules, thereby generating at least 2-10,000 concatemer molecules that correspond to at least 2-10,000 target RNA molecules. In some embodiments, the rolling circle amplification can be conducted in the presence of a plurality of compaction oligonucleotides. In some embodiments, each compaction oligonucleotide can comprise a single stranded oligonucleotide. In some embodiments, the single stranded oligonucleotide can have a first region at one end and a second region at the other end. In some embodiments, the first region can hybridize to a portion of the concatemer molecule and the second region can hybridize to another portion of the concatemer molecule. In some embodiments, the hybridization of the first region and the second region of the compaction oligonucleotide can compact the concatemer molecule. In some embodiments, the compaction oligonucleotide can compact the size of the concatemer molecule. In some embodiments, the compaction oligonucleotide can compact the shape of the concatemer molecule. In some embodiments, the compaction oligonucleotide can compact the size and shape of the concatemer molecule. In some embodiments, the compaction oligonucleotide can compact the concatemer molecule to form a compact nanoball.

In some embodiments, compositions as disclosed herein may further comprise one or more reagents or components for sequencing the plurality of concatemer molecules inside the cellular sample. The sequencing may comprise sequencing the first concatemer molecule by conducting no more than 2-30 sequencing cycles to generate a plurality of first sequencing read products, and sequencing the second concatemer molecule by conducting no more than 2-30 sequencing cycles to generate a plurality of second sequencing read products. In some embodiments, sequencing the plurality of concatemer molecules may be conducted inside the cellular sample. In some embodiments, sequencing the plurality of concatemer molecules inside the cellular sample can comprise sequencing the first concatemer molecule by conducting no more than 2-30 sequencing cycles to generate a plurality of first sequencing read products. In some embodiments, sequencing can comprise sequencing the plurality of concatemer molecules inside the cellular sample. In some embodiments, sequencing the plurality of concatemer molecules inside the cellular sample can comprise sequencing the second concatemer molecule by conducting no more than 2-30 sequencing cycles to generate a plurality of second sequencing read products. In some embodiments, the sequencing of can comprise sequencing no more than 2-30 bases of the first concatemer molecules to generate a plurality of first sequencing read products, and which can comprise sequencing no more than 2-30 bases of the second concatemer molecules to generate a plurality of second sequencing read products. In some embodiments, the sequencing of (f) can comprise sequencing no more than 2-30 bases of the first concatemer molecules to generate a plurality of first sequencing read products. In some embodiments, the sequencing of (f) can comprise sequencing no more than 2-30 bases of the second concatemer molecules to generate a plurality of second sequencing read products. In some embodiments, the method can comprise sequencing the at least 2-10,000 concatemer molecules inside the cellular sample, which can comprise conducting no more than 2-30 sequencing cycles on the 2-10,000 concatemer molecules to generate a plurality of sequencing read products.

In some embodiments, only the first target barcode region of the first concatemer molecules are sequenced (e.g., FIG. 2). In some embodiments, at least a portion or the full length of the first target barcode of the first concatemer molecules are sequenced (e.g., FIG. 2). In some embodiments, the first target barcode is sequenced and a portion of the first cDNA region of the first concatemer molecules are sequenced (e.g., FIG. 3). In some embodiments, at least a portion of the first cDNA region of the first concatemer molecules are sequenced (e.g., FIG. 4 or 5).

In some embodiments, only the second target barcode region of the second concatemer molecules are sequenced (e.g., FIG. 2). In some embodiments, at least a portion or the full length of the second target barcode of the second concatemer molecules are sequenced (e.g., FIG. 2). In some embodiments, the second target barcode is sequenced and a portion of the second cDNA region of the second concatemer molecules are sequenced (e.g., FIG. 3). In some embodiments, at least a portion of the second cDNA region of the second concatemer molecules are sequenced (e.g., FIG. 4 or 5).

In some embodiments, the reiterative sequencing can be conducting using a sequencing-by-binding procedure, labeled and/or non-labeled chain-terminating nucleotides, or multivalent molecules. Descriptions of these three sequencing compositions is described below.

In some embodiments, the plurality of universal sequencing primers can be hybridized to concatemer template molecules with a hybridization reagent comprising an SSC buffer (e.g., 2× saline-sodium citrate) buffer with formamide (e.g., 10-20% formamide). The hybridization conditions comprise a temperature of about 20-30° C., for about 10-60 minutes.

In some embodiments, the plurality of sequencing read products can be removed from the concatemers and the plurality of concatemers can be retained inside the cellular sample using a de-hybridization reagent comprising an SSC buffer (e.g., saline-sodium citrate) buffer, with or without formamide, at a temperature that promotes nucleic acid denaturation such as for example 30-90° C.

In some embodiments, the plurality of nucleotide reagents comprise a plurality of nucleotides that are detectably labeled or non-labeled. In some embodiments, individual nucleotides are linked to a detectable reporter moiety. In some embodiments, the detectable reporter moiety comprises a fluorophore. In some embodiments, the plurality of detectably labeled nucleotide analogs comprise a plurality of chain terminating nucleotides, where the chain terminating moiety is linked to the 3′ nucleotide sugar position to form a 3′ blocked nucleotide analog. In some embodiments, the chain terminating moiety can be removed to convert the 3′ blocked nucleotide analog to an extendible nucleotide having a 3′ OH group on the sugar. In some embodiments, the labeled nucleotide analogs are linked to a different fluorophore that corresponds to the nucleobases adenine, cytosine, guanine, thymine or uracil, where the different fluorophores emit a fluorescent signal during the sequencing of step (f). In some embodiments, a sequencing cycle comprises (1) contacting the concatemer/sequencing primer duplex with a sequencing polymerase and a detectably labeled chain terminating nucleotide under a condition suitable for polymerase-catalyzed incorporation of the detectably labeled chain terminating nucleotide into the terminal end of the sequencing primer, (2) detecting and imaging the fluorescent signal and color emitted by the incorporated chain terminating nucleotide, and (3) removing the chain terminating moiety (e.g., unblocking) and retaining the concatemer/sequencing primer duplex. In some embodiments, no more than 2-30 sequencing cycles can be conducted on the plurality of concatemers inside the cellular sample to generate a plurality of first sequencing read products and a plurality of second sequencing read products.

In some embodiments, out-of-sync phasing and/or pre-phasing events can occur during synchronized sequencing reactions on clonally amplified template amplicons, where the sequencing reactions comprise polymerase-catalyzed sequencing reactions employing detectably labeled chain terminator nucleotides. In some embodiments, a sequencing reaction on one template molecule in the clonally-amplified template molecules moves ahead (e.g., pre-phasing) or fall behind (e.g., phasing) of the sequencing of the other template molecules within the clonally-amplified template molecules. During sequencing, a fluorescent signal is typically detected which corresponds to incorporation of a labeled chain terminator nucleotide. Thus, phasing and pre-phasing events can be detected and monitored using incorporation of a labeled chain terminator nucleotide.

In some embodiments, the plurality of nucleotide reagents of step (f) comprise a plurality of multivalent molecules each comprising a core attached to a plurality of nucleotide-arms, wherein the nucleotide-arms are attached to a nucleotide unit. In some embodiments, individual multivalent molecules are labeled with a detectably reporter moiety. In some embodiments, the detectable reporter moiety comprises a fluorophore. In some embodiments, the core of the multivalent molecule is labeled with a fluorophore, and wherein the fluorophore which is attached to a given core of the multivalent molecule corresponds to the nucleotide base (e.g., adenine, guanine, cytosine, thymine or uracil) of the nucleotide arm. In some embodiments, at least one of the nucleotide arms of the multivalent molecule comprises a linker and/or nucleotide base that is attached to a fluorophore, and wherein the fluorophore which is attached to a given nucleotide base corresponds to the nucleotide base (e.g., adenine, guanine, cytosine, thymine or uracil) of the nucleotide arm. In some embodiments, a sequencing cycle can comprise contacting the concatemer/sequencing primer duplex with a first sequencing polymerase to form a complexed polymerase. In some embodiments, a sequencing cycle can comprise contacting the complexed polymerase with a detectably labeled multivalent molecule under a condition suitable for binding a complementary nucleotide unit of the multivalent molecule to the complexed polymerase thereby forming a multivalent-binding complex, and the condition is suitable for inhibiting incorporation of the complementary nucleotide unit into the terminal end of the sequencing primer.

In situ Batch Sequencing RNA and Polypeptides

The present disclosure provides compositions for conducting in situ multiplex and multi-omics detection and identification using coded padlocks probes. The padlock probes are designed to selectively detect target RNA or polypeptides. The padlock probe may comprise one or more batch-specific primer-binding sites, which are specific to a primer. The primer may be spatially localized, and the padlock probe may therefore not require a barcode sequence. In some embodiments, however, the padlock probe may comprise a barcode sequence.

The RNA-specific padlock probes selectively hybridize to cDNA that corresponds to target RNA. The RNA-specific probes carry barcodes that uniquely identify the cDNA. The RNA-specific padlock probes also carry batch-specific sequencing primer binding sites. The padlock probe may comprise one or more batch-specific primer-binding sites, which are specific to a primer. The primer may be spatially localized, and the padlock probe may therefore not require a barcode sequence. In some embodiments, however, the padlock probe may comprise a barcode sequence.

The target polypeptides are detected using antibody-oligonucleotide conjugates and polypeptide-specific padlock probes which selectively hybridize to the oligonucleotide which is conjugated to the antibody. The polypeptide-specific padlock probes carry barcodes that uniquely identify the antibody that selectively binds a target polypeptides. The polypeptide-specific padlock probes also carry a batch-specific sequencing primer which is the same batch-specific sequencing primer carried by a corresponding RNA-specific padlock probe to enable simultaneous sequencing and detection of a target RNA and the polypeptide encoded by that target RNA. Thus, the padlock probes enable simultaneous detection and identification of RNA and their encoded polypeptides.

### Reverse Transcriptase

The present disclosure provides compositions for conducting in situ analysis of a nucleic acid. The analysis may comprise a sequencing of the nucleic acid. In some embodiments, the nucleic acid may be an RNA. The analysis of the RNA may comprise reverse transcribing the RNA into a complementary DNA (cDNA). A reverse transcriptase may be used to transcribe the RNA into a cDNA.

Compositions as described herein may further comprise a reverse transcriptase. In some embodiments, the reverse transcriptase enzyme can exhibit RNA-dependent DNA polymerase activity. In some embodiments, the reverse transcriptase enzyme can comprise a reverse transcriptase enzyme from AMV (avian myeloblastosis virus), M-MuLV (moloney murine leukemia virus), or HIV (human immunodeficiency virus). In some embodiment, the reverse transcriptase enzyme can comprise a recombinant enzyme that exhibits reduced RNase H activity, for example REVERTAID (e.g., from Thermo Fisher Scientific, catalog No. EP0441). In some embodiments, the reverse transcriptase can be a commercially-available enzyme, including MULTISCRIBE (e.g., from Thermo Fisher Scientific, catalog #4311235), THERMOSCRIPT (e.g., from Thermo Fisher Scientific, catalog #12236-014), or ARRAYSCRIPT (e.g., from Ambion, catalog No. AM2048). In some embodiments, the reverse transcriptase enzyme can comprise SUPERSCRIPT II (e.g., catalog No. 18064014), SUPERSCRIPT III (e.g., catalog No. 18080044), or SUPERSCRIPT IV enzymes (e.g., catalog No. 18090010) (all SUPERSCRIPT enzymes from Invitrogen). In some embodiments, the reverse transcription reaction can include an RNase inhibitor.

In some embodiments, the reverse transcription primers comprise a single-stranded oligonucleotide comprising DNA, RNA, or chimeric DNA/RNA. In some embodiments, the reverse transcription primers Any combination of adenine (A), thymine (T), guanine (G), cytosine (C), uracil (L . . . 1) and/or inosine (I). In some embodiments, the reverse transcription primers can be any length, for example 5-25 bases, or 25-50 bases, or 50-75 bases, or 75-100 bases in length or longer. The reverse transcription primers each comprise a 5′ end and 3′ end. In some embodiments, the 3′ end of the reverse transcription primers can include a 3′ OH moiety which serves as a nucleotide polymerization initiation site in a polymerase-catalyzed primer extension reaction. In some embodiments, the 3′ end of the reverse transcription primers have a chain terminating moiety which blocks a polymerase-catalyzed primer extension reaction. The chain terminating moiety can be removed to convert the 3′ sugar position to an extendible 3′OH.

In some embodiments, the reverse transcription primers are modified to confer resistance to nuclease degradation (e.g., ribonuclease degradation). For example, the reverse transcription primers comprise at least one phosphorothioate diester bond at their 5′ ends which can render the reverse transcription primers resistant to nuclease degradation. In some embodiments, the reverse transcription primers comprise 2-5 or more consecutive phosphorothioate diester bonds at their 5′ ends. In some embodiments, the plurality of reverse transcription primers comprise at least one ribonucleotide and/or at least one 2′-O-methyl, 2′-O-methoxyethyl (MOE), 2′ fluoro-base nucleotide. In some embodiments, the reverse transcription primers comprise phosphorylated 3′ ends. In some embodiments, the reverse transcription primers comprise locked nucleic acid (LNA) bases. In some embodiments, the reverse transcription primers comprise a phosphorylated 5′ end (e.g., using a polynucleotide kinase).

In some embodiments, the entire length of a reverse transcription primer can hybridize to a portion of an RNA molecule. In some embodiments, individual reverse transcription primers comprise a 3′ region having a sequence that hybridizes to a portion of an RNA molecule and a 5′ region that carries a tail that does not hybridize to an RNA molecule. In some embodiments, the 5′ tail comprises a universal adaptor sequence including any one or any combination of two or more of a sample barcode sequence, an amplification primer binding site, a sequencing primer binding site, a compaction oligonucleotide binding site and/or a surface capture primer binding site. In some embodiments, the 5′ tail comprises a unique identification sequence (e.g., unique molecular index (UMI). In some embodiments, the 5′ tail comprises a restriction enzyme recognition sequence. In some embodiments, individual reverse transcription primers comprise at least a portion of the 3′ region having a homopolymer sequence, for example poly-A, poly-T, poly-C, poly-G or poly-U. In some embodiments, the reverse transcription primers can hybridize to any portion of an RNA molecule, including the 5′ or the 3′ end of the RNA molecule, or an internal portion of the RNA molecule.

In some embodiments, the plurality of reverse transcription primers comprises a first sub-population of target-specific reverse transcription primers that hybridize selectively to the first target RNA (e.g., targeted transcriptomics). In some embodiments, the plurality of reverse transcription primers further comprise a second sub-population of target-specific reverse transcription primers that hybridize selectively to the second target RNA. In some embodiments, the target-specific reverse transcription primers comprise a pre-determined sequence at the 3′ region which hybridizes to a target RNA molecule. In some embodiments, the pre-determined sequence portion of the reverse transcription primers can be 4-20 bases, or 20-40 bases, or 40-50 bases in length.

In some embodiments, the first sub-population of target-specific reverse transcription primers can selectively hybridize to an RNA transcribed in the cellular sample by a housekeeping gene. In some embodiments, selection of the housekeeping gene may be dependent upon the type of cellular sample to be used for the in situ compositions described herein. Non-limiting example housekeeping genes include glyceraldehyde-3-phosphate dehydrogenase (GAPDH), beta-actins (ACTB), tubulins, PPIA (peptidyl-prolyl cis-trans isomerase), NME4 (NME/NM23 nucleoside diphosphate kinase 4), SMARCAL1 (SWI/SNF related matrix associated actin dependent regulator of chromatin, subfamily A like 1), and POMK (protein-O-mannose kinase). The skilled artisan can design the first sub-population of target-specific reverse transcription primers to hybridize to RNA transcripts from any of the numerous housekeeping genes.

In some embodiments, the second sub-population of target-specific reverse transcription primers can selectively hybridize to an RNA transcribed from a gene that is expressed in the cellular sample being examined (e.g., a cell-specific or tissue-specific RNA).

In some embodiments, the plurality of reverse transcription primers comprises a first sub-population of random-sequence reverse transcription primers that hybridize to the first target RNA (e.g., whole transcriptomics). In some embodiments, the plurality of reverse transcription primers further comprises a second sub-population of random-sequence reverse transcription primers that hybridize to the second target RNA. In some embodiments, the reverse transcription primers comprise a random and/or degenerate sequence at the 3′ region which hybridizes to an RNA molecule. In some embodiments, the random-sequence or the degenerate-sequence portion of the reverse transcription primers can be 4-20 bases, or 20-40 bases, or 40-50 bases in length.

### Rolling Circle Amplification (RCA)

Compositions as disclosed herein may comprise a plurality of concatemer molecules. The plurality of concatemer molecules may be generated via RCA. The plurality of concatemer molecules each having two or more tandem copies of a unit wherein the unit comprises a target sequence that corresponds to a target RNA molecules and any additional sequence(s) carried by the padlock probes including universal adaptor sequence(s), unique molecular index sequence(s) and/or restriction enzyme recognition sequence(s). In some embodiments, a unit can comprise a target sequence that can correspond to a target RNA molecule. In some embodiments, a unit can comprise additional sequence(s) carried by the padlock probes. In some embodiments, the additional sequences can include universal adaptor sequence(s), unique molecular index sequence(s), restriction enzyme recognition sequence(s), or any combination thereof.

In some embodiments, when the rolling circle amplification reaction includes a plurality of nucleotide which includes dUTP, the resulting concatemer can be cross-linked to a cross-linking reactive group by treating the cellular sample with a succininuide ester (NHS), maleimide (Sulfo-SMCC), imidoester (DMP), carbodiimide (DCC, EDC) or phenyl azide. in some embodiments, polymerization of the cross-linking reactive group can be initiated with light or UV light. in some embodiments, the resulting concatemer can be cross-linked to a matrix by treating the cellular sample with a cross-linked agarose, cross-linked dextran or cross-linked polyethylene glycol (PEG), polyacrylamide, cellulose alginate or polyamide. In some embodiments, the PEG comprises a sulfo-NHS ester moiety at one or both ends, for example a PEGylated bis(sulfosuccinimidyl)suberate) (e.g., BS(PEG)9 from Thermo Fisher Scientific, catalog No. 21582).

In some embodiments, the rolling circle amplification reaction can be conducted at a constant temperature (e.g., isothermal) wherein the constant temperature is at room temperature to about 30° C., or about 30-40° C., or about 40-50° C., or about 50-65° C.

In some embodiments, the DNA polymerase having a strand displacing activity can be selected from a group consisting of phi29 DNA polymerase, large fragment of Bst DNA polymerase, large fragment of Bsu DNA polymerase, and Bca (exo-) DNA polymerase, Klenow fragment of E. coli DNA polymerase, T5 polymerase, M-MuLV reverse transcriptase, HIV viral reverse transcriptase, or Deep Vent DNA polymerase. In some embodiments, the phi29 DNA polymerase can be wild type phi29 DNA polymerase (e.g., MagniPhi from Expedeon), or variant EquiPhi29 DNA polymerase (e.g., from Thermo Fisher Scientific), and chimeric QualiPhi DNA polymerase (e.g., from 4basebio).

In some embodiments, the rolling circle amplification primers comprise at least one phosphorothioate diester bond at their 5′ ends which can render the amplification primers resistant to exonuclease degradation. In some embodiments, the rolling circle amplification primers comprise 2-5 or more consecutive phosphorothioate diester bonds at their 5′ ends. In some embodiments, the rolling circle amplification primers comprise at least one ribonucleotide and/or at least one 2′-O-methyl or 2′-O-methoxyethyl (MOE) nucleotide.

In some embodiments, the composition further comprises a plurality of compaction oligonucleotides which, when hybridized to a concatemer molecule, can compact the size, the shape, or a combination of the size and the shape of the concatemer to form a compact nanoball. In some embodiments, rolling circle amplification reaction can be conducted in the presence of one or more compaction oligonucleotides.

The compaction oligonucleotide may include a 5′ region, an optional internal region (intervening region), a 3′ region, or any combination thereof. A 5′ and 3′ region of the compaction oligonucleotide can hybridize to different portions of the concatemer to pull together distal portions of the concatemer causing compaction of the concatemer to form a DNA nanoball. For example, the 5′ region of the compaction oligonucleotide can be designed to hybridize to a first portion of the concatemer molecule (e.g., a universal compaction oligonucleotide binding site), and the 3′ region of the compaction oligonucleotide is designed to hybridized to a second portion of the concatemer molecule (e.g., a universal compaction oligonucleotide binding site). Inclusion of compaction oligonucleotides during RCA can promote formation of DNA nanoballs having tighter size and shape compared to concatemers generated in the absence of the compaction oligonucleotides. The compact and stable characteristics of the DNA nanoballs improves in situ sequencing accuracy by increasing signal intensity and the nanoballs retain their shape and size during multiple sequencing cycles.

In some embodiments, the compaction oligonucleotides comprise single stranded oligonucleotides comprising DNA, RNA, or a combination of DNA and RNA. The compaction oligonucleotides can be any length, including 20-150 nucleotides, or 30-100 nucleotides, or 40-80 nucleotides in length.

In some embodiments, the compaction oligonucleotide comprises a 5′ region and a 3′ region, and optionally an intervening region between the 5′ and 3′ regions. The intervening region can be any length, for example about 2-20 nucleotides in length. The intervening region comprises a homopolymer having consecutive identical bases (e.g., AAA, GGG, CCC, TTT or UUU). The intervening region comprises a non-homopolymer sequence.

The 5′ region of the compaction oligonucleotide can be wholly complementary or partially complementary along its length to a first portion of a concatemer molecule. The 5′ region of the compaction oligonucleotides can be wholly complementary along its length to a first portion of a concatemer molecule. The 5′ region of the compaction oligonucleotides can be partially complementary along its length to a first portion of a concatemer molecule. The 3′ region of the compaction oligonucleotides can be wholly complementary or partially complementary along its length to a second portion of a concatemer molecule. The 3′ region of the compaction oligonucleotides can be wholly complementary along its length to a second portion of a concatemer molecule. The 3′ region of the compaction oligonucleotides can be partially complementary along its length to a second portion of a concatemer molecule. The 5′ region of the compaction oligonucleotides can hybridize to a first universal sequence portion of a concatemer molecule. The 3′ region of the compaction oligonucleotides can hybridize to a second universal sequence portion of a concatemer molecule . . . .

In some embodiments, the 5′ region of the compaction oligonucleotide can have the same sequence as the 3′ region. The 5′ region of the compaction oligonucleotide can have a sequence that is different from the 3′ region. In some embodiments, the 3′ region of the compaction oligonucleotide can have a sequence that is a reverse sequence of the 5′ region. In some embodiments, the 5′ region of the compaction oligonucleotide can have a sequence that is a reverse sequence of the 3′ region.

In some embodiments, the 3′ region of any of the compaction oligonucleotides can include an additional three bases at the terminal 3′ end which comprises 2′-O-methyl RNA bases (e.g., designated mUmUmU) or the terminal 3′ end lacks additional 2′-O-methyl RNA bases.

In some embodiments, the compaction oligonucleotides comprise one or more modified bases or linkages at their 5′ or 3′ ends to confer certain functionalities. In some embodiments, the compaction oligonucleotides comprise at least one phosphorothioate linkages at their 5′ and/or 3′ ends to confer exonuclease resistance. In some embodiments, at least one nucleotide at or near the 3′ end comprises a 2′ fluoro base which confers exonuclease resistance. In some embodiments, the 3′ end of the compaction oligonucleotides comprise at least one 2′-O-methyl RNA base which blocks polymerase-catalyzed extension. For example, the 3′ end of the compaction oligonucleotide comprises three bases comprising 2′-O-methyl RNA base (e.g., designated mUmUmU). In some embodiments, the compaction oligonucleotides comprise a 3′ inverted dT at their 3′ ends which blocks polymerase-catalyzed extension. In some embodiments, the compaction oligonucleotides comprise 3′ phosphorylation which blocks polymerase-catalyzed extension. In some embodiments, the internal region of the compaction oligonucleotides comprise at least one locked nucleic acid (LNA) which increases the thermal stability of duplexes formed by hybridizing a compaction oligonucleotide to a concatemer molecule. In some embodiments, the compaction oligonucleotides comprise a phosphorylated 5′ end (e.g., using a polynucleotide kinase).

In some embodiments, the compaction oligonucleotide comprises the sequence 5′-CATGTAATGCACGTACTTTCAGGGTAAACATGTAATGCACGTACTTTCAGGGT-3′ (SEQ ID NO: 14). In some embodiments, the compaction oligonucleotides includes an additional three bases at the terminal 3′ end which comprises 2′-O-methyl RNA bases (e.g., designated mUmUmU) or the terminal 3′ end lacks additional 2′-O-methyl RNA bases.

In some embodiments, the compaction oligonucleotides can include at least one region having consecutive guanines. For example, the compaction oligonucleotides can include at least one region having 2, 3, 4, 5, 6 or more consecutive guanines. In some embodiments, the compaction oligonucleotides comprise four consecutive guanines which can form a guanine tetrad structure (see FIG. 18). The guanine tetrad structure can be stabilized via Hoogsteen hydrogen bonding. The guanine tetrad structure can be stabilized by a central cation including potassium, sodium, lithium, rubidium or cesium.

At least one compaction oligonucleotide can form a guanine tetrad (FIG. 18) and hybridize to the universal binding sequences in a concatemer which can cause the concatemer to fold to form an intramolecular G-quadruplex structure (FIG. 19). The concatemers can self-collapse to form compact nanoballs. Formation of the guanine tetrads and G-quadruplexes in the nanoballs may increase the stability of the nanoballs to retain their compact size and shape which can withstand changes in pH, temperature and/or repeated flows of reagents during sequencing inside the cellular sample.

In some embodiments, the plurality of compaction oligonucleotides in the rolling circle amplification reaction have the same sequence. Alternatively, the plurality of compaction oligonucleotides in the rolling circle amplification reaction comprise a mixture of two or more different populations of compaction oligonucleotides having different sequences.

In some embodiment, the immobilized concatemer template molecule can self-collapse into a compact nucleic acid nanoball. The nanoballs can be imaged and a FWHM measurement can be obtained to give the shape/size of the nanoballs.

In some embodiments, inclusion of compaction oligonucleotides in the rolling circle amplification reaction can promote collapsing of a concatemer into a DNA nanoball. Conducting RCA with compaction oligonucleotides helps retain the compact size and shape of a DNA nanoball during multiple sequencing cycles which can improve FWHM (full width half maximum) of a spot image of the DNA nanoball inside a cellular sample. In some embodiments, the DNA nanoball does not unravel during multiple sequencing cycles. In some embodiments, the spot image of the DNA nanoball does not enlarge during multiple sequencing cycles. In some embodiments, the spot image of the DNA nanoball remains a discrete spot during multiple sequencing cycles. The spot image can be represented as a Gaussian spot and the size can be measured as a FWHM. A smaller spot size as indicated by a smaller FWHM typically correlates with an improved image of the spot. In some embodiments, the FWHM of a nanoball spot can be about 10 μm or smaller.

### Antibody-Oligonucleotide Conjugates

The present disclosure provides a protein-binding composition. The protein-binding composition may be specific to a target polypeptide or protein corresponding to a target RNA. The target RNA may then be sequenced. In this manner, the protein-binding composition may increase the specificity of the methods of sequencing as disclosed herein.

In some embodiments, protein-binding compositions as disclosed herein may comprise an antibody and an oligonucleotide tag. The antibody and oligonucleotide tag may be joined via a linker moiety. The antibody may comprise a plurality of oligonucleotide tags linked via a plurality of linker moieties. The antibody may be specific to a target protein or polypeptide corresponding to a target RNA. The oligonucleotide tag may correspond to a sequence of a padlock probe as disclosed herein. The successful binding of the oligonucleotide tag to a padlock probe as disclosed herein may lock the topological configuration of the padlock probe. In some embodiments, the oligonucleotide tag sequence is designed to provide a sequencing read-out that is associated with binding between the antibody-oligonucleotide conjugate and the target polypeptide. In some embodiments, the sequence of the oligonucleotide tag is designed to exhibit minimal hybridization to RNA (e.g., target RNA) in the cellular sample.

The present disclosure provides one or more sets of antibody-oligonucleotide conjugates, comprising at least a first and a second antibody-oligonucleotide conjugate. A set of antibody-oligonucleotide conjugates can be used to conduct any of the detecting and/or sequencing methods described herein.

In some embodiments, a set comprises at least a first antibody-oligonucleotide conjugate comprising a first antibody which selectively binds a first target polypeptide. The first antibody is linked to a first oligonucleotide carrying an oligonucleotide tag sequence which uniquely identifies the first antibody which selectively binds the first target polypeptide.

In some embodiments, the set comprises at least a second antibody-oligonucleotide conjugate comprising a second antibody which selectively binds a second target polypeptide. The second antibody is linked to a second oligonucleotide carrying an oligonucleotide tag sequence which uniquely identifies the second antibody which selectively binds the second target polypeptide.

In some embodiments, the first antibody-oligonucleotide conjugate and the second antibody-oligonucleotide conjugate are the same (e.g., FIG. 28). In some embodiments, the first antibody-oligonucleotide conjugate and the second antibody-oligonucleotide conjugate are different (e.g., FIG. 29).

In some embodiments, the first and second oligonucleotide tag sequences are designed to selectively bind to left and right binding arms of a padlock probe. In some embodiments, the first and second oligonucleotide tag sequences differ from each other.

In some embodiments, a set of antibody-oligonucleotide conjugates comprises 2-20 antibody-oligonucleotide conjugates, wherein each antibody-oligonucleotide conjugate binds its respective target polypeptide. In some embodiments, the set comprises 20-100 antibody-oligonucleotide conjugates, or 100-500 antibody-oligonucleotide conjugates, or 500-1000 antibody-oligonucleotide conjugate, or more antibody-oligonucleotide conjugates.

The present disclosure provides antibody-oligonucleotide conjugates each comprising an antibody linked to an oligonucleotide. In some embodiments, the antibody comprises an intact immunoglobulin, antibody fragment, an antigen binding portion of an antibody, or single-chain antibody. The antibodies can be monoclonal or polyclonal antibodies. The antibodies are capable of binding specifically to a target analyte. The target analyte includes polypeptides, polynucleotides, carbohydrates, saccharides and lipids. In some embodiments, target analytes comprise intact polypeptides or peptide fragments. The antibodies comprises an antigen-binding region (e.g., paratope) that binds specifically to a target analyte.

An immunoglobulin is typically a tetrameric molecule comprising two identical pairs of polypeptide chains where each pair includes a light chain and a heavy chain. The amino portion of the heavy and light chains each comprise a variable region which associate with each other to form an antigen binding region (e.g., paratope). Thus, a typical immunoglobulin can bind two antigens or can bind two target analytes. The carboxyl portion of the heavy chain comprise a constant region which associate with each other to form an Fc region for effector function. The Fc portion of the heavy chains can define the class of antibody which includes IgG, IgM, IgD, IgA or IgE isotype. The heavy and/or light chains can be prepared using recombinant techniques or by immunizing an animal with an antigen of interest.

The antibody fragment generally comprises a portion of an intact immunoglobulin that can bind an antigen. Examples of antibody fragments include but are not limited to Fv, Fab, Fab′, Fab′-SH, F(ab′)₂, and Fd.

In some embodiments, an Fv fragment comprises a variable light chain region (VL) and variable heavy chain region (VH).

In some embodiments, an Fab fragment comprises a monovalent antibody fragment having a variable light chain region (VL), constant light chain region (CL), variable heavy chain region (VH), and first constant region (CH1).

In some embodiments, an Fab′ fragment comprises a monovalent antibody fragment having a variable light chain region (VL), constant light chain region (CL), variable heavy chain region (VH), first constant region (CH1), hinge region, and at least a portion of a second constant region (CH2).

In some embodiments, an F(ab′)₂ fragment comprises a bivalent antibody fragment having two Fab fragments linked via a disulfide bridge at the hinge region.

A single-chain antibody (scFv) typically comprises a single polypeptide chain (e.g., a monovalent antibody molecule) having a variable light chain region (VL) and variable heavy chain region (VH) joined by a polypeptide linker (see, e.g., Bird et al., 1988, Science 242:423-26 and Huston et al., 1988, Proc. Natl. Acad. Sci. USA 85:5879-83). The amino-terminal end of the single-chain antibody comprises either the variable light chain region (VL) or the variable heavy chain region (VH). In some embodiments, the single-chain antibody comprises an scFv-Fc antibody which further comprises an antibody hinge region, and at least a portion of the Fc region including the CH2 and/or the CH3 region. In some embodiments, the single-chain antibody comprises an scFv-CH antibody which further comprises an antibody hinge region, and at least a portion of the CH3 region.

In some embodiments, the antibody-oligonucleotide conjugates comprise an antibody linked to an oligonucleotide by a linker moiety. In some embodiments, the linker moiety comprises streptavidin (or an avidin-like moiety), biotin, an amine group, or a disulfide group. In some embodiments, the linker moiety is not cleavable or not removable. In some embodiments, the linker moiety is cleavable or removable. For example, the linker moiety can be cleavable with light (e.g., UV light), chemically-cleavable (e.g., dithiothreitol), heat-cleavable, or enzymatically cleavable.

In some embodiments, antibody-oligonucleotide conjugates can be prepared by cross-linking amino groups on the antibody and oligonucleotide using glutaraldehyde. The lysine side chain epsilon-amide is commonly targeted to conjugate to oligonucleotides. In some embodiments, maleimide-modified antibodies can be reacted with sulfhydryl-modified oligonucleotides. In some embodiments, heterobifunctional cross-linkers are introduced as bridges to link together the antibody and oligonucleotide.

In some embodiments, the antibody-oligonucleotide conjugates comprise an antibody linked to an oligonucleotide. In some embodiments, the oligonucleotide comprises a nucleic acid comprising DNA, RNA, or chimeric DNA and RNA. In some embodiments, the oligonucleotide comprises canonical nucleotides or nucleotide analogs such as locked nucleic acids (LNA). In some embodiments, the length of the oligonucleotide can be 10-50 nucleotides in length, or 50-100 nucleotides in length, or 100-200 nucleotides in length.

In some embodiments, the oligonucleotide is designed to carry at least one tag sequence, where the tag sequence selectively binds to left and right binding arms of a padlock probe. In some embodiments, the oligonucleotide tag sequence also uniquely identifies the antibody to which it is conjugated, where the antibody selectively binds a target polypeptide. In some embodiments, the oligonucleotide tag sequence is designed to provide a sequencing read-out that is associated with binding between the antibody-oligonucleotide conjugate and the target polypeptide. In some embodiments, the sequence of the oligonucleotide tag is designed to exhibit minimal hybridization to RNA (e.g., target RNA) in the cellular sample.

In some embodiments, a set of antibody-oligonucleotide conjugates comprises at least a first and a second antibody-oligonucleotide conjugate. In some embodiments, a set of oligonucleotide tags comprises at least a first and a second oligonucleotide tag. In some embodiments, the set of oligonucleotide tags comprises 2-20 oligonucleotide tags wherein each oligonucleotide tag in the set uniquely identifies its respective conjugated antibody where the antibody selectively binds a target polypeptide. The set of oligonucleotide tags comprises 20-100 oligonucleotide tags, or 100-500 oligonucleotide tags, or 500-1000 oligonucleotide tags, or more oligonucleotide tags.

As depicted in the non-limiting embodiment of FIG. 28, a first antibody-nucleotide conjugate and a second antibody-nucleotide conjugate may be used against a target protein encoded by a target RNA (in FIG. 28, “Protein-1 encoded by RNA-1”). The first antibody-nucleotide conjugate, the second antibody-nucleotide conjugate, or both antibody-nucleotide conjugates may comprise a linker moiety. A first oligonucleotide tag may be linked to the first antibody-oligonucleotide conjugate via a first linker moiety. The first oligonucleotide tag may conjugate to a padlock probe. The first oligonucleotide tag of the first antibody-oligonucleotide conjugate may, e.g., ligate, chemically conjugate, or have proximity crosslinking to the padlock probe. A second oligonucleotide tag of the second antibody-oligonucleotide conjugate may be linked to the second antibody via a second linker moiety.

As depicted in the non-limiting embodiment of FIG. 29, a first antibody-oligonucleotide conjugate and a second antibody-oligonucleotide conjugate may be used against a target protein (in FIG. 29, “Protein-1 encoded by RNA-1”). The first antibody-oligonucleotide conjugate may comprise a first antibody and a first oligonucleotide tag. The first oligonucleotide tag may be linked to the first antibody via a first linker moiety. The second antibody-oligonucleotide tag may comprise a second antibody and a second oligonucleotide tag. The second oligonucleotide tag may be linked to the second antibody via a second linker moiety. The first antibody-oligonucleotide conjugate and the second antibody-oligonucleotide conjugate may be specific to the same target protein encoded by the same target RNA.

In some embodiments, the target analytes comprise polypeptides or peptide fragments. In some embodiments, a target polypeptide is encoded by a target RNA in the cellular sample. Target polypeptides include polypeptides having post-translationally modified forms, including methylation, phosphorylation, glycosylation, hydroxylation, ubiquitination, nitrosylation, acetylation, lipidation, ADP-ribosylation, carbonylation, SUMOylation and/or disulfide bond formation. The target polypeptides can be subjected to proteolysis for example by a protease or cleavage due to ribosomal skipping. Target polypeptides also include precursor molecules that have not yet been subjected to post-translation modification. Target polypeptides include muteins, variants, chimeric proteins and fusion proteins. Target polypeptides can be labeled with a binding partner molecule having an affinity moiety, such as for example biotin (or its derivatives), digoxigenin, fluorescein, cholesterol, maltose, or any of the affinity molecules described below.

In some embodiments, polysaccharides inside the cellular sample can be detected by contacting the cellular sample with lectin conjugated to an oligonucleotide carrying a tag sequence that is designed to bind the left and right arms of a padlock probe. Lectin-oligonucleotide conjugates can permit detection of polysaccharides using a sequencing readout.

In some embodiments, lipids inside the cellular sample can be detected by contacting the cellular sample with a lipid-specific binding protein or an amphipathic polypeptide which are conjugated to an oligonucleotide carrying a tag sequence that is designed to bind the left and right arms of a padlock probe. Lipid-specific binding proteins and amphipathic polypeptides that are conjugated to tagged oligonucleotides can permit detection of lipids using a sequencing readout.

### Sequencing Polymerases

In any of the compositions described herein, sequencing polymerases can be used for conducting sequencing reactions. In some embodiments, the sequencing polymerase(s) is/are capable of binding and incorporating a complementary nucleotide opposite a nucleotide in a concatemer template molecule. In some embodiments, the sequencing polymerase(s) is/are capable of binding a complementary nucleotide unit of a multivalent molecule opposite a nucleotide in a concatemer template molecule. In some embodiments, the plurality of sequencing polymerases comprise recombinant mutant polymerases.

Examples of suitable polymerases for use in sequencing with nucleotides and/or multivalent molecules include but are not limited to: Klenow DNA polymerase; Thermus aquaticus DNA polymerase I (Taq polymerase); KlenTaq polymerase; Candidatus altiarchaeales archaeon; Candidatus Hadarchaeum Yellowstonense; Hadesarchaea archaeon; Euryarchaeota archaeon; Thermoplasmata archaeon; Thermococcus polymerases such as Thermococcus litoralis, bacteriophage T7 DNA polymerase; human alpha, delta and epsilon DNA polymerases; bacteriophage polymerases such as T4, RB69 and phi29 bacteriophage DNA polymerases; Pyrococcus furiosus DNA polymerase (Pfu polymerase); Bacillus subtilis DNA polymerase III; E. coli DNA polymerase III alpha and epsilon; 9 degree N polymerase; reverse transcriptases such as HIV type M or 0 reverse transcriptases; avian myeloblastosis virus reverse transcriptase; Moloney Murine Leukemia Virus (MMLV) reverse transcriptase; or telomerase. Further non-limiting examples of DNA polymerases include those from various Archaea genera, such as, Aeropyrum, Archaeoglobus, Desulfurococcus, Pyrobaculum, Pyrococcus, Pyrolobus, Pyrodictium, Staphylothermus, Stetteria, Sulfolobus, Thermococcus, and Vulcanisaeta and the like or variants thereof, including such polymerases as are known in the art such as 9 degrees N, VENT, DEEP VENT, THERMINATOR, Pfu, KOD, Pfx, Tgo and RB69 polymerases.

Sequencing cDNA Amplicons

In any of the methods described herein, the sequencing comprises conducting sequencing reactions inside a cellular sample, where the cDNA amplicons are the concatemer molecules. In some embodiments, the composition comprises a non-labeled chain-terminating nucleotide. In some embodiments, the composition comprises a primed concatemer (e.g., a concatemer annealed to a plurality of sequencing primers). In some embodiments, the composition comprises the primed concatemer contacted with at least two separate mixtures under ternary complex stabilizing conditions. In some embodiments, the at least two separate mixtures can each include a polymerase and a nucleotide. In some embodiments, the primed concatemer is contacted with nucleotide cognates for first, second and third base type base types in the template. Compositions as disclosed herein are described in U.S. Pat. Nos. 10,246,744 and 10,731,141 (where the contents of both patents are hereby incorporated by reference in their entireties).

### Padlock Probes

The present disclosure provides a padlock probe for use in any of the methods as described herein. A padlock probe may be designed to selectively detect a target molecule. For example, the padlock probe may be designed to selectively detect a target nucleic acid or polypeptide. The target nucleic acid may be a target DNA or a target RNA. By binding the padlock probe to the target nucleic acid or protein/polypeptide, one can learn information about one or more properties of the target nucleic acid or protein/polypeptide. For example, a location of the target nucleic acid or protein/polypeptide can be ascertained. The padlock probe may comprise one or more batch-specific primer-binding sites, which are specific to a primer. The primer may be spatially localized, and the padlock probe may therefore not require a barcode sequence. The location of the target can give information about the sample, e.g., cellular origin. In such instances, it may not be necessary for the padlock probe to carry a barcode. However, in other non-limiting instances, the padlock probe may carry a barcode.

The present disclosure provides a padlock probe that is specific to a target RNA. The RNA-specific padlock probe may selectively hybridize to a cDNA that corresponds to the target RNA. The RNA-specific probe may lack a barcode that uniquely identifies the cDNA. In such instances, a spatial information may be obtained with respect to the RNA-specific probe. The RNA-specific probe may carry a barcode that uniquely identifies the cDNA. The RNA-specific padlock probes may also carry a sequencing primer binding site. The sequencing primer binding site may be a batch-specific primer binding site—in other words, a primer may bind to the primer binding site that is unique to the probe. One or more padlock probes may be used. Each probe may have a unique batch-specific primer binding site. The use of a batch-specific primer binding site may enable the spatial localization of the padlock probe, thereby obviating the need for a barcode sequence on the padlock probe.

In any of the compositions described herein, a plurality of cDNA inside the cellular sample can be amplified to generate cDNA amplicons (e.g., concatemers). In some embodiments, the plurality of cDNA molecules can be amplified by conducting a padlock probe circularization and rolling circle amplification workflow. In some embodiments, the compositions comprise contacting the plurality of cDNA molecules with a plurality of padlock probes, including a first plurality of target-specific padlock probes that hybridize with first target cDNA molecules, and a second plurality of target-specific padlock probes that hybridize with second target cDNA molecules.

In some embodiments, the padlock probes comprise single-stranded oligonucleotides. In some embodiments, the padlock probes comprise DNA, RNA, DNA and RNA. In some embodiments, the padlock probes comprise canonical nucleotides and/or nucleotide analogs. In some embodiments, the padlock probes are modified to confer resistance to nuclease degradation (e.g., ribonuclease degradation). For example, the padlock probes comprise at least one phosphorothioate diester bond at their 5′ ends which can render the padlock probes resistant to nuclease degradation. In some embodiments, the padlock probes comprise 2-5 or more consecutive phosphorothioate diester bonds at their 5′ ends. In some embodiments, the padlock probes comprise at least one ribonucleotide and/or at least one 2′-O-methyl, 2′-O-methoxyethyl (MOE), 2′ fluoro-base nucleotide. In some embodiments, the padlock probes comprise phosphorylated 3′ ends. In some embodiments, the padlock probes comprise at least one locked nucleic acid (LNA) base. In some embodiments, the padlock probes comprise a phosphorylated 5′ end (e.g., using a polynucleotide kinase).

In some embodiments, the plurality of padlock probes can include a first plurality of target-specific padlock probes. In some embodiments, the first plurality of target-specific padlock probes can hybridize with the first target cDNA molecules. In some embodiments, the plurality of padlock probes can include a second plurality of target-specific padlock probes. In some embodiments, the second plurality of target-specific padlock probes can hybridize with the second target cDNA molecules.

In some embodiments, individual padlock probes comprise first and second terminal regions that hybridize to portions of cDNA molecules to form a plurality of cDNA-padlock probe complexes, wherein individual complexes have the first and second terminal probe regions hybridized to proximal regions of a cDNA molecule to form a nick or gap between the first and second terminal probe ends. In some embodiments, the first terminal region of an individual padlock probe has a first target-specific sequence that selectively hybridizes to a first region of a target cDNA molecule, and the second terminal region of the individual padlock probe has a second target-specific sequence that selectively hybridizes to a second region of the same target cDNA molecule, where a nick or gap is formed between the hybridized first and second terminal regions, thereby circularizing the padlock probe. In some embodiments, the first terminal region of an individual padlock probe can have a first target-specific sequence that can selectively hybridize to a first region of a target cDNA molecule. In some embodiments, the second terminal region of an individual padlock probe can have a second target-specific sequence that can selectively hybridize to a second region of a target cDNA molecule. In some embodiments, a nick or gap can be formed between the hybridized first and second terminal regions. In some embodiments, the hybridization of the first terminal region and the second terminal region can circularize the padlock probe.

In some embodiments, individual padlock probes in a set of padlock probes (e.g., a plurality of padlock probes) comprise first and second terminal regions that hybridize to the same target regions of the target cDNA molecules to form a plurality of cDNA-padlock probe complexes having the same cDNA sequence.

In some embodiments, a set of padlock probes (e.g., a plurality of padlock probes) comprise at least two sub-sets of padlock probes. In some embodiments, individual padlock probes in a first sub-set of padlock probes comprise first and second terminal regions that hybridize to the same target regions (e.g., a first target region) of the target cDNA molecules to form a first plurality of cDNA-padlock probe complexes having the same cDNA sequence. In some embodiments, individual padlock probes in a second sub-set of padlock probes comprise first and second terminal regions that hybridize to the same target regions (e.g., a second target region) of the target cDNA molecules to form a second plurality of cDNA-padlock probe complexes having the same cDNA sequence. In some embodiments, the first and second sub-sets of padlock probes hybridize to different target regions of the same target cDNA molecules. In some embodiments, the first and second sub-sets of padlock probes hybridize to different target regions of different target cDNA molecules. In some embodiments, the set of padlock probes comprise 2-10 sub-sets of padlock probes, or 10-25 sub-sets of padlock probes, or 25-50 sub-sets of padlock probes, or up to 100 sub-sets of padlock probes. In some embodiments, the set of padlock probes comprise at least 100 sub-sets of padlock probes, at least 500 sub-sets of padlock probes, at least 1000 sub-sets of padlock probes, at least 10,000 sub-sets of padlock probes, or more sub-sets of padlock probes.

In some embodiments, the nicks can be enzymatically ligated to generate covalently closed circular padlock probes. In some embodiments, the ligase enzyme can discriminate between matched and mis-matched hybridized ends to ensure target-specific hybridization. In some embodiments, the ligation reaction comprises use of a ligase enzyme, including a T3, T4, T7 or Taq DNA ligase enzyme.

In some embodiments, the size of the gap between the hybridized first and second terminal regions is 1-25 bases. The 3′OH end of hybridized padlock probe can serve as an initiation site for a polymerase-catalyzed fill-in reaction (e.g., gap fill-in reaction) using the target cDNA molecule as a template. After the fill-in reaction, the remaining nick can be enzymatically ligated to generate covalently closed circular padlock probes.

In some embodiments, the gap-filling reaction comprises contacting the circularized padlock probe with a DNA polymerase and a plurality of nucleotides. In some embodiments, the DNA polymerase comprises E. coli DNA polymerase I, Klenow fragment of E. coli DNA polymerase I, T7 DNA polymerase, or T4 DNA polymerase. In some embodiments, the ligase enzyme can discriminate between matched and mis-matched hybridized ends to ensure target-specific hybridization. In some embodiments, the ligation reaction comprises use of a ligase enzyme, including a T3, T4, T7 or Taq DNA ligase enzyme.

In some embodiments, the padlock probes comprise at least one universal adaptor sequence including a sample barcode sequence, an amplification primer binding site, a sequencing primer binding site, a compaction oligonucleotide binding site and/or a surface capture primer binding site. In some embodiments, the padlock probes comprise at least one unique identification sequence (e.g., unique molecular index (UMI). In some embodiments, the padlock probes comprise at least one restriction enzyme recognition sequence.

### Nucleotides and Chain-Terminating Nucleotides

The present disclosure provides methods for detecting the sequence of a nucleic acid in a biological sample. The nucleic acid may be, e.g., DNA or RNA. The biological sample may be a cellular sample.

The present disclosure provides compositions for detecting in situ at least two different target RNA molecules in a cellular sample, which can include conducting sequencing reactions inside the cellular sample, where the cDNA amplicons can be the concatemer molecules.

In any of the compositions described herein, any of the compositions described herein can comprise at least one nucleotide. The nucleotides comprise a base, sugar and at least one phosphate group. In some embodiments, at least one nucleotide in the plurality comprises an aromatic base, a five carbon sugar (e.g., ribose or deoxyribose), and one or more phosphate groups (e.g., 1-10 phosphate groups). The plurality of nucleotides can comprise at least one type of nucleotide selected from a group consisting of dATP, dGTP, dCTP, dTTP and dUTP. The plurality of nucleotides can comprise at a mixture of any combination of two or more types of nucleotides selected from a group consisting of dATP, dGTP, dCTP, dTTP and/or dUTP. In some embodiments, at least one nucleotide in the plurality is not a nucleotide analog. In some embodiments, at least one nucleotide in the plurality comprises a nucleotide analog.

In some embodiments, in any of the compositions described herein, at least one nucleotide in the plurality of nucleotides comprise a chain of one, two or three phosphorus atoms where the chain is typically attached to the 5′ carbon of the sugar moiety via an ester or phosphoramide linkage. In some embodiments, at least one nucleotide in the plurality is an analog having a phosphorus chain in which the phosphorus atoms are linked together with intervening O, S, NH, methylene or ethylene. In some embodiments, the phosphorus atoms in the chain include substituted side groups including 0, S or BH3. In some embodiments, the chain includes phosphate groups substituted with analogs including phosphoramidate, phosphorothioate, phosphordithioate, and O-methyl phosphoramidite groups.

In some embodiments, in any of the compositions described herein, at least one nucleotide in the plurality of nucleotides comprises a terminator nucleotide analog having a chain terminating moiety (e.g., blocking moiety) at the sugar 2′ position, at the sugar 3′ position, or at the sugar 2′ and 3′ position. In some embodiments, the chain terminating moiety can inhibit polymerase-catalyzed incorporation of a subsequent nucleotide unit or free nucleotide in a nascent strand during a primer extension reaction. In some embodiments, the chain terminating moiety is attached to the 3′ sugar hydroxyl position where the sugar comprises a ribose or deoxyribose sugar moiety. In some embodiments, the chain terminating moiety is removable/cleavable from the 3′ sugar hydroxyl position to generate a nucleotide having a 3′OH sugar group which is extendible with a subsequent nucleotide in a polymerase-catalyzed nucleotide incorporation reaction. In some embodiments, the chain terminating moiety comprises an alkyl group, alkenyl group, alkynyl group, allyl group, aryl group, benzyl group, azide group, amine group, amide group, keto group, isocyanate group, phosphate group, thiol group, disulfide group, carbonate group, urea group, or silyl group. In some embodiments, the chain terminating moiety is cleavable/removable from the nucleotide, for example by reacting the chain terminating moiety with a chemical agent, pH change, light or heat. In some embodiments, the chain terminating moieties alkyl, alkenyl, alkynyl and allyl are cleavable with tetrakis(triphenylphosphine)palladium(0) (Pd(PPh3)4) with piperidine, or with 2,3-Dichloro-5,6-dicyano-1,4-benzo-quinone (DDQ). In some embodiments, the chain terminating moieties aryl and benzyl are cleavable with H2 Pd/C. In some embodiments, the chain terminating moieties amine, amide, keto, isocyanate, phosphate, thiol, disulfide are cleavable with phosphine or with a thiol group including beta-mercaptoethanol or dithiothritol (DTT). In some embodiments, the chain terminating moiety carbonate is cleavable with potassium carbonate (K₂CO₃) in MeOH, with triethylamine in pyridine, or with Zn in acetic acid (AcOH). In some embodiments, the chain terminating moieties urea and silyl are cleavable with tetrabutylammonium fluoride, pyridine-HF, with ammonium fluoride, or with triethylamine trihydrofluoride.

In some embodiments, in any of the compositions described herein, at least one nucleotide in the plurality of nucleotides comprises a terminator nucleotide analog having a chain terminating moiety (e.g., blocking moiety) at the sugar 2′ position, at the sugar 3′ position, or at the sugar 2′ and 3′ position. In some embodiments, the chain terminating moiety comprises an azide, azido or azidomethyl group. In some embodiments, the chain terminating moiety comprises a 3′-O-azido or 3′-O-azidomethyl group. In some embodiments, the chain terminating moieties azide, azido and azidomethyl group are cleavable/removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (BS-TPP) or Tri(hydroxyproyl)phosphine (THPP). In some embodiments, the cleaving agent comprises 4-dimethylaminopyridine (4-DMAP).

In some embodiments, in any of the compositions described herein, the nucleotide comprises a chain terminating moiety which is selected from a group consisting of 3′-deoxy nucleotides, 2′,3′-dideoxynucleotides, 3′-methyl, 3′-azido, 3′-azidomethyl, 3′-O-azidoalkyl, 3′-O-ethynyl, 3′-O-aminoalkyl, 3′-O-fluoroalkyl, 3′-fluoromethyl, 3′-difluoromethyl, 3′-trifluoromethyl, 3′-sulfonyl, 3′-malonyl, 3′-amino, 3′-O-amino, 3′-sulfhydral, 3′-aminomethyl, 3′-ethyl, 3′butyl, 3′-tert butyl, 3′-Fluorenylmethyloxycarbonyl, 3′ tert-Butyloxycarbonyl, 3′-O-alkyl hydroxylamino group, 3′-phosphorothioate, and 3-O-benzyl, or derivatives thereof.

In some embodiments, in any of the compositions described herein, the plurality of nucleotides comprises a plurality of nucleotides labeled with detectable reporter moiety. The detectable reporter moiety comprises a fluorophore. In some embodiments, the fluorophore is attached to the nucleotide base. In some embodiments, the fluorophore is attached to the nucleotide base with a linker which is cleavable/removable from the base. In some embodiments, at least one of the nucleotides in the plurality is not labeled with a detectable reporter moiety. In some embodiments, a particular detectable reporter moiety (e.g., fluorophore) that is attached to the nucleotide can correspond to the nucleotide base (e.g., dATP, dGTP, dCTP, dTTP or dUTP) to permit detection and identification of the nucleotide base.

In some embodiments, in any of the compositions described herein, the cleavable linker on the nucleotide base comprises a cleavable moiety comprising an alkyl group, alkenyl group, alkynyl group, allyl group, aryl group, benzyl group, azide group, amine group, amide group, keto group, isocyanate group, phosphate group, thiol group, disulfide group, carbonate group, urea group, or silyl group. In some embodiments, the cleavable linker on the base is cleavable/removable from the base by reacting the cleavable moiety with a chemical agent, pH change, light or heat. In some embodiments, the cleavable moieties alkyl, alkenyl, alkynyl and allyl are cleavable with tetrakis(triphenylphosphine)palladium(0) (Pd(PPh3)4) with piperidine, or with 2,3-Dichloro-5,6-dicyano-1,4-benzo-quinone (DDQ). In some embodiments, the cleavable moieties aryl and benzyl are cleavable with H2 Pd/C. In some embodiments, the cleavable moieties amine, amide, keto, isocyanate, phosphate, thiol, disulfide are cleavable with phosphine or with a thiol group including beta-mercaptoethanol or dithiothritol (DTT). In some embodiments, the cleavable moiety carbonate is cleavable with potassium carbonate (K₂CO₃) in MeOH, with triethylamine in pyridine, or with Zn in acetic acid (AcOH). In some embodiments, the cleavable moieties urea and silyl are cleavable with tetrabutylammonium fluoride, pyridine-HF, with ammonium fluoride, or with triethylamine trihydrofluoride.

In some embodiments, in any of the compositions described herein, the cleavable linker on the nucleotide base comprises cleavable moiety including an azide, azido or azidomethyl group. In some embodiments, the cleavable moieties azide, azido and azidomethyl group are cleavable/removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (BS-TPP) or Tri(hydroxyproyl)phosphine (THPP). In some embodiments, the cleaving agent comprises 4-dimethylaminopyridine (4-DMAP).

In some embodiments, in any of the compositions described herein, the chain terminating moiety (e.g., at the sugar 2′ and/or sugar 3′ position) and the cleavable linker on the nucleotide base have the same or different cleavable moieties. In some embodiments, the chain terminating moiety (e.g., at the sugar 2′ and/or sugar 3′ position) and the detectable reporter moiety linked to the base are chemically cleavable/removable with the same chemical agent. In some embodiments, the chain terminating moiety (e.g., at the sugar 2′ and/or sugar 3′ position) and the detectable reporter moiety linked to the base are chemically cleavable/removable with different chemical agents.

### Multivalent Molecules

Compositions as described herein may employ at least one multivalent molecule. A multivalent binding complex may be formed between one or more polymer-nucleotide conjugates, one or more polymerases, one or more plurality of primed target nucleic acid molecules, or any combination thereof. The multivalent binding complex may be stable, and may be used in any sequencing method as described herein. For example, the multivalent binding complex may allow a detection or base-calling step in a sequencing cycle to be separated from the nucleotide incorporation step.

Compositions as described herein may comprise least one multivalent molecule which comprises a plurality of nucleotide arms attached to a core and having any configuration including a starburst, helter skelter, or bottle brush configuration (e.g., FIG. 7). The multivalent molecule comprises: (1) a core; and (2) a plurality of nucleotide arms which comprise (i) a core attachment moiety, (ii) a spacer comprising a PEG moiety, (iii) a linker, and (iv) a nucleotide unit, wherein the core is attached to the plurality of nucleotide arms, wherein the spacer is attached to the linker, wherein the linker is attached to the nucleotide unit. In some embodiments, the multivalent molecule can comprise a core. In some embodiments, the multivalent molecule can comprise one or more linker moieties (e.g., FIG. 26). In some embodiments, the multivalent molecule can comprise a plurality of nucleotide arms. In some embodiments, a nucleotide arm from the plurality of nucleotide arms can comprise a core In some embodiments, a nucleotide arm can comprise a spacer. In some embodiments, the spacer can comprise a PEG moiety. In some embodiments, a nucleotide arm can comprise a linker. In some embodiments, a nucleotide arm can comprise a nucleotide unit. In some embodiments, the spacer can be attached to the linker. In some embodiments, the linker can be attached to the nucleotide unit. In some embodiments, the core can be attached to the plurality of nucleotide arms. In some embodiments, the nucleotide unit can comprise a base, sugar and at least one phosphate group, and the linker can be attached to the nucleotide unit through the base. In some embodiments, the linker can comprise an aliphatic chain or an oligo ethylene glycol chain where both linker chains can have 2-6 subunits. In some embodiments, the linker can also include an aromatic moiety. A non-limiting example nucleotide arm is shown in FIG. 11. Non-limiting example multivalent molecules are shown in FIGS. 7-10. A non-limiting example spacer is shown in FIG. 12 (top) and non-limiting example linkers are shown in FIG. 12 (bottom) and FIG. 13. Non-limiting example nucleotides attached to a linker are shown in FIGS. 14-16. A non-limiting example biotinylated nucleotide arm is shown in FIG. 17.

In some embodiments, a multivalent molecule comprises a core attached to multiple nucleotide arms, and wherein the multiple nucleotide arms have the same type of nucleotide unit which is selected from a group consisting of dATP, dGTP, dCTP, dTTP and dUTP.

In some embodiments, a multivalent molecule comprises a core attached to multiple nucleotide arms, where each arm includes a nucleotide unit. The nucleotide unit comprises an aromatic base, a five carbon sugar (e.g., ribose or deoxyribose), and one or more phosphate groups (e.g., 1-10 phosphate groups). The plurality of multivalent molecules can comprise one type multivalent molecule having one type of nucleotide unit selected from a group consisting of dATP, dGTP, dCTP, dTTP and dUTP. The plurality of multivalent molecules can comprise at a mixture of any combination of two or more types of multivalent molecules, where individual multivalent molecules in the mixture comprise nucleotide units selected from a group consisting of dATP, dGTP, dCTP, dTTP and/or dUTP.

In some embodiments, the nucleotide unit comprises a chain of one, two or three phosphorus atoms where the chain is typically attached to the 5′ carbon of the sugar moiety via an ester or phosphoramide linkage. In some embodiments, at least one nucleotide unit is a nucleotide analog having a phosphorus chain in which the phosphorus atoms are linked together with intervening O, S, NH, methylene or ethylene. In some embodiments, the phosphorus atoms in the chain include substituted side groups including 0, S or BH3. In some embodiments, the chain includes phosphate groups substituted with analogs including phosphoramidate, phosphorothioate, phosphordithioate, and O-methyl phosphoramidite groups.

In some embodiments, the multivalent molecule comprises a core attached to multiple nucleotide arms, and wherein individual nucleotide arms comprise a nucleotide unit which is a nucleotide analog having a chain terminating moiety (e.g., blocking moiety) at the sugar 2′ position, at the sugar 3′ position, or at the sugar 2′ and 3′ position. In some embodiments, the nucleotide unit comprises a chain terminating moiety (e.g., blocking moiety) at the sugar 2′ position, at the sugar 3′ position, or at the sugar 2′ and 3′ position. In some embodiments, the chain terminating moiety can inhibit polymerase-catalyzed incorporation of a subsequent nucleotide unit or free nucleotide in a nascent strand during a primer extension reaction. In some embodiments, the chain terminating moiety is attached to the 3′ sugar hydroxyl position where the sugar comprises a ribose or deoxyribose sugar moiety. In some embodiments, the chain terminating moiety is removable/cleavable from the 3′ sugar hydroxyl position to generate a nucleotide having a 3′OH sugar group which is extendible with a subsequent nucleotide in a polymerase-catalyzed nucleotide incorporation reaction. In some embodiments, the chain terminating moiety comprises an alkyl group, alkenyl group, alkynyl group, allyl group, aryl group, benzyl group, azide group, amine group, amide group, keto group, isocyanate group, phosphate group, thiol group, disulfide group, carbonate group, urea group, or silyl group. In some embodiments, the chain terminating moiety is cleavable/removable from the nucleotide unit, for example by reacting the chain terminating moiety with a chemical agent, pH change, light or heat. In some embodiments, the chain terminating moieties alkyl, alkenyl, alkynyl and allyl are cleavable with tetrakis(triphenylphosphine)palladium(0) (Pd(PPh3)4) with piperidine, or with 2,3-Dichloro-5,6-dicyano-1,4-benzo-quinone (DDQ). In some embodiments, the chain terminating moieties aryl and benzyl are cleavable with H2 Pd/C. In some embodiments, the chain terminating moieties amine, amide, keto, isocyanate, phosphate, thiol, disulfide are cleavable with phosphine or with a thiol group including beta-mercaptoethanol or dithiothritol (DTT). In some embodiments, the chain terminating moiety carbonate is cleavable with potassium carbonate (K₂CO₃) in MeOH, with triethylamine in pyridine, or with Zn in acetic acid (AcOH). In some embodiments, the chain terminating moieties urea and silyl are cleavable with tetrabutylammonium fluoride, pyridine-HF, with ammonium fluoride, or with triethylamine trihydrofluoride.

In some embodiments, the nucleotide unit comprises a chain terminating moiety (e.g., blocking moiety) at the sugar 2′ position, at the sugar 3′ position, or at the sugar 2′ and 3′ position. In some embodiments, the chain terminating moiety comprises an azide, azido or azidomethyl group. In some embodiments, the chain terminating moiety comprises a 3′-O-azido or 3′-O-azidomethyl group. In some embodiments, the chain terminating moieties azide, azido and azidomethyl group are cleavable/removable with a phosphine compound. In some embodiments, the phosphine compound comprises a derivatized tri-alkyl phosphine moiety or a derivatized tri-aryl phosphine moiety. In some embodiments, the phosphine compound comprises Tris(2-carboxyethyl)phosphine (TCEP) or bis-sulfo triphenyl phosphine (B S-TPP) or Tri(hydroxyproyl)phosphine (THPP). In some embodiments, the cleaving agent comprises 4-dimethylaminopyridine (4-DMAP).

In some embodiments, the nucleotide unit comprising a chain terminating moiety which is selected from a group consisting of 3′-deoxy nucleotides, 2′,3′-dideoxynucleotides, 3′-methyl, 3′-azido, 3′-azidomethyl, 3′-O-azidoalkyl, 3′-O-ethynyl, 3′-O-aminoalkyl, 3′-O-fluoroalkyl, 3′-fluoromethyl, 3′-difluoromethyl, 3′-trifluoromethyl, 3′-sulfonyl, 3′-malonyl, 3′-amino, 3′-O-amino, 3′-sulfhydral, 3′-aminomethyl, 3′-ethyl, 3′butyl, 3′-tert butyl, 3′-Fluorenylmethyloxycarbonyl, 3′ tert-Butyloxycarbonyl, 3′-O-alkyl hydroxylamino group, 3′-phosphorothioate, and 3-O-benzyl, or derivatives thereof.

In some embodiments, the multivalent molecule comprises a core attached to multiple nucleotide arms, wherein the nucleotide arms comprise a spacer, linker and nucleotide unit, and wherein the core, linker and/or nucleotide unit is labeled with detectable reporter moiety. In some embodiments, the detectable reporter moiety comprises a fluorophore. In some embodiments, a particular detectable reporter moiety (e.g., fluorophore) that is attached to the multivalent molecule can correspond to the base (e.g., dATP, dGTP, dCTP, dTTP or dUTP) of the nucleotide unit to permit detection and identification of the nucleotide base.

In some embodiments, at least one nucleotide arm of a multivalent molecule has a nucleotide unit that is attached to a detectable reporter moiety. In some embodiments, the detectable reporter moiety is attached to the nucleotide base. In some embodiments, the detectable reporter moiety comprises a fluorophore. In some embodiments, a particular detectable reporter moiety (e.g., fluorophore) that is attached to the multivalent molecule can correspond to the base (e.g., dATP, dGTP, dCTP, dTTP or dUTP) of the nucleotide unit to permit detection and identification of the nucleotide base.

In some embodiments, the core of a multivalent molecule comprises an avidin-like or streptavidin-like moiety and the core attachment moiety comprises biotin. In some embodiments, the core of a multivalent molecule can comprise an avidin-like moiety. In some embodiments, the core of a multivalent molecule can comprise a streptavidin-like moiety. In some embodiments, the core of a multivalent molecule can comprise the core attachment moiety can comprise biotin. In some embodiments, the core comprises an streptavidin-type or avidin-type moiety which includes an avidin protein, as well as any derivatives, analogs and other non-native forms of avidin that can bind to at least one biotin moiety. Other forms of avidin moieties include native and recombinant avidin and streptavidin as well as derivatized molecules, e.g. non-glycosylated avidin and truncated streptavidins. For example, avidin moiety includes de-glycosylated forms of avidin, bacterial streptavidin produced by Streptomyces (e.g., Streptomyces avidinii), as well as derivatized forms, for example, N-acyl avidins, e.g., N-acetyl, N-phthalyl and N-succinyl avidin, and the commercially-available products EXTRAVIDIN, CAPTAVIDIN, NEUTRAVIDIN and NEUTRALITE AVIDIN.

In some embodiments, any of the compositions for sequencing nucleic acid molecules described herein can comprise a binding complex, where the binding complex comprises (i) a polymerase, a nucleic acid concatemer molecule duplexed with a primer, and a nucleotide, or the binding complex comprises (ii) a polymerase, a nucleic acid concatemer molecule duplexed with a primer, and a nucleotide unit of a multivalent molecule. In some embodiments, the binding complex can comprise (i) a polymerase, a nucleic acid concatemer molecule duplexed with a primer, and a nucleotide. In some embodiments, the binding complex can comprise (ii) a polymerase, a nucleic acid concatemer molecule duplexed with a primer, and a nucleotide unit of a multivalent molecule. In some embodiments, the binding complex has a persistence time of greater than about 0.1, 0.2, 0.3, 0.4, 0.5, 0.6, 0.7, 0.8, 0.9 or 1 second. The binding complex has a persistence time of greater than about 0.1-0.25 seconds, or about 0.25-0.5 seconds, or about 0.5-0.75 seconds, or about 0.75-1 second, or about 1-2 seconds, or about 2-3 seconds, or about 3-4 second, or about 4-5 seconds, and/or wherein the method is or may be carried out at a temperature of at or above 15° C., at or above 20° C., at or above 25° C., at or above 35° C., at or above 37° C., at or above 42° C. at or above 55° C. at or above 60° C., or at or above 72° C., or at or above 80° C., or within a range defined by any of the foregoing, or a combination of the binding complex can have a persistence time of greater that about 0.1-0.25 seconds, or about 0.25-0.5 seconds, or about 0.5-0.75 seconds, or about 0.75-1 second, or about 1-2 seconds, or about 2-3 seconds, or about 3-4 second, or about 4-5 seconds and wherein the methodcan beor may be carried out at a temperature of at or above 15° C., at or above 20° C., at or above 25° C., at or above 35° C., at or above 37° C., at or above 42° C. at or above 55° C. at or above 60° C., or at or above 72° C., or at or above 80° C., or within a range defined by any of the foregoing . . . . The binding complex (e.g., ternary complex) remains stable until subjected to a condition that causes dissociation of interactions between any of the polymerase, template molecule, primer and/or the nucleotide unit or the nucleotide. For example, a dissociating condition comprises contacting the binding complex with any one or any combination of a detergent, EDTA and/or water. In some embodiments, the present disclosure provides said method wherein the binding complex is deposited on, attached to, or hybridized to, a surface showing a contrast to noise ratio in the detecting step of greater than 20. In some embodiments, the present disclosure provides said method wherein the contacting is performed under a condition that stabilizes the binding complex when the nucleotide or nucleotide unit is complementary to a next base of the template nucleic acid, and destabilizes the binding complex when the nucleotide or nucleotide unit is not complementary to the next base of the template nucleic acid.

### Avidity Complexes

Compositions as disclosed herein may comprise one or more avidity complexes. Compositions as disclosed herein may comprise one or more of a first nucleic acid primer, a first sensing polymerase and a first multivalent molecule. The first nucleic acid primer, the first sequencing polymerase, the first multivalent molecule may be bound to a first portion of a concatemer template molecule thereby forming a first binding complex, wherein a first nucleotide unit of the first multivalent molecule can bind to the first sequencing polymerase. Compositions as disclosed herein may comprises a second nucleic acid primer and a second sequencing polymerase. The second nucleic acid primer, second polymerase, and first multivalent molecule may be bound to a second portion of the same concatemer template molecule thereby forming a second binding complex, wherein a second nucleotide unit of the first multivalent molecule can bind to the second sequencing polymerase, wherein the first and second binding complexes which can include the same multivalent molecule forms an avidity complex. In some embodiments, the first multivalent molecule can be detectably-labeled multivalent molecule. In some embodiments, a first nucleotide unit of the first multivalent molecule can bind to the first sequencing polymerase . . . . In some embodiments, a second nucleotide unit of the first multivalent molecule can bind to the second sequencing polymerase, wherein the first and second binding complexes which can include the same multivalent molecule forms an avidity complex. In some embodiments, the first multivalent molecule can comprise a core attached to a plurality of nucleotide arms. In some embodiments, each nucleotide arm can be attached to a nucleotide unit. In some embodiments, the binding can be conducted under a condition suitable to inhibit polymerase-catalyzed incorporation of the first nucleotide unit and the second nucleotide unity in the first binding complex and the second binding complex. In some embodiments, the first sequencing polymerase can comprise any wild type or mutant polymerase described herein. In some embodiments, the second sequencing polymerase can comprise any wild type or mutant polymerase described herein. The concatemer template molecule can comprise tandem repeat sequences of a sequence of interest and at least one universal sequencing primer binding site. The first and second nucleic acid primers can bind to a sequencing primer binding site along the concatemer template molecule. Multivalent molecules are shown in FIGS. 7-10. The composition may comprise a plurality of sequencing polymerases and a plurality of nucleic acid primers with different portions of a concatemer nucleic acid concatemer molecule to form at least first and second complexed polymerases on the same concatemer template molecule. In some embodiments, at least a first nucleotide unit of the single multivalent molecule can be bound to the first complexed polymerase. In some embodiments, the first complexed polymerase can include a first primer hybridized to a first portion of the concatemer template molecule thereby forming a first binding complex (e.g., first ternary complex). In some embodiments, at least a second nucleotide unit of the single multivalent molecule can be bound to the second complexed polymerase. In some embodiments, the second complexed polymerase can include a second primer hybridized to a second portion of the concatemer template molecule thereby forming a second binding complex (e.g., second ternary complex). In some embodiments, the first and second binding complexes which can be bound to the same multivalent molecule forms an avidity complex. In some embodiments, the composition may comprise a plurality of concatemer molecules, a plurality of sequencing polymerases and a plurality of soluble sequencing primers. In some embodiments, the plurality of sequencing polymerases can comprise a plurality of first sequencing polymerases. In some embodiments, the plurality of sequencing polymerases can comprise a plurality of second sequencing polymerases. In some embodiments, each complexed sequencing polymerase can comprise a sequencing polymerase bounds to a nucleic acid duplex. In some embodiments, the nucleic acid duplex can comprise a concatemer molecule hybridized to a soluble sequencing primer. In some embodiments, the plurality of nucleotides can comprise at least one nucleotide analog. In some embodiments, the at least one nucleotide analog can be labeled with a fluorophore. In some embodiments, the at least one nucleotide analog can have a removable chain terminating moiety at the sugar 3′position. In some embodiments, the at least one nucleotide analog can be labeled with a fluorophore and can have a removable chain terminating moiety at the sugar 3′ position. In some embodiments, the method can further comprise incorporating the at least nucleotide analog into the 3′ end of the plurality of soluble sequencing primers, thereby generating a plurality of nascent extended sequencing primers. In some embodiments, the method can further comprise detecting the incorporated at least one nucleotide analog. In some embodiments, the method can further comprise identifying the nucleobase of the incorporated at least one nucleotide analog.

Compositions as disclosed herein may comprise a plurality of concatemer molecules and a plurality of sequencing polymerases. In some embodiments, the plurality of sequencing polymerases can comprise a plurality of first sequencing polymerases. In some embodiments, the plurality of sequencing polymerases can comprise a plurality of second sequencing polymerases. In some embodiments, each complexed sequencing polymerase can comprise a sequencing polymerase bounds to a nucleic acid duplex. In some embodiments, the nucleic acid duplex can comprise a concatemer molecule hybridized to a soluble sequencing primer. In some embodiments, individual multivalent molecules in a plurality of detectably labeled multivalent molecules can comprise a core attached to a nucleotide unit. In some embodiments, a second complexed sequencing polymerase can comprise a second sequencing polymerase bound to the nucleic acid duplex. In some embodiments, the concatemer template molecule comprises tandem repeat sequences of a sequence of interest and at least one universal sequencing primer binding site. The plurality of nucleic acid primers can bind to a sequencing primer binding site along the concatemer template molecule. Non-limiting example multivalent molecules are shown in FIGS. 7-10.

### Systems

### Computer Systems

The present disclosure provides computer systems that are programmed to implement methods of the disclosure. Disclosed herein, in some embodiments, are computer-implemented methods of performing a nucleic acid processing and/or analysis utilizing the computer systems disclosed herein. FIG. 26 shows a computer system 2601 that is programmed or otherwise configured to implement methods and systems as disclosed herein. The computer system 2601 can regulate various aspects of the present disclosure. The computer system 2601 can be an electronic device of a user or a computer system that is remotely located with respect to the electronic device. The electronic device can be a mobile electronic device.

The computer system 2601 includes a central processing unit (CPU, also “processor” and “computer processor” herein) 2605, which can be a single core or multi core processor, or a plurality of processors for parallel processing. The computer system 2601 also includes memory or memory location 2610 (e.g., random-access memory, read-only memory, flash memory), electronic storage unit 2615 (e.g., hard disk), communication interface 2620 (e.g., network adapter) for communicating with one or more other systems, and peripheral devices 2625, such as cache, other memory, data storage and/or electronic display adapters. The memory 2610, storage unit 2615, interface 2620 and peripheral devices 2625 are in communication with the CPU 2605 through a communication bus (solid lines), such as a motherboard. The storage unit 2615 can be a data storage unit (or data repository) for storing data. The computer system 2601 can be operatively coupled to a computer network (“network”) 2630 with the aid of the communication interface 2620. The network 2630 can be the Internet, an internet and/or extranet, or an intranet and/or extranet that is in communication with the Internet. The network 2630 In some embodiments is a telecommunication and/or data network. The network 2630 can include one or more computer servers, which can enable distributed computing, such as cloud computing. The network 2630, In some embodiments with the aid of the computer system 2601, can implement a peer-to-peer network, which may enable devices coupled to the computer system 2601 to behave as a client or a server.

The CPU 2605 can execute a sequence of machine-readable instructions, which can be embodied in a program or software. The instructions may be stored in a memory location, such as the memory 2610. The instructions can be directed to the CPU 2605, which can subsequently program or otherwise configure the CPU 2605 to implement methods of the present disclosure.

Examples of operations performed by the CPU 2605 can include fetch, decode, execute, and writeback.

The CPU 2605 can be part of a circuit, such as an integrated circuit. One or more other components of the system 2601 can be included in the circuit. In some embodiments, the circuit is an application specific integrated circuit (ASIC).

The storage unit 2615 can store files, such as drivers, libraries and saved programs. The storage unit 2615 can store user data, e.g., user preferences and user programs. The computer system 2601 In some embodiments can include one or more additional data storage units that are external to the computer system 2601, such as located on a remote server that is in communication with the computer system 2601 through an intranet or the Internet.

The computer system 2601 can communicate with one or more remote computer systems through the network 2630. For instance, the computer system 2601 can communicate with a remote computer system of a user. Examples of remote computer systems include personal computers (e.g., portable PC), slate or tablet PC's (e.g., Apple® iPad, Samsung@Galaxy Tab), telephones, Smart phones (e.g., Apple® iPhone, Android-enabled device, Blackberry®), or personal digital assistants. The user can access the computer system 2601 via the network 2630.

Methods as described herein can be implemented by way of machine (e.g., computer processor) executable code stored on an electronic storage location of the computer system 2601, such as, for example, on the memory 2610 or electronic storage unit 2615. The machine executable or machine readable code can be provided in the form of software. During use, the code can be executed by the processor 2605. In some embodiments, the code can be retrieved from the storage unit 2615 and stored on the memory 2610 for ready access by the processor 2605. In some situations, the electronic storage unit 2615 can be precluded, and machine-executable instructions are stored on memory 2610.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 2601, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 2601 can include or be in communication with an electronic display 2635 that comprises a user interface (UI) 2640 for providing, for example, information on a property of a nucleic acid, e.g., sequence. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 2605. The algorithm can, for example, perform or analyze methods of nucleic acid processing as disclosed herein.

### Optical Systems

The present disclosure provides optical systems for detecting a sequence of a nucleic acid in a biological sample. Systems as disclosed herein may be used to conduct in situ sequencing of the nucleic acid. The biological sample may be, e.g., a cellular sample. The nucleic acid may be DNA or RNA. Optical systems as disclosed herein may be used for imaging a nucleic acid, e.g., for high performance fluorescence imaging. Optical systems as disclosed herein may comprise an illumination and imaging module. The illumination and imaging module may be used for multi-channel fluorescence imaging. The illumination and imaging module may include an objective lens, illumination source, a plurality of detection channels, and a dichroic filter comprising a dichroic reflector or beam splitter, or any combination thereof. Optical systems as disclosed herein may comprise an autofocus system, which may include an autofocus laser, for example, that projects a spot the size of which is monitored to determine when the imaging system is in-focus may be included in some designs. One or more components of an illumination and imaging module as disclosed herein may be coupled to a baseplate. In some embodiments, optical systems in accordance with the disclosure are provided in US Publication No. US2022-0136047 and PCT Publication No. PCT/US2022/37831, which are incorporated herein by reference in their entirety.

### Nucleic Acid Processing Systems

The present disclosure provides systems for detecting a sequence of a nucleic acid in a biological sample. Systems as disclosed herein may be used to conduct in situ sequencing of the nucleic acid. The biological sample may be, e.g., a cellular sample. The nucleic acid may be DNA or RNA.

Systems may comprise compositions as disclosed herein. The composition may comprise a reverse transcriptase. The composition may comprise a padlock probe. The composition may comprise a protein-binding composition, e.g., an antibody-oligonucleotide conjugate. The composition may comprise a sequence polymerase. The composition may comprise a nucleotide, e.g., a chain-terminating nucleotide. The composition may comprise a multivalent molecule. The composition may comprise an avidity complex.

The present disclosure provides a system for detecting in situ at least two target nucleic acid sequences in a biological sample, wherein the at least two target nucleic acid sequences comprise a first target nucleic acid sequence and a second target nucleic acid sequence. The system may comprise a biological sample comprising a first nucleic acid molecule and a second nucleic acid molecule, wherein the first nucleic acid molecule comprises the first target nucleic acid sequence or a reverse complement thereof, or a portion thereof, and wherein the second nucleic acid molecule comprises the second target nucleic acid sequence or a reverse complement thereof, or a portion thereof. The system may comprise a first oligonucleotide comprising a first end portion and a second end portion, wherein the first end portion and second end portion of the first oligonucleotide are complementary and bind to two neighboring segments of the first target nucleic acid sequence or the reverse complement thereof, or the portion thereof, so that the first oligonucleotide forms a first circular oligonucleotide within the biological sample, wherein the first circular oligonucleotide comprises a gap between the first end portion and the second end portion. The system may comprise a second oligonucleotide comprising a first end portion and a second end portion, wherein the first end portion and second end portion of the second oligonucleotide are complementary and bind to two neighboring segments of the second target nucleic acid sequence or the reverse complement thereof, or the portion thereof, so that the second oligonucleotide forms a second circular oligonucleotide within the biological sample, wherein the second circular oligonucleotide comprises a gap between the first end portion and the second end portion.

In some embodiments, the first circular oligonucleotide further comprises a first nucleic acid enzyme configured to join the first and second end portions of the first oligonucleotide, and the second circular oligonucleotide further comprises a second nucleic acid enzyme configured to join the first and second end portions of the second oligonucleotide. In some embodiments, the first and second nucleic acid enzymes comprises a nucleic acid ligase, a nucleic acid ligation enzyme, a nucleic acid polymerase, a nucleic acid polymerization enzyme, or combinations thereof. In some embodiments, the system further comprises a first amplicon of the first circular oligonucleotide, and a second amplicon of the second circular oligonucleotide. In some embodiments, the first amplicon comprises a first concatemer and/or the second amplicon comprises a second concatemer. In some embodiments, the first concatemer comprises at least two repeats of a first unit nucleic acid sequence comprising the first target nucleic acid sequence or a portion thereof, or the reverse complement thereof or a portion thereof; and/or the second concatemer comprises at least two repeats of a second unit nucleic acid sequence comprising the second target nucleic acid sequence or a portion thereof, or the reverse complement thereof or a portion thereof. In some embodiments, the first concatemer further comprises the first identification sequence that identifies the first target nucleic acid sequence, or a sequencing primer or a reverse complement thereof, wherein the second concatemer further comprises the second identification sequence that identifies the second target nucleic acid sequence, or a sequencing primer or a reverse complement thereof. In some embodiments, the first concatemer further comprises a first compaction oligonucleotide, wherein a first segment of the first compaction oligonucleotide is complementary and binds to a first portion of the first concatemer. In some embodiments, second segment of the first compaction oligonucleotide is complementary and binds to a second portion of the first concatemer. In some embodiments, the binding of the first segment of the first compaction oligonucleotide to a first portion of the first concatemer, and/or the binding of the second segment of the first compaction oligonucleotide to a second portion of the first concatemer results in a reduction in the size or a change in the shape of the first concatemer. In some embodiments, the second concatemer further comprises a second compaction oligonucleotide, wherein a first segment of the second compaction oligonucleotide is complementary and binds to a first portion of the second concatemer. In some embodiments, a second segment of the second compaction oligonucleotide is complementary and binds to a second portion of the second concatemer. In some embodiments, the binding of the first segment of the second compaction oligonucleotide to a first portion of the second concatemer, and/or the binding of the second segment of the second compaction oligonucleotide to a second portion of the second concatemer results in a reduction in the size or a change in the shape of the second concatemer. In some embodiments, the system further comprises a polymerizing enzyme, a plurality of nucleotides, and a primer sequence that is complementary to at least a portion of the first concatemer or the second concatemer under conditions sufficient to form a binding complex. In some embodiments, the system further comprises an agent configured to remove a blocking group from a nucleotide of the plurality of nucleotides and generate a 3′ OH group on a sugar moiety of the nucleotide. In some embodiments, the blocking group comprises an alkyl group, an alkenyl group, an alkynyl group, an allyl group, an aryl group, a benzyl group, an azide group, an azido group, an O-azidomethyl group, an amine group, an amide group, a keto group, an isocyanate group, a phosphate group, a thiol group, a disulfide group, a carbonate group, a urea group, or a silyl group. In some embodiments, the plurality of nucleotides comprises a fluorescent label. In some embodiments, the plurality of nucleotides consists of at least two of the same type of nucleotide, wherein the same type of nucleotide is selected from the group comprising dATP, dGTP, dCTP, dTTP, and dUTP, wherein the fluorescent label of one type of nucleotide of the group emits light at a wavelength that is different from the wavelength of light emitted from the fluorescent label of another type of nucleotide of the group. In some embodiments, the plurality of nucleotides comprises at least two types of nucleotides. In some embodiments, the at least two types of nucleotides are selected from the group comprising dATP, dGTP, dCTP, dTTP, and dUTP. In some embodiments, the fluorescent label of one type of nucleotide of the group emits light at a wavelength that is different from the wavelength of light emitted from the fluorescent label of another type of nucleotide of the group. In some embodiments, the system further comprises a plurality of nucleotide conjugates. In some embodiments, a nucleotide conjugate of the plurality of nucleotide conjugates is configured to form a multivalent binding complex comprising two or more of the polymerizing enzyme, the nucleotide conjugate, and the at least two target nucleic acid sequences. In some embodiments, the nucleotide conjugate comprises a label and at least two nucleotide moieties that are each complementary and bind to a nucleotide of each of the at least two target nucleic acid sequences. In some embodiments, the system further comprises a first oligonucleotide conjugate comprising a first short nucleic acid and a first binding moiety that binds specifically to a first target polypeptide. In some embodiments, the first short nucleic acid comprises a first tag sequence and a second tag sequence. In some embodiments, the first oligonucleotide conjugate binds specifically to the first target polypeptide in the biological sample through the first binding moiety to form a first binding complex. In some embodiments, the first and second tag sequences identify the first binding moiety in a nucleic acid sequence reaction. In some embodiments, the system further comprises a second oligonucleotide conjugate comprising a second short nucleic acid and a second binding moiety that binds specifically to a second target polypeptide, wherein the second short nucleic acid comprises a third tag sequence and a fourth tag sequence, wherein the second oligonucleotide conjugate binds specifically to the second target polypeptide in the biological sample through the second binding moiety to form a second binding complex, wherein the third and fourth tag sequences identify the second binding moiety in a nucleic acid sequence reaction. In some embodiments, the first or second binding moiety is an antibody or an antigen-binding fragment thereof. In some embodiments, the system further comprises a third oligonucleotide comprising a first end portion and a second end portion, wherein the first end portion and second end portion of the third oligonucleotide are complementary and bind to the first tag sequence and the second tag sequence of the first oligonucleotide conjugate so that the third oligonucleotide forms a circular structure with a gap between the first end portion and the second end portion. In some embodiments, the system further comprises a fourth oligonucleotide comprising a first end portion and a second end portion, wherein the first end portion and second end portion of the fourth oligonucleotide are complementary and bind to the third tag sequence and the fourth tag sequence so that the fourth oligonucleotide forms a circular structure with a gap between the first end portion and the second end portion. In some embodiments, the system further comprises a third circular oligonucleotide that results from joining the first and second end portions of the third oligonucleotide and a fourth circular oligonucleotide that results from joining the first and second end portions of the fourth oligonucleotide, wherein the first circular oligonucleotide and the third circular oligonucleotide comprise a first sequencing primer or the reverse complement thereof, wherein the second circular oligonucleotide and the fourth circular oligonucleotide comprise a second sequencing primer or the reverse complement thereof, wherein the sequences of the first and second sequencing primers have at least one nucleotide of difference, wherein the joining is carried out by the first or second nucleic acid enzyme. In some embodiments, the system further comprises a third amplicon of the third circular oligonucleotide, and a fourth amplicon of the fourth circular oligonucleotide. In some embodiments, the third amplicon comprises a third concatemer and/or the fourth amplicon comprises a fourth concatemer. In some embodiments, the third concatemer comprises at least two repeats of a third unit nucleic acid sequence comprising the third circular oligonucleotide or a portion thereof, or the reverse complement thereof or a portion thereof. In some embodiments, the fourth concatemer comprises at least two repeats of a second unit nucleic acid sequence comprising the fourth circular oligonucleotide or a portion thereof, or the reverse complement thereof or a portion thereof. In some embodiments, the third concatemer further comprises the first or second tag sequence or a reverse complement thereof that identifies the first binding moiety. In some embodiments, the fourth concatemer further comprises the third or fourth tag sequence or a reverse complement thereof that identifies the second binding moiety. In some embodiments, the third concatemer further comprises a third compaction oligonucleotide, wherein a first segment of the third compaction oligonucleotide is complementary and binds to a first portion of the third concatemer. In some embodiments, a second segment of the third compaction oligonucleotide is complementary and binds to a second portion of the third concatemer. In some embodiments, the binding of the first segment of the third compaction oligonucleotide to the first portion of the third concatemer and/or the binding of the second segment of the third compaction oligonucleotide to the second portion of the third concatemer results in a reduction in the size or a change in the shape of the third concatemer. In some embodiments, the fourth concatemer further comprises a fourth compaction oligonucleotide, wherein a first segment of the fourth compaction oligonucleotide is complementary and binds to a first portion of the fourth concatemer. In some embodiments, a second segment of the fourth compaction oligonucleotide is complementary and binds to a second portion of the fourth concatemer. In some embodiments, the binding of the first segment of the fourth compaction oligonucleotide to the first portion of the fourth concatemer and/or the binding of the second segment of the fourth compaction oligonucleotide to the second portion of the fourth concatemer results in a reduction in the size or a change in the shape of the fourth concatemer. In some embodiments, the system further comprises a second polymerizing enzyme, a second plurality of nucleotides, and a second primer sequence that is complementary to at least a portion of the third concatemer or the fourth concatemer under conditions sufficient to form a binding complex comprising the third concatemer hybridized to the second primer sequence, the second polymerizing enzyme, and a second nucleotide of the second plurality of nucleotides that is complementary and binds to the a nucleotide of the third or fourth concatemer. In some embodiments, the system further comprises a second agent configured to remove a second blocking group from a second nucleotide of the second plurality of nucleotides, and generate a 3′ OH group on a sugar moiety of the second nucleotide. In some embodiments, the second blocking group comprises an alkyl group, an alkenyl group, an alkynyl group, an allyl group, an aryl group, a benzyl group, an azide group, an azido group, an O-azidomethyl group, an amine group, an amide group, a keto group, an isocyanate group, a phosphate group, a thiol group, a disulfide group, a carbonate group, a urea group, or a silyl group. In some embodiments, the second plurality of nucleotides comprises a second fluorescent label. In some embodiments, the second plurality of nucleotides consists of at least two of the same type of nucleotide, wherein the same type of nucleotide is selected from the group comprising dATP, dGTP, dCTP, dTTP, and dUTP, wherein the second fluorescent label of one type of nucleotide of the group emits light at a wavelength that is different from the wavelength of light emitted from the second fluorescent label of another type of nucleotide of the group. In some embodiments, the second plurality of nucleotides comprises at least two types of nucleotides, wherein the at least two types of nucleotides are selected from the group comprising dATP, dGTP, dCTP, dTTP, and dUTP, wherein the second fluorescent label of one type of nucleotide of the group emits light at a wavelength that is different from the wavelength of light emitted from the second fluorescent label of another type of nucleotide of the group. In some embodiments, the system further comprises a second plurality of nucleotide conjugates. In some embodiments, a second nucleotide conjugate of the second plurality of nucleotide conjugates is configured to form a multivalent binding complex comprising two or more of the second polymerizing enzyme, the second nucleotide conjugate of the second plurality of nucleotide conjugates plurality of nucleotide conjugates, and the at least two of the first short nucleic acid or the short nucleic acid or a portion thereof. In some embodiments, the second nucleotide conjugate comprises a label and at least two nucleotide moieties that are each complementary and bind to a nucleotide of each of the at least two of the first short nucleic acid or the short nucleic acid or a portion thereof. In some embodiments, the system further comprises a solid surface comprising the biological sample immobilized to the solid surface. In some embodiments, the biological sample is permeabilized. In some embodiments, the solid surface further comprises a hydrophilic polymer coating layer coupled thereto. In some embodiments, the hydrophilic polymer coating layer has a water contact angle that is less than 50 degrees. In some embodiments, the system further comprises an optical imaging module configured to image the biological sample coupled to the solid surface to detecting in situ the at least two target nucleic acid sequences and/or the at least two target polypeptides in the biological sample. In some embodiments, the first target polypeptide is encoded by the first target nucleic acid molecule or a reverse complement thereof and the second target polypeptide is encoded by the second target nucleic acid molecule or a reverse complement thereof.

### Flow Cells

In any of the systems described herein, the cellular sample can be deposited onto a solid support (e.g., a flow cell). In some embodiments, the cellular sample is deposited onto a flow cell having walls (e.g., top or first wall, and bottom or second wall) and a gap in-between, where the gap can be filled with a fluid, where the flow cell is positioned in a fluorescence optical imaging system. The cellular sample has a thickness that may require using the imaging system to focus separately on the first and second surfaces of the flow cell, when using a traditional imaging system. For improved imaging of the sequencing reaction of the concatemers in the cellular sample, the flow cell can be positioned in a high performance fluorescence imaging system, which comprises two or more tube lenses which are designed to provide optimal imaging performance for the first and second surfaces of the flow cell at two or more fluorescence wavelengths. In some embodiments, the high-performance imaging system further comprises a focusing mechanism configured to refocus the optical system between acquiring images of the first and second surfaces of the flow cell. In some embodiments, the high performance imaging system is configured to image two or more fields-of-view on at least one of the first flow cell surface or the second flow cell surface.

### Supports and Coatings

In any of the systems described herein, the solid support comprises a flow cell having a coating that promotes cell adhesion. In some embodiments, the flow cell comprises a support which can be a planar or non-planar support. The support can be solid or semi-solid. In some embodiments, the support can be porous, semi-porous or non-porous. The support can be made of any material such as glass, plastic or a polymer material. In some embodiments, the surface of the support can be coated with one or more compounds to produce a passivated layer on the support. In some embodiments, the passivated layer forms a porous or semi-porous layer. In some embodiments, the support is coated with a lysine compound, poly-lysine compound, arginine compound or an amino-terminated compound. The support can be coated with an unbranched compound, a branched compound, or a mixture of unbranched and branched compounds. In some embodiments, the support is coated with surface primers for capturing nucleic acids from the cellular sample. Alternatively, the support lacks surface primers.

### Automated Mode

In any of the systems described herein, any combination of the steps for conducting in situ reiterative short read sequencing can be performed in an automated mode using the fluid dispensing system, including cell seeding, cell fixation, cell permeabilization, reverse transcription reactions, padlock probe hybridization, padlock probe ligation reaction, rolling circle amplification, and sequencing.

The present disclosure provides systems for growing/culturing a cellular sample on a flow cell and conducting nucleic acid workflows of the cultured cellular sample on the flow cell.

In some embodiments, the cellular sample is deposited on the flow cell. The flow cell can be coated with a reagent that promotes cell adhesion to the flow cell. The flow cell, having a cell sample adhered thereon, can be placed onto a sequencing apparatus having a flow cell holder/cradle which is fluidically connected to an automated fluid dispensing system and configured on a fluorescent microscope. In some embodiments, the sequencing apparatus can be configured with at least one fluidic delivery device, at least one fluidics device (e.g., microfluidics device), at least one imaging device and/or at least one sensor to detect signals from the sequencing reactions.

In some embodiments, the automated fluid dispensing system can be used to deliver simple or complex cell culture media to the cellular sample on the flow cell. In some embodiments, the cellular sample can be cultured/expanded on the flow cell for 2-10 generations or more. In some embodiments, the cellular sample can be expanded to confluence or non-confluence.

In some embodiments, the automated fluid dispensing system can be used to deliver fixation reagents to the expanded cellular sample on the flow cell, and the cellular sample can be incubated under conditions suitable for cell fixation.

In some embodiments, the automated fluid dispensing system can be used to deliver permeabilization reagents to the fixed cellular sample on the flow cell, and the cellular sample can be incubated under conditions suitable for cell permeabilization.

In some embodiments, the automated fluid dispensing system can be used to deliver reagents for conducting reverse transcription of RNA inside the fixed and permeabilized cellular sample under a condition suitable for generating a plurality of cDNA inside the cellular sample.

In some embodiments, the automated fluid dispensing system can be used to deliver reagents for conducting padlock probe hybridization, circularization and ligation under a condition suitable for generating a plurality of covalently closed circular padlock probes inside the cellular sample.

In some embodiments, the automated fluid dispensing system can be used to deliver reagents for conducting rolling circle amplification under a condition suitable for generating a plurality of concatemer molecules inside the cellular sample.

In some embodiments, the automated fluid dispensing system can be used to deliver sequencing reagents for conducting sequencing cycles of the concatemer molecules under a condition suitable for generating a plurality of sequencing read products inside the cellular sample. In some embodiments, individual cycle times can be achieved in less than 30 minutes. In some embodiments, the field of view (FOV) can exceed 1 mm² and the cycle time for scanning large area (>10 mm²) can be less than 5 minutes.

In some embodiments, the automated fluid dispensing system can be used to deliver reagents for removing the plurality of sequencing read products from the concatemer molecules and retaining the concatemer molecules inside the cellular sample.

In some embodiments, the automated fluid dispensing system can be used to deliver sequencing reagents for conducting sequencing cycles of the concatemer molecules under a condition suitable for generating another plurality of sequencing read products inside the cellular sample. In some embodiments, individual cycle times can be achieved in less than 30 minutes. In some embodiments, the field of view (FOV) can exceed 1 mm² and the cycle time for scanning large area (>10 mm²) can be less than 5 minutes.

### EXAMPLES

The following examples are meant to be illustrative and can be used to further understand embodiments of the present disclosure and should not be construed as limiting the scope of the present teachings in any way.

### Example 1: Reiterative Insitu Sequencing

Preparing Coated Flow cells

Glass 2-lane flow cells were washed with poly-L-lysine (e.g., 0.01% w/v in water) for about 2-30 minutes to coat the flow cell. The coated flow cell was rinsed with cell culture media prior to cell seeding. The poly-lysine coating lacked capture primers that could hybridize to nucleic acids having a universal capture primer sequence.

Seeding and Culturing Cells on Coated Flow cells

Human embryonic kidney 293 cells (HEK 293 cells) in fresh cell culture media were seeded on the coated flow cells. The cell culture media included D-MEM high glucose (Thermo Fisher Scientific, catalog No. 11965118), fetal bovine serum (10% FBS; Thermo Fisher Scientific, catalog No. A3160402), MEM non-essential amino acids (0.1 mM MEM, Thermo Fisher Scientific, catalog No. 11140050), L-glutamine (6 mM L-glutamine, Thermo Fisher Scientific, catalog No. A2916801), MEM sodium pyruvate (1 mM sodium pyruvate, Thermo Fisher Scientific, catalog No. 11360070), and an antibiotic (1% penicillin-streptomycin-glutamine, Thermo Fisher, catalog No. 10378016). The seeded cells were cultured for 2 days at 37° C. with a humidified atmosphere of approximately 5-10% carbon dioxide in air. The cells remained as whole cells on the flow cell, and were not embedded in paraffin or sliced.

### Cell Fixation and Permeabilization

The cells were washed with DPBS and then fixed with 4% paraformaldehyde in PBS and water for about 30 minutes at room temperature. The cells were washed twice with PBS.

The fixed cells were permeabilized with 70% ethanol for about 30 minutes at room temperature, then washed with PBS. The fixed and permeabilized cells remained whole and were not sectioned into slices. The HEK 293 were not transgenic cells.

### Reverse Transcription

Each lane of the flow cells were flowed with 20 μL of reverse transcription reagents. The reverse transcription reagents included 84 uL RevertAid reverse transcription buffer Thermo Fischer Scientific, catalog No. EP0441), 1.0.5 uL dNTPs, 4.2 ul., BSA., 4.2 uL reverse transcription primers, 16.8 uL RiboLock RNase inhibitor (Thermo Fisher Scientific, catalog No. E00381), 10.08 uL, RevertAid H-minus (Thermo Fisher Scientific, catalog No. EP0451), and 290.22 uL, water. The flow cells were placed in a moisturized dish which was sealed with parafilm, and the dish was placed in a 37° C. incubator for about 16 hours. The cells were fixed again by washing the cells 5 times with PBS-T (PBS with Tween-20), then treated with a post-fixation reagent at room temperature for about 30 minutes. The post-fixation reagent included 3% paraformaldehyde and 0.1% glutaraldehyde, in PBS. The cells were washed 5 times with PBS-T.

The reverses transcription primers were designed to selectively hybridize to RNA transcribed from a housekeeping gene, human GAPDH. (glyceraldehyde-3-phosphate dehydrogenase). The sequence of a cDNA primer for human GAPDH was 5′-CAGAGAGCGAAGCGGGAGGCTGCGGGCTCAATTTATAGAA-3′ (SEQ ID NO:1).

### Padlock Probe Circularization

The PBS-T was replaced with 100 μL of padlock probe reagent which included 28 uL AmpLigase buffer (e.g., Lucigen, catalog No. A1905B), 22.4 uL RNase H (e.g., New England Biolabs, catalog No. M0297S, 2.8 uL BSA, 0.7 uL padlock probe, 28 uL AmpLigase (e.g., Lucigen, catalog No. A0110K), and 198.1 uL water. The flow cells were incubated at 37° C. for about 10 minutes, then at 45° C. for about 90 minutes. The flow cells were washed twice with PBS-T.

For a first experiment, the sequence of the GAPDH padlock probe was 5′ [P]-GCCTCCCGCTTCGCTCTCTGCATGTAATGCACGTACTTTCAGGTATGTCGG AAGGTGTGCAGGCTACCGCTTGTCAACTGGAGAATGTTCTATAAATTGAGCCC GCA-3′ (SEQ ID NO:2) (see also FIG. 1). This padlock probe carried a universal binding site for a compaction oligonucleotide which was 5′-CATGTAATGCACGTACTTTCAGGT-3′ (SEQ ID NO:3). This padlock probe also carried a universal binding site for an RCA primer and a sequencing primer which was 5′-ATGTCGGAAGGTGTGCAGGCTACCGCTTGTCAACT-3′ (SEQ ID NO:4). This padlock probe also carried a target barcode sequence which was 5′-GGAGAATG-3′ (SEQ ID NO:5).

For a second experiment, the sequence of another GAPDH padlock probe was 5′ [P]-CAGCCGCATCTTCTTTTGCGTCCTCTATGATTACTGACTGCGTCTATTTAGT GGAGCCXXXXXXXXXXXCTTTTGCTCCTCCTGTTCGACAGT (SEQ ID NO:6), wherein the 11-mer ‘X’ sequence (underlined sequence) represents the target barcode sequence that corresponds to GAPDH transcripts. This padlock probe carried a universal binding site for an RCA primer and a sequencing primer which was 5′-TCCTCTATGATTACTGACTGCGTCTA TTTAGTGGAGCC-3′ (SEQ ID NO:7). A set of this padlock probe was designed to include one of four different target barcode sequences which included: Target Barcode 1: GCTACTATCTT (SEQ ID NO:8); Target Barcode 2: TTAGTAGATAA (SEQ ID NO:9);Target Barcode 3: CACCAGCGAGG (SEQ ID NO:10); and Target Barcode 4: AGGTGCTCGCC (SEQ ID NO: 11). Both padlock probes lacked a universal capture primer sequence.

For the first experiment, the cDNA was annealed to one type of padlock probe specific for GAPDH (as described above), rather than annealed to a set of different GAPDH-specific padlock probes which could hybridize to different regions along the length of the GAPDH cDNA. For the second experiment, the cDNA was annealed to a set of four different padlock probes, each probe carrying a different target barcode sequence but the left and right padlock arms were designed to hybridize to the same target sequences in the cDNA. Thus, for the second experiment, the padlock probes were designed to hybridize to one target region in the cDNA.

### Rolling Circle Amplification

Rolling circle amplification was conducted by flowing onto the flow cells 100 μL of a primer hybridization reagent which included 2×SSC, 20% formamide, and 1 uM RCA primer. The flow cells were incubated at room temperature for about 30 minutes. The flow cells were washed twice with PBS-T buffer. The rolling circle amplification reaction was conducted by flowing 100 μL of RCA reagent which included 1× Phi29 buffer, 250 uM of dNTPs, 0.2 mg/mL BSA, 5% glycerol, and 1 Unit/uL Phi29 DNA polymerase. The flow cells were incubated at 30° C. for about 16 hours. The flow cells were washed twice with PBS-T.

In situ Sequencing Using 2-Stage Sequencing Method

The flow cells were washed with sequencing primer reagent which included 2×SSC, 20% formamide, and 1 uM universal sequencing primer. The sequence of the sequencing primer for the first experiment was 5′-ATGTCGGAAGGTGTGCAGGCTACCGCTTGTCAACT-3′ (SEQ ID NO:12). The sequence of the sequencing primer for the second experiment was 5′-TTACTGACTGCGTCTATTTAGTGGAGCC-3′ (SEQ ID NO: 13).

The flow cells were washed twice with a wash buffer which included 10 mM Tris (pH 8), 100 mM NaCl, 0.4 mM EDTA and 0.3% Tween-20.

A two-stage sequencing method was used to sequence the primer-concatemers inside the cells. Each sequencing cycle included a first stage and a second stage. The first stage employed a first sequencing polymerase and fluorescently labeled multivalent molecules. The second stage employed a second sequencing polymerase and non-labeled nucleotide analogs.

The first stage sequencing was conducted with fluorescently labeled multivalent molecules. An image of the cells on the flow cells was obtained prior to the start of sequencing. A solution of a first sequencing polymerase was flowed onto the flow cells and incubated at 42° C. for about 10 minutes to form complexed polymerases on the concatemers inside the cells. A trap reagent was flowed onto the flow cells. The trap reagent included a mixture of fluorescently labeled multivalent molecules (e.g., about 40-100 nM) (see FIGS. 7-10) and a non-catalytic cation (e.g., strontium, barium or calcium). The mixture of fluorescently labeled multivalent molecules included dATP, dGTP, dCTP and dUTP. The flow cells were incubated at 42° C. for about 10 minutes to permit the multivalent molecules to bind the complexed polymerases, without incorporation of the nucleotide units, and form avidity complexes on the concatemers inside the cells. The flow cells were washed at room temperature with a trap reagent that lacked the fluorescently labeled multivalent molecules. The flow cells were washed with an imaging buffer. An image was obtained using a fluorescent microscope: 1 minute/FOV, 4 channels, 200 ms/frame, using Z-stack imaging. The multivalent molecules and first sequencing polymerases were removed by washing the flow cells twice at 42° C. with a removal reagent. The flow cells were washed four times at 52° C. with a wash buffer (see formulation above).

The second stage sequencing was conducted with non-labeled nucleotide analogs. A solution of a second sequencing polymerase, a mixture of non-labeled nucleotide analogs and a catalytic divalent cation (e.g., magnesium) was flowed onto the flow cells and incubated at 52° C. for about 2-5 minutes to permit incorporation of the nucleotide analogs. The mixture of non-labeled nucleotide analogs included 3′O-methylazido nucleotides with dATP, dGTP, dCTP and dTTP. The flow cells were washed twice with a removal reagent at 51° C. for about 20 seconds. The 3′ blocking moieties were removed from the incorporated nucleotide by washing the flow cells twice with a cleaving reagent at 51° C. for about 40 seconds. The flow cells were washed twice with a wash buffer (see formulation above). The next sequencing cycle was conducted using the two-stage sequencing method described above. The cells on the flow cells were subjected to 30 sequencing cycles which generated a first plurality of sequencing read products.

In a separate experiment, the workflow was conducted under automated mode by placing a flow cell on an apparatus configured with an automated fluidics delivery system. Similar to the manual workflow described above, the automated workflow employed a glass 2-lane flow cell which was coated with poly-L-lysine and lacked capture primers that could hybridize to nucleic acids having a universal capture primer sequence. The automated workflow conducted cell fixation, cell permeabilization, reverse transcription, post-fixation, padlock probe hybridization, and rolling circle amplification. The concatemers inside the cells were sequenced under automated mode using the two-stage sequencing method as described above for the manual workflow. The formulations for the various steps in the automated workflow were the same as described above for the manual workflow.

In a separate experiment, the workflow was conducted under automated mode by placing a flow cell on an apparatus configured with an automated fluidics delivery system. Similar to the manual workflow described above, the automated workflow employed a glass 2-lane flow cell which was coated with poly-L-lysine and lacked capture primers that could hybridize to nucleic acids having a universal capture primer sequence. The automated workflow conducted cell fixation, cell permeabilization, reverse transcription, post-fixation, padlock probe hybridization, and rolling circle amplification. The concatemers inside the cells were sequenced under automated mode using the two-stage sequencing method as described above for the manual workflow. The formulations for the various steps in the automated workflow were the same as described above for the manual workflow.

FIG. 6 shows images of fluorescent sequencing signals emitted from the fixed and permeabilized HEK 293 cells on the poly-lysine coated flow cell using the automated workflow mode. Thirty cycles of the two-stage sequencing reactions were conducted using multivalent molecules labeled with one of four fluorophores and unlabeled nucleotide analogs. FIG. 6 shows fluorescent signals from cycles 20-25 where the sequence reads CCTCCT. A total of 4620 fluorescent spots were detected with significant signals in all cycles. More than 90% of the fluorescent spots detected the target barcode sequences.

The images in FIG. 6 were collected using an Olympus IX83 inverted microscope configured with an Olympus UPlanFL N, 20×0.7NA objective and an Andor Zyla 4.2 SCMOS camera. The filters used included Semrock LPD02-532RU-25, Semrock LP03-532RU-25, Chroma T6471pxr, Semrock LP02-647RU-25, Semrock FFO1-562/40-25, Semrock FF01-600/37-25, Semrock FF01-719/60-25, and Semrock FF01-660/30-25.

In some embodiments, reiterative in situ sequencing can be conducted. For example, after conducting up to 30 sequencing cycles using the two-stage sequencing method as described above, the first plurality of sequencing read products can be removed from the concatemers while retaining the concatemers inside the cells by washing the flow cells with a de-hybridization reagent. In some embodiments, the de-hybridization reagent comprises an SSC buffer (e.g., saline-sodium citrate) buffer, with or without formamide, at a temperature that promotes nucleic acid denaturation such as for example 30-90° C., for about 2-10 minutes.

The concatemers, now being denatured from the first sequencing read products, can be re-hybridized with universal sequencing primers to start another round of up to 30 cycles of the two-stage sequencing method. For example, the flow cells can be washed with sequencing primer reagent which can include 2×SSC, 20% formamide, and 1 uM universal sequencing primer. The sequence of the sequencing primer can be 5′-ATGTCGGAAGGTGTGCA GGCTACCGCTTGTCAACT-3′ (SEQ ID NO:12). The flow cells can be washed twice with a wash buffer which can include 10 mM Tris (pH 8), 100 mM NaCl, 0.4 mM EDTA and 0.3% Tween-20. Thirty cycles of the two-stage sequencing method can be conducted as described above. In some embodiments, one or more rounds of up to 30 cycles of the two-stage sequencing method can be conducted on the cells adhered to the flow cells.

All sequences used in the foregoing examples are depicted in Table 2.

TABLE 2
Sequences used in examples

| SEQ | |
|---|---|
| ID NO: | SEQUENCE |
| 1 | CAGAGAGCGAAGCGGGAGGCTGCGGGCTCAATTTATAG |
| | AA |
| 2 | GCCTCCCGCTTCGCTCTCTGCATGTAATGCACGTACTTTC |
| | AGGTATGTCGGAAGGTGTGCAGGCTACCGCTTGTCAACT |
| | GGAGAATGTTCTATAAATTGAGCCCGCA |
| 3 | CATGTAATGCACGTACTTTCAGGT |
| 4 | ATGTCGGAAGGTGTGCAGGCTACCGCTTGTCAACT |
| 5 | GGAGAATG |
| 6 | CAGCCGCATCTTCTTTTGCGTCCTCTATGATTACTGACTG |
| | CGTCTATTTAGTGGAGCCXXXXXXXXXXXCTTTTGCTCC |
| | TCCTGTTCGACAGT |
| 7 | TCCTCTATGATTACTGACTGCGTCTATTTAGTGGAGCC |
| 8 | GCTACTATCTT |
| 9 | TTAGTAGATAA |
| 10 | CACCAGCGAGG |
| 11 | AGGTGCTCGCC |
| 12 | ATGTCGGAAGGTGTGCAGGCTACCGCTTGTCAACT |
| 13 | TTACTGACTGCGTCTATTTAGTGGAGCC |
| 14 | CATGTAATGCACGTACTTTCAGGGTAAACATGTAATGCA |
| | CGTACTTTCAGGGT |

While preferred embodiments of the present composition, apparatuses, and methods have been shown and described herein, it will be obvious to those skilled in the art that such embodiments are provided by way of example only. Numerous variations, changes, and substitutions will now occur to those skilled in the art without departing from the composition, apparatuses, and methods. It should be understood that various alternatives to the embodiments of the composition, apparatuses, and methods described herein may be employed in practicing the inventive concepts. It is intended that the following claims define the scope of the composition, apparatuses, and methods and that methods, compositions, and apparatuses within the scope of these claims and their equivalents be covered thereby.

1-33. (canceled)

34. A method of detecting a ribonucleic acid (RNA) sequence and a polypeptide in a cellular sample, the method comprising:
(a) providing the cellular sample on a solid support, wherein the cellular sample comprises:
(i) a first concatemer comprising at least at portion of a complementary deoxyribonucleic acid (cDNA) sequence that corresponds to an RNA sequence, wherein the first concatemer further comprises a first barcode sequence that corresponds to the cDNA sequence; and
(ii) a second concatemer comprising a second barcode sequence that corresponds to the polypeptide; and
(b) sequencing the first concatemer or a portion thereof and the second concatemer or a portion thereof to identify the RNA sequence and the polypeptide.

35. The method of claim 34, wherein the cellular sample further comprises:
(a) a third concatemer comprising at least a portion of a second cDNA sequence that corresponds to an RNA sequence, wherein the third concatemer further comprises a third barcode sequence that corresponds to the second cDNA sequence; and
(b) a fourth concatemer comprising a fourth barcode sequence that corresponds to a second polypeptide.

36. The method of claim 35, wherein (b) comprises sequencing under conditions that inhibit sequencing of the third and fourth concatemers.

37. The method of claim 36, wherein the conditions that inhibit sequencing of the third and fourth concatemers comprise a non-catalytic divalent cation including strontium, barium, calcium, or any combination thereof.

38. The method of claim 34, wherein (b) comprises contacting the first concatemer with a plurality of fluorophore-labeled molecules, wherein the fluorophore-labeled molecules comprise nucleotides, under conditions suitable to form a binding complex between the first concatemer and at least two nucleotides of the fluorophore-labeled molecules.

39. The method of claim 38, wherein a fluorophore-labeled molecule of the plurality of fluorophore-labeled molecules comprises a core attached to a plurality of nucleotide arms and wherein a nucleotide arm of the plurality of nucleotide arms is attached to at least one nucleotide.

40. The method of claim 39, wherein the nucleotide is selected from the group consisting of dATP, dGTP, dCTP, dTTP, and dUTP.

41. The method of claim 38, wherein the first concatemer is primed.

42. The method of claim 38, wherein the binding complex further comprises a polymerase.

43. The method of claim 38, wherein the conditions suitable to form the binding complex comprise magnesium, manganese, or a combination thereof.

44. The method of claim 34, wherein (b) comprises contacting the second concatemer with a plurality of fluorophore-labeled molecules, wherein the fluorophore-labeled molecules comprise nucleotides, under conditions suitable to form a binding complex between the second concatemer and at least two nucleotides of the fluorophore-labeled molecules.

45. The method of claim 44, wherein a fluorophore-labeled molecule of the plurality of fluorophore-labeled molecules comprises a core attached to a plurality of nucleotide arms and wherein a nucleotide arm of the plurality of nucleotide arms is attached to at least one nucleotide.

46. The method of claim 45, wherein the nucleotide is selected from the group consisting of dATP, dGTP, dCTP, dTTP, and dUTP.

47. The method of claim 44, wherein the second concatemer is primed.

48. The method of claim 44, wherein the binding complex further comprises a polymerase.

49. The method of claim 44, wherein the conditions suitable to form the binding complex comprise magnesium, manganese, or a combination thereof.

50. The method of claim 34, wherein the RNA sequence comprises a messenger RNA sequence.

51. The method of claim 34, wherein the polypeptide is encoded by the RNA sequence.

52. The method of claim 34, wherein prior to (a), generating the cDNA sequence using reverse transcription.

53. The method of claim 34, wherein prior to (a), contacting the cellular sample with an antibody-oligonucleotide conjugate, wherein the antibody-oligonucleotide conjugate binds to the polypeptide, and wherein the antibody-oligonucleotide conjugate comprises a tag.

54. The method of claim 53, further comprising
(a) contacting the cellular sample with a padlock probe, wherein the padlock probe comprises a sequence that hybridizes to the tag; and
(b) performing rolling circle amplification to generate the second concatemer.

55. The method of claim 34, wherein the solid support is an interior of a flow cell.

56. The method of claim 55, wherein the interior surface has a water contact angle of less than or equal to 45 degrees.

57. The method of claim 34, wherein the solid support comprises a hydrophilic coating layer coupled thereto.

58. The method of claim 34, wherein (b) comprises performing 2-30 sequencing cycles.

59. The method of claim 34, wherein the first concatemer comprises tandem repeats of a sequence corresponding to the RNA sequence.

60. The method of claim 34, wherein (b) is performed within the cellular sample.

61. The method of claim 34, wherein (b) is performed outside of the cellular sample using a sequencing system.

62. The method of claim 34, wherein the cellular sample comprises a cell, wherein the cell comprises:
a first concatemer comprising at least at portion of a complementary deoxyribonucleic acid (cDNA) sequence that corresponds to an RNA sequence, wherein the first concatemer further comprises a first barcode sequence that corresponds to the cDNA sequence; and a second concatemer comprising a second barcode sequence that corresponds to the polypeptide.

63. The method of claim 34, wherein the cellular sample comprises a cell, wherein the cell comprises:
a first concatemer comprising at least at portion of a complementary deoxyribonucleic acid (cDNA) sequence that corresponds to an RNA sequence, wherein the first concatemer further comprises a first barcode sequence that corresponds to the cDNA sequence; and a second concatemer comprising a second barcode sequence that corresponds to the polypeptide.
